SEQUENCE LISTING (49 sequences)

SEQ ID NO: 1 - protein, 596 aa, Homo sapiens
MAGNCGARGALSAHTLLFDLPPALLGELCAVLDSCDGALGWRGLAERLSSSWLDVRHIEKYVDQGKSGTRELLWSWAQKNKTIGDLLQVLQEMGHRRAIHLITNYGAVLSPSEKSYQEGGFPNILFKETANVTVDNVLIPEHNEKGILLKSSISFQNIIEGTRNFHKDFLIGEGEIFEVYRVEIQNLTYAVKLFKQEKKMQCKKHWKRFLSELEVLLLFHHPNILELAAYFTETEKFCLIYPYMRNGTLFDRLQCVGDTAPLPWHIRIGILIGISKAIHYLHNVQPCSVICGSISSANILLDDQFQPKLTDFAMAHFRSHLEHQSCTINMTSSSSKHLWYMPEEYIRQGKLSIKTDVYSFGIVIMEVLTGCRVVLDDPKHIQLRDLLRELMEKRGLDSCLSFLDKKVPPCPRNFSAKLFCLAGRCAATRAKLRPSMDEVLNTLESTQASLYFAEDPPTSLKSFRCPSPLFLENVPSIPVEDDESQNNNLLPSDEGLRIDRMTQKTPFECSQSEVMFLSLDKKPESKRNEEACNMPSSSCEESWFPKYIVPSQDLRPYKVNIDPSSEAPGHSCRSRPVESSCSSKFSWDEYEQYKKE

SEQ ID NO: 2 - protein, 12 aa, synthetic construct
GRKKRRQRRPPQ

SEQ ID NO: 3 - protein, 9 aa, synthetic construct
  SITE 1..9: note = D-Arginine
RRRRRRRRR

SEQ ID NO: 4 - protein, 13 aa, synthetic construct
RLVSYNGIIFFLK

SEQ ID NO: 5 - protein, 12 aa, synthetic construct
FNLPLPSRPLLR

SEQ ID NO: 6 - protein, 12 aa, synthetic construct
  SITE 12: note = D-Arginine
KGKPILFFRLKR

SEQ ID NO: 7 - protein, 10 aa, synthetic construct
KPILFFRLGK

SEQ ID NO: 8 - protein, 11 aa, synthetic construct
KGSALISWIKR

SEQ ID NO: 9 - protein, 27 aa, synthetic construct
  SITE 27: note = D-Arginine
GRKKRRQRRPPQGGSKGKPILFFRLKR

SEQ ID NO: 10 - protein, 25 aa, synthetic construct
GRKKRRQRRPPQGGSKPILFFRLGK

SEQ ID NO: 11 - protein, 26 aa, synthetic construct
GRKKRRQRRPPQGGSKGSALISWIKR

SEQ ID NO: 12 - protein, 24 aa, synthetic construct
  SITE 1..9: note = D-Arginine
  SITE 24: note = D-Arginine
RRRRRRRRRGGSKGKPILFFRLKR

SEQ ID NO: 13 - protein, 22 aa, synthetic construct
  SITE 1..9: note = D-Arginine
RRRRRRRRRGGSKPILFFRLGK

SEQ ID NO: 14 - protein, 23 aa, synthetic construct
  SITE 1..9: note = D-Arginine
RRRRRRRRRGGSKGSALISWIKR

SEQ ID NO: 15 - protein, 28 aa, synthetic construct
  SITE 28: note = D-Arginine
RLVSYNGIIFFLKGGSKGKPILFFRLKR

SEQ ID NO: 16 - protein, 26 aa, synthetic construct
RLVSYNGIIFFLKGGSKPILFFRLGK

SEQ ID NO: 17 - protein, 27 aa, synthetic construct
RLVSYNGIIFFLKGGSKGSALISWIKR

SEQ ID NO: 18 - protein, 27 aa, synthetic construct
  SITE 27: note = D-Arginine
FNLPLPSRPLLRGGSKGKPILFFRLKR

SEQ ID NO: 19 - protein, 25 aa, synthetic construct
FNLPLPSRPLLRGGSKPILFFRLGK

SEQ ID NO: 20 - protein, 26 aa, synthetic construct
FNLPLPSRPLLRGGSKGSALISWIKR

SEQ ID NO: 21 - protein, 24 aa, synthetic construct
  SITE 24: note = D-Arginine
GRKKRRQRRPPQKGKPILFFRLKR

SEQ ID NO: 22 - protein, 22 aa, synthetic construct
GRKKRRQRRPPQKPILFFRLGK

SEQ ID NO: 23 - protein, 23 aa, synthetic construct
GRKKRRQRRPPQKGSALISWIKR

SEQ ID NO: 24 - protein, 21 aa, synthetic construct
  SITE 1..9: note = D-Arginine
  SITE 21: note = D-Arginine
RRRRRRRRRKGKPILFFRLKR

SEQ ID NO: 25 - protein, 19 aa, synthetic construct
  SITE 1..9: note = D-Arginine
RRRRRRRRRKPILFFRLGK

SEQ ID NO: 26 - protein, 20 aa, synthetic construct
  SITE 1..9: note = D-Arginine
RRRRRRRRRKGSALISWIKR

SEQ ID NO: 27 - protein, 25 aa, synthetic construct
  SITE 25: note = D-Arginine
RLVSYNGIIFFLKKGKPILFFRLKR

SEQ ID NO: 28 - protein, 23 aa, synthetic construct
RLVSYNGIIFFLKKPILFFRLGK

SEQ ID NO: 29 - protein, 24 aa, synthetic construct
RLVSYNGIIFFLKKGSALISWIKR

SEQ ID NO: 30 - protein, 24 aa, synthetic construct
  SITE 24: note = D-Arginine
FNLPLPSRPLLRKGKPILFFRLKR

SEQ ID NO: 31 - protein, 22 aa, synthetic construct
FNLPLPSRPLLRKPILFFRLGK

SEQ ID NO: 32 - protein, 23 aa, synthetic construct
FNLPLPSRPLLRKGSALISWIKR

SEQ ID NO: 33 - protein, 7 aa, synthetic construct
PKKKRKV

SEQ ID NO: 34 - DNA, 19 bp, synthetic construct
  source 1..19: mol_type = other DNA
tgtggccaggcggacgcag

SEQ ID NO: 35 - DNA, 19 bp, synthetic construct
  source 1..19: mol_type = other DNA
aggtcgaacagcagcgtgt

SEQ ID NO: 36 - DNA, 20 bp, synthetic construct
  source 1..20: mol_type = other DNA
gaccagctccaacccaaact

SEQ ID NO: 37 - DNA, 20 bp, synthetic construct
  source 1..20: mol_type = other DNA
gccaccgccggtcatattta

SEQ ID NO: 38 - DNA, 20 bp, synthetic construct
  source 1..20: mol_type = other DNA
cccactcccttggcacattc

SEQ ID NO: 39 - DNA, 20 bp, synthetic construct
  source 1..20: mol_type = other DNA
agcatggttgaacgttgtgc

SEQ ID NO: 40 - DNA, 22 bp, synthetic construct
  source 1..22: mol_type = other DNA
cagaggtggaacagctatcaag

SEQ ID NO: 41 - DNA, 22 bp, synthetic construct
  source 1..22: mol_type = other DNA
cattgggcaagaagccataaac

SEQ ID NO: 42 - DNA, 22 bp, synthetic construct
  source 1..22: mol_type = other DNA
aaaggacaggacatccgtaatg

SEQ ID NO: 43 - DNA, 20 bp, synthetic construct
  source 1..20: mol_type = other DNA
tcgctggactctacacttct

SEQ ID NO: 44 - DNA, 20 bp, synthetic construct
  source 1..20: mol_type = other DNA
acggtctgatccgcaaatac

SEQ ID NO: 45 - DNA, 22 bp, synthetic construct
  source 1..22: mol_type = other DNA
atccattcaaggtcgcttagtc

SEQ ID NO: 46 - DNA, 885 bp, Homo sapiens
  source 1..885: mol_type = genomic DNA
ttagagtgtgatgggctgagtggggttgtgagtgattatcttcttttttcagtttttttctgggttttccaagtgttcctcgatgaacatggatagtttttctgacaggataaaaaagaagtagtccgggacagtggctaacaccccgaatcccagcactttgggaagccggaggtgggaggatcgcttgaggccaggagtttgaaaccagcctgggcagcataacgacactccctctctacgaaaaacgaaaaaaaataattagccggacgtggtggcgtgcgactgtggtcccagctactcgggaggctgaggtgggaggatcgcttgagcccaataggtggaggctccgtgagctgagatagcgccactgcgctcctgcctgggcgacagagtgagaacctgactcaaaacaaagaaaaaaggaagaaaagaaaggaagggaagaaggaaggaagggagaagctttcaaaaataaacttttgtaagaagtaatgacaccgctagccgtccacaccaggagaccgcctagccgtggggcacggtgggctcctgggagctctgagctctgggctttctccagttcgcactctgcttgtctcggcagctccgtccccaccgcagaggtgtgaaggggcgcaaagccagcgaagggagaacccgggtcgggtaacccccaggcctggccaggcggacgcaggggcatctcgggcgaggcgcgccttgcgtcacgtgggcaccgcccctgcagtgaccggagaacggcgtgttcctagggctctgctgccgtcgtggaagcaggatttccgcggttgtgtaacggcctgtcgcaggcgtgcagggacctggactccgcctcgtccccggggctcgggcagccgagcc

SEQ ID NO: 47 - DNA, 1365 bp, Homo sapiens
  source 1..1365: mol_type = genomic DNA
cacctctggtgttttcacttgatggccactgcccactcttccagtcactaaggctgagatttttctcaccactttcaaaacaactttttgtcactggtaacatgcctcatgtgacctagaagattttgaaacaacaattttacttaaactccgtgaagatttcaaaacaacaattttacttaaactccatgttgaaggcaaaaaggaagattgtgtgaagcactttgaggaaggaaaaaatgatggaacgatatttcatcaaggaagcccatgaggaagaaaatttattaaaaccaagtaggtgtatggaggagaggccgcctcgtgaagaaaacgaacataattttacgctgatttggtgtcttttctgtttttctcttgttggtatctacattcatcttttccaataataatcccatatatgtacatttttatctgtttaaattcagtaaaagttgggaggaaaatgtgtcaaaacttttagagtgtgatgggctgagtggggttgtgagtgattatcttcttttttcagtttttttctgggttttccaagtgttcctcgatgaacatggatagtttttctgacaggataaaaaagaagtagtccgggacagtggctaacaccccgaatcccagcactttgggaagccggaggtgggaggatcgcttgaggccaggagtttgaaaccagcctgggcagcataacgacactccctctctacgaaaaacgaaaaaaaataattagccggacgtggtggcgtgcgactgtggtcccagctactcgggaggctgaggtgggaggatcgcttgagcccaataggtggaggctccgtgagctgagatagcgccactgcgctcctgcctgggcgacagagtgagaacctgactcaaaacaaagaaaaaaggaagaaaagaaaggaagggaagaaggaaggaagggagaagctttcaaaaataaacttttgtaagaagtaatgacaccgctagccgtccacaccaggagaccgcctagccgtggggcacggtgggctcctgggagctctgagctctgggctttctccagttcgcactctgcttgtctcggcagctccgtccccaccgcagaggtgtgaaggggcgcaaagccagcgaagggagaacccgggtcgggtaacccccaggcctggccaggcggacgcaggggcatctcgggcgaggcgcgccttgcgtcacgtgggcaccgcccctgcagtgaccggagaacggcgtgttcctagggctctgctgccgtcgtggaagcaggatttccgcggttgtgtaacggcctgtcgcaggcgtgcagggacctggactccgcctcgtccccggggctcgggcagccgagcc

SEQ ID NO: 48 - DNA, 1605 bp, Homo sapiens
  source 1..1605: mol_type = genomic DNA
gaaggaaggaaggaaggtaggtaggtgtacagtgtgtacagattgacaataaaaccttgaacaacgggatatcgaaattcttgcccacacagtatgggctttgccagttttcagatacatttttatgaagtattttcgctggcaaaaccattcaggtaggggcatgaaatgatagtgtttcatcactcgtgatctctaggggcagctattccaaagactacaatctgagaggtttcaaaacacctctggtgttttcacttgatggccactgcccactcttccagtcactaaggctgagatttttctcaccactttcaaaacaactttttgtcactggtaacatgcctcatgtgacctagaagattttgaaacaacaattttacttaaactccgtgaagatttcaaaacaacaattttacttaaactccatgttgaaggcaaaaaggaagattgtgtgaagcactttgaggaaggaaaaaatgatggaacgatatttcatcaaggaagcccatgaggaagaaaatttattaaaaccaagtaggtgtatggaggagaggccgcctcgtgaagaaaacgaacataattttacgctgatttggtgtcttttctgtttttctcttgttggtatctacattcatcttttccaataataatcccatatatgtacatttttatctgtttaaattcagtaaaagttgggaggaaaatgtgtcaaaacttttagagtgtgatgggctgagtggggttgtgagtgattatcttcttttttcagtttttttctgggttttccaagtgttcctcgatgaacatggatagtttttctgacaggataaaaaagaagtagtccgggacagtggctaacaccccgaatcccagcactttgggaagccggaggtgggaggatcgcttgaggccaggagtttgaaaccagcctgggcagcataacgacactccctctctacgaaaaacgaaaaaaaataattagccggacgtggtggcgtgcgactgtggtcccagctactcgggaggctgaggtgggaggatcgcttgagcccaataggtggaggctccgtgagctgagatagcgccactgcgctcctgcctgggcgacagagtgagaacctgactcaaaacaaagaaaaaaggaagaaaagaaaggaagggaagaaggaaggaagggagaagctttcaaaaataaacttttgtaagaagtaatgacaccgctagccgtccacaccaggagaccgcctagccgtggggcacggtgggctcctgggagctctgagctctgggctttctccagttcgcactctgcttgtctcggcagctccgtccccaccgcagaggtgtgaaggggcgcaaagccagcgaagggagaacccgggtcgggtaacccccaggcctggccaggcggacgcaggggcatctcgggcgaggcgcgccttgcgtcacgtgggcaccgcccctgcagtgaccggagaacggcgtgttcctagggctctgctgccgtcgtggaagcaggatttccgcggttgtgtaacggcctgtcgcaggcgtgcagggacctggactccgcctcgtccccggggctcgggcagccgagcc

SEQ ID NO: 49 - DNA, 777 bp, Homo sapiens
  source 1..777: mol_type = genomic DNA
gataaaaaagaagtagtccgggacagtggctaacaccccgaatcccagcactttgggaagccggaggtgggaggatcgcttgaggccaggagtttgaaaccagcctgggcagcataacgacactccctctctacgaaaaacgaaaaaaaataattagccggacgtggtggcgtgcgactgtggtcccagctactcgggaggctgaggtgggaggatcgcttgagcccaataggtggaggctccgtgagctgagatagcgccactgcgctcctgcctgggcgacagagtgagaacctgactcaaaacaaagaaaaaaggaagaaaagaaaggaagggaagaaggaaggaagggagaagctttcaaaaataaacttttgtaagaagtaatgacaccgctagccgtccacaccaggagaccgcctagccgtggggcacggtgggctcctgggagctctgagctctgggctttctccagttcgcactctgcttgtctcggcagctccgtccccaccgcagaggtgtgaaggggcgcaaagccagcgaagggagaacccgggtcgggtaacccccaggcctggccaggcggacgcaggggcatctcgggcgaggcgcgccttgcgtcacgtgggcaccgcccctgcagtgaccggagaacggcgtgttcctagggctctgctgccgtcgtggaagcaggatttccgcggttgtgtaacggcctgtcgcaggcgtgcagggacctggactccgcctcgtccccggggctcgggcagccgagcc

(19) United States
(12) Patent Application Publication
(10) Pub. No.: US 2025/0177573 A1
(43) Pub. Date: Jun. 5, 2025

(54) Materials & Methods for Treatment of Macular Degeneration

(71) Applicant: The University of Bristol, Bristol, GBN (GB)

(72) Inventors: Ying Kai Chan, Bristol (GB); Andrew Dick, Bristol (GB); Jian Liu, Bristol (GB)

(73) Assignee: The University of Bristol, Bristol, GBN (GB)

(21) Appl. No.: 18/710,972

(22) PCT Filed: Nov. 18, 2022
(86) PCT No.: PCT/EP2022/082518

§ 371 Date: May 16, 2024

Related U.S. Application Data
(60) Provisional application No. 63/281,360, filed on Nov. 19, 2021.

(51) Int. Cl. A61K 48/00; A61K 9/00; A61K 38/17; A61P 27/02; C12N 9/22; C12N 15/11; C12N 15/115; C12N 15/86

(52) U.S. Cl. CPC A61K 48/0066; A61K 9/0048; A61K 38/1709; A61K 48/0075; A61P 27/02; C12N 9/22; C12N 15/111; C12N 15/115; C12N 15/86; C07K 2319/09; C07K 2319/85; C12N 2310/16; C12N 2310/20; C12N 2750/14143

(57) ABSTRACT

The present invention relates to an agent that increases expression of IRAK-M and/or activity of IRAK-M for use in a method of treatment or prophylaxis of macular degeneration in a subject. The agent may be one or more of a small molecule, a nucleic acid, a vector virion, a polypeptide, a nucleic acid system, a viral vector system, or a pharmaceutical composition.

### CROSS-REFERENCE

This application is a 371 National Stage filing and claims the benefit under 35 U.S.C. § 120 of International Application No. PCT/EP2022/082518, filed 18 Nov. 2022, which claims priority to U.S. Provisional Application 63/281,360, filed 19 Nov. 2021, each of which is incorporated herein by reference in its entirety.

### SEQUENCE LISTING INCORPORATION BY REFERENCE

The application herein incorporates by reference in its entirety the sequence listing material submitted concurrently with the specification as an XML file, with a filename of “Sequence Listing”, a production date of Nov. 28, 2022, and a size of 57,344 bytes. The ST26 Sequence Listing is part of the specification and is incorporated in its entirety by reference herein.

### FIELD OF THE INVENTION

The present invention relates to an agent that increases expression of IRAK-M and/or activity of IRAK-M for use in a method of treatment or prophylaxis of macular degeneration in a subject. The agent may be one or more of a small molecule, a nucleic acid, a vector virion, a polypeptide, a nucleic acid system, a viral vector system, or a pharmaceutical composition.

### BACKGROUND

Alongside other cell-autonomous responses such as metabolic regulation and autophagy, immune-mediated inflammation initiated by noxious stress (environmental factors) is at the frontline to maintain and restore homeostasis (1-3). Not only does insufficiency or failure in the immune response results in tissue damage, but also excessive immune response, and particularly chronic inflammation or divergent or defective responses are detrimental. All may be accentuated with age (“inflammageing”) and contributes to age-related degenerative disorders (4, 5).

Age-related macular degeneration (AMD) is the leading cause of blindness in the elderly, and the prevalence gradually increases with age. With increasing life expectancy, AMD has become a major public health issue as the global AMD burden is projected to reach 288 million people by 2040 (6, 7). In the US, approximately 11 million people are affected by AMD, a prevalence that is similar to that of all invasive cancers combined, and over double of that of Alzheimer's disease (6). The global cost of visual impairment due to AMD alone is substantial, estimated to be US$343 billion including 74% in direct healthcare costs (AMD Alliance International).

Clinically, AMD is characterized by deposits of lipoproteinaceous drusen and pigmentary abnormalities in the RPE (early AMD), an insidious lesion of RPE frequently but not exclusively preceding photoreceptor loss (geographic atrophy, dry AMD, late form) or, in 10-15% cases, choroidal neovascularization (CNV, wet AMD, late form). Due to a current lack of early intervention options for dry AMD, a substantial proportion of AMD eventually leads to severe visual impairment or blindness (6, 8). In addition to the association with immune response-related genotypes and complement (9-12), unchecked inflammatory responses from immune cells (such as microglia/macrophages), and immuno-competent tissue-resident cells (such as RPE) form a crucial driving force to accelerate tissue ageing towards AMD (13-17). Notwithstanding, mechanisms behind the defective immunoregulation with ageing remain elusive.

Among various retinal cell types, RPE is regarded as the most susceptible to ageing with the highest number of differential expression genes (DEGs) overlapping with genes associated with ageing and age-related retinal diseases (18, 19). Oxidative changes in the ageing RPE predisposes AMD by triggering altered mitochondrial metabolism, impaired intracellular RPE processing pathways (autophagy, phagolysosome and protein trafficking) and senescence, all tractable pathways that cross-regulate and also determine appropriate immune responses (20-24). Inflammatory responses can be initiated by pathogen-induced reactive oxygen species (ROS) through signalling transduction pathways mediated by Toll-like receptors (TLRs) (25-27), which detect a variety of pathogen-associated molecular patterns (PAMPs) and damage-associated molecular patterns (DAMPs). Nevertheless, excessive or persistent TLR-mediated inflammation disrupts cell and tissue homeostasis.

The magnitude of inflammatory responses is balanced by tonic inhibitory mechanisms, including Interleukin 1 receptor-associated kinase-M (IRAK-M), encoded by IRAK3 gene, a unique IRAK family member that lacks kinase activity and serves as an anti-inflammatory molecule (28). IRAK-M suppresses TLRs or Interleukin 1 receptor (IL-1R)-transduced inflammation cascade by impeding the uncoupling of IRAK1/4 from IRAK-MyD88 complex (Myddosome) (28-30). Dysregulated IRAK signalling contributes to metabolic insulin resistance in diabetes and obesity (30, 31). IRAK-M expression is downregulated in monocytes and adipose tissues of obese subjects, associated with exaggerated oxidative stress, elevated systemic inflammation and features of metabolic syndrome (31). It was previously found that IRAK-M was expressed in a murine RPE cell line and the expression was reduced by wortmannin (a PI3K inhibitor) or following a prolonged period of culture when the cells demonstrated increased mitochondrial superoxide and impaired autophagy (13). However, the expression, role, and significance of the regulation of IRAK-M in retinal health and diseases have not been defined.

### SUMMARY OF THE INVENTION

The present inventors have identified that IRAK-M expression within the RPE in human and mouse retinas declines with age and oxidative stress. Data mining of an RNA-Seq study further divulged lower IRAK-M expression in AMD eyes than those from age-matched controls. Immunohistochemistry staining analysis of human eye sections additionally confirmed the decline of ocular IRAK-M expression with age and AMD. The decreased IRAK-M expression may undermine ability to maintain RPE function and health. For example, IRAK-M knockout mice developed outer retinal and RPE pathology, and this was accentuated following oxidative insult. The present inventors have showed that augmentation of IRAK-M expression provided protection to the RPE and retina. By introducing human IRAK-M transgene to mouse RPE, mitochondrial activity was retained and cell survival under oxidative stress was promoted. The present inventors have also shown the prevention of AMD-like phenotype in two animal models (i.e., prevention of light-induced retinal damage (LIRD) in wild-type mice and prevention of age-related retinal damage in IRAK-M knockout mice).

Accordingly, the invention relates to an agent for increasing IRAK-M expression in a target cell and/or increasing IRAK-M activity in a target cell, for use in a method of treatment or prophylaxis of macular degeneration in a subject.

IRAK-M in a target cell may be increased by introducing exogenous IRAK-M to a target cell. Thus, the agent may be an IRAK-M polypeptide or a nucleic acid encoding IRAK-M. The following aspects relate to approaches for increasing IRAK-M in a target cell by introducing exogenous IRAK-M to a target cell.

In an aspect of the invention, provided is a nucleic acid for use in a method of treatment or prophylaxis of macular degeneration in a subject. The nucleic acid may comprise a nucleic acid sequence encoding IRAK-M. The nucleic acid may be capable of driving expression of IRAK-M in a target cell.

In some embodiments, a promoter is operably linked to the nucleic acid sequence. The promoter may be an RPE-specific promoter. The RPE-specific promoter may be selected from the group consisting of a RPE65 promoter, a NA65 promoter, a VMD2 promoter (also known as Best1 promoter), and a Synpiii promoter. In alternative embodiments, the promoter is a ubiquitous promoter. The ubiquitous promoter may be selected from the group consisting of a CMV promoter, a CAG promoter, a GAPDH promoter, a UbiC promoter, and an EF-1α promoter. In other embodiments, the promoter is the native promoter for IRAK3 or a functional fragment thereof.

In some embodiments, IRAK-M expression is increased in the target cell comprising the nucleic acid compared to an equivalent cell not comprising the nucleic acid. Autophagic flux may be maintained or increased in the target cell comprising the nucleic acid compared to an equivalent cell not comprising the nucleic acid. Mitochondrial activity may be maintained or increased in the target cell comprising the nucleic acid compared to an equivalent cell not comprising the nucleic acid. Proinflammatory cytokine production may be reduced in the target cell comprising the nucleic acid compared to an equivalent target cell not comprising the nucleic acid. The proinflammatory cytokines may be selected from the group consisting of GM-CSF and MCP-1.

The nucleic acid may be suitable for integration into the genome of the target cell by an RNA-guided endonuclease system. The RNA-guided endonuclease may be a CRISPR-Cas system.

In some embodiments, the nucleic acid is DNA. The nucleic acid may be an episome. In some embodiments, the nucleic acid is a plasmid or a minicircle. In some embodiments, the nucleic acid is RNA. The nucleic acid may be messenger RNA or circular RNA.

In some embodiments, the nucleic acid is delivered to a target cell via a non-viral carrier. The non-viral carrier may be selected from the group consisting of nanoparticles, liposomes, cationic polymer, and calcium phosphate particles.

In some embodiments, the nucleic acid is delivered to a target cell via a viral vector. The viral vector may be selected from the group consisting of an adeno-associated virus vector, an adenovirus vector, a retrovirus vector, an orthomyxovirus vector, a paramyxovirus vector, a papovavirus vector, a picornavirus vector, a lentivirus vector, a herpes simplex virus vector, a vaccinia virus vector, a pox virus vector, an anellovirus vector, and an alphavirus vector.

The nucleic acid may be a viral vector genome. The viral vector genome may be selected from the group consisting of an adeno-associated virus vector genome, an adenovirus vector genome, a retrovirus vector genome, an orthomyxovirus vector genome, a paramyxovirus vector genome, a papovavirus vector genome, a picornavirus vector genome, a lentivirus vector genome, a herpes simplex virus vector genome, a vaccinia virus vector genome, a pox virus vector genome, an anellovirus vector genome, and an alphavirus vector genome.

The macular degeneration may be age-related macular degeneration (AMD). The age-related macular degeneration (AMD) may be dry AMD. In some embodiments, the dry AMD is selected from the group consisting of early dry AMD, intermediate dry AMD, and advanced dry AMD.

The target cell may be a cell of the retina or the choroid. In some embodiments, the target cell is a cell of the retina. The target cell may be a cell of the ganglion cell layer (GCL), the inner plexiform layer (IPL), the inner nuclear layer (INL), the outer plexiform layer (OPL), the outer nuclear layer (ONL), the photoreceptor outer segment (POS), or the retinal pigmental epithelium (RPE). In some embodiments, the target cell is a cell of the RPE.

The target cell may be a myeloid cell. The myeloid cell may be a retinal myeloid cell. In some embodiments, the target cell is a CD11b+ myeloid cell.

The nucleic acid may be administered intraocularly, intravitreally, subretinally, or periocularly to a subject. In some embodiments, the nucleic acid is administered subretinally. The nucleic acid may be administered by injection or infusion. In some embodiments, the nucleic acid is administered by subretinal injection. In some embodiments, the subject is human. The subject may be affected by or at risk of developing macular degeneration.

In some embodiments, the nucleic acid sequence encodes a polypeptide comprising an amino acid sequence having at least 60% sequence identity to the amino acid sequence of SEQ ID NO: 1. In some embodiments, the nucleic acid sequence encodes a functional polypeptide. The nucleic acid sequence may encode a polypeptide capable of preventing dissociation of IRAK-1 and/or IRAK-4 from MyD88 in a target cell. The nucleic acid sequence may encode a polypeptide capable of preventing formation of an IRAK-1-TRAF6 complex.

In another aspect, provided is a vector virion for use in a method of treatment or prophylaxis of macular degeneration in a subject, the vector virion comprising the nucleic acid described herein.

In some embodiments, IRAK-M expression is increased in the target cell comprising the vector virion compared to an equivalent cell not comprising the vector virion. Autophagic flux may be maintained or increased in the target cell comprising the vector virion compared to an equivalent cell not comprising the vector virion. Mitochondrial activity may be maintained or increased in the target cell comprising the nucleic acid compared to an equivalent cell not comprising the vector virion. Proinflammatory cytokine production may be reduced in the target cell comprising the vector virion compared to an equivalent target cell not comprising the vector virion. The proinflammatory cytokines may be selected from the group consisting of GM-CSF and MCP-1.

The vector virion may be selected from the group consisting of adeno-associated virus, adenovirus, retrovirus, orthomyxovirus, paramyxovirus, papovavirus, picornavirus, lentivirus, herpes simplex virus, vaccinia virus, pox virus, anellovirus, and alphavirus. In some embodiments, the vector virion is an adeno-associated virus (AAV). The AAV may be selected from the group consisting of AAV type 1 (AAV-1), AAV type 2 (AAV-2), AAV type 3 (AAV-3), AAV type 4 (AAV-4), AAV type 5 (AAV-5), AAV type 6 (AAV6), AAV type 7 (AAV-7), AAV type 8 (AAV-8), and AAV type 9 (AAV9). In some embodiments, the AAV is AAV2. In some embodiments, the AAV is AAV8.

The vector virion may be administered intraocularly, intravitreally, subretinally, or periocularly to a subject. In some embodiments, the vector virion is administered subretinally. The vector virion may be administered by injection or infusion. In some embodiments, the vector virion is administered by subretinal injection. In some embodiments, the subject is human. The subject may be affected by or at risk of developing macular degeneration.

In some embodiments, the vector virion comprises a nucleic acid sequence encoding a polypeptide comprising an amino acid sequence having at least 60% sequence identity to the amino acid sequence of SEQ ID NO: 1. The vector virion may comprise a nucleic acid sequence encoding a polypeptide capable of preventing dissociation of IRAK-1 and/or IRAK-4 from MyD88 in a target cell. The vector virion may comprise a nucleic acid sequence encoding a polypeptide capable of preventing formation of an IRAK-1-TRAF6 complex.

A further aspect provides an IRAK-M polypeptide for use in a method of treatment or prophylaxis of macular degeneration in a subject. In some embodiments, the polypeptide comprises an amino acid sequence having at least 60% sequence identity to the amino acid sequence of SEQ ID NO: 1.

The polypeptide may be capable of preventing dissociation of IRAK-1 and/or IRAK-4 from MyD88 in a target cell. The polypeptide may be capable of preventing formation of an IRAK-1-TRAF6 complex.

In some embodiments, the polypeptide further comprises a cell penetrating peptide (CPP). In some embodiments, the polypeptide further comprises a peptide-based cleavable linker (PCL). The CPP may be conjugated to the N-terminus of the PCL. The amino acid sequence having at least 60% sequence identity to the amino acid sequence of SEQ ID NO: 1 may be conjugated to the C-terminus of the PCL. In some embodiments, the PCL is a peptide sequence that is cleavable by cathepsin D.

Autophagic flux may be maintained or increased in the target cell comprising the polypeptide compared to an equivalent cell not comprising the polypeptide. Mitochondrial activity may be maintained or increased in the target cell comprising the polypeptide compared to an equivalent cell not comprising the polypeptide. Proinflammatory cytokine production may be reduced in the target cell comprising the polypeptide compared to an equivalent target cell not comprising the polypeptide. The proinflammatory cytokines may be selected from the group consisting of GM-CSF and MCP-1.

In an aspect of the invention, a nucleic acid system is provided comprising one or more nucleic acids, comprising:
a) a nucleic acid sequence encoding an RNA-guided endonuclease;
b) a nucleic acid sequence encoding a guide RNA complementary to a target sequence associated with an insertion site in the genome of the target cell and capable of directing said RNA-guided endonuclease to said target sequence; and
c) a nucleic acid sequence encoding IRAK-M,
   for use in a method of treatment or prophylaxis of macular degeneration in a subject. The nucleic acid sequence encoding IRAK-M is capable of driving expression of IRAK-M in a target cell of the subject and the nucleic acid system is suitable for directed insertion of the nucleic acid sequence encoding IRAK-M at the insertion site in the genome of the target cell.

The nucleic acid sequence encoding IRAK-M may be flanked by 5′ homology arm and a 3′ homology arm. In some embodiments, the 5′ homology arm is homologous to a DNA sequence 5′ of the target sequence from the insertion site and the 3′ homology arm is homologous to a DNA sequence 3′ of the target sequence from the insertion site. The nucleic acid sequence encoding IRAK-M may further comprise a 5′ flanking sequence comprising a target sequence and a 3′ flanking sequence comprising a target sequence. In some embodiments, the 5′ flanking sequence is 5′ of the 5′ homology arm and the 3′ flanking sequence is 3′ of the 3′ homology arm.

In alternative embodiments, the nucleic acid sequence encoding IRAK-M is flanked by a 5′ target sequence and a 3′ target sequence. The 5′ target sequence and the 3′ target sequence may be identical to target sequence from an insertion site in the genome.

In some embodiments, the one or more nucleic acids are one or more viral vector genomes. The one or more viral vector genomes may be one or more adeno-associated virus vector genomes.

Further provided is a viral vector system comprising the nucleic acid system as described herein. In some embodiments, the viral vector system is an adeno-associated virus vector system.

Another aspect provides a pharmaceutical composition comprising a nucleic acid, a vector virion, a polypeptide, a nucleic acid system, or a viral vector system as described herein. In some embodiments, the pharmaceutical composition is formulated for ocular delivery.

IRAK-M expression may be increased by increasing endogenous IRAK-M expression in a target cell. Thus, the agent may be capable of increasing endogenous IRAK-M expression. The following aspects relate to agents capable of increasing endogenous IRAK-M expression in a target cell.

Accordingly, provided is a nucleic acid system comprising one or more nucleic acids, comprising:
a) a nucleic acid sequence encoding a deactivated RNA-guided endonuclease fused to one or more transcriptional activators; and
b) a nucleic acid sequence encoding a guide RNA complementary to a target sequence in the promoter or regulatory sequences for the IRAK3 gene and capable of directing said RNA-guided endonuclease to said target sequence,
   for use in a method of treatment or prophylaxis of macular degeneration in a subject, where the nucleic acid system increases IRAK-M expression in a target cell of the subject.

The transcriptional activator may be VP64.

Accordingly, provided is a nucleic acid system comprising one or more nucleic acids, comprising:
a) a nucleic acid sequence encoding a deactivated RNA-guided endonuclease; and
b) a nucleic acid sequence encoding a guide RNA complementary to a target sequence in the promoter or regulatory sequences for IRAK3 gene and capable of directing said RNA-guided endonuclease to said target sequence, wherein said guide RNA further comprises an aptamer capable of specifically binding to a transcriptional activator, for use in a method of treatment or prophylaxis of macular degeneration in a subject, wherein the nucleic acid system increases IRAK-M expression in a target cell of the subject.

An aspect of the invention, also provides a nucleic acid system comprising one or more nucleic acids, comprising:
a) a nucleic acid sequence encoding a deactivated RNA-guided endonuclease;
b) a nucleic acid sequence encoding an RNA binding protein fused to one or more transcriptional activators; and
c) a nucleic acid sequence encoding a guide RNA complementary to a target sequence in the promoter or regulatory sequences for the IRAK3 gene and capable of directing said RNA-guided endonuclease to said target sequence, wherein said guide RNA further comprises an RNA aptamer capable of specifically binding to the RNA binding protein,
   for use in a method of treatment or prophylaxis of macular degeneration in a subject, wherein the nucleic acid system increases IRAK-M expression in a target cell of the subject.

The one or more transcriptional activators may be selected from the group consisting of VP64, p65 and HSF1. The RNA aptamer may be capable of binding to an RNA binding protein dimer. The RNA binding protein may be MS2. In some embodiments, the deactivated RNA-guided endonuclease is fused to an additional transcriptional activator. The additional transcriptional activator may be VP64.

A further aspect provides a nucleic acid system comprising one or more nucleic acids, comprising:
a) a nucleic acid sequence encoding a deactivated RNA-guided endonuclease fused to an epitope repeat array comprising one or more epitopes;
b) one or more nucleic acid sequences encoding an epitope binding molecule fused to one or more transcriptional activators, wherein said epitope binding molecule is capable of specifically binding to an epitope of the epitope repeat array; and
c) a nucleic acid sequence encoding a guide RNA complementary to a target sequence in the promoter or regulatory sequences for the IRAK3 gene and capable of directing said RNA-guided endonuclease to said target sequence,
   for use in a method of treatment or prophylaxis of macular degeneration in a subject, wherein the nucleic acid system increases IRAK-M expression in a target cell of the subject.

The epitope binding molecule may comprise a nuclear localisation sequence (NLS). The epitope binding molecule may be an antibody or antibody-like molecule. In some embodiments, the one or more transcriptional activators are selected from the group consisting of VP64, p65 and Rta.

The one or more nucleic acids may be one or more viral vector genomes. The one or more viral vector genomes may be one or more adeno-associated virus vector genomes.

Another aspect provides a nucleic acid system comprising one or more nucleic acids, comprising:
a) a nucleic acid sequence encoding a deactivated RNA-guided endonuclease fused to one or more DNA demethylating agents; and
b) a nucleic acid sequence encoding a guide RNA complementary to (i) a target sequence in the promoter sequence for the IRAK3 gene, (ii) a target sequence in the regulatory sequences for the IRAK3 gene or (iii) a target sequence in the IRAK3 gene, and capable of directing said RNA-guided endonuclease to said target sequence,
   for use in a method of treatment or prophylaxis of macular degeneration in a subject, wherein the nucleic acid system increases IRAK-M expression in a target cell of the subject.

Further provided is a nucleic acid system comprising one or more nucleic acids, comprising:
a) a nucleic acid sequence encoding a deactivated RNA-guided endonuclease; and
b) a nucleic acid sequence encoding a guide RNA complementary to (i) a target sequence in the promoter sequence for the IRAK3 gene, (ii) a target sequence in the regulatory sequences for the IRAK3 gene or (iii) a target sequence in the IRAK3 gene, and capable of directing said RNA-guided endonuclease to said target sequence, wherein said guide RNA further comprises an aptamer capable of specifically binding to a DNA demethylating agent,
   for use in a method of treatment or prophylaxis of macular degeneration in a subject, wherein the nucleic acid system increases IRAK-M expression in a target cell of the subject.

An aspect provides a nucleic acid system comprising one or more nucleic acids, comprising:
a) a nucleic acid sequence encoding a deactivated RNA-guided endonuclease;
b) a nucleic acid sequence encoding an RNA binding protein fused to one or more DNA demethylating agents;
c) a nucleic acid sequence encoding a guide RNA complementary to (i) a target sequence in the promoter sequence for the IRAK3 gene, (ii) a target sequence in the regulatory sequences for the IRAK3 gene or (iii) a target sequence in the IRAK3 gene, and capable of directing said RNA-guided endonuclease to said target sequence, wherein said guide RNA further comprises an RNA aptamer capable of specifically binding to the RNA binding protein,
   for use in a method of treatment or prophylaxis of macular degeneration in a subject, wherein the nucleic acid system increases IRAK-M expression in a target cell of the subject.

The RNA aptamer may be capable of binding to an RNA binding protein dimer. The RNA binding protein may be MS2. In some embodiments, the deactivated RNA-guided endonuclease is fused to an additional transcriptional activator.

A further aspect provides a nucleic acid system comprising one or more nucleic acids, comprising:
a) a nucleic acid sequence encoding a deactivated RNA-guided endonuclease fused to an epitope repeat array comprising one or more epitopes;
b) one or more nucleic acid sequences encoding an epitope binding molecule fused to one or more DNA demethylating agents, wherein said epitope binding molecule is capable of specifically binding to an epitope of the epitope repeat array; and
c) a nucleic acid sequence encoding a guide RNA complementary to (i) a target sequence in the promoter sequence for the IRAK3 gene, (ii) a target sequence in the regulatory sequences for the IRAK3 gene or (iii) a target sequence in the IRAK3 gene, and capable of directing said RNA-guided endonuclease to said target sequence,
   for use in a method of treatment or prophylaxis of macular degeneration in a subject, wherein the nucleic acid system increases IRAK-M expression in a target cell of the subject.

The epitope binding molecule may comprise a nuclear localisation sequence (NLS). The epitope binding molecule may be an antibody or antibody-like molecule.

In some embodiments, the DNA demethylating agent is TET1. In some embodiments, the DNA demethylating agent is LESD1.

The one or more nucleic acids may be one or more viral vector genomes. The one or more viral vector genomes may be one or more adeno-associated virus vector genomes.

Also provided is a viral vector system comprising the nucleic acid systems as described herein. In some embodiments, the viral vector system is an adeno-associated virus vector system.

An alternative targeted approach for increasing endogenous expression of IRAK-M in a target cell may use a nucleic acid binding molecule (e.g., nucleic acid binding portion) capable of binding to a target sequence.

Thus, an aspect provides a nucleic acid comprising a nucleic acid sequence encoding a fusion protein, the fusion protein comprising:
a) a nucleic acid binding molecule capable of binding to a target sequence in the promoter or regulatory sequences of the IRAK3 gene; and
b) one or more transcriptional activators,
   for use in a method of treatment or prophylaxis of macular degeneration in a subject, wherein the fusion protein is capable of increasing IRAK-M expression in a target cell of the subject.

An aspect of the invention provides a nucleic acid system comprising:
a) a nucleic acid sequence encoding a fusion protein, wherein the fusion protein comprises (i) a nucleic acid binding molecule capable of binding to a target sequence in the promoter or regulatory sequences of the IRAK3 gene and (ii) an epitope repeat array; and
b) one or more nucleic acid sequences encoding an epitope binding molecule fused to one or more transcriptional activators, wherein said epitope binding molecule is capable of specifically binding to an epitope of the epitope repeat array,
   for use in a method of treatment or prophylaxis of macular degeneration in a subject, wherein the nucleic acid system is capable of increasing IRAK-M expression in a target cell of the subject.

A further aspect provides a nucleic acid sequence encoding a fusion protein, the fusion protein comprising:
a) a nucleic acid binding molecule capable of binding to (i) a target sequence in the promoter sequence for the IRAK3 gene, (ii) a target sequence in the regulatory sequences for the IRAK3 gene or (iii) a target sequence in the IRAK3 gene; and
b) one or more DNA demethylating agents,
   for use in a method of treatment or prophylaxis of macular degeneration in a subject, wherein the fusion protein is capable of increasing IRAK-M expression in a target cell of the subject.

Also provided is a nucleic acid system comprising:
a) a nucleic acid sequence encoding a fusion protein, wherein the fusion protein comprises (i) nucleic acid binding molecule capable of binding to (1) a target sequence in the promoter sequence for the IRAK3 gene, (2) a target sequence in the regulatory sequences for the IRAK3 gene or (3) a target sequence in the IRAK3 gene and (ii) an epitope repeat array; and
b) one or more nucleic acid sequences encoding an epitope binding molecule fused to one or more DNA demethylating agents, wherein said epitope binding molecule is capable of specifically binding to an epitope of the epitope repeat array,
   for use in a method of treatment or prophylaxis of macular degeneration in a subject, wherein the nucleic acid system is capable of increasing IRAK-M expression in a target cell of the subject.

The nucleic acid binding molecule may be a TAL effector repeat array or a zinc finger array.

The transcriptional activator may be the transactivation domain, VP64.

The DNA demethylating agent may be a DNA demethylating enzyme or a fragment thereof. In further embodiments, the DNA demethylating agent is the catalytic domain of TET1. In some embodiments, the DNA demethylating agent is TET1. In some embodiments, the DNA demethylating agent is LESD1.

Another aspect provides a small molecule for use in a method of treatment or prophylaxis of macular degeneration in a subject, where the small molecule increases endogenous IRAK-M expression in a target cell of the subject.

The molecule may reduce DNA methylation in the promoter sequence for the IRAK3 gene, the regulatory sequence for the IRAK3 gene and/or in the IRAK3 gene sequence. In some embodiments, the small molecule is EPZ-6438. In some embodiments, the small molecule is azacytidine.

In some embodiments, the small molecule is ibudilast.

The small molecule may enhance the transcription-activating activity of a factor (such as a polypeptide) that promotes transcription from the IRAK3 promoter. In some embodiments, the small molecule is a glucocorticoid. In some embodiments, the small molecule is cortisol.

The small molecule may reduce the N⁶-methyladenosine (m⁶A) modification of IRAK-M mRNA transcripts, which reduces the mRNA degradation and increases IRAK-M expression. In some embodiments, the small molecule is a METTL3 inhibitor. In some embodiments, the small molecule is STM2457. In some embodiments, the small molecule is Cpd-564. In some embodiments, the small molecule is UZH2.

A further aspect provides a nucleic acid for use in a method of treatment or prophylaxis of macular degeneration in a subject, wherein the nucleic acid increases endogenous IRAK-M expression in a target cell of the subject.

In some embodiments, the nucleic acid inhibits METTL3 expression. The nucleic acid may be capable of binding to a target sequence in METTL3 mRNA and downregulating it, thereby increasing IRAK-M expression.

A further aspect provides a peptide or polypeptide for use in a method of treatment or prophylaxis of macular degeneration in a subject, where the peptide or polypeptide increases endogenous IRAK-M in a target cell of the subject.

The peptide or polypeptide may activate ERK1/2 and/or activate PI3K and Akt1. In some embodiments, the peptide or polypeptide is adiponectin. In some embodiments, the peptide or polypeptide is globular adiponectin.

In some embodiments, IRAK-M expression is increased in the target cell comprising the agent capable of increasing endogenous IRAK-M expression compared to an equivalent cell not comprising the agent. Autophagic flux may be maintained or increased in the target cell comprising the agent capable of increasing endogenous IRAK-M expression compared to an equivalent cell not comprising the agent. Mitochondrial activity may be maintained or increased in the target cell comprising the nucleic acid compared to an equivalent cell not comprising the agent capable of increasing endogenous IRAK-M expression Proinflammatory cytokine production may be reduced in the target cell comprising the agent compared to an equivalent target cell not comprising the agent. The proinflammatory cytokines may be selected from the group consisting of GM-CSF and MCP-1.

Alternatively, or additionally, IRAK-M activity could be increased in a target cell. Thus, the agent may be capable of increasing IRAK-M activity. The following aspects relate to increasing IRAK-M activity in a target cell.

The agent may promote IRAK-M binding to IRAK-1 and/or IRAK-4. Alternatively, or additionally, the agent may promote IRAK-M binding to MyD88.

A small molecule for use in a method of treatment or prophylaxis of macular degeneration in a subject is provided, where the small molecule increases IRAK-M activity in a target cell of the subject.

The small molecule is capable of stimulating guanylate cyclase (GC), thereby increasing cellular CGMP. In some embodiments, the small molecule is nitric oxide (NO) donor. In some embodiments, the small molecule is nitric oxide. In some embodiments, the small molecule is riociguat. Alternatively, the small molecule may be cGMP.

Further provided is a peptide or polypeptide for use in a method of treatment or prophylaxis of macular degeneration in a subject, where the peptide or polypeptide increases IRAK-M activity in a target cell of a subject. In some embodiments, the agent is a polypeptide.

The peptide or polypeptide may promote IRAK-M binding to IRAK-1. In some embodiments, the peptide or polypeptide is α-MSH or a fragment thereof.

In some embodiments, IRAK-M activity is increased in the target cell comprising the agent capable of increasing IRAK-M activity compared to an equivalent cell not comprising the agent. Autophagic flux may be maintained or increased in the target cell comprising the agent capable of increasing IRAK-M activity compared to an equivalent cell not comprising the agent. Mitochondrial activity may be maintained or increased in the target cell comprising the nucleic acid compared to an equivalent cell not comprising the agent capable of increasing IRAK-M activity. Proinflammatory cytokine production may be reduced in the target cell comprising the agent capable of increasing IRAK-M activity compared to an equivalent target cell not comprising the agent capable of increasing IRAK-M activity.

The proinflammatory cytokines may be selected from GM-CSF and MCP-1. The macular degeneration may be age-related macular degeneration (AMD). The age-related macular degeneration (AMD) may be dry AMD. In some embodiments, the dry AMD is selected from the group consisting of early dry AMD, intermediate dry AMD, and advanced dry AMD.

The target cell may be a cell of the retina or the choroid. In some embodiments, the target cell is a cell of the retina. The target cell may be a cell of the ganglion cell layer (GCL), the inner plexiform layer (IPL), the inner nuclear layer (INL), the outer plexiform layer (OPL), the outer nuclear layer (ONL), the photoreceptor outer segment (POS), or the retinal pigmental epithelium (RPE). In some embodiments, the target cell is a cell of the RPE.

The target cell may be a myeloid cell. The myeloid cell may be a retinal myeloid cell. In some embodiments, the target cell is a CD11b+ myeloid cell.

A further aspect provides a pharmaceutical composition comprising one or more of the agents as described herein. In some embodiments, the pharmaceutical composition is formulated for ocular delivery.

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures in which:

FIG. 1. IRAK-M is predominantly expressed by RPE in the retina and its expression is reduced with age and in AMD. (A&B) Representative confocal images of human retinal sections from a 20-year-old donor (without recorded ocular disease) demonstrate a predominant immunopositivity of IRAK-M at the pigmented RPE layer (anti-RPE65 stain). DAPI and anti-Rhodopsin were used to stain nuclei and photoreceptor outer segments (POS), respectively. (C&D) Representative confocal images of retinal sections of adult mice showing IRAK-M immunostaining largely colocalized with RPE65. (E&F) Representative western blotting and densitometry quantification demonstrate reduced levels of IRAK-M expression with old age in human RPE/choroidal lysates (E, n=4-6) and mouse RPE lysates (F, n=4-6). (G) Data mining of transcriptome data (GSE99248) shows significantly decreased IRAK-M mRNA level in RPE/Choroid/Sclera of AMD donors, compared to age-matched normal donors. Antisense RNA of IRAK-M showed an insignificant increase in AMD samples (n=7-8).

FIG. 2. In vitro oxidative treatment downregulates IRAK-M expression in RPE cells. (A) A human RPE cell line (ARPE-19) was treated with various concentrations of a prooxidant, paraquat (PQ), for up to 72 h. LDH release demonstrate dose-dependent cytotoxic effect of PQ after 72 h (n=3-4). (B) A sub-toxic concentration of PQ (0.25 mM) was used to stress ARPE-19 and led to diminished IRAK-M expression in ARPE19 cells after 72 h (Western blot), which is accompanied by (C) increased secretion of pro-inflammatory cytokines HMGB1 (EIA, n=4-6), IL-18 and GM-CSF, and decreased anti-inflammatory cytokine IL-11 (multiplex cytokine array, n=4). (D) LDH cytotoxicity assay demonstrates sub-toxic doses of PQ on human iPSC-derived RPE after 72 h treatment (n=5). (E) Western blot shows downregulated IRAK-M expression by 72 h treatment of subtoxic PQ (0.25-0.5 mM). LDH assay (F, n=3-6) and representative western blot (G) show curtailed IRAK-M expression in human primary RPE cell culture by 72 h treatment of sub-toxic PQ (0.25 mM).

FIG. 3. In vivo oxidative insults leads to declined IRAK-M level in the RPE. Retinal oxidative stresses were induced in C57BL/6J mice by either fundus-light induction (100kLux for 20 min, A-C) or intravitreal administration of PQ (2 μl at 1.5 mM, D-F). Western blot analyses of IRAK-M expression in RPE lysate on day 7 post oxidative damage (A&D, n=4 or 5). Representative fundoscopy and OCT images obtained on day 14 demonstrate appearance of retinal lesions (red arrows, B&E) and a surrogate of cell loss as demonstrated by reduced thickness of outer retina (light model, C, n=8) or both outer and inner retina (PQ model, F, n=9-11).

FIG. 4. IRAK-M expression is regulated by AP-1 transcription factor in RPE. (A) ChIP assay of ARPE-19 demonstrates the binding of AP-1 subunits c-Jun and c-Fos proteins to IRAK-M promoter under resting condition, and more pronounced by LPS stimulation for 24 h. (B) Western blot and densitometry analysis show reduced c-Jun expression in mouse RPE at the age of 13 and 19m compared to 3m (n=4). (C) Downregulations of c-Jun and c-Fos phosphorylation in ARPE-19 treated with PQ for 72 h, examined by western blot. (D) c-Jun and c-Fos inhibitors downregulates IRAK-M expression. (E) Augmentation of c-Jun expression by CRISPR/Cas9 activation plasmid induces total c-Jun and phosphorylated c-Jun, as well as IRAK-M expression in ARPE-19. (F) LDH assay shows increased susceptibility of ARPE19 in response to PQ when c-Jun or c-Fos is inhibited (n=4). (G) IRAK-M knockdown by siRNA exacerbates the effect of PQ in inducing ARPE-19 toxicity (n=4).

FIG. 5. Irak3^{-/-} mice spontaneously exhibit early retinal abnormalities. (A) Representative fundal and OCT images show increased incidence of retina presenting scattered white spots (red line arrow) in Irak3^{-/-} mice aged 5m, which is not evident at 2m. (B) Representative fundal images showing time course of appearance of white spots in Irak3^{-/-} mouse retinas. (C) Time course of incidence of flecked retina shows earlier appearance of retinal white spots in Irak3^{-/-} mice. Each value is a ratio of number of flecked retina to total number of retina at each time point. (D) Representative fundal and OCT images demonstrate that the white spots (red line arrow) are associated with outer retinal abnormalities (red arrow) in 5m-old Irak3^{-/-} mice.

FIG. 6. Irak3^{-/-} mice develop AMD-like pathologies and are more vulnerable to oxidative stress. (A) Z-stacks confocal images of retinal flatmounts demonstrate abnormal CD11b+ myeloid populations in the outer retina in 5m-old Irak3^{-/-} mice, which is not seen in WT counterparts. (B) Subretinal accumulation of CD11b+ cells was assessed by immune-staining on RPE/choroidal flatmounts of Irak3^{-/-} mice (n=3-10). (C) TUNEL staining on flatmounts reveals elevated number of apoptotic cells in both retinal and RPE/choroidal tissues of Irak3^{-/-} mice (5m) (n=7-10). (D) Quantification of OCT images indicates a surrogate of retinal cell loss as demonstrated by outer retinal thinning in Irak3^{-/-} mice aged 12-13m (n=6-12). (E) Multiplex cytokine array demonstrates an overall higher level of serum cytokines in KO vs. WT mice (12-13m), where the increases in TNF-α, MCP-1 and IL-10 were statistically significant (n=5-6). (F&G) Eight-week-old WT and KO mice were subjected to retina oxidative insults through light induction (F) or intravitreal PQ injection (G). Quantification of retinal thickness by OCT demonstrates exaggerated thinning of outer and inner retinal layers in KO mice by light induction (F) or reduced inner retinal thickness in KO mice by PQ after 14 days (G) (n=8-24).

FIG. 7. Loss of IRAK-M in RPE cells leads to impaired RPE cell homeostasis. Primary RPE cells isolated from WT or Irak3^{-/-} mice (5m old) were subjected to Mitochondrial Stress Test using a Seahorse XFp Analyzer, and metabolic parameters calculated from OCR (A) and ECAR (B) profiles demonstrate decreased mitochondrial basal respiration (BR) and ATP production in KO-RPE, despite no significant differences in maximal respiration (MR), proton leak (H+), non-mitochondrial respiration (NMR), and basal (BG) and maximal glycolytic capacity (MGC), between WT and KO-RPE cells (n=3). (C) Mouse primary RPE cells subjected to a pulsed PQ or H₂O₂ treatment (repeated 2 h treatment per day for a total of 7 days) were analyzed for induction of senescence using a fluorescence-based SA-β-Gal assay. Mean fluorescence intensity quantified by ImageJ demonstrates induced SA-β-Gal signal in KO cells (n=9). Oxidative stress-induced, and Irak3^{-/-} promoted RPE senescence was also confirmed by increased p21 and decreased Lamin-B1 (D), and enhanced secretion of proinflammatory cytokines IL-6 (E, n=4) and HMGB1 (F, n=4).

FIG. 8. Inducing IRAK-M expression in RPE cells retains cell homeostasis and function against stresses. OCR (A) and ECAR (B) analyses show that increasing endogenous IRAK-M expression in human iPSC-RPE cells by CRISPR/Cas9 activation plasmid maintains both mitochondrial respiration and glycolytic capacity upon 24 h treatment with 30 μM H₂O₂ or 1 μg/ml LPS (n=3-7). (C) Stable transfectant cell lines selected from mouse B6-RPE07 were established to persistently express human IRAK-M. Measurement of LDH release from the cells over 5 days since confluence shows sustained cell viability by human IRAK-M transgene expression. (D) Human IRAK-M expression also reduces chronic treatment (72 h) of PQ (125 μM) or LPS (40 ng/ml)-induced cytotoxicity (n=2-4). (E&F) Primary murine Irak3^{-/-} RPE cells were subjected to transient transfection for human IRAK-M expression and 48 h later, the cells were stressed with 60 μM H₂O₂ for another 24 h. OCR (E) and ECAR (F) analyses show maintained maximal respiration by human IRAK-M. No significant change in glycolysis capacity was found. (G) Multiplex cytokine array demonstrated that the stable expression of human IRAK-M in B6-RPE07 cells inhibited proinflammatory cytokine secretion in response to stresses, including LPS or PQ-induced GM-CSF, and LPS-induced MCP-1 (n=3).

FIG. 9. The second western blot of human RPE/choroidal lysates showing IRAK-M expression at different ages. For samples (marked in red) included in both blots (FIG. 1E and FIG. 9), the average of IRAK-M expression level was used for quantitative analysis.

FIG. 10. Enrichment analysis of DEGs of AMD-derived RPE/choroid/sclera by Metascape. Heatmaps show up to top 20 enriched clusters in downregulated mRNA (A), upregulated mRNA (B), downregulated antisense RNA (C), and upregulated antisense RNA (D). Downregulated mRNA or upregulated antisense RNA (A and D) suggests decreased gene expression, and upregulated mRNA or downregulated antisense RNA (B and C) suggests increased gene expression. Enriched terms can be GO/KEGG terms, canonical pathways, reactome gene sets and WikiPathways. The enriched clusters are listed in order from the greatest statistical significance.

FIG. 11. Data mining of RNA-Seq dataset (GSE99248) to compare normalized counts of mRNA (A) and antisense RNA (B) of IRAK family members (IRAK1, IRAK2 and IRAK4) in RPE/choroid/sclera tissues between AMD and controls. A gene is considered differentially expressed (DEG) if it has a P<0.05 and a fold-change>±2.

FIG. 12. Data mining of RNA-Seq dataset (GSE99248) to compare normalized counts of mRNA (A) and antisense RNA (B) of JUN in RPE/choroid/sclera tissues between AMD and controls. A gene is considered differentially expressed (DEG) if it has a P<0.05 and a fold-change>±2.

FIG. 13. (A) Overexpression of c-Jun in ARPE-19 cells does not protect the cells from PQ-induced cell damage. The cells were transfected with c-Jun or IRAK-M CRISPR/Cas9 activation plasmid, or control plasmid for 48 h, followed by treatment with a toxic dose of PQ (1 mM) for a further 48 h. Cell culture supernatants were collected for measurement of LDH release as an indicator of cytotoxicity (n=3-4). (B) Increasing endogenous IRAK-M expression in human iPSC-RPE cells by CRISPR/Cas9 based activation plasmid. The cells were transfected with the plasmid for 48 h and cell lysate prepared for western blotting analysis of protein expression. Transfection of vehicle CRISPR/Cas9 plasmid was used as control.

FIG. 14. Inducing IRAK-M expression in ARPE-19 cells maintains cell homeostasis against stresses. (A) Western blot analysis of IRAK-M in the cells following transfection with CRISPR/Cas9 activation plasmid for 48 h. (B) OCR profile and parameter analysis showing partly inhibition of LPS-caused reduction in maximal respiration (MR) by IRAK-M overexpression (n=3 or 4). (C) ECAR profile and parameter analysis showing induced basal and maximal glycolytic activity (BG and MGC) by IRAK-M overexpression when the cells are stressed with H₂O₂ (n=3 or 4). (D) Autophagy Tandem LC3B-GFP-RFP sensor assay and confocal imaging demonstrating that IRAK-M overexpression enhances the formation of LC3B-autophagosome (green) and LC3B-autolysosome (red) to combat H₂O₂ or LPS-induced stresses (n=20-25). PQ-induced cell senescence is inhibited by IRAK-M overexpression, evidenced by reduced SA-β-Gal activity (n=20, E) and HMGB1 release (n=6, F). (G) LDH cytotoxicity assay showing that IRAK-M overexpression inhibits PQ (1 mM)-induced cytotoxicity (n=4).

FIG. 15. Stable transfected cell colonies were selected from mouse B6-RPE07 cell line, and two new cell lines were established to persistently express human and mouse IRAK-M, respectively. Another new cell line bears vehicle plasmid (pUNO1) as a transfection control. (A) qRT-PCR analysis demonstrates strong expression of human or mouse IRAK-M gene in the cell lines, with no changes in the expression of IRAK1 and IRAK4. (B) The cell lines were stimulated with 1 μg/ml LPS for 30 min to activate TLR4 signalling. NF-κB activity assay shows reduced nuclear NF-κB activity in cells expressing human or mouse IRAK-M (n=2).

FIG. 16. (A) Western blot analysis of time course of total and phosphorylated c-Jun expression in ARPE-19 treated with PQ, H2O2, or a c-Jun inhibitor SP600125. (B) Dot plot of BLAST sequence alignment showed a close similarity in the sequences between human IRAK-M (Q9Y616) and mouse IRAK-M (Q8K4B2).

FIG. 17. IHC analysis of human retinal sections reveals reduced IRAK-M expression primarily at the macular RPE and choroid in old age and AMD. Mean staining intensity of IHC images of human retinal sections from two non-AMD donor eyes (59-year old female and 97-year old female, respectively), an early AMD donor (95-year old female) and a mild AMD donor demonstrate that the expression of IRAK-M is more severely reduced in the macula RPE in both ageing and AMD, while the reduced expression in choroid is only significant in old ages and the change in retina is not significant (n=2 for young control, n=5 for old control and n=11 for AMD). Scale bars=100 μm.

FIG. 18. AAV2 serotype dose-dependently transduces retina following subretinal injection. A total of 2×10⁹ or 4×10⁸ genome copies (gc) of AAV2 encoding EGFP driven by the CMV promoter were injected into the subretinal space per eye in 8-week-old mice. At 1-11 weeks post injection, viral dose-dependent retinal transduction was examined by in vivo fundal fluorescence imaging using Micron IV.

FIG. 19. Subretinal delivery of AAV2.CMV.hIRAK3 induces human IRAK3 expression in mouse RPE. (A) Two weeks post subretinal injection of AAV2.CMV.hIRAK3 or null AAV2.CMV (2×10⁹ or 4×10⁸ gc/eye), RPE/choroid and retina tissues were analysed for human and mouse IRAK3 mRNA expression by quantitative RT-PCR. The relative quantification (RQ, Log10 transformed) of gene expression was normalized by mouse RPS29 mRNA (n=5). (B) Mouse retinal cryosections were examined for virus-induced human IRAK-M protein expression by immunofluorescence staining using either an antibody specific to human IRAK-M only, or an antibody recognizing both human and mouse IRAK-M. Representative confocal images confirm the augmentation of IRAK-M protein in the RPE. Scale bars=50 μm.

FIG. 20. Subretinal delivery of AAV2.CMV.hIRAK3 suppresses light-induced outer retinal thinning. Two weeks after subretinal injection of the hIRAK3 or control virus (2×10⁹ gc/eye), mice were subjected to light-induced retinal degeneration in one eye of each mouse and left thereafter for a further two weeks for assessment of retinal damage and response to the therapy. (A) Representative fundoscopy and OCT images obtained on day 14 after light challenge show light-induced focal outer retinal lesions. (B) Each value of outer retinal thickness was an averaged thickness between 200 and 800 μm distant to ONH covering the light affected area, measured from OCT images using an ImageJ macro. Quantification of the data shows the protective effect of AAV2.CMV.hIRAK3 treatment on light-induced photoreceptor loss as demonstrated by suppressing the outer retinal thinning (n=10-11).

FIG. 21. AAV2.CMV.hIRAK3 gene therapy prevents light-induced retinal cell death. Two weeks after subretinal injection of hIRAK3 or null AAV2.CMV vectors (2×10⁹ gc/eye), mice were subjected to light-induced retinal degeneration in one eye and kept for another two weeks. Eyes were collected and sections were processed for TUNEL staining and analysis. DAPI was used to stain cell nuclei. The number of TUNEL+ cells (identified with confocal images showing apoptotic cells (TUNEL-positive) in the retina of different groups) was quantified and averaged from 3 sections of each eye from 3 or 6 mice per group. Scale bars=100 μm.

FIG. 22. AAV2.CMV.hIRAK3 gene therapy protects photoreceptor mitochondria against light damage. Two weeks after light induction, mouse retinal cryosections were fixed for MitoView Green staining to assess the mitochondrial content. Slides were counterstained with DAPI. MFI analysis of confocal images for MitoView Green stain demonstrated a reduction of mitochondria content in PR inner segment by light damage in control AAV2-injected eyes, which is significantly inhibited by AAV2.CMV.hIRAK3 treatment. The graph shows the average of MFI measured in three different fields from two sections of 3-6 mice. Scale bars=100 μm.

FIG. 23. AAV2.CMV.hIRAK3 gene therapy reduces retinal spots in Irak3^{-/-} mice. 2×10⁹ gc of AAV2.CMV.hIRAK3 was injected into the subretinal space of one eye of each Irak3^{-/-} mouse (2-4m old), with control AAV2 injection to the contralateral eye. The mice were then kept under normal conditions for 6 months and retinas were assessed using fundoscopy and OCT at indicated time points. (A) Representative fundal images show retinal white spots in Irak3^{-/-} mice (8m old) with AAV administration at age of 2m. Lines separate the retina into two sides based on the site of injection. (B) Time course of incidence of flecked retina (number of spots>3) shows IRAK3 gene delivery significantly decelerated the appearance of retinal spots in IRAK3-KO mice during ageing. Each value is a ratio of number of flecked retina to total number (n=15 or 16) of retina at each time point. (C) Number of retinal spots in the whole retina, or (D) at injection side, was blind-counted for comparison between AAV2.CMV.hIRAK3 and null vector-treated eyes of in aged KO mice (8-10m).

FIG. 24. AAV2.CMV.hIRAK3 gene therapy inhibits retinal thinning in aged IRAK3^{-/-} mice. Irak3^{-/-} mouse (2-4m old) were administered subretinally with 2×10⁹ gc of AAV2.CMV.hIRAK3 or null AAV2.CMV. The outer retinal thickness was averaged from temporal and nasal sides of OCT images for all animals in each group. Quantification of data indicates a reduction in outer retinal thickness at the centre region, 200 μm distant from optic nerve head, in old Irak3^{-/-} mice (8-10m old) compared with age-matched WT mice, which was significantly revoked by IRAK3 gene therapy.

FIG. 25. Putative endogenous promoter sequences for human IRAK3 gene. Three fragments in front of exon 1 of human IRAK-M gene (ENSG00000090376) were selected. (A) The 0.88 kb fragment is the predicted “core promoter” which includes the CpG island and H3K methylation marks. (B) The 1.36 kb fragment is predicted as the maximum promoter size, which the AAV backbone CMV.GFP.WPRE.io2 can accommodate given IRAK3 gene size plus WPRE and io2 elements. (C) The 1.6 kb fragment is the maximum promoter size that AAV vectors in general can accommodate given IRAK3 gene size.

FIG. 26. Comparison of different promoters for AAV2-mediated IRAK3 gene delivery and protective effect in RPE cells and LIRD. (A-C) Mouse B6-RPE07 cells were transduced with null AAV2.CMV, AAV2.CMV.hIRAK3, AAV2.Best1.hIRAK3, AAV2.Endo1.hIRAK3, AAV2.Endo2.hIRAK3 or AAV2.Endo3.hIRAK3 (MOI 50,000 gc). Three days post gene transfer, mRNA expression of (A) exogenous human IRAK3 and (B) endogenous mouse IRAK3 was analysed by qPCR, and (C) cytotoxicity was measured by LDH assay (n=4). (D) Given the greater transgene expression by Endo3 promoter compared to the Endo1 and Endo2 promoters, AAV2.Endo3.hIRAK3, with comparison to AAV2.CMV.hIRAK3 and AAV2.Best1.hIRAK3, was transduced to human ARPE-19 cells, followed by paraquat treatment for 4 days. LDH assay was used to assessment of cytotoxicity in response to the oxidative stressor (n=4). (E) Subretinal delivery of AAV2.hIRAK3 with different promoters suppresses light-induced outer retinal thinning. Three-five weeks after subretinal injection of indicated AAV2s (2×109 gc/eye), mice were subjected to LIRD and left thereafter for up to two weeks for OCT assessment. Data shows the percentage of ORL thinning relative to control retina without light challenge (averaged from 8-9 eyes/group).

FIG. 27. AAV5 dose-dependently induces human IRAK-M gene expression in RPE cells. Mouse B6-RPE07 cells were transduced with null AAV5.CMV or AAV5.CMV.hIRAK3 at an MOI of 50,000 or 100,000 gc. Three days after transduction, mRNA expression of (A) exogenous human IRAK3 and (B) endogenous mouse IRAK3 was analysed by qPCR (n=3).

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

Age-related macular degeneration (AMD) is a progressive degenerative disease. Impairment of the nourishing, immune and metabolic function of the retinal pigment epithelium (RPE) and the surrounding microenvironment typically leads to macular disorder, RPE and photoreceptor (PR) loss and gradual loss of the central visual acuity (94, 95). The accumulation of lipoproteinaceous drusen deposit at early non-exudative (dry) AMD can develop into geographic atrophy (late dry) or neovascular exudative (wet) stage (96). There is a lack of treatment options to prevent RPE and PR loss in AMD, despite an increasing burden with the rapidly rising prevalence of AMD due to population ageing. The unmet need for therapies of dry AMD is urgent.

Alongside chronological ageing, the interplay of oxidative stress and chronic inflammation resulting from genotype-predisposed susceptibility and environmental stressors are the primary contributors to AMD. Inflammation-induced drusen genesis is not only the hallmark of early AMD but also the critical origin of proinflammatory factors that trigger the disruption of macular function (97). The central inflammatory players that participate in AMD progress include dysregulated complement cascade components, inflammasome activation, cytokines and immune-responsive cells including dendritic cells, microglia, macrophages and RPE (97, 98).

RPE is highly susceptible to the perturbance of ageing (19) and inflammatory stressors. When the disturbance in RPE intracellular process pathways including autophagy, phagolysosome, mitochondrial metabolism, protein trafficking and senescence is compounded by oxidative stress, further inflammation is elaborated (23). The magnitude of inflammatory responses is balanced by inhibitory mechanisms such as regulation by Interleukin 1 receptor-associated kinase-M (IRAK-M), encoded by IRAK3 gene (28). Dysregulation and impairment of IRAK-M signalling is associated with oxidative stress and systemic inflammation implicated in metabolic disorders such as insulin resistance and obesity, all associated with age-related eye diseases including the AMD (31, 99, 100).

The present invention is based on finding that IRAK-M expression within the RPE declines with age and following oxidative stress. A decrease in IRAK-M expression was also identified in eyes of patients with AMD. The present inventors have found that decreased IRAK-M expression undermines the ability to maintain cellular function and health. Augmentation of IRAK-M expression provides protection to RPE and retina. Accordingly, the invention relates to increasing IRAK-M expression for the prophylaxis of and treatment of macular degeneration.

Several strategies can be used to achieve increased IRAK-M expression in a subject. These are discussed in detail below.

### Medical Uses

Accordingly, the invention relates to agents as described herein for increasing IRAK-M expression in a target cell and/or increasing IRAK-M activity in a target cell, for use in a method of treatment or prophylaxis of macular degeneration in a subject.

An option for increasing IRAK-M expression in a target cell may involve introducing exogenous IRAK-M to a target cell. Thus, the agent may be an IRAK-M polypeptide or a nucleic acid encoding IRAK-M. In some embodiments, the agent is heterologous. Another option for increasing IRAK-M expression involves increasing endogenous IRAK-M expression in a target cell. Thus, the agent may be capable of increasing endogenous IRAK-M expression.

Alternatively, the agent may be capable of increasing IRAK-M activity in a target cell.

Further provided is a method of treatment or prophylaxis of macular degeneration in a subject comprising administering an agent as described herein to the subject. Additionally provided is the use of an agent as described herein for the manufacture of a medicament for the treatment or prophylaxis of macular degeneration in a subject.

The present invention also relates to a nucleic acid, a vector virion, a polypeptide, a nucleic acid system, a viral vector system, or a pharmaceutical composition, for use in a method of treatment or prophylaxis of macular degeneration in a subject, where the nucleic acid increases IRAK-M expression in a target cell of the subject.

Also provided is a method of treatment or prophylaxis of macular degeneration in a subject comprising administering a nucleic acid, a vector virion, a polypeptide, a nucleic acid system, a viral vector system, or a pharmaceutical composition as described herein to the subject. In another aspect, provided is the use of a nucleic acid, a vector virion, a polypeptide, a nucleic acid system, a viral vector system, or a pharmaceutical composition as described herein, for the manufacture of a medicament for the treatment or prophylaxis of macular degeneration in a subject.

### Macular Degeneration

Macular degeneration is a medical condition which may result in deterioration of vision, resulting in blurred or no vision in the centre of the visual field. The term “macular degeneration” refers to any of a number of conditions in which the retinal macula degenerates or becomes dysfunctional, e.g. as a result of decreased growth of cells of the macula, increased death or rearrangement of the cells of the macula (e.g. RPE cells), loss of normal biological function, or a combination of these events.

In particular, the present invention relates to age-related macular degeneration. As used herein, “age-related macular degeneration” or “AMD” includes early, intermediate, and advanced/late AMD and includes both dry AMD such as geographic atrophy and wet AMD, also known as neovascular or exudative AMD. Degeneration/dysregulation of the retinal pigment epithelium (RPE), a supportive monolayer of cells underlying the photoreceptors, is commonly seen in patients with AMD. The retinal pigment epithelium (RPE) is a multifunctional monolayer of neuroepithelium-derived cells, flanked by photoreceptor (PR) cells and the choroid complex. The RPE is typically composed of a single layer of hexagonal cells that are densely packed with pigment granules.

In some embodiments, the macular degeneration is age-related macular degeneration.

Typically, in AMD there is a progressive accumulation of characteristic yellow deposits, called drusen in the macula (a part of the retina) between the RPE and the underlying choroid. Drusen are formed of extracellular proteins and lipids. The accumulation of drusen damages the retina over time. AMD can be divided into 3 stages: early, intermediate, and late, based partially on the extent (size and number) of drusen. In some embodiments, administration of the therapy of the invention results in a reduction of drusen in a target cell compared to a cell not comprising the therapy of the invention. In some embodiments, administration of therapy of the invention to the target cell prevents the formation of drusen in a target cell compared to a cell not comprising the therapy of the invention.

In some embodiments, the age-related macular degeneration (AMD) is early AMD. Early AMD is typically diagnosed based on the presence of medium-sized drusen. Early AMD tends to be asymptomatic. In some embodiments, the age-related macular degeneration (AMD) is intermediate AMD. Intermediate AMD is typically diagnosed by large drusen and/or any retinal pigment abnormalities. Intermediate AMD can lead to some vision loss, but generally is asymptomatic. In some embodiments, the age-related macular degeneration (AMD) is late AMD (also known as advanced AMD). Typically, in late AMD, patients experience symptomatic central vision loss caused by retinal damage. This damage can be caused by atrophy or by the onset of neovascular disease. Late AMD is further divided into two subtypes based on the type of damage. These are called geographic atrophy/dry AMD and wet AMD/neovascular AMD. In some embodiments, the AMD is selected from the group consisting of early AMD, intermediate AMD, and late AMD.

In some embodiments, the age-related macular degeneration (AMD) is dry AMD. Dry AMD encompasses all forms of AMD that are not wet AMD, including early and intermediate forms of AMD as well as the advanced form of dry AMD, called geographic atrophy. In some embodiments, the age-related macular degeneration (AMD) is geographic atrophy. Geographic atrophy, also known as atrophic AMD, is an advanced form of dry AMD. It is characterised by progressive and irreversible loss of retinal cells leading to a loss of visual function. Typically, in geographic atrophy, three areas of the retina undergo atrophy. These are the choriocapillaris, retinal pigment epithelium, and the overlying photoreceptors.

In contrast, wet AMD (also called neovascular or exudative AMD) is the wet form of advanced AMD. It is characterised as vision loss due to abnormal blood vessel growth (choroidal neovascularization) in the choriocapillaris, through Bruch's membrane. It is usually, but not always, preceded by the dry form of AMD. The proliferation of abnormal blood vessels in the retina is stimulated by vascular endothelial growth factor (VEGF). These abnormal blood vessels are more fragile than typical blood vessels, and so lead to blood and protein leakage below the macula. Bleeding, leaking, and scarring from these blood vessels eventually cause irreversible damage to the photoreceptors and rapid vision loss if left untreated. In some embodiments, the age-related macular degeneration (AMD) is not wet AMD. In some embodiments, the age-related macular degeneration (AMD) is dry AMD and excludes wet AMD.

Typically, in patients with macular degeneration the retina and the choroid are affected. Thus, in some embodiments, the target cell is a cell of the retina or the choroid. In some embodiments, the target cell is a cell of the retina. The retina is the innermost, light-sensitive tissue of the eye. The retina comprises several layers, including a layer comprising photoreceptors. The principal functional layers of the retina comprise the ganglion cell layer (GCL), the inner plexiform layer (IPL), the inner nuclear layer (INL), the outer plexiform layer (OPL), the outer nuclear layer (ONL), the photoreceptor outer segment (POS), and supporting the retina, the retinal pigmental epithelium (RPE). In some embodiments, the target cell is a cell of the ganglion cell layer (GCL), the inner plexiform layer (IPL), the inner nuclear layer (INL), the outer plexiform layer (OPL), the outer nuclear layer (ONL), the photoreceptor outer segment (POS), or the retinal pigmental epithelium (RPE). In some embodiments, the target cell is a cell of the retinal pigmental epithelium (RPE).

In some embodiments, the target cell is a myeloid cell. In some embodiments, the myeloid cell is a retinal myeloid cell. In some embodiments, the target cell is a CD11b+ myeloid cell. CD11b+ cells include the yolk-sac derived tissue resident microglia. These cells are inherent to maintaining retinal tissue and neuronal cell homeostasis.

In some embodiments, the method of treatment or prophylaxis of macular degeneration in a subject includes the step of contacting a target cell or tissue with the agent as described herein. In some embodiments, the method of treatment or prophylaxis of macular degeneration in a subject includes the step of contacting a target cell or tissue with the nucleic acid, a vector virion, a polypeptide, a nucleic acid system, a viral vector system, or a pharmaceutical composition described herein.

### IRAK-M

IRAK-M is an inactive kinase encoded by the IRAK3 gene. Specifically, IRAK-M (also known as IRAK3) is a cytoplasmic pseudo-kinase, belonging to the IRAK family. The IRAK family consists of two active kinases (IRAK-1 and IRAK-4) and two inactive kinases (IRAK-2 and IRAK-M).

IRAK-M is a negative regulator for TLR/IL-1R-induced proinflammatory cascade. IRAK-M prevents dissociation of IRAK-1 and IRAK-4 from MyD88 as well as formation of IRAK-1-TRAF6 complexes. Thus, preventing downstream TLR/IL-1R signalling.

Exogenous IRAK-M polypeptide and/or peptide may be directly delivered into the cytoplasm of ocular cells. Accordingly, in an aspect of the invention provides an IRAK-M polypeptide and/or peptide for use in a method of treatment or prophylaxis of macular degeneration in a subject.

The human sequence of IRAK-M is provided below (UniProt Q9Y616).

SEQ ID NO: 1:
MAGNCGARGALSAHTLLFDLPPALLGELCAVLDSCDGALGWRGLAERLSS
SWLDVRHIEKYVDQGKSGTRELLWSWAQKNKTIGDLLQVLQEMGHRRAIH
LITNYGAVLSPSEKSYQEGGFPNILFKETANVTVDNVLIPEHNEKGILLK
SSISFQNIIEGTRNFHKDFLIGEGEIFEVYRVEIQNLTYAVKLFKQEKKM
QCKKHWKRFLSELEVLLLFHHPNILELAAYFTETEKFCLIYPYMRNGTLF
DRLQCVGDTAPLPWHIRIGILIGISKAIHYLHNVQPCSVICGSISSANIL
LDDQFQPKLTDFAMAHFRSHLEHQSCTINMTSSSSKHLWYMPEEYIRQGK
LSIKTDVYSFGIVIMEVLTGCRVVLDDPKHIQLRDLLRELMEKRGLDSCL
SFLDKKVPPCPRNFSAKLFCLAGRCAATRAKLRPSMDEVLNTLESTQASL
YFAEDPPTSLKSFRCPSPLFLENVPSIPVEDDESQNNNLLPSDEGLRIDR
MTQKTPFECSQSEVMFLSLDKKPESKRNEEACNMPSSSCEESWFPKYIVP
SQDLRPYKVNIDPSSEAPGHSCRSRPVESSCSSKFSWDEYEQYKKE

An aspect provides a polypeptide for use in a method of treatment or prophylaxis of macular degeneration in a subject. The polypeptide comprising an amino acid sequence having at least 60% sequence identity to the amino acid sequence of SEQ ID NO: 1.

In some embodiments, the polypeptide has an amino acid sequence having at least 65%, at least 70%, at least 75%, at least 80%, at least 85%, at least 90%, at least 95%, at least 96%, at least 97%, at least 98%, at least 99%, or 100% sequence identity to the amino acid sequence of SEQ ID NO: 1. In some embodiments, the amino acid sequence of polypeptide consists of SEQ ID NO: 1.

In some embodiments, the nucleic acid or vector virion comprising said nucleic acid comprises a nucleic acid sequence encoding a polypeptide having at least 60% sequence identity to the amino acid sequence of SEQ ID NO: 1. In some embodiments, the nucleic acid or vector virion comprising said nucleic acid comprises a nucleic acid sequence encoding a polypeptide having at least 65%, at least 70%, at least 75%, at least 80%, at least 85%, at least 90%, at least 95%, at least 96%, at least 97%, at least 98%, at least 99%, or 100% sequence identity to the amino acid sequence of SEQ ID NO: 1. In some embodiments, the nucleic acid or vector virion comprising said nucleic acid comprises a nucleic acid sequence consisting of SEQ ID NO: 1.

Percent (%) amino acid sequence identity with respect to a reference sequence is defined as the percentage of amino acid residues in a candidate sequence that are identical with the amino acid residues in the reference sequence, after aligning the sequences and introducing gaps, if necessary, to achieve the maximum percent sequence identity, and not considering any conservative substitutions as part of the sequence identity. Various known tools can be used to measure sequence identity, including but not limited to Clustal Omega, Multiple Sequence Alignment (EMBL-EBI).

In some embodiments, the polypeptide of the invention is formulated for ocular delivery. In some embodiments, the polypeptide according to the invention is functional. In some embodiments, the polypeptide is capable of preventing dissociation of IRAK-1 and/or IRAK-4 from MyD88 in a target cell. In some embodiments, the polypeptide is capable of preventing formation of an IRAK-1-TRAF6 complex.

The present inventors have found that augmentation of IRAK-M expression provided protection to the RPE and retina more generally. By introducing human IRAK-M transgene to mouse RPE, mitochondrial activity was retained and cell survival under oxidative stress was promoted. The inventors have also identified an increase in autophagic flux associated with an increase in IRAK-M expression.

In some embodiments, autophagic flux is increased in the target cell relative to a cell that is not modified with the agent described herein (e.g., the small molecule, nucleic acid, vector virion, polypeptide, nucleic acid system, viral vector system, or pharmaceutical composition described herein). Autophagic flux can be used to determine autophagic activity within a cell. By “autophagic flux” is meant the amount of autophagic degradation occurring in a target cell. Autophagic flux can be measured using many different techniques as described in Yoshii S. R., and Mizushima N. Int J Mol Sci. 2017 September; 18 (9): 1865. For example, autophagic flux can be measured by immunoblotting for LC3-II. LC3-II is commonly used as an autophagosome marker because the amount of LC3-II reflects the number of autophagosomes and autophagy-related structures. Similarly, the amount of p62 in tissues can be measured. Degradation of p62 is another widely used marker to monitor autophagic activity because p62 directly binds to LC3-II and is selectively degraded by autophagy. The autophagy pathway dynamics can be also measured by fluorescent live cell imaging using LC3-RFP-GFP tandem sensor combined with a lysosome probe such as Lysotracker or labelling of LAMP1/2, which enables the detection of LC3+ neutral pH autophagosomes, LC3+ acidic pH autolysosome, and lysosomal activities.

In some embodiments, mitochondrial activity is maintained or increased in the target cell relative to a cell that is not modified with the agent described herein. Mitochondria are organelles of eukaryotes and have their own mitochondrial DNA. Oxygen respiration (aerobic respiration) and production of ATP occur in the mitochondria. The synthesis of ATP via oxidative phosphorylation is the most common function ascribed to mitochondria. This process is typically determined indirectly through measurement of mitochondrial oxygen (O₂) consumption, or respiration. Mitochondria play a central role in establishing and regulating cellular redox homeostasis. Generally, maintaining or increasing mitochondrial activity means maintaining or increasing the expression or activity of electron transport components, ATP synthesis proteins, TCA cycle components, or coproporphyrinogen oxidase (CPOX). It also includes maintaining or increasing ATP synthesis.

Methods for measuring mitochondrial activity include but are not limited to measuring the rate of mitochondrial respirometric O₂ flux (including O₂-dependent quenching of porphyrin-based phosphors and amperometric O₂ sensors), oxidant emission (e.g. fluorescent-, chemiluminescent-, and, electrochemical/nanoparticle-based approaches to detect oxidants), measuring mitochondrial membrane potential, ATP production via bioluminescence, calcium retention capacity, mitochondrial NAD(P)H, etc.

Inflammation may be reduced in the target tissue relative to target tissue that is not modified with the agent described herein. Proinflammatory cytokine production may be reduced in the target cell comprising the agent compared to an equivalent target cell not comprising the agent. In some embodiments, IRAK-M expressed in a target cell inhibits production of proinflammatory cytokines (e.g., under stresses). The proinflammatory cytokines may be selected from the group consisting of GM-CSF and MCP-1. The levels of these proinflammatory cytokines may be measured using techniques such as flow cytometry or western blot. The concentration of proinflammatory cytokines in the target cell may be reduced relative to a target cell that is not modified with the agent described herein

In some embodiments, the polypeptide is a recombinant polypeptide modified for delivery to a target cell. In an example, IRAK-M may be conjugated to peptides that are described in Bhattacharya et al. to mediate delivery into RPE cells. Bhattacharya 2017, Journal of Controlled Release 251, 37-48 describes a peptide-based delivery system that allows for controlled cargo release in RPE cells. The described system is typically used for intravitreal administration. Other possible routes of delivery are described herein. The peptide-based delivery system comprises a peptide-based cleavable linker (PCL) with a cell penetrating peptide (CPP) conjugated to the N-terminus and the cargo (e.g. IRAK-M) is conjugated to the C-terminus. Example PCLs include peptide sequences sensitive to cathepsin D. Cathepsin D, a lysosomal enzyme has relatively high expression in RPE cells. CPPs are charged peptide sequences capable of intracellular delivery of molecular cargo.

A cell penetrating peptide (CPP) is typically a short peptide that facilitates cellular intake and uptake of molecules (e.g. polypeptides). CPPs typically deliver cargo into cells via endocytosis. CPPs generally have an amino acid composition comprising a high abundance of positively charged amino acids (e.g. lysine or arginine) or comprising sequence containing an alternating pattern of polar, charged amino acids and non-polar, hydrophobic amino acids. Example CPPs include but are not limited to, penetratin peptide, Tat peptide (48-60), VP22 peptide, Mouse PrP peptide, pVEC peptide, Transportan peptide, TP10 peptide, Polyarginine peptide, etc. Example CPPs for RPE cells are provided in Bhattacharya 2017, Journal of Controlled Release 251, 37-48. Non-limiting examples include GRKKRRQRRPPQ (SEQ ID NO: 2), rrrrrrrrr (SEQ ID NO: 3), RLVSYNGIIFFLK (SEQ ID NO: 4), FNLPLPSRPLLR (SEQ ID NO: 5), where “r” is D-Arg. In some embodiments, the CPP further comprises a short flexible linker in between the CPP and PCL. In some embodiments, the short flexible linker has the amino acid sequence GGS. A PCL is a peptide-based cleavable linker. In the context of the invention other cleavage linkers may be used. Example PCLs cleavable by cathepsin D are described in Bhattacharya 2017, Journal of Controlled Release 251, 37-48. Non-limiting examples include KGKPILFFRLKr (SEQ ID NO: 6), KPILFFRLGK (SEQ ID NO: 7), and KGSALISWIKR (SEQ ID NO: 8), where “r” is D-Arg.

Accordingly, example CPPs conjugated to PCLs include but are not limited to

(SEQ ID NO: 9)
GRKKRRQRRPPQGGSKGKPILFFRLKr,
(SEQ ID NO: 10)
GRKKRRQRRPPQGGSKPILFFRLGK,
(SEQ ID NO: 11)
GRKKRRQRRPPQGGSKGSALISWIKR,
(SEQ ID NO: 12)
rrrrrrrrrGGSKGKPILFFRLKr,
(SEQ ID NO: 13)
rrrrrrrrrGGSKPILFFRLGK,
(SEQ ID NO: 14)
rrrrrrrrrGGSKGSALISWIKR,
(SEQ ID NO: 15)
RLVSYNGIIFFLKGGSKGKPILFFRLKr,
(SEQ ID NO: 16)
RLVSYNGIIFFLKGGSKPILFFRLGK,
(SEQ ID NO: 17)
RLVSYNGIIFFLKGGSKGSALISWIKR,
(SEQ ID NO: 18)
FNLPLPSRPLLRGGSKGKPILFFRLKr,
(SEQ ID NO: 19)
FNLPLPSRPLLRGGSKPILFFRLGK,
(SEQ ID NO: 20)
FNLPLPSRPLLRGGSKGSALISWIKR,
(SEQ ID NO: 21)
GRKKRRQRRPPQKGKPILFFRLKr,
(SEQ ID NO: 22)
GRKKRRQRRPPQKPILFFRLGK,
(SEQ ID NO: 23)
GRKKRRQRRPPQKGSALISWIKR,
(SEQ ID NO: 24)
rrrrrrrrrKGKPILFFRLKr,
(SEQ ID NO: 25)
rrrrrrrrrKPILFFRLGK,
(SEQ ID NO: 26)
rrrrrrrrrKGSALISWIKR,
(SEQ ID NO: 27)
RLVSYNGIIFFLKKGKPILFFRLKr,
(SEQ ID NO: 28)
RLVSYNGIIFFLKKPILFFRLGK,
(SEQ ID NO: 29)
RLVSYNGIIFFLKKGSALISWIKR,
(SEQ ID NO: 30)
FNLPLPSRPLLRKGKPILFFRLKr,
(SEQ ID NO: 31)
FNLPLPSRPLLRKPILFFRLGK,
and
(SEQ ID NO: 32)
FNLPLPSRPLLRKGSALISWIKR.

Any one of the above peptides may be conjugated to the IRAK-M polypeptide, directly or indirectly.

In some embodiments, provided is a molecule comprising a PCL with a CPP conjugated to the N-terminus and an IRAK-M polypeptide or peptide conjugated to the C-terminus of the PCL (e.g. CPP-PCL-IRAK-M). In some embodiments, provided is a molecule comprising a PCL with a CPP conjugated to the N-terminus and an IRAK-M polypeptide having at least 60% identity to the amino acid sequence of SEQ ID NO: 1 conjugated to the C-terminus of the PCL (e.g. CPP-PCL-IRAK-M).

### Gene Therapy

Gene therapy involves introducing genetic material into target cells for the purpose of modulating the expression of specific proteins which are altered, thus reversing the biological disorder causing the alteration thereof. The present invention contemplates a nucleic acid sequence encoding IRAK-M protein for use in a method of treatment or prophylaxis of macular degeneration in a subject.

In human cells, IRAK-M is encoded by the IRAK3 gene. In the context of the present invention “IRAK3 gene” refers to the DNA sequence encoding IRAK-M (e.g., found in the genome). The IRAK3 gene may be operably linked to any suitable transcriptional and/or translational regulatory sequences in the nucleic acid and vector systems described herein.

The term “nucleic acid” herein is meant either DNA or RNA, or molecules which contain both ribo- and deoxyribonucleotides. The nucleic acids include genomic DNA, cDNA and oligonucleotides including sense and anti-sense nucleic acids. The nucleic acid may be double stranded, single stranded, or contain portions of both double stranded or single stranded sequence. In some embodiments, the nucleic acid is a recombinant nucleic acid.

In some embodiments, the nucleic acid sequence encoding IRAK-M is exogenous. In some embodiments, the nucleic acid sequence encoding IRAK-M is heterologous. The term “exogeneous” herein is meant nucleic acid which encodes proteins not ordinarily made in appreciable or therapeutic amounts in ocular cells. Exogeneous nucleic acid also includes nucleic acid which is ordinarily found within the genome of the ocular cell, but which is no longer being expressed or is being expressed at a reduced amount compared to non-diseased tissue. Thus, the genetically engineered ocular cell may contain extra copies of a gene ordinarily found within its genome. The term “heterologous” with reference to a nucleic acid refers to a nucleic acid that does not naturally occur in the target cell.

In some embodiments, the nucleic acid is an episome. An episome is a genetic element that can replicate independently of the target cell and also in association with a chromosome with which it becomes integrated. The nucleic acid may be a plasmid or a minicircle. A plasmid is a small, extrachromosomal DNA molecule within a cell that is physically separated from chromosomal DNA and can replicate independently. A minicircle is a small (˜4 kb) circular replicon. In some embodiments, the nucleic acid is messenger RNA or circular RNA.

In some embodiments, the nucleic acid can be integrated into the host's genome. In alternative embodiments, the nucleic acid is not inserted into the host's genome. A nucleic acid randomly integrating into the host's genome can cause adverse events following insertional mutagenesis. A nucleic acid that is not randomly inserted into the host's genome advantageously avoids any insertional mutagenesis.

As will be understood by those of skill in the art, nucleic acids for gene therapy contain the necessary elements for the transcription and translation of the inserted coding sequence (and may include, for example, a promoter, an enhancer, and other regulatory elements). Promoters can be constitutive or inducible. Promoters can be selected to target preferential gene expression in a target tissue, such as the RPE (Sutanto et al., 2005, “Development and evaluation of the specificity of a cathepsin D proximal promoter in the eye” Curr Eye Res. 30:53-61; Zhang et al., 2004, “Concurrent enhancement of transcriptional activity and specificity of a retinal pigment epithelial cell-preferential promoter” Mol Vis. 10:208-14; Esumi et al., 2004, “Analysis of the VMD2 promoter and implication of E-box binding factors in its regulation” J Biol Chem 279:19064-73; Camacho-Hubner et al., 2000, “The Fugu rubripes tyrosinase gene promoter targets transgene expression to pigment cells in the mouse” Genesis. 28:99-105; and references therein). Promoters can also be active in any cell or tissue type.

The nucleic acid encoding IRAK-M is typically operably linked to regulatory elements, such as promoters and enhancers, which drive transcription of the DNA in the target cells of an individual. The promoter may drive expression of IRAK-M in all cell types. Alternatively, the promoter may drive expression of the IRAK-M only in specific cell types, for example, in cells of the retina, e.g. RPE. In some embodiments, the promoter is a ubiquitous promoter. The term “ubiquitous promoter” means a promoter that is active in any cell, tissue, and/or cell cycle stage. Typically, the ubiquitous promoter is strongly active in a wide range of cells, tissues, and/or cell cycle stages. In further embodiments, the ubiquitous promoter is selected from the group consisting of CMV promoter, CAGGS promoter (aka CBA or CAG), mini CAG (SV40 Intron) promoter, SV40 promoter, CBA/CB7 promoter, smCBA promoter, CBh promoter, MeCP2 promoter, shCMV promoter, CMVd2 promoter, core CMV promoter, SV40mini promoter, SCP3 promoter, EF1-α promoter, PGK promoter, GAPDH promoter, and UbC promoter.

In some embodiments, the promoter is an RPE-specific promoter. In further embodiments, the RPE-specific promoter is selected from the group consisting of a RPE65 promoter, NA65 promoter, VMD2 promoter (also known as Best1 promoter) and Synpiii promoter. Suitable promoters, in particular retina-specific promoters are described in Buck et al. Int. J. Mol. Sci. 2020, 21, 4197. Synthetic promoters for RPE are also described in Johari et al. 2021 “Design of synthetic promoters for controlled expression of therapeutic genes in retinal pigment epithelial cells”, Biotechnology and Bioengineering.

In yet another embodiment, the promoter is the native promoter for IRAK3 or a functional fragment thereof. Preferably, the native promoter is the promoter region spanning −1 to −1698 from the transcription start site of IRAK3 (as identified in Pino-Yanes et al. (2011) Am J Respir Cell Mol Biol Vol 45. pp 740-745). The inventors have identified and tested three fragments upstream of the first exon of human IRAK3 gene (Ensembl ID: ENSG00000090376). These fragments were selected as putative endogenous IRAK3 promoters and shown to drive robust expression in cells. These three fragments are shown as SEQ ID NOs 46-48 below.

0.88 kb fragment (“Endo1” in this specification)-SEQ ID
NO: 46:
TTAGAGTGTGATGGGCTGAGTGGGGTTGTGAGTGATTATCTTCTTTTTTCAGTTTTTTTC
TGGGTTTTCCAAGTGTTCCTCGATGAACATGGATAGTTTTTCTGACAGGATAAAAAAGAA
GTAGTCCGGGACAGTGGCTAACACCCCGAATCCCAGCACTTTGGGAAGCCGGAGGTGGGA
GGATCGCTTGAGGCCAGGAGTTTGAAACCAGCCTGGGCAGCATAACGACACTCCCTCTCT
ACGAAAAACGAAAAAAAATAATTAGCCGGACGTGGTGGCGTGCGACTGTGGTCCCAGCTA
CTCGGGAGGCTGAGGTGGGAGGATCGCTTGAGCCCAATAGGTGGAGGCTCCGTGAGCTGA
GATAGCGCCACTGCGCTCCTGCCTGGGCGACAGAGTGAGAACCTGACTCAAAACAAAGAA
AAAAGGAAGAAAAGAAAGGAAGGGAAGAAGGAAGGAAGGGAGAAGCTTTCAAAAATAAAC
TTTTGTAAGAAGTAATGACACCGCTAGCCGTCCACACCAGGAGACCGCCTAGCCGTGGGG
CACGGTGGGCTCCTGGGAGCTCTGAGCTCTGGGCTTTCTCCAGTTCGCACTCTGCTTGTC
TCGGCAGCTCCGTCCCCACCGCAGAGGTGTGAAGGGGCGCAAAGCCAGCGAAGGGAGAAC
CCGGGTCGGGTAACCCCCAGGCCTGGCCAGGCGGACGCAGGGGCATCTCGGGCGAGGCGC
GCCTTGCGTCACGTGGGCACCGCCCCTGCAGTGACCGGAGAACGGCGTGTTCCTAGGGCT
CTGCTGCCGTCGTGGAAGCAGGATTTCCGCGGTTGTGTAACGGCCTGTCGCAGGCGTGCA
GGGACCTGGACTCCGCCTCGTCCCCGGGGCTCGGGCAGCCGAGCC
1.36 kb fragment (“Endo2” in this specification)-SEQ ID
NO: 47:
CACCTCTGGTGTTTTCACTTGATGGCCACTGCCCACTCTTCCAGTCACTAAGGCTGAGAT
TTTTCTCACCACTTTCAAAACAACTTTTTGTCACTGGTAACATGCCTCATGTGACCTAGA
AGATTTTGAAACAACAATTTTACTTAAACTCCGTGAAGATTTCAAAACAACAATTTTACT
TAAACTCCATGTTGAAGGCAAAAAGGAAGATTGTGTGAAGCACTTTGAGGAAGGAAAAAA
TGATGGAACGATATTTCATCAAGGAAGCCCATGAGGAAGAAAATTTATTAAAACCAAGTA
GGTGTATGGAGGAGAGGCCGCCTCGTGAAGAAAACGAACATAATTTTACGCTGATTTGGT
GTCTTTTCTGTTTTTCTCTTGTTGGTATCTACATTCATCTTTTCCAATAATAATCCCATA
TATGTACATTTTTATCTGTTTAAATTCAGTAAAAGTTGGGAGGAAAATGTGTCAAAACTT
TTAGAGTGTGATGGGCTGAGTGGGGTTGTGAGTGATTATCTTCTTTTTTCAGTTTTTTTC
TGGGTTTTCCAAGTGTTCCTCGATGAACATGGATAGTTTTTCTGACAGGATAAAAAAGAA
GTAGTCCGGGACAGTGGCTAACACCCCGAATCCCAGCACTTTGGGAAGCCGGAGGTGGGA
GGATCGCTTGAGGCCAGGAGTTTGAAACCAGCCTGGGCAGCATAACGACACTCCCTCTCT
ACGAAAAACGAAAAAAAATAATTAGCCGGACGTGGTGGCGTGCGACTGTGGTCCCAGCTA
CTCGGGAGGCTGAGGTGGGAGGATCGCTTGAGCCCAATAGGTGGAGGCTCCGTGAGCTGA
GATAGCGCCACTGCGCTCCTGCCTGGGCGACAGAGTGAGAACCTGACTCAAAACAAAGAA
AAAAGGAAGAAAAGAAAGGAAGGGAAGAAGGAAGGAAGGGAGAAGCTTTCAAAAATAAAC
TTTTGTAAGAAGTAATGACACCGCTAGCCGTCCACACCAGGAGACCGCCTAGCCGTGGGG
CACGGTGGGCTCCTGGGAGCTCTGAGCTCTGGGCTTTCTCCAGTTCGCACTCTGCTTGTC
TCGGCAGCTCCGTCCCCACCGCAGAGGTGTGAAGGGGCGCAAAGCCAGCGAAGGGAGAAC
CCGGGTCGGGTAACCCCCAGGCCTGGCCAGGCGGACGCAGGGGCATCTCGGGCGAGGCGC
GCCTTGCGTCACGTGGGCACCGCCCCTGCAGTGACCGGAGAACGGCGTGTTCCTAGGGCT
CTGCTGCCGTCGTGGAAGCAGGATTTCCGCGGTTGTGTAACGGCCTGTCGCAGGCGTGCA
GGGACCTGGACTCCGCCTCGTCCCCGGGGCTCGGGCAGCCGAGCC
1.6 kb fragment (“Endo3” in this specification)-SEQ ID
NO: 48:
GAAGGAAGGAAGGAAGGTAGGTAGGTGTACAGTGTGTACAGATTGACAATAAAACCTTGA
ACAACGGGATATCGAAATTCTTGCCCACACAGTATGGGCTTTGCCAGTTTTCAGATACAT
TTTTATGAAGTATTTTCGCTGGCAAAACCATTCAGGTAGGGGCATGAAATGATAGTGTTT
CATCACTCGTGATCTCTAGGGGCAGCTATTCCAAAGACTACAATCTGAGAGGTTTCAAAA
CACCTCTGGTGTTTTCACTTGATGGCCACTGCCCACTCTTCCAGTCACTAAGGCTGAGAT
TTTTCTCACCACTTTCAAAACAACTTTTTGTCACTGGTAACATGCCTCATGTGACCTAGA
AGATTTTGAAACAACAATTTTACTTAAACTCCGTGAAGATTTCAAAACAACAATTTTACT
TAAACTCCATGTTGAAGGCAAAAAGGAAGATTGTGTGAAGCACTTTGAGGAAGGAAAAAA
TGATGGAACGATATTTCATCAAGGAAGCCCATGAGGAAGAAAATTTATTAAAACCAAGTA
GGTGTATGGAGGAGAGGCCGCCTCGTGAAGAAAACGAACATAATTTTACGCTGATTTGGT
GTCTTTTCTGTTTTTCTCTTGTTGGTATCTACATTCATCTTTTCCAATAATAATCCCATA
TATGTACATTTTTATCTGTTTAAATTCAGTAAAAGTTGGGAGGAAAATGTGTCAAAACTT
TTAGAGTGTGATGGGCTGAGTGGGGTTGTGAGTGATTATCTTCTTTTTTCAGTTTTTTTC
TGGGTTTTCCAAGTGTTCCTCGATGAACATGGATAGTTTTTCTGACAGGATAAAAAAGAA
GTAGTCCGGGACAGTGGCTAACACCCCGAATCCCAGCACTTTGGGAAGCCGGAGGTGGGA
GGATCGCTTGAGGCCAGGAGTTTGAAACCAGCCTGGGCAGCATAACGACACTCCCTCTCT
ACGAAAAACGAAAAAAAATAATTAGCCGGACGTGGTGGCGTGCGACTGTGGTCCCAGCTA
CTCGGGAGGCTGAGGTGGGAGGATCGCTTGAGCCCAATAGGTGGAGGCTCCGTGAGCTGA
GATAGCGCCACTGCGCTCCTGCCTGGGCGACAGAGTGAGAACCTGACTCAAAACAAAGAA
AAAAGGAAGAAAAGAAAGGAAGGGAAGAAGGAAGGAAGGGAGAAGCTTTCAAAAATAAAC
TTTTGTAAGAAGTAATGACACCGCTAGCCGTCCACACCAGGAGACCGCCTAGCCGTGGGG
CACGGTGGGCTCCTGGGAGCTCTGAGCTCTGGGCTTTCTCCAGTTCGCACTCTGCTTGTC
TCGGCAGCTCCGTCCCCACCGCAGAGGTGTGAAGGGGCGCAAAGCCAGCGAAGGGAGAAC
CCGGGTCGGGTAACCCCCAGGCCTGGCCAGGCGGACGCAGGGGCATCTCGGGCGAGGCGC
GCCTTGCGTCACGTGGGCACCGCCCCTGCAGTGACCGGAGAACGGCGTGTTCCTAGGGCT
CTGCTGCCGTCGTGGAAGCAGGATTTCCGCGGTTGTGTAACGGCCTGTCGCAGGCGTGCA
GGGACCTGGACTCCGCCTCGTCCCCGGGGCTCGGGCAGCCGAGCC

It is anticipated that fragments of SEQ ID NOs: 46-48 may also be functional as a promoter for IRAK3. An example functional fragment of SEQ ID NOs: 46-48 is shown in SEQ ID NO: 49 below. SEQ ID NO: 49 comprises H3K methylation marks and a CpG island, and also comprises a predicted TATA box (GATAAA), which are all typical hallmarks of a promoter.

SEQ ID NO: 49:
GATAAAAAAGAAGTAGTCCGGGACAGTGGCTAACACCCCGAATCCCAGC
ACTTTGGGAAGCCGGAGGTGGGAGGATCGCTTGAGGCCAGGAGTTTGAA
ACCAGCCTGGGCAGCATAACGACACTCCCTCTCTACGAAAAACGAAAAA
AAATAATTAGCCGGACGTGGTGGCGTGCGACTGTGGTCCCAGCTACTCG
GGAGGCTGAGGTGGGAGGATCGCTTGAGCCCAATAGGTGGAGGCTCCGT
GAGCTGAGATAGCGCCACTGCGCTCCTGCCTGGGCGACAGAGTGAGAAC
CTGACTCAAAACAAAGAAAAAAGGAAGAAAAGAAAGGAAGGGAAGAAGG
AAGGAAGGGAGAAGCTTTCAAAAATAAACTTTTGTAAGAAGTAATGACA
CCGCTAGCCGTCCACACCAGGAGACCGCCTAGCCGTGGGGCACGGTGGG
CTCCTGGGAGCTCTGAGCTCTGGGCTTTCTCCAGTTCGCACTCTGCTTG
TCTCGGCAGCTCCGTCCCCACCGCAGAGGTGTGAAGGGGCGCAAAGCCA
GCGAAGGGAGAACCCGGGTCGGGTAACCCCCAGGCCTGGCCAGGCGGAC
GCAGGGGCATCTCGGGCGAGGCGCGCCTTGCGTCACGTGGGCACCGCCC
CTGCAGTGACCGGAGAACGGCGTGTTCCTAGGGCTCTGCTGCCGTCGTG
GAAGCAGGATTTCCGCGGTTGTGTAACGGCCTGTCGCAGGCGTGCAGGG
ACCTGGACTCCGCCTCGTCCCCGGGGCTCGGGCAGCCGAGCC

In some embodiments, the promoter comprises the nucleic acid sequence of SEQ ID NO: 49 or functional fragment thereof. In some embodiments, the promoter comprises the nucleic acid sequence of SEQ ID NO: 49. In some embodiments, the promoter consists of the nucleic acid sequence of SEQ ID NO: 49. In some embodiments, the promoter comprises the nucleic acid sequence of SEQ ID NO: 46 or a functional fragment thereof. In some embodiments, the promoter consists of the nucleic acid sequence of SEQ ID NO: 46. In some embodiments, the promoter comprises the nucleic acid sequence of SEQ ID NO: 47 or a functional fragment thereof. In some embodiments, the promoter consists of nucleic acid sequence of SEQ ID NO: 47. In some embodiments, the promoter comprises the nucleic acid sequence of SEQ ID NO: 48 or a functional fragment thereof. In some embodiments, the promoter consists of the nucleic acid sequence of SEQ ID NO: 48.

In this specification the term “operably linked” may include the situation where a selected nucleotide sequence and regulatory nucleotide sequence, such as a promoter sequence are covalently linked in such a way as to place the expression of a nucleotide coding sequence under the influence or control of the regulatory sequence. Thus, a regulatory sequence is operably linked to a selected nucleotide sequence if the regulatory sequence is capable of effecting transcription of a nucleotide coding sequence which forms part or all of the selected nucleotide sequence. Where appropriate, the resulting transcript may then be translated into a desired protein or polypeptide.

In some embodiments, introduction of a nucleic acid encoding IRAK-M results in a genetically engineered target cell or tissue. By the term “genetically engineered” herein is meant a cell or tissue that has been subjected to recombinant DNA manipulations, such as the introduction of exogeneous nucleic acid. For example, the cell contains exogeneous nucleic acid. Generally, the exogeneous nucleic acid is made using recombinant DNA techniques.

Therapeutic nucleic acid can be delivered in vivo. Alternatively, therapeutic nucleic acid can be delivered ex vivo, whereby cells of a patient are extracted and cultured outside of the body. The cells are then genetically modified by introduction of a therapeutic nucleic acid and then re-introduced back into the patient. In preferred embodiments, the nucleic acid is delivered in vivo. In the context of the invention, it is preferable that expression of the nucleic acid encoding IRAK-M lasts for as long as possible. It is also preferable that there is low immunogenicity since the host's immune response can determine transgenic expression.

Gene delivery into target tissue/cells is a key step in gene therapy. This step may be carried out by gene delivery vehicles called vectors. Vectors for gene therapy are vehicles that carry the gene of interest to the target cell. There are two types of vector, viral and non-viral. In some embodiments, the vector is a viral vector. In alternative embodiments, the vector is a non-viral vector.

### Viral Vector Gene Delivery Systems

Recombinant viral vectors that are preferably replication deficient have been used as vehicles to deliver transgenes into target cells.

In some embodiments, the nucleic acid is delivered to the target cell via a viral vector. Viral gene delivery vectors include, but are not limited to nucleic acid sequences from the following viruses: RNA viruses such as a retrovirus, adenovirus, adeno-associated virus, SV40-type viruses, polyoma viruses, Epstein-Barr viruses, papilloma viruses, herpes virus, vaccinia virus, polio virus, orthomyxovirus, paramyxovirus, papovavirus, picornavirus, lentivirus, pox virus, anellovirus, and alphavirus. In some embodiments, the viral vector is selected from the group consisting of adeno-associated virus vector, adenovirus vector, retrovirus vector, orthomyxovirus vector, paramyxovirus vector, papovavirus vector, picornavirus vector, lentivirus vector, herpes simplex virus vector, vaccinia virus vector, pox virus vector, anellovirus virus vector, and alphavirus vector.

In some embodiments, the viral vector is an adeno-associated virus vector. In some embodiments, the nucleic acid is a viral vector genome.

An aspect of the invention provides a vector virion for use in a method of treatment or prophylaxis of macular degeneration in a subject. The vector virion comprises a nucleic acid comprising a nucleic acid sequence encoding IRAK-M and is capable of driving expression of IRAK-M in a target cell. In some embodiments, the vector virion is a recombination vector virion.

Virion particles comprising vector genomes of the invention are typically generated in packing cells capable of replicating viral genomes, expressing viral proteins (e.g. structural virion proteins and associated enzymes), and assembling virion particles. Also provided is a packaging cell comprising a nucleic acid construct encoding a vector genome described herein. Packing cells may also require helper virus functions, e.g. from adenovirus, E1-deleted adenovirus or herpes virus. Techniques for producing virion particles are well known in art. The packaging cell is typically a eukaryotic cell, such as a mammalian cell, e.g., a primate cell, e.g. a human cell. In some embodiments, a cell line is used. In some embodiments, the packaging cells may be stably transformed cells such as HeLa cells, 293 cells (HEK293, HEK293T or HEK293ET cells) and PerC.6 cells. Other cell lines include MRC-5 cells, WI-38 cells, Vero cells and FRhL-2 cells. The invention also provides method of producing a vector virion.

The size of the transgene which can be incorporated into the viral vector will depend on various factors, such as the specific virus on which the vector is based, the packaging capacity of the virion and which (if any) of the native viral genes have been deleted from the vector.

Non-limiting examples of viral vectors are provided below.

### Adenovirus:

Adenoviruses are commonly used in gene therapy because of their ability to be successfully transduced into a large number of cell types. They generally have a packaging ability of about packaging ability of 30 to 40 kb nucleic acid.

To improve safety different generations of adenoviral vectors have been generated. First generation adenoviral vectors were engineered by removing the E1 region making them replication defective and removing the E3 region. Newer second-generation adenoviruses have been engineered with additional deletions or mutations in the viral E2 and E4 regions, preventing transcriptional control of viral gene expression and viral genome replication, respectively. Further improvement in the safety and efficacy of adenoviral vectors has come with the development of “gutless” or “helper-dependent” adenoviral vectors that have all viral sequences deleted except for the inverted terminal repeat (ITRs) and the packing signal allowing for around 36 kb of space for cargo genes. This third-generation virus requires an additional adenoviral helper virus that is similar in composition to the first general virus (except they contain loxP sites inserted to flank the packing signal) to help with replication and packaging. These third-generation vectors retain the advantages of the first-generation adenoviral vectors in terms of high efficiency in in vivo transduction and transgene expression, and can mediate high-level, long-term transgene expression in the absence of toxicity.

In some embodiments, the viral vector is an adenoviral vector. In some embodiments, the viral vector is a first-generation adenoviral vector, a second-generation adenoviral vector, or a third-generation adenoviral vector.

### Lentivirus:

Lentiviruses are RNA viruses of the retrovirus family. The packaging capacity of this viral vector ranges from 8-9 kb nucleic acid. They possess a reverse transcriptase through which they can integrate their retrotranscribed proviral DNA into the chromosomes of host cells. Lentiviruses are able to integrate their genome into the host cell, resulting in stable expression. However, genome integration can result in insertional mutagenesis. Accordingly, non-integral lentiviral vectors have been developed, typically by making them deficient in integrases. These persist as episomal dsDNA circles capable of transducing nondividing cells. These non-integral vectors allow for efficient and sustained transgenic expression in post-mitotic tissues.

In some embodiments, the viral vector is a lentiviral vector. In some embodiment, the viral vector is a non-integral lentiviral vector.

Adeno-associated viruses:

Adeno-associated virus is a replication-deficient parvovirus having a single stranded DNA genome of which is about 4.7 kb in length, including 145 nucleotide ITRs. Several features render them suitable for retinal gene therapy, such as lack of pathogenicity, minimal immunogenicity, ability to transduce nondividing cells, and capacity to mediate sustained levels of therapeutic gene expression. Adeno-associated viruses are among the smallest viruses, with an uncoiled icosahedral capsid of about 22 nm. Since they require the presence of a helper virus for replication to occur, adeno-associated viruses are classified as dependoviruses that are naturally deficient in replication and nonpathogenic. Importantly, AAV recombinant genomes persist as episomes in transduced cells, leading to long-lasting expression of the transgene in nondividing retinal cells (Bordet T et al. Drug Discovery Today, Volume 24, Number 8, August 2019). AAVs have also been routinely used in ocular gene therapy (Buck T. M. Int. J. Mol. Sci. 2020, 21, 4197).

Preferred viral gene delivery vectors are AAV vectors. “AAV” is an abbreviation for adeno-associated virus and may be used to refer to the virus itself or derivatives thereof. The term covers all serotypes and variants both naturally occurring and engineered forms. The abbreviation “rAAV” refers to recombinant adeno-associated virus, also referred to as a recombinant AAV vector (or “rAAV vector”). The rAAV may comprise the polynucleotide of interest (e.g. the nucleic acid sequence encoding IRAK-M). In general, the rAAV vectors contain 5′ and 3′ adeno-associated virus inverted terminal repeats (ITRs), and the polynucleotide of interest operatively linked to sequences which regulate its expression in a target cell.

The term “AAV” includes but is not limited to AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), and AAV type 9 (AAV9). The genomic sequences of various serotypes of AAV, as well as the sequences of the native terminal repeats (TRs), Rep proteins, and capsid subunits are known in the art. Such sequences may be found in the literature or in public databases, for example, such as GenBank. See, e.g., GenBank Accession Numbers NC_002077 (AAV-1), AF063497 (AAV-1), NC_001401 (AAV-2), AF043303 (AAV-2), NC_001729 (AAV-3), NC_001829 (AAV-4), U89790 (AAV-4), NC_006152 (AAV-5), AF513851 (AAV-7), AF513852 (AAV-8), and NC_006261 (AAV-8).

An AAV is able to infect both dividing and nondividing cells and has a broad tropism that allows it to infect many cell types depending on the particular serotype. The recombinant vectors of AAV (rAAV) used for gene therapy are mainly based on serotype 2 (AAV2); this was the first human serotype described and the best characterized AAV serotype. Since the AAV capsid protein is responsible for its tropism and, therefore, for its efficacy, a pseudotyped strategy has previously been developed in which pseudotyped or hybrid AAV vectors encode a serotype rep, usually AAV2, and the cap gene of a different serotype.

The vector may be a pseudotyped AAV vector. The phrase “pseudotyped AAV vector”, herein designates a vector particle comprising a native AAV capsid including an rAAV vector genome and AAV Rep proteins, wherein Cap, Rep and the ITRs of the vector genome come from at least 2 different AAV serotypes. Examples of AAV chimeric vectors include but are not limited to AAV2/5, AAV2/6, and AAV2/8.

As the signals directing AAV replication, genome encapsulation and integration are contained within the ITRs of the AAV genome, some or all of the internal sequence of the genome (encoding replication and structural capsid proteins, rep-cap) may be replaced with foreign DNA such as an expression cassette, with the rep and cap proteins provided in trans. The sequence located between the ITRs of an AAV vector may be referred to as a “payload”. In some embodiments, the payload is a nucleic acid comprising a nucleic acid sequence encoding IRAK-M. The actual capacity of any particular AAV particle may vary depending on the viral proteins employed.

The vector may be an engineered AAV vector. For example, the engineered AAV vector is the SH10 vector as described in Klimczak R R, et al. 2009. PLOS One 4 (10): e7467. The AAV engineered vector may have a mutated capsid, in particular a tyrosine mutated capsid. Other known suitable engineered capsids include AAV2tYF, AAV2.7m8, R100, AAV2.GL, AAV2.NN, AAV44.9, and AAV44.9 (E531D).

Techniques to produce AAV vector particles in packaging cells are standard in the art. For example, production of pseudotyped AAV is disclosed in WO 01/83692. In various embodiments, AAV capsid proteins may be modified to enhance delivery of the recombinant vector. Modifications to capsid proteins are generally known in the art. See, for example, US 2005/0053922 and US 2009/0202490.

A non-limiting example method of generating a packaging cell is to create a cell line that stably expresses all the necessary components for AAV particle production. For example, a plasmid (or multiple plasmids) comprising an AAV genome lacking AAV rep and cap genes, AAV rep and cap genes separate from the AAV genome, and a selectable marker, such a neomycin resistance gene, are integrated into the genome of the cell. The packaging cell line is then infected with a helper virus such as adenovirus. The advantages of this method are that the cells are selected and are suitable for large-scale production of AAV. This can also be achieved using an adenovirus or baculovirus instead of plasmids for introducing AAV genomes and/or rep and cap genes into packaging cells.

In some embodiments, the viral vector is an adeno-associated virus vector (AAV). In some embodiments, the AAV is selected from the group consisting of AAV type 1 (AAV-1), AAV type 2 (AAV-2), AAV type 3 (AAV-3), AAV type 4 (AAV-4), AAV type 5 (AAV-5), AAV type 6 (AAV6), AAV type 7 (AAV-7), AAV type 8 (AAV-8), and AAV type 9 (AAV9). In some embodiments, the AAV is AAV2. In some embodiments, the AAV is AAV8. In some embodiments, the AAV is Anc80. In some embodiments, the AAV is AAV44.9. In some embodiments, the AAV is AAV44.9 (E531D).

### Nonviral Gene Transfer:

Nonviral systems typically comprise all the physical and chemical systems except viral systems and generally include either chemical methods, such as cationic liposomes and polymers, or physical methods such as gene gun, electroporation, particle bombardment, ultrasound utilisation, and magnetofection.

Nonviral gene transfer has the benefit that it is typically more cost-effective, has reduced induction of the immune system and has no limitation in the size of the transgenic DNA. Nonviral DNA vectors can include a plasmid or minicircle. Nonviral RNA vectors can include a messenger RNA or circular RNA.

In some embodiments, the non-viral carrier is selected from the group consisting of nanoparticles, liposomes, cationic polymer, and calcium phosphate particles.

In some embodiments, the nucleic acid is delivered to the target cell via a non-viral delivery system. In some embodiments, the non-viral delivery system is selected from the group consisting of nanoparticles, liposomes, cationic polymer, calcium phosphate particles, gene gun, electroporation, particle bombardment, ultrasound utilisation, and magnetofection.

Nanoparticles (NPs) can be used to provide plasmid DNA containing a functional copy of a gene into target tissues, for example, the retina. NPs are usually engulfed by the target cells via phagocytosis or endocytosis. Typically, nanoparticle compositions can pass through the plasma membrane, escape endosomes, and transport the plasmid DNA to the nucleus (Sahu B et al. Biomolecules 2021, 11, 1135).

Generally, nanoparticles wrap or adsorb DNA or RNA on the surface. Nanoparticle uptake by target cells depends on their composition and net charge. There are many different types of nanoparticle, including but not limited to, lipid-based NPs, peptide-based NPs, polymer-based NPs, and metal-NPs.

Lipidic nanoparticles are stable and biocompatible, and do not cause immune responses after administration (e.g. to the eye). Typically, lipid-based NPs are composed of a cationic lipid (having a positive charge, a hydrophilic head, and a hydrophobic tail, such as DOTAP) and a helper lipid (such as cholesterol). The positively charged head binds to a negatively charged phosphate group in the DNA to form a compact structure of lipoplexes. When DNA is enclosed in lipoplexes, it is protected from degradation. The lipid-DNA complex enters the cell by endocytosis.

Peptide-based NPs generally comprise a cationic peptide, enriched in lysine/arginine forming a tight compact structure with the DNA. Polymer-based NPs generally comprise a cationic polymer mixed with DNA to form nanosized polyplexes. Some examples of polymer-based vectors are polyethylene (PEI), dendrimers, and polyphosphoesters. Example synthetic polymers include but are not limited to Poly (L-ornithine), polyethyleneimine, and poly(amidoamine) dendrimers. Some example natural polymers include but are not limited to chitosan, dextran, and gelatin. An example of a metal NP is a gold NP (AuNP). DNA-gold nanoparticles are easy to generate and have high tolerability and low toxicity. Other nanoparticles considered are calcium-phosphorus silicate nanoparticles, calcium phosphate nanoparticles, silicon dioxide nanoparticles.

In some embodiments, the nucleic acid according to the invention is delivered to a target cell using nanoparticles. In some embodiments, the nanoparticle is a lipid-based nanoparticle. In some embodiments, the nanoparticle is a peptide-based nanoparticle. In some embodiments, the nanoparticle is a polymer-based nanoparticle. In some embodiments, the nanoparticle is a metal nanoparticle, optionally a gold nanoparticle.

The positive charge on the surface of the cationic polymer can form a positive complex with the negatively charged gene. The complex can be absorbed onto the cell surface by electrostatic action, and the gene is introduced naturally into the cell and subsequently expressed through endocytosis. Cationic polymers can be divided into polypeptides such as polylysine and polyglutamic acid, synthetic polymer material such as polyethylenimine (PEI) and polypropylene imine, and natural polymers such as chitosan, gelatin, and cyclodextrin.

In some embodiments, the nucleic acid according to the invention is delivered to a target cell using a cationic polymer. In some embodiments, the cationic polymer is a polypeptide polymer. In further embodiments, the polypeptide polymer is selected from the group consisting of polylysine and polyglutamic acid. In some embodiments, the cationic polymer is a synthetic polymer. In further embodiments, the synthetic polymer is selected from the group consisting of polyethylenimine (PEI) and polypropylene imine. In some embodiments, the cationic polymer is a natural polymer. In further embodiments, the natural polymer is selected from the group consisting of chitosan, gelatin, and cyclodextrin.

Also considered are calcium phosphate particles. These are biocompatible and biodegradable. Calcium plays a vital role in endocytosis and has the advantage of being readily absorbed and it poses high binding affinity. In some embodiments, the non-viral delivery system is calcium phosphate nucleotide-mediated nucleotide delivery.

Liposomes can be used for delivery of the nucleic acid of the invention into a target cell. A liposome is an artificial membrane with a thickness of 5-7 nm and a diameter of 25-500 nm. It has favourable biocompatibility and almost has no inhibition and no significant damage to normal tissues and cells such that it can exist around the target cells for a long time, enabling the target gene to be fully transfected into the target cells. Liposomes can be digested by lysosomes to release the nucleic acid in the natural mechanism, and therefore it entails a fast and convenient drug delivery, high transdermal absorption efficiency, low drug toxicity, and high stability. In some embodiments, the nucleic acid according to the invention is delivered to a target cell using liposomes. Also anticipated are nanolipsomes. Nanolipsomes are submicro bilayer lipid vesicle. Examples include but are not limited to ceramide-containing nanoliposomes and proteoliposomes.

Physical methods include but are not limited to, iontophoresis, bioballistic delivery, electrotransfection, magnetofection, sonoporation, and optoporation. Electrotransfection has been demonstrated as being particular useful for gene delivery to the eye. It is also known as electroporation or electro-permeabilization, involves applying a local and short external electric field to the cell to transiently modify the permeability of the cell membrane, facilitate the penetration of naked plasmid DNA, and promote its intracellular trafficking through electrophoresis (Bordet T et al. Drug Discovery Today, Volume 24, Number 8, August 2019). In some embodiments, the nucleic acid according to the invention is delivered to a target cell by iontophoresis, bioballistic delivery, electrotransfection, magnetofection, sonoporation, or optoporation. In some embodiments, the nucleic acid according to the invention is delivered to a target cell by electrotransfection.

In naked plasmid vector delivery, a clinical-grade plasmid DNA is prepared to transfer the gene to the tissue. Cells can be injected or electroporated with naked plasmid DNA. This method is typically considered to be safe and biocompatible. Additionally, the method is associated with a low risk of inducing immune responses. There is also no limit of the size of coding sequences. In some embodiments, the nucleic acid according to the invention is delivered to a target cell as naked DNA.

In some embodiments, the non-viral carrier is selected from the group consisting of liposomes, nanoliposomes, ceramide-containing nanoliposomes, proteoliposomes, nanoparticles, calcium-phosphorus silicate nanoparticles, calcium phosphate nanoparticles, silicon dioxide nanoparticles, Microparticles, poly (D-arginine), nano-dendrimers, and calcium phosphate nucleotide-mediated nucleotide delivery

### Genome Editing System

The agent described herein may also be a genome editing system.

In an aspect, a nucleic acid system is provided comprising one or more nucleic acids, comprising:
a) a nucleic acid sequence encoding an RNA-guided endonuclease;
b) a nucleic acid sequence encoding a guide RNA complementary to a target sequence associated with an insertion site in the genome of the target cell and capable of directing said RNA-guided endonuclease to said target sequence; and
c) a nucleic acid sequence encoding IRAK-M,
   for use in a method of treatment or prophylaxis of macular degeneration in a subject. The nucleic acid sequence encoding IRAK-M is capable of driving expression of IRAK-M in a target cell of the subject and the nucleic acid system is suitable for directed insertion of the nucleic acid sequence encoding IRAK-M at the insertion site in the genome of the target cell.

Also provided is a system comprising:
a) an RNA-guided endonuclease;
b) a guide RNA complementary to a target sequence associated with an insertion site in the genome of the target cell and capable of directing said RNA-guided endonuclease to said target sequence; and
c) a nucleic acid sequence encoding IRAK-M,
   for use in a method of treatment of prophylaxis of macular degeneration in a subject, where the nucleic acid sequence encoding IRAK-M is capable of driving expression of IRAK-M in a target cell of the subject and the system is suitable for directed insertion of the nucleic acid sequence encoding IRAK-M at the insertion site in the genome of the target cell.

The present invention may also use a CRISPR (“clustered regularly interspaced short palindromic repeats”) system to modulate expression of target genes.

The CRISPR or CRISPR-Cas system is derived from a prokaryotic RNA-guided defence system. There are at least eleven different CRISPR-Cas systems, which have been grouped into three major types (I-III). Type II CRISPR-Cas systems have been adapted as a genome-engineering tool.

Typically, most naturally occurring type II CRISPR-Cas systems employ three components:
a protein endonuclease Cas (CRISPR-associated protein) having DNA nickase activity which is referred to in this specification as an RNA-guided endonuclease (or an RNA-guided DNA endonuclease),
a “targeting” or “guide” RNA (CRISPR-RNA or crRNA) comprising a short sequence, typically of approximately 20 nucleotides, complementary to a target sequence (“protospacer”) in the genome, and
a “scaffold” RNA (trans-acting CRISPR RNA or tracrRNA) which interacts with the crRNA and recruits the Cas endonuclease.

Typically, assembly of these components and hybridisation of the crRNA with its target sequence in the chromosome results in cleavage of the chromosome by the endonuclease, at or close to the target sequence. Cleavage also requires that the target DNA contains a recognition site for the Cas enzyme (protospacer adjacent motif, or PAM) located sufficiently close to the crRNA target sequence, typically immediately adjacent the 3′ end of the target sequence. Cellular repair of the DNA break can lead to the insertion/deletion/mutation of bases and mutation at the target locus.

This three-component system has been simplified by fusing together crRNA and tracrRNA, to create a chimeric single guide RNA (sgRNA or gRNA). Hybridisation of the gRNA with the target sequence leads to cleavage of the target DNA at an adjacent/upstream PAM site. An gRNA can therefore be regarded as comprising a crRNA component (which determines the target sequence) and a tracrRNA component (which recruits the endonuclease).

The protein component of the CRISPR system is referred to as an endonuclease and may have enzymatic activity (i.e. DNA nickase activity) when associated with the appropriate RNA factors. Typically, the endonuclease will cleave chromosomal DNA. In some embodiments, the endonuclease is a Cas9 protein. Examples include Staphylococcus aureus (SaCas9), Streptococcus pyogenes (SpCas9), Neisseria meningitidis (NM Cas9), Streptococcus thermophilus (ST Cas9), Treponema denticola (TD Cas9), or variants thereof. The PAM sequences recognised by these enzymes are well known in the art. Beneficially, the new generation of SaCas9, CjCas9, and NmCas9 (2.9-3.3 kb) allows for the packaging of both Cas9 and gRNA in a single AAV vector. In some embodiments, the endonuclease is a Cas12a protein.

When using a catalytically active endonuclease, the target sequence recognised by the guide RNA may be upstream of a suitable site for insertion.

However, the endonuclease protein need not be enzymatically active. Catalytically inactive or (“dead”) endonuclease proteins may also be used in the context of the present invention, as they retain their ability to bind at the protospacer site targeted by the gRNA. A catalytically dead endonuclease may be indicated by the prefix “d”, e.g. dCas or dCas9.

The term “endonuclease” is therefore used to encompass both catalytically active and catalytically dead proteins unless the context demands otherwise.

The endonuclease may comprise a nuclear localisation sequence (NLS) effective in mammalian cells, such as the SV40 large T antigen NLS, which has the sequence PKKKRKV (SEQ ID NO: 33). Other mammalian NLS sequences are known to the skilled person. The endonuclease may comprise multiple copies of an NLS, e.g. two or three copies of an NLS. Where multiple NLS sequences are present, they are typically repeats of the same NLS.

In some embodiments, a gene encoding the endonuclease component of the nucleic acid system will be under transcriptional control of an RNA polymerase II promoter e.g. a viral or human RNA polymerase II promoter. Examples include CMV or SV40 promoter, or a mammalian “housekeeping” promoter. Genes encoding any RNA components (gRNA, crRNA or tracrRNA) will typically be under the transcriptional control of an RNA polymerase III promoter (e.g. a human RNA polymerase UII promoter) such as the U6 or H1 promoter, or variants thereof which retain or have enhanced activity.

In some embodiments, the gene editing system described herein (e.g., a nucleic acid system or CRISPR-based system) is used to increase expression of IRAK-M.

In some embodiments, it can be beneficial to employ multiple vectors and/or virions carrying different payloads. For example, for targeting integration of IRAK-M into the genome of the target cell, it may be necessary to employ one or more vectors. In one example, an AAV comprising Cas9 and the gRNA is used and a second AAV vector comprising the transgene of interest (e.g. IRAK-M). In an embodiment, one or more virions may each comprise at least one of the relevant components. The gene editing system as described herein can be used to introduce exogenous IRAK-M into the genome of a target cell. This process of introducing an exogenous gene is known as “knocking-in” or a “knock-in”. In this way exogenous IRAK-M is introduced into the target cell to increase expression.

Typically, the guide RNA directs the endonuclease (e.g. Cas9) to the target site to create a double-strand DNA break (DSB). Cleaved ends produced by nuclease cleavage are mainly repaired by non-homologous end joining (NHEJ) or homology-directed repair (HDR). Broadly, an exogenous DNA sequence or gene can then be incorporated into the target sequencing using HDR or NHEJ. The term “homology-directed repair” or “HDR” refers to a mechanism in cells to accurately and precisely repair double-strand DNA breaks using a homologous template to guide repair. The most common form of HDR is homologous recombination (HR), a type of genetic recombination in which nucleotide sequences are exchanged between two similar or identical molecules of DNA. The term “nonhomologous end joining” or “NHEJ” refers to a pathway that repairs double-strand DNA breaks in which the break ends are directly ligated without the need for a homologous template.

In some embodiments, the nucleic acid sequence encoding IRAK-M is inserted into the genome at the insertion site through homology-directed repair. In some embodiments, the nucleic acid sequence encoding IRAK-M is flanked by 5′ homology arm and a 3′ homology arm, wherein the 5′ homology arm is homologous to a DNA sequence 5′ of the target sequence from the insertion site and the 3′ homology arm is homologous to a DNA sequence 3′ of the target sequence from the insertion site. The term “homologous nucleic acid” as used herein includes a nucleic acid sequence that is either identical or substantially similar to a known reference sequence. In one embodiment, the term “homologous nucleic acid” is used to characterise a sequence that is at least 70%, at least 75%, at least 80%, at least 85%, at least 90%, at least 95%, at least 96%, at least 97%, at least 98%, at least 99%, or even 100% identical to a known reference sequence.

In some embodiments, the nucleic acid sequence encoding IRAK-M further comprises a 5′ flanking sequence comprising a target sequence and a 3′ flanking sequence comprising a target sequence. In some embodiments, the 5′ flanking sequence is 5′ of the 5′ homology arm and wherein the 3′ flanking sequence is 3′ of the 3′ homology arm. In some embodiments, the guide RNA recognises the target sequence from the insertion site, the 5′ flanking sequence, and the 3′ flanking sequence. In some embodiments, the RNA-guided endonuclease cleaves the genome at the insertion site. In some embodiments, the RNA-guided endonuclease cleaves the nucleic acid comprising the nucleic acid sequence encoding IRAK-M at the 5′ flanking sequence and the 3′ flanking sequence.

In some embodiments, the nucleic acid comprising the nucleic acid sequence encoding IRAK-M is a plasmid. This may be called a “donor plasmid”. Typically, this produces a linear nucleic acid comprising the nucleic acid sequence encoding IRAK-M. In some embodiments, the linear nucleic acid comprising the nucleic acid sequence encoding IRAK-M is inserted into the genome at the insertion site through homology-directed repair.

In alternative embodiments, the nucleic acid system further comprises at least a second a nucleic acid sequence encoding a guide RNA. In some embodiments, the second gRNA recognises the 5′ flanking sequence, and the 3′ flanking sequence only. In some embodiments, the first gRNA recognises the target sequence from the insertion site.

In some embodiments, provided is an engineered CRISPR-Cas vector system, comprising one or more vectors, comprising:
a) a nucleic acid sequence encoding a Cas endonuclease;
b) a nucleic acid sequence encoding a guide RNA complementary to a target sequence in a suitable site for insertion and capable of directing said RNA-guided endonuclease to said target sequence; and
c) a nucleic acid sequence encoding IRAK-M, for use in a method of treatment or prophylaxis of macular degeneration in a subject, wherein the nucleic acid increases IRAK-M expression in a target cell of the subject and wherein the nucleic acid encoding IRAK-M is inserted in the genome of the target cell.

An engineered CRISPR-Cas system is also provided, where the system comprises:
a) a Cas endonuclease;
b) a guide RNA complementary to a target sequence in a suitable site for insertion and capable of directing said RNA-guided endonuclease to said target sequence; and
c) a nucleic acid sequence encoding IRAK-M, for use in a method of treatment or prophylaxis of macular degeneration in a subject, wherein the nucleic acid increases IRAK-M expression in a target cell of the subject and wherein the nucleic acid encoding IRAK-M is inserted in the genome of the target cell.

In some embodiments, the suitable site for insertion is the AAVS1 site. The AAVS1 site or locus is also known as the “safe harbour” site or locus. The AAVS1 locus, in the intron of PPP1R12C, provides a “safe harbour” locus because disruption of this site by the introduction of an exogenous gene does not have adverse effects on the cell. Moreover, this site is associated with robust transcription, maintaining expression of an exogenously inserted gene. Accordingly, the AAVS1 is a well-validated “safe harbour” for hosting exogenous genes, thus making it a suitable target site in the context of the present invention.

Photoreceptors and RPE are postmitotic. Accordingly, these cells lack the homology-directed repair (HDR) mechanism (Ziccardi L., Int. J. Mol. Sci. 2019, 20, 5722). Site-specific transgene integration typically requires the HDR pathway. However, recent studies have identified methods for performing targeting integration using CRISPR systems in non-dividing cells. An example is described in Suzuki K., et al. 2016, Nature, Vol540, 144-149 and WO2018013932. The method described in Suzuki et al. employs a homology-independent targeted integration (HITI) strategy, which allows for robust DNA knock-in in both dividing and non-dividing cells. The HITI is based on non-homologous end joining (NHEJ) and so can be carried out in non-dividing cells. The method as described in Suzuki et al. can be readily applied to the present invention. For example, a nucleic acid encoding IRAK-M can be knocked-in the genome of the subject's ocular cells through CRISPR/Cas9-mediated homology-independent targeted integration (Suzuki 2016), which has been demonstrated to work in vivo in non-dividing cells such as RPE.

This method allows for directional insertion of exogenous DNA in non-dividing cells. This is achieved by employing a nucleic acid sequence comprising the gene of interest, flanked by two target sequences (e.g. a target sequence 5′ of the nucleic acid sequence encoding IRAK-M and a target sequence 3′ of the nucleic acid sequence encoding IRAK-M). The target sequences in the nucleic acid sequence comprising the gene of interest are typically in the reverse direction. The target sequence in the genome is cleaved by the RNA-endonuclease forming a first half and second half of the sequence. The target sequences in the nucleic acid sequence comprising the gene of interest are also cleaved by the RNA-guided endonuclease forming a first half and second half of each target sequence. This forms a nucleic acid sequence, in the forward direction comprising a first half of a target sequence, a nucleic acid sequence comprising the gene of interest, and a second half of a target sequence. If this nucleic acid is correctly inserted in the genome it will form a sequence in the genome comprising a first half of the target sequence in the genome, a first half of the target sequence in the nucleic acid, a nucleic acid sequence comprising the gene of interest, a second half of the target sequence in the nucleic acid, and a second half of the target sequence in the genome. However, if the nucleic acid is incorrectly inserted in the genome it will form a sequence in the genome comprising a first half of target sequence in the genome, a second half of target sequence in the nucleic acid, a nucleic acid sequence comprising the gene of interest, a first half of the target sequence in the nucleic acid, and a second half of the target sequence in the genome. Thus, reforming the complete target sequence at each end of the gene of interest that has been incorrectly inserted (i.e. the gene of interest is present in the reverse orientation). HITI is expected to occur more frequently in the forward direction than the reverse direction as an intact guide RNA (gRNA) target sequence remains in the latter, which is subjected to additional endonuclease cutting until forward transgene insertion or insertions and deletions (indels) occur that prevent further gRNA binding.

In some embodiments, the nucleic acid sequence encoding IRAK-M is flanked by a 5′ target sequence and a 3′ target sequence. In some embodiments, the 5′ target sequence and the 3′ target sequence are the same as the target sequence from an insertion site in the genome. In some embodiments, the nucleic acid sequence encoding a guide RNA is complementary to the 5′ target sequence and the 3′ target sequence. In some embodiments, the nucleic acid sequence encoding a guide RNA is complementary to target sequence in the genome, the 5′ target sequence and the 3′ target sequence. In some embodiments, the target sequence is no longer present once the nucleic acid sequence encoding IRAK-M has been integrated into the genome in the correct orientation. In some embodiments, the target sequences present in the nucleic acid encoding IRAK-M are present in the opposite orientation to the target sequence from an insertion site in the genome. In some embodiments, the target sequences present in the nucleic acid encoding IRAK-M are present in the reverse direction. Typically, the target sequence in the genome is in the forward direction. In some embodiments, the first half and the second half of the target sequence have been cleaved by a nuclease and the first half and second half of the target sequence are inserted into the genome upstream and downstream of the exogenous DNA sequence. In this embodiment, there are no homology arms present in the nucleic acid comprising a nucleic acid sequence encoding IRAK-M.

“Target sequences” herein are nucleic acid sequences recognised and cleaved by an endonuclease disclosed herein in a sequence specific manner. In some embodiments, the target sequence comprises a nuclease binding site. In some embodiments, the target sequence comprises a nick/cleavage site. In some embodiments, the target sequence comprises a protospacer adjacent motif (PAM) sequence. The target sequences include the target sequence in the genome, the 5′ target sequence and the 3′ target sequence.

In some embodiments, the suitable site for insertion is the AAVS1 site.

The viral delivery system described herein, or the non-viral delivery system described herein, may be used to introduce the nucleic acid systems described herein to a target cell.

CRISPR/Cas system components may be delivered to a target cell as a ribonucleoprotein (RNP) complex comprising a Cas9 protein and a gRNA (as described in Zhang et al., Theranostics 2021, Vol. 11, Issue 2). Thus, a system comprising an RNA-guided endonuclease, a guide RNA, and a nucleic acid encoding IRAK-M as described herein may be delivered to a target cell as a complex.

Zhang et al, Theranostics 2021, Vol. 11, Issue 2, describes various methods for delivering such complexes to target cells. The complex described herein may be delivered to a target cell by direct penetration, such as microinjection of a target cell or biolistics. A target cell membrane may be disrupted by electroporation. Electroporation may disrupt the target cell membrane, temporarily forming “nanopores” which the complex can transport across. Before electroporation, the complexes may be stabilised using an anionic polymer (e.g., polyglutamic acid). Alternatively, virus-like particles (VLPs) may be used to deliver the complex. For example, an RNA-guided endonuclease may be incorporated into lentriviral particles. Banskota et al., 2022 Cell 185, 250-265, describes the use of engineered-DNA-free-virus-like particles (eVLPs) that are able to package and deliver complexes, such as a Cas9 RNP, to target cells (for example in the retina).

Zhang et al., 2022 also describes the use of lipid nanoparticles to deliver the complex as described herein. The lipid nanoparticles may include cell-derived extracellular vesicles (EVs) and synthetic lipid nanoparticles. An example of a synthetic lipid nanoparticle includes, CRISPR^{MAX} (Thermo-Fisher) which has been described as successfully delivering complexes to the human retinal pigment epithelial cells (Yu et al, Biotechnol Lett (2016) 38:919-929). Alternatively, Zhang et al. 2022, describes the use of CPPs to enable the delivery of the complex. Also described are methods in which lipid moieties are added to the complex to increase the membrane permeability. Further described are polymers such as dendrimers, PBAEs, PEGylated PLL, and Chitosan nanoparticules for delivering complexes as described herein. The use of nanogels is also described in Zhang et al. Chen et al., Nat Nanotechnol (2019); 14:974-80 describes the delivery of a complex in a nanogel to mouse retina/RPE in vivo. Nanoparticles could also be used to deliver such complexes (Zhang et al., 2022). For example, inorganic nanoparticles, such as gold nanoparticles, metal-organic frameworks (MOFs), graphene oxide, black phosphorous (BP) nanosheets, or calcium phosphate nanoparticles. In an example, Wang et al., J Controlled Release. 2020; 324:194-203, describes delivery of a complex (e.g., RNP) to mouse retina in vivo using a nanoparticle (a pH-responsive silica-metal-organic framework hybrid nanoparticle). Other methods described in Zhang et al., may also be used in the context of the present invention.

Any of the methods described herein may be used to deliver a complex as described herein (i.e., a system comprising an RNA-guided endonuclease, a guide RNA, and a nucleic acid encoding IRAK-M as described herein).

Delivery of the systems described herein as complexes (e.g., the protein/RNA/DNA complexes) may result in transient genome editing and thus reducing off-target effects, insertional mutagenesis, and immune responses. Delivery as a complex may also result in faster genome editing because it eliminates the need for intracellular transcription and translation.

### Increasing Endogenous Gene Expression

The following agents are capable of increasing endogenous IRAK-M expression.

### Systems for Increasing Endogenous IRAK-M Expression

A nucleic acid system (e.g. CRISPR activation system) can be employed to increase endogenous IRAK-M expression. An example is a nucleic acid activation system (e.g., a CRISPR/Cas9 activation system).

Transcriptional activators are protein domains or whole proteins (which may be linked to deactivated endonuclease) that assist in the recruitment of co-factors, transcription factors and/or RNA polymerase for transcription of the target gene.

In an aspect, a nucleic acid system is provided comprising one or more nucleic acids, comprising:
a) a nucleic acid sequence encoding a deactivated RNA-guided endonuclease fused to one or more transcriptional activators; and
b) a nucleic acid sequence encoding a guide RNA complementary to a target sequence in the promoter or regulatory sequences for the IRAK3 gene and capable of directing said RNA-guided endonuclease to said target sequence, for use in a method of treatment or prophylaxis of macular degeneration in a subject, where the nucleic acid system increases IRAK-M expression in a target cell of the subject.

In some embodiments, the deactivated RNA-guided endonuclease is fused to a single transcriptional activator. For example, the deactivated RNA-guided endonuclease may be fused to VP64.

The one or more transcriptional activators may be joined to the N-terminus of the deactivated RNA-guided endonuclease. The one or more transcriptional activators may be joined to the C-terminus of the deactivated RNA-guided endonuclease. For example, VP64 may be fused to the C-terminus of the deactivated RNA-guided endonuclease. The VP64 may be fused to the deactivated RNA-guided endonuclease via a linker.

In some embodiments, the deactivated RNA-guided endonuclease is fused to more than one transcriptional activator. For example, the deactivated RNA-guided endonuclease may be fused to three transcriptional activators. In some embodiments, the transcriptional activators may be VP64, p65 and Rta. VP64 may be joined to the C-terminus of the deactivated RNA-guided endonuclease, p65 may be joined to the C-terminus of VP64, and Rta may be joined to the C-terminus of p65. An example of this system is the VP64-p65-Rta system, which is also known as VPR.

Also provided is a nucleic acid system comprising one or more nucleic acids, comprising:
a) a nucleic acid sequence encoding a deactivated RNA-guided endonuclease; and
b) a nucleic acid sequence encoding a guide RNA complementary to a target sequence in the promoter or regulatory sequences for IRAK3 gene and capable of directing said RNA-guided endonuclease to said target sequence, wherein said guide RNA further comprises an aptamer capable of specifically binding to a transcriptional activator,
   for use in a method of treatment or prophylaxis of macular degeneration in a subject, where the nucleic acid system increases IRAK-M expression in a target cell of the subject.

In some embodiments, the aptamer is an RNA aptamer. The transcriptional activator may be endogenous to the cell. Additionally, or alternatively, the transcriptional activator may be exogenous to the cell.

Also provided is a nucleic acid system comprising one or more nucleic acids, comprising:
a) a nucleic acid sequence encoding a deactivated RNA-guided endonuclease;
b) a nucleic acid sequence encoding an RNA binding protein fused to one or more transcriptional activators; and
c) a nucleic acid sequence encoding a guide RNA complementary to a target sequence in the promoter or regulatory sequences for the IRAK3 gene and capable of directing said RNA-guided endonuclease to said target sequence, wherein said guide RNA further comprises an RNA aptamer capable of specifically binding to the RNA binding protein,
   for use in a method of treatment or prophylaxis of macular degeneration in a subject, where the nucleic acid system increases IRAK-M expression in a target cell of the subject.

The one or more transcriptional activators may be selected from the group consisting of VP64, p65 and HSF1. In some embodiments, the p65 and HSF1 are fused to an RNA binding protein.

The RNA binding protein may be MS2 (also known as a MS2 bacteriophage coat protein) and the RNA aptamer may be capable of binding to MS2. The RNA aptamer may be capable of binding to dimerised RNA binding proteins (such as dimerised MS2). Without wishing to be bound by theory, one or more RNA binding proteins are anticipated to bind to the RNA aptamer, thereby providing the one or more transcriptional activators at the target site (via the gRNA). For example, a MS2-p65-HSF1 complex guided by target-specific MS2-mediated gRNA is anticipated to enhance the binding of transcription factors to the promoter for IRAK3. In some embodiments, the gRNA comprises a hairpin aptamer capable of binding MS2 (e.g., an MS2 fusion protein).

The tetraloop and stem-loop 2 of gRNA typically protrude outside of the Cas9-gRNA complex. It is also believed that these regions of the gRNA do not affect endonuclease activity. Thus, the tetraloop and/or the stem-loop 2 of gRNA may each be modified with RNA aptamers. In some embodiments, the RNA aptamer is a minimal hairpin aptamer. The minimal hairpin aptamer may be appended to the tetraloop and/or the stem loop 2 of gRNA. In some embodiments, the minimal hairpin aptamer specifically binds MS2. In some embodiments, the minimal hairpin aptamer specifically binds a MS2 dimer.

In some embodiments, the deactivated RNA-guided endonuclease is fused to an additional transcriptional activator. For example, the deactivated RNA-guided endonuclease may be fused to a single additional transcriptional activator. The deactivated RNA-guided endonuclease may be fused to VP64. An example nucleic acid system is the Synergistic Activation Mediator (SAM) system.

In another aspect, a nucleic acid system is provided comprising one or more nucleic acids, comprising:
a) a nucleic acid sequence encoding a deactivated RNA-guided endonuclease fused to an epitope repeat array comprising one or more epitopes;
b) one or more nucleic acid sequences encoding an epitope binding molecule fused to one or more transcriptional activators, wherein said epitope binding molecule is capable of specifically binding to an epitope of the epitope repeat array; and
c) a nucleic acid sequence encoding a guide RNA complementary to a target sequence in the promoter or regulatory sequences for the IRAK3 gene and capable of directing said RNA-guided endonuclease to said target sequence,
   for use in a method of treatment or prophylaxis of macular degeneration in a subject, where the nucleic acid system increases IRAK-M expression in a target cell of the subject.

In some embodiments, the epitope binding molecule is an antibody or an antibody-like molecule. The one or more transcriptional activators may be fused to a single-chain variable fragment (scFV). In some embodiments, a VP64 is fused to a scFv.

A single transcriptional activator may be fused to an epitope binding molecule. For example, VP64 may be fused to an antibody or antibody-like molecule. In some embodiments, more than one transcriptional activator may be fused to an epitope binding molecule. In some embodiments, the transcriptional activators may be selected from the group consisting of VP64, p65 and Rta.

The epitope repeat array may be capable of binding multiple epitope binding molecules fused to one or more transcriptional activators. Thus, the system described herein is capable of amplifying the number of transcriptional activators at the target site. The epitope sequence may be unique (i.e., it is different to naturally occurring sequences in the target cell).

The epitope binding molecule may comprise a nuclear localization sequence (NLS). The NLS can facilitate the transport of the epitope binding molecule to the nucleus of a target cell. In some embodiments, the NLS comprises an amino acid sequence comprising SEQ ID NO: 33.

An example of this system is known as a SunTag system, where GCN4 antibodies were fused to an NLS and VP64.

Also provided are the following systems.

An aspect of the invention provides a system comprising:
a) a deactivated RNA-guided endonuclease fused to one or more transcriptional activators; and
b) a guide RNA complementary to a target sequence in the promoter or regulatory sequences for the IRAK3 gene and capable of directing said RNA-guided endonuclease to said target sequence, for use in a method of treatment or prophylaxis of macular degeneration in a subject, where the system increases IRAK-M expression in a target cell of the subject.

Another aspect of the invention provides a system comprising:
a) a deactivated RNA-guided endonuclease; and
b) a guide RNA complementary to a target sequence in the promoter or regulatory sequences for IRAK3 gene and capable of directing said RNA-guided endonuclease to said target sequence, wherein said guide RNA further comprises an aptamer capable of specifically binding to a transcriptional activator,
   for use in a method of treatment or prophylaxis of macular degeneration in a subject, where the system increases IRAK-M expression in a target cell of the subject.

A further aspect provides a system comprising:
a) a deactivated RNA-guided endonuclease;
b) an RNA binding protein fused to one or more transcriptional activators;
c) a guide RNA complementary to a target sequence in the promoter or regulatory sequences for the IRAK3 gene and capable of directing said RNA-guided endonuclease to said target sequence, wherein said guide RNA comprises an RNA aptamer capable of specifically binding to the RNA binding protein,
   for use in a method of treatment or prophylaxis of macular degeneration in a subject, where the system increases IRAK-M expression in a target cell of the subject

Also provided is a system comprising:
a) a deactivated RNA-guided endonuclease fused to an epitope repeat array comprising one or more epitopes;
b) one or more epitope binding molecules fused to one or more transcriptional activators, wherein said epitope binding molecule is capable of specifically binding to an epitope of the epitope repeat array; and
c) a guide RNA complementary to a target sequence in the promoter or regulatory sequences for the IRAK3 gene and capable of directing said RNA-guided endonuclease to said target sequence,
   for use in a method of treatment or prophylaxis of macular degeneration in a subject, where the system increases IRAK-M expression in a target cell of the subject.

As described above, the above systems may be delivered to a target cell as a complex (e.g., a protein/RNA complex). In some embodiments, the RNA-guided endonuclease is a Cas endonuclease.

Described herein are activation systems. Example activation systems include but are not limited to, VP64-p65-Rta or VPR, deactivated endonuclease-SAM system, and deactivated endonuclease-SunTag system. Any of these activation systems can be used in the context of the present invention.

An example CRISPR activation system is described in Konermann S. et al. Nature 2015: 517 (7536), 583-588.

In some embodiments, provided is an engineered CRISPR-Cas vector system, comprising one or more vectors, comprising:
a) a nucleic acid sequence encoding a deactivated Cas endonuclease fused to one or more transcriptional activators; and
b) a nucleic acid sequence encoding a guide RNA complementary to a target sequence in the promoter or regulatory sequences for the IRAK3 gene and capable of directing said Cas endonuclease to said target sequence,
   for use in a method of treatment or prophylaxis of macular degeneration in a subject, where the CRISPR-Cas vector system increases IRAK-M expression in a target cell of the subject.

In some embodiments, an engineered CRISPR-Cas vector system comprising one or more nucleic acids is provided, where the system comprises:
a) a nucleic acid sequence encoding a deactivated Cas endonuclease; and
b) a nucleic acid sequence encoding a guide RNA complementary to a target sequence in the promoter or regulatory sequences for IRAK3 gene and capable of directing said Cas endonuclease to said target sequence, wherein said guide RNA further comprises an aptamer capable of specifically binding to a transcriptional activator,
   for use in a method of treatment or prophylaxis of macular degeneration in a subject, where the CRISPR-Cas vector system increases IRAK-M expression in a target cell of the subject.

An embodiment provides an engineered CRISPR-Cas vector system, comprising one or more vectors, comprising:
a) a nucleic acid sequence encoding a deactivated Cas endonuclease;
b) a nucleic acid sequence encoding an RNA binding protein fused to one or more transcriptional activators; and
c) a nucleic acid sequence encoding a guide RNA complementary to a target sequence in the promoter or regulatory sequences for the IRAK3 gene and capable of directing said Cas endonuclease to said target sequence, wherein said guide RNA further comprises an RNA aptamer capable of specifically binding to the RNA binding protein,
   for use in a method of treatment or prophylaxis of macular degeneration in a subject, where the CRISPR-Cas vector system increases IRAK-M expression in a target cell of the subject.

In another embodiment, provided is an engineered CRISPR-Cas vector system, comprising one or more vectors, comprising:
a) a nucleic acid sequence encoding a deactivated Cas endonuclease fused to an epitope repeat array comprising one or more epitopes;
b) one or more nucleic acid sequences encoding an epitope binding molecule fused to one or more transcriptional activators, wherein said epitope binding molecule is capable of specifically binding to an epitope of the epitope repeat array; and
c) a nucleic acid sequence encoding a guide RNA complementary to a target sequence in the promoter or regulatory sequences of the IRAK3 gene and capable of directing said Cas endonuclease to said target sequence,
   for use in a method of treatment or prophylaxis of macular degeneration in a subject, where the CRISPR-Cas vector system increases IRAK-M expression in a target cell of the subject.

Another example of an agent capable of increasing endogenous IRAK-M expression in a target cell is a nucleic acid demethylation system (e.g., a CRISPR/Cas9 demethylation system).

DNA methylation is an epigenetic process which occurs by the addition of a methyl group to DNA, typically cytosine bases. In mammals, DNA methylation regulates gene expression by acting to repress gene transcription. Without wishing to be bound by theory, it is anticipated by that the use of a demethylating system would increase accessibility of the IRAK3 gene or its promoter/regulatory sequences, allowing transcription.

Thus, another aspect provides a nucleic acid system comprising one or more nucleic acids, comprising:
a) a nucleic acid sequence encoding a deactivated RNA-guided endonuclease fused to one or more DNA demethylating agents; and
b) a nucleic acid sequence encoding a guide RNA complementary to (i) a target sequence in the promoter sequence for the IRAK3 gene, (ii) a target sequence in the regulatory sequences for the IRAK3 gene or (iii) a target sequence in the IRAK3 gene and capable of directing said RNA-guided endonuclease to said target sequence,
   for use in a method of treatment or prophylaxis of macular degeneration in a subject, where the nucleic acid system increases IRAK-M expression in a target cell of the subject.

The one or more DNA demethylating agents may be one or more DNA demethylating enzymes or a fragment thereof. For example, ten-eleven translocation methylcytosine dioxygenases (TET enzymes) mediate DNA demethylation by oxidizing 5-methylcytosine (5mC) in DNA to 5-hydroxymethylcytosine (5hmC), 5-formylcytosine (5fC), and 5-carboxylcytosine (5caC). In further embodiments, the DNA demethylating agent is the catalytic domain of TET1. In some embodiments, the DNA demethylating agent is TET1. Lysine-specific demethylase 1 (LESD1, also known as KDM1A) is a lysine demethylase acting on histones H3K4me1/2 and H3K9me1/2. In some embodiments, the DNA demethylating agent is LESD1.

In some embodiments, the deactivated RNA-guided endonuclease is fused to a single DNA demethylating agent. The one or more DNA demethylating agents may be fused to the C-terminus of the deactivated RNA-guided endonuclease. Alternatively, the one or more DNA demethylating agents may be fused to the N-terminus of the deactivated RNA-guided endonuclease.

Also provided is a nucleic acid system comprising one or more nucleic acids, comprising:
a) a nucleic acid sequence encoding a deactivated RNA-guided endonuclease; and
b) a nucleic acid sequence encoding a guide RNA complementary to (i) a target sequence in the promoter sequence for the IRAK3 gene, (ii) a target sequence in the regulatory sequences for the IRAK3 gene or (iii) a target sequence in the IRAK3 gene and capable of directing said RNA-guided endonuclease to said target sequence, wherein said guide RNA further comprises an aptamer capable of specifically binding to a demethylating agent,
   for use in a method of treatment or prophylaxis of macular degeneration in a subject, where the nucleic acid system increases IRAK-M expression in a target cell of the subject.

In some embodiments, the aptamer is an RNA aptamer. The demethylating agent may be endogenous to the cell. Additionally, or alternatively, the demethylating agent may be exogenous to the cell.

Also provided is a nucleic acid system is provided comprising one or more nucleic acids, comprising:
a) a nucleic acid sequence encoding a deactivated RNA-guided endonuclease;
b) a nucleic acid sequence encoding an RNA binding protein fused to one or more DNA demethylating agents;
c) a nucleic acid sequence encoding a guide RNA complementary to (i) a target sequence in the promoter sequence for the IRAK3 gene, (ii) a target sequence in the regulatory sequences for the IRAK3 gene or (iii) a target sequence in the IRAK3 gene and capable of directing said RNA-guided endonuclease to said target sequence, wherein said guide RNA further comprises an RNA aptamer capable of specifically binding to the RNA binding protein,
   for use in a method of treatment or prophylaxis of macular degeneration in a subject, where the nucleic acid system increases IRAK-M expression in a target cell of the subject.

The one or more DNA demethylating agents may be as described above. The RNA binding protein and/or the gRNA may be as described for the transcriptional activating system described above, with the exception that one or more DNA demethylating agents are fused to the RNA binding protein.

In some embodiments, the deactivated RNA-guided endonuclease is fused to an additional DNA demethylating agent. The additional DNA demethylating may be different to the one or more DNA demethylating agents.

In another aspect, a nucleic acid system is provided comprising one or more nucleic acids, comprising:
a) a nucleic acid sequence encoding a deactivated RNA-guided endonuclease fused to an epitope repeat array comprising one or more epitopes;
b) one or more nucleic acid sequences encoding an epitope binding molecule fused to one or more DNA demethylating agents, wherein said epitope binding molecule is capable of specifically binding to an epitope of the epitope repeat array; and
c) a nucleic acid sequence encoding a guide RNA complementary to (i) a target sequence in the promoter sequence for the IRAK3 gene, (ii) a target sequence in the regulatory sequences for the IRAK3 gene or (iii) a target sequence in the IRAK3 gene and capable of directing said RNA-guided endonuclease to said target sequence,
   for use in a method of treatment or prophylaxis of macular degeneration in a subject, where the nucleic acid system increases IRAK-M expression in a target cell of the subject.

The epitope repeat array may be as described for the transcriptional activation system described above. The epitope binding molecule may be as described above, with the exception that one or more DNA demethylating agents are fused to the epitope binding molecule.

The invention also provides the following aspects.

An aspect provides a system comprising:
a) a deactivated RNA-guided endonuclease fused to one or more DNA demethylating agents; and
b) a guide RNA complementary to (i) a target sequence in the promoter sequence for the IRAK3 gene, (ii) a target sequence in the regulatory sequences for the IRAK3 gene or (iii) a target sequence in the IRAK3 gene and capable of directing said RNA-guided endonuclease to said target sequence,
   for use in a method of treatment or prophylaxis of macular degeneration in a subject, where the system increases IRAK-M expression in a target cell of the subject.

A further aspect provides a system comprising:
a) a deactivated RNA-guided endonuclease; and
b) a guide RNA complementary to (i) a target sequence in the promoter sequence for the IRAK3 gene, (ii) a target sequence in the regulatory sequences for the IRAK3 gene or (iii) a target sequence in the IRAK3 gene and capable of directing said RNA-guided endonuclease to said target sequence, wherein said guide RNA further comprises an aptamer capable of specifically binding to a DNA demethylating agent,
   for use in a method of treatment or prophylaxis of macular degeneration in a subject, where the system increases IRAK-M expression in a target cell of the subject.

Further provided is a system comprising:
a) a deactivated RNA-guided endonuclease;
b) an RNA binding protein fused to one or more DNA demethylating agents;
c) a guide RNA complementary to (i) a target sequence in the promoter sequence for the IRAK3 gene, (ii) a target sequence in the regulatory sequences for the IRAK3 gene or (iii) a target sequence in the IRAK3 gene and capable of directing said RNA-guided endonuclease to said target sequence, wherein said guide RNA further comprises an RNA aptamer capable of specifically binding to the RNA binding protein,
   for use in a method of treatment or prophylaxis of macular degeneration in a subject, where the system increases IRAK-M expression in a target cell of the subject.

Another aspect, provides a system comprising:
a) a deactivated RNA-guided endonuclease fused to an epitope repeat array comprising one or more epitopes;
b) one or more epitope binding molecules fused to one or more DNA demethylating agents, wherein said epitope binding molecule is capable of specifically binding to an epitope of the epitope repeat array; and
c) a guide RNA complementary to (i) a target sequence in the promoter sequence for the IRAK3 gene, (ii) a target sequence in the regulatory sequences for the IRAK3 gene or (iii) a target sequence in the IRAK3 gene and capable of directing said RNA-guided endonuclease to said target sequence,
   for use in a method of treatment or prophylaxis of macular degeneration in a subject, where the system increases IRAK-M expression in a target cell of the subject.

As described above, the above systems may be delivered to a target cell as a complex (e.g., a protein/RNA complex). In some embodiments, the RNA-guided endonuclease is a Cas endonuclease.

A CRISPR-based approach for targeting DNA demethylation may allow for targeting epigenetic editing. For example, the demethylation system may comprise a deactivated endonuclease (e.g., a Cas9 nuclease) fused to a demethylation agent (e.g., TET1), and at least one IRAK-M-specific guide RNA. As with the CRISPR activation system, a CRISPR demethylation system uses modified versions of CRISPR effectors without endonuclease activity, with transcriptional activators on dCas or the gRNA. As with the system described above, the demethylation system comprises a deactivated endonuclease (e.g., dCas9), a gRNA and a DNA demethylating agent fused to the deactivated endonuclease or gRNA. Approaches for targeting DNA demethylation using CRISPR are described in Xu et a., 2016, Cell Discovery (2016) 2, 16009; doi: 10.1038/celldisc.2016.9.

In some embodiments, provided is an engineered CRISPR-Cas vector system, comprising one or more vectors, comprising:
a) a nucleic acid sequence encoding a deactivated Cas endonuclease fused to one or more DNA demethylating agents; and
b) a nucleic acid sequence encoding a guide RNA complementary to (i) a target sequence in the promoter sequence for the IRAK3 gene, (ii) a target sequence in the regulatory sequences for the IRAK3 gene or (iii) a target sequence in the IRAK3 gene, and capable of directing said Cas endonuclease to said target sequence, for use in a method of treatment or prophylaxis of macular degeneration in a subject, where the CRISPR-Cas vector system increases IRAK-M expression in a target cell of the subject.

In some embodiments, provided is an engineered CRISPR-Cas vector system comprising one or more nucleic acids, comprising:
a) a nucleic acid sequence encoding a deactivated Cas endonuclease; and
b) a nucleic acid sequence encoding a guide RNA complementary to (i) a target sequence in the promoter sequence for the IRAK3 gene, (ii) a target sequence in the regulatory sequences for the IRAK3 gene or (iii) a target sequence in the IRAK3 gene and capable of directing said Cas endonuclease to said target sequence, wherein said guide RNA further comprises an aptamer capable of specifically binding to a DNA demethylating agent,
   for use in a method of treatment or prophylaxis of macular degeneration in a subject, where the CRISPR-Cas vector system increases IRAK-M expression in a target cell of the subject.

An embodiment provides an engineered CRISPR-Cas vector system, comprising one or more vectors, comprising:
a) a nucleic acid sequence encoding a deactivated Cas endonuclease;
b) a nucleic acid sequence encoding an RNA binding protein fused to one or more DNA demethylating agents; and
c) a nucleic acid sequence encoding a guide RNA complementary to (i) a target sequence in the promoter sequence for the IRAK3 gene, (ii) a target sequence in the regulatory sequences for the IRAK3 gene or (iii) a target sequence in the IRAK3 gene, and capable of directing said Cas endonuclease to said target sequence, wherein said guide RNA further comprises an RNA aptamer capable of specifically binding to the RNA binding protein,
   for use in a method of treatment or prophylaxis of macular degeneration in a subject, where the CRISPR-Cas vector system increases IRAK-M expression in a target cell of the subject.

In another embodiment, provided is an engineered CRISPR-Cas vector system, comprising one or more vectors, comprising:
a) a nucleic acid sequence encoding a deactivated Cas endonuclease fused to an epitope repeat array comprising one or more epitopes;
b) one or more nucleic acid sequences encoding an epitope binding molecule fused to one or more DNA demethylating agents, wherein said epitope binding molecule is capable of specifically binding to an epitope of the epitope repeat array; and
c) a nucleic acid sequence encoding a guide RNA complementary to (i) a target sequence in the promoter sequence for the IRAK3 gene, (ii) a target sequence in the regulatory sequences for the IRAK3 gene or (iii) a target sequence in the IRAK3 gene, and capable of directing said Cas endonuclease to said target sequence,
   for use in a method of treatment or prophylaxis of macular degeneration in a subject, where the CRISPR-Cas vector system increases IRAK-M expression in a target cell of the subject.

As described herein, the deactivated endonuclease is a mutant form of endonuclease where the endonuclease activity has been removed by point mutations in the endonuclease domain. Although deactivated endonuclease lacks endonuclease activity, it is still able to bind gRNAs and the target DNA. The deactivated endonuclease described herein may be a dCas. Typically, Cas9 is used, but other endonucleases can be used, for example, Cas12a.

The viral delivery system described herein, or the non-viral delivery system described herein, may be used to introduce the nucleic acid systems described herein to a target cell.

In some embodiments, it can be beneficial to employ multiple vectors and/or virions carrying different payloads. For example, the nucleic acid sequences described above may be delivered via the same vector. Alternatively, the nucleic acid sequences may be delivered via multiple vectors. In an embodiment, one or more virions may each comprise at least one of the relevant components.

In some embodiments, the nucleic acid is DNA. In some embodiments, the nucleic acid is RNA.

### Additional Targeted Approaches

An aspect of the invention provides a nucleic acid comprising a nucleic acid sequence encoding a fusion protein, the fusion protein comprising:
(a) a nucleic acid binding molecule capable of binding to a target sequence in the promoter or regulatory sequences of the IRAK3 gene; and
(b) one or more transcriptional activators,
   for use in a method of treatment or prophylaxis of macular degeneration in a subject, where the fusion protein is capable of increasing IRAK-M expression in a target cell of the subject.

Also provided is a nucleic acid system comprising:
a) a nucleic acid sequence encoding a fusion protein, wherein the fusion protein comprises (i) a nucleic acid binding molecule capable of binding to a target sequence in the promoter or regulatory sequences of the IRAK3 gene and (ii) an epitope repeat array; and
b) one or more nucleic acid sequences encoding an epitope binding molecule fused to one or more transcriptional activators, wherein said epitope binding molecule is capable of specifically binding to an epitope of the epitope repeat array,
   for use in a method of treatment or prophylaxis of macular degeneration in a subject, where the nucleic acid system is capable of increasing IRAK-M expression in a target cell of the subject.

Another aspect provides a nucleic acid comprising a nucleic acid sequence encoding a fusion protein, the fusion protein comprising:
a) a nucleic acid binding molecule capable of binding to (i) a target sequence in the promoter sequence for the IRAK3 gene, (ii) a target sequence in the regulatory sequences for the IRAK3 gene or (iii) a target sequence in the IRAK3 gene; and
b) one or more DNA demethylating agents,
   for use in a method of treatment or prophylaxis of macular degeneration in a subject, where the fusion protein is capable of increasing IRAK-M expression in a target cell of the subject.

Also provided is a nucleic acid system comprising:
a) a nucleic acid sequence encoding a fusion protein, wherein the fusion protein comprising (i) nucleic acid binding molecule capable of binding to (1) a target sequence in the promoter sequence for the IRAK3 gene, (2) a target sequence in the regulatory sequences for the IRAK3 gene or (3) a target sequence in the IRAK3 gene and (ii) an epitope repeat array; and
b) one or more nucleic acid sequences encoding an epitope binding molecule fused to one or more DNA demethylating agents, wherein said epitope binding molecule is capable of specifically binding to an epitope of the epitope repeat array,
   for use in a method of treatment or prophylaxis of macular degeneration in a subject, where the nucleic acid system is capable of increasing IRAK-M expression in a target cell of the subject.

The invention also provides the following fusion proteins.

An aspect of the invention provides a fusion protein comprising:
(a) a nucleic acid binding molecule capable of binding to a target sequence in the promoter or regulatory sequences of the IRAK3 gene; and
(b) one or more transcriptional activators,
   for use in a method of treatment or prophylaxis of macular degeneration in a subject, where the fusion protein is capable of increasing IRAK-M expression in a target cell of the subject.

Another aspect provides a fusion protein comprising:
a) a nucleic acid binding molecule capable of binding to (i) a target sequence in the promoter sequence for the IRAK3 gene, (ii) a target sequence in the regulatory sequences for the IRAK3 gene or (iii) a target sequence in the IRAK3 gene; and
b) one or more DNA demethylating agents,
   for use in a method of treatment or prophylaxis of macular degeneration in a subject, where the fusion protein is capable of increasing IRAK-M expression in a target cell of the subject.

The following systems are also provided.

An aspect provides a system comprising:
a) a fusion protein, wherein the fusion protein comprises (i) a nucleic acid binding molecule capable of binding to a target sequence in the promoter or regulatory sequences of the IRAK3 gene and (ii) an epitope repeat array; and
b) one or more epitope binding molecules fused to one or more transcriptional activators, wherein said epitope binding molecule is capable of specifically binding to an epitope of the epitope repeat array,
   for use in a method of treatment or prophylaxis of macular degeneration in a subject, where the system is capable of increasing IRAK-M expression in a target cell of the subject.

Also provided is system comprising:
a) a fusion protein, wherein the fusion protein comprising (i) nucleic acid binding molecule capable of binding to (1) a target sequence in the promoter sequence for the IRAK3 gene, (2) a target sequence in the regulatory sequences for the IRAK3 gene or (3) a target sequence in the IRAK3 gene and (ii) an epitope repeat array; and
b) one or more epitope binding molecules fused to one or more DNA demethylating agents, wherein said epitope binding molecule is capable of specifically binding to an epitope of the epitope repeat array,
   for use in a method of treatment or prophylaxis of macular degeneration in a subject, where the system is capable of increasing IRAK-M expression in a target cell of the subject.

The fusion proteins and systems may be delivered to a target cell as a complex. The delivery methods described for the gene editing systems may also apply to the fusion proteins and systems described herein.

The fusion protein may further comprise a linker between the nucleic acid binding molecule and (i) the one or more transcriptional activators, (ii) the one or more demethylating agents, or (iii) the epitope repeat array.

In some embodiments, the nucleic acid binding molecule is a transcription activator-like (TAL) effector (also known as TALEs) repeat array. A fusion protein comprising a TAL effector repeat array and either (i) a transcriptional activator or (ii) a DNA demethylating agent can be used to increase endogenous expression of IRAK-M in a target cell.

TALEs are proteins secreted by some β- and γ-proteobacteria. TALEs have a modular DNA-binding domains (DBD) consisting of repetitive sequences of residues. Each repeat region comprises around 34 amino acids. The residues at position 12 and 13 determine the nucleotide specificity and are known as the Repeat Variable Diresidue (RVD). The RVD is highly variable and shows a strong correlation with specific nucleotide recognition.

TAL effector repeat domains can be engineered to each bind to one nucleotide of DNA with the specificity determined by the identities of the two hypervariable residues. To construct a protein capable of recognizing a specific DNA sequence, repeats with different specificities are simply joined together into a TAL effector repeat array. Accordingly, the TAL effector repeat array can be used to bind to target sequences in IRAK3 gene or the promoter/regulatory sequence(s) for the IRAK3 gene.

The TAL effector repeat array may be fused to either (i) a transcriptional activator or (ii) a DNA demethylating agent. Alternatively, the TAL effector repeat array may be fused to an epitope repeat array as described above.

In some embodiments, provided is a nucleic acid comprising a nucleic acid sequence encoding a fusion protein, the fusion protein comprising:
a) a TAL effector repeat array capable of binding to a target sequence in the promoter or regulatory sequences of the IRAK3 gene; and
b) one or more transcriptional activators,
   for use in a method of treatment or prophylaxis of macular degeneration in a subject, where the fusion protein is capable of increasing IRAK-M expression in a target cell of the subject.

In some embodiments, provided is a fusion protein comprising:
a) a TAL effector repeat array capable of binding to a target sequence in the promoter or regulatory sequences of the IRAK3 gene; and
b) one or more transcriptional activators,
   for use in a method of treatment or prophylaxis of macular degeneration in a subject, where the fusion protein is capable of increasing IRAK-M expression in a target cell of the subject.

In some embodiments, provided is a nucleic acid comprising a nucleic acid sequence encoding a fusion protein, the fusion protein comprising:
a) a TAL effector repeat array capable of binding to (i) a target sequence in the promoter sequence for the IRAK3 gene, (ii) a target sequence in the regulatory sequences for the IRAK3 gene or (iii) a target sequence in the IRAK3 gene; and
b) one or more DNA demethylating agents,
   for use in a method of treatment or prophylaxis of macular degeneration in a subject, where the fusion protein is capable of increasing IRAK-M expression in a target cell of the subject.

In some embodiments, provided is a fusion protein comprising:
a) a TAL effector repeat array capable of binding to (i) a target sequence in the promoter sequence for the IRAK3 gene, (ii) a target sequence in the regulatory sequences for the IRAK3 gene or (iii) a target sequence in the IRAK3 gene; and
b) one or more DNA demethylating agents,
   for use in a method of treatment or prophylaxis of macular degeneration in a subject, where the fusion protein is capable of increasing IRAK-M expression in a target cell of the subject.

In some embodiments, the nucleic acid binding molecule is a zinc finger array. A fusion protein comprising a zinc finger (ZNF) array and either (i) a transcriptional activator or (ii) a DNA demethylating agent may be used to increase endogenous IRAK-M expression.

Zinc-finger motifs are maintained by a zinc ion, which coordinates cysteine and histidine in different combinations allowing ZNFs to have the ability to interact with DNA and/or RNA. The ZNFs can be engineered to alter the DNA-binding specificity of the zinc-fingers. Tandem repeats of the zinc-finger domains (and/or engineered zinc-finger domains) can be used to target specific DNA (or RNA) sequence. Engineered zinc finger arrays may have between 3 and 6 individual zinc finger motifs and are capable of binding target sites ranging from 9 base pairs to 18 base pairs in length. Arrays with at least 6 zinc finger motifs may be preferred because they are capable of binding longer a target sequences, which increases specificity.

The zinc finger array may be fused to either (i) a transcriptional activator or (ii) a DNA demethylating agent. Alternatively, the zinc finger array may be fused to an epitope repeat array as described above. In some embodiments, the zinc finger array comprises at least 3 zinc finger motifs. In some embodiments, the zinc finger array comprises at least 6 zinc finger motifs. The zinc finger array may be capable of binding to target sequences in IRAK3 gene or the promoter/regulatory sequence(s) for the IRAK3 gene.

In some embodiments, provided is a nucleic acid comprising a nucleic acid sequence encoding a fusion protein, the fusion protein comprising:
a) a zinc finger array capable of binding to a target sequence in the promoter or regulatory sequences of the IRAK3 gene; and
b) one or more transcriptional activators,
   for use in a method of treatment or prophylaxis of macular degeneration in a subject, where the fusion protein is capable of increasing IRAK-M expression in a target cell of the subject.

In some embodiments, provided is a fusion protein comprising:
a) a zinc finger array capable of binding to a target sequence in the promoter or regulatory sequences of the IRAK3 gene; and
b) one or more transcriptional activators,
   for use in a method of treatment or prophylaxis of macular degeneration in a subject, where the fusion protein is capable of increasing IRAK-M expression in a target cell of the subject.

In some embodiments, provided is a nucleic acid comprising a nucleic acid sequence encoding a fusion protein, the fusion protein comprising:
a) a zinc finger array capable of binding to (i) a target sequence in the promoter sequence for the IRAK3 gene, (ii) a target sequence in the regulatory sequences for the IRAK3 gene or (iii) a target sequence in the IRAK3 gene; and
b) one or more DNA demethylating agents,
   for use in a method of treatment or prophylaxis of macular degeneration in a subject, where the fusion protein is capable of increasing IRAK-M expression in a target cell of the subject.

In some embodiments, provided is a fusion protein comprising:
a) a zinc finger array capable of binding to (i) a target sequence in the promoter sequence for the IRAK3 gene, (ii) a target sequence in the regulatory sequences for the IRAK3 gene or (iii) a target sequence in the IRAK3 gene; and
b) one or more DNA demethylating agents,
   for use in a method of treatment or prophylaxis of macular degeneration in a subject, where the fusion protein is capable of increasing IRAK-M expression in a target cell of the subject.

The transcriptional activator may be any of the transcriptional activators as described herein. Similarly, the DNA demethylating agent may any of the DNA demethylating agents as described herein.

In some embodiments, the nucleic acid is DNA. In some embodiments, the nucleic acid is RNA.

In some embodiments, the epitope binding molecule is an antibody or antibody-like molecule.

The viral delivery system described herein, or the non-viral delivery system described herein, may be used to introduce the nucleic acids encoding a fusion protein described herein to a target cell.

### Small Molecule Agents

Small molecule and peptide agents may be used to increase endogenous expression of IRAK-M in target cells.

In this specification, the term small molecule refers to a low molecular weight organic compound. Small molecules are able to bind specific biological macromolecules and act as an effector, altering the activity or function of the target. Due to their small size, small molecules may have the benefit of being able to pass across cell membranes to reach targets in the cell.

An aspect of the invention provides a small molecule for use in a method of treatment or prophylaxis of macular degeneration in a subject, where the small molecule increases endogenous IRAK-M expression in a target cell of the subject.

Small molecules may be used to reduce DNA methylation in the promoter sequence for the IRAK3 gene or the IRAK3 gene itself, thereby increasing accessibility of the IRAK3 gene or its promoter. Thus, in some embodiments, the small molecule reduces DNA methylation in the promoter sequence for the IRAK3 gene. In some embodiments, the small molecule reduces DNA methylation in the IRAK3 gene.

Example of small molecules that are capable of reducing DNA methylation in the promoter sequence for the IRAK3 gene or the IRAK3 gene include EPZ-6438 and azacytidine. Geng et al., 2020, Communications Biology, 3:306 describes the use of EPZ-6438 and azacytidine to induce IRAK-M expression in target cells by increasing IRAK-M transcripts. In some embodiments, the small molecule is EPZ-6438. In some embodiments, the small molecule is azacytidine.

Another small molecule shown to increase endogenous IRAK-M expression is ibudilast. Oliveros et al., 2022, Brain. awac136. (doi: 10.1093/brain/awac136) describes that ibudilast increased IRAK3 transcripts. Ibudilast is a multi-target drug, as it is a phosphodiesterase inhibitor and toll-like receptor 4 (TLR4) antagonist and has also been shown to inhibit IRAK1 activity by increasing expression of its negative regulator IRAK-M. Therefore, in some embodiments, the small molecule is ibudilast.

The small molecule may increase IRAK-M expression by recruiting one or more polypeptides that promote transcription to promoter for IRAK3. For example, Miyata et al. 2015, Nature Communications, 6:6062 show that glucocorticoids are able to upregulate IRAK-M by recruiting the glucocorticoid receptor (GR) to the IRAK-M promoter. Without wishing to be bound by theory, GR along with p65 binding to the promoter is able to result in the induction of IRAK-M transcription. In some embodiments, the small molecule is a glucocorticoid. In some embodiments, the small molecule is cortisol. In some embodiments, the small molecule is dexamethasone.

The small molecule may increase IRAK-M expression by reducing degradation of IRAK3 RNA transcripts. For example, Tong et al 2021, Science Advances, Vol 7. No. 18 show that IRAK3 mRNA transcripts are highly decorated by m⁶A modification, which promotes degradation of IRAK3 mRNA. Loss of the major m⁶A “writers”, such as the N6-adenosine-methyltransferase-like 3 (METTL3), may reduce m⁶A modification of IRAK3 mRNA, leading to reduced mRNA degradation and increased IRAK-M expression. In some embodiments, the small molecule is a METTL3 inhibitor. In some embodiments, the small molecule is STM2457 (Yankova, et al., (2021) Nature, 593, 597-601). In some embodiments, the small molecule is Cpd-564 (Wang et al., Science Translational Medicine, 2022, Vol. 14 No. 640). In some embodiments, the small molecule is UZH2 (Dolbois et al., J. Med. Chem. 2021, 64, 17, 12738-12760).

### Nucleic Acid Agents

A further aspect provides a nucleic acid for use in a method of treatment or prophylaxis of macular degeneration in a subject, where the nucleic acid increases endogenous IRAK-M expression in a target cell of a subject.

As described herein, IRAK3 mRNA transcripts are highly decorated by m⁶A modification, which promotes degradation. Loss of the major m6A “writers”, such as the methyltransferase METTL3, can reduce m⁶A modification of IRAK3 mRNA. Thus, a nucleic acid targeting METTL3 may also be used to increase expression of IRAK-M.

The nucleic acid may inhibit expression of METTL3. The nucleic acid may be capable of binding to METTL3 mRNA. In some embodiments, the nucleic acid is capable of hybridising to a target sequence in METTL3 mRNA. The nucleic acid may comprise a nucleic acid sequence which is at least partially complementary to a sequence in METTL3 mRNA. The nucleic acid may downregulate METTL3 expression, thereby increasing IRAK-M expression.

In some embodiments, the nucleic acid may be an inhibitory nucleic acid, such as antisense or small interfering RNA, including but not limited to shRNA or siRNA. In some embodiments, the nucleic acid is selected from the group consisting of an siRNA, an shRNA, a miRNA, and an ASO.

“Short or small interfering RNAs” (siRNAs) or microRNAs” (miRNAs) depending on their origin may be used to down-regulate gene expression by binding to complementary RNAs and either triggering mRNA elimination (RNAi) or arresting mRNA translation into protein. siRNA are derived by processing of long double stranded RNAs and when found in nature are typically of exogenous origin. Micro-interfering RNAs (miRNA) are endogenously encoded small non-coding RNAs, derived by processing of short hairpins. Both siRNA and miRNA can inhibit the translation of mRNAs bearing partially complimentary target sequences without RNA cleavage and degrade mRNAs bearing fully complementary sequences.

An antisense oligonucleotide (ASO) is an oligonucleotide, preferably single stranded, that targets and binds, by complementary sequence binding, to a target oligonucleotide, e.g., mRNA. Where the target oligonucleotide is an mRNA, binding of the antisense to the mRNA blocks translation of the mRNA and expression of the gene product. Antisense oligonucleotides may be designed to bind sense genomic nucleic acid and inhibit transcription or promote degradation of a target nucleotide sequence.

Another alternative is the expression of a short hairpin RNA molecule (shRNA) in the cell. shRNAs are more stable than synthetic siRNAs. A shRNA consists of short, inverted repeats separated by a small loop sequence. One inverted repeat is complimentary to the gene target. In the cell the shRNA is processed by DICER into a siRNA which degrades the target gene mRNA and suppresses expression. In an embodiment, the shRNA is produced endogenously (within a cell) by transcription from a vector. shRNAs may be produced within a cell by transfecting the cell with a vector encoding the shRNA sequence under control of an RNA polymerase III promoter such as the human H1 or 7SK promoter or a RNA polymerase II promoter. Alternatively, the shRNA may be synthesised exogenously (in vitro) by transcription from a vector. The shRNA may then be introduced directly into the cell.

Example nucleic acids which reduce expression of METTL3 can be found in CN111676222A and CN114438085A.

### Peptide and Polypeptide Agents

The agent may be a peptide or polypeptide. The term “peptide” is used herein to refer to short chains of amino acids consisting of 40 or fewer amino acids linked by peptide bonds. The term “polypeptide” is used herein to refer to large biomolecules, or macromolecules, consisting of one or more long chains of amino acid residues, each being more than 40 amino acids in length.

A peptide or polypeptide may be used to increase endogenous expression of IRAK-M in target cells.

Accordingly, another aspect provides a peptide or polypeptide for use in a method of treatment or prophylaxis of macular degeneration in a subject, where the peptide or polypeptide increases endogenous IRAK-M expression in a target cell of the subject. The agent may be polypeptide.

For example, Zacharioudaki et al., 2009, The Journal of Immunology, 182:6444-6451, describes increasing IRAK-M expression using adiponectin (globular adiponectin (gAd)). Globular adiponectin was shown to activate the Tpl2/ERK and PI3K/Akt1 signalling pathways. In particular, Zacharioudaki reported that Tlp2 mediates adiponectin signals to activate ERK1/2 and induce IRAK-M, and that activation of PI3K and its downstream effector Akt1 is also implication in the induction of IRAK-M expression. In some embodiments, the peptide or polypeptide activates ERK1/2 and/or activates PI3K and Akt1. In some embodiments, the peptide or polypeptide is adiponectin. In some embodiments, the peptide or polypeptide is globular adiponectin.

The small molecules, peptides, and polypeptides as described herein can be introduced to the target cell using any of the delivery methods described herein.

### Augmenting IRAK-M Activity

Some agents according to the invention may augment IRAK-M activity.

As described herein, IRAK-M is a negative regulator for TLR/IL-1R-induced proinflammatory cascade. IRAK-M prevents dissociation of IRAK-1 and IRAK-4 from MyD88 as well as formation of IRAK-1-TRAF6 complexes. Thus, in some embodiments the agent promotes IRAK-M binding to IRAK-1 and/or IRAK-4. In some embodiments, the agent promotes IRAK-M binding to MyD88.

An aspect provides a small molecule for use in a method of treatment or prophylaxis of macular degeneration in a subject, where the small molecule increases IRAK-M activity in a target cell of the subject.

Nguyen et al., 2022, Int. J. Mol. Sci. 2022, 23, 2552 reported that cyclic guanosine monophosphate (cGMP) is able to modulate IRAK-M activity without changing expression levels of IRAK-M. The authors report that the pseudokinase domain of IRAK-M comprises a guanylate cyclase (GC) centre that generates cGMP. The cGMP then associates with IRAK-M and contributes to mediating its anti-inflammatory activity. Accordingly, IRAK-M activity may be increased by increasing cellular cGMP levels. Cellular cGMP levels may be increased by using a nitric oxide donor (Nguyen et al., 2022). It known that the cGMP synthesis by guanylate cyclase (GC) is enhanced in response to nitric oxide (NO). Cellular cGMP levels may be increased by using riociguat. Riociguat is describes as a soluble guanylate cyclase stimulator (Lian et al. 2017. Drug Design, Development and Therapy: 11 1195-1207. In some embodiments, the small molecule is capable of stimulating guanylate cyclase (GC). In some embodiments, the small molecule increases cellular cGMP. In some embodiments, the small molecule is nitric oxide (NO) donor. In some embodiments, the small molecule is nitric oxide. In some embodiments, the small molecule is riociguat. Alternatively, the small molecule may be cGMP.

Another aspect provides a peptide or polypeptide for use in a method of treatment or prophylaxis of macular degeneration in a subject, where the peptide or polypeptide increases IRAK-M activity in a target cell of a subject. The agent may be polypeptide.

An example peptide/polypeptide is described in Taylor, J Neuroimmunol. 2005 May; 162 (0): 43-50. Taylor 2005, describes that the neuropeptide alpha-melanocyte-stimulating hormone (α-MSH) can promote IRAK-M binding to IRAK-1. In some embodiments, the peptide or polypeptide promotes IRAK-M binding to IRAK-1 and/or IRAK-4. In some embodiments, the peptide or polypeptide is α-MSH.

The small molecules, peptides, and polypeptides as described herein can be introduced to the target cell using any of the delivery methods described herein.

### Additional Therapeutic Agents

Additional therapeutic agents may also be used in the treatment or prophylaxis of macular degeneration in a subject alongside or in combination with the agents described elsewhere in this specification. These additional therapeutic agents may target other signalling pathways or processes involved in macular degeneration. Thus, the medical uses described herein may further comprise the administration of one or more additional therapeutic agents to a subject.

For example, complement activation has been strongly implicated in AMD risk and pathogenesis, particularly dry AMD risk and pathogenesis. Accordingly, the additional therapeutic agent may be an inhibitor of the complement system, such as a regulator, e.g., complement factor H (CFH) or complement factor I (CFI). The additional therapeutic agent may be a biologic that inhibits C1q, C3, C5, complement factor B (CFB), or complement factor D (CFD). Example C3 inhibitors include Pegcetacoplan (Apellis) and NGM621 (NGM Bio). An example C5 inhibitor is Avacincaptad pegol (IVERIC Bio). An example CFD inhibitor is Lampalizumab (Novartis). Gene therapy may also be used. For example, GT005 (Gyroscope), which is a CFI gene therapy. Some patients with AMD have been shown to have less CD59 present in the retina to protect cells from damage as a result of complement. Thus, in some embodiments, the additional therapeutic agent increases a soluble form of CD59 (sCD59) in target cells. An example is HMR59 (Hemera/J&J), which is a sCD59 gene therapy.

Antagonists of VEGF are also commonly used to treat AMD, particularly wet AMD. Thus, in some embodiments, the additional therapeutic agent is an anti-VEGF therapeutic effector. Anti-VEGF therapeutic agents may include ranibizumab, aflibercept, bevacizumab, brolucizumab or faricimab.

Inflammasome activation has been implicated in AMD, particularly in patients with dry AMD. In some embodiments, the additional therapeutic agent is an inhibitor of the inflammasome pathway. Examples of inhibitors of the inflammasome pathway include anakinra and canakinumab. The additional therapeutic agent may be a serine protease HtrA (gene name: HTRA1), which has been shown to be reduced in the retina of patients with dry AMD (Williams, et al. PNAS 2021 Vol. 118 No. 30 e2103617118).

The additional therapeutic agent may be a ciliary neurotrophic factor (CNTF), a neuroprotective factor.

The additional therapeutic agent may be a mitochondrial-targeted peptide antioxidant, which may inhibit oxidative stress associated with AMD, particularly dry AMD. Example a mitochondrial-targeted peptide antioxidants include SS-31 (also known as elamipretide) and SS-20 (Szeto. The AAPS Journal 2006; 8 (2) Article 32).

The additional therapeutic agent may be a senolytic molecule. For example, a senolytic molecule may be an inhibitor of Bcl-XL protein. Bcl-XL protein has been found to be upregulated in senescent retinal cells to evade apoptosis (Crespo-Garcia et a., 2021, Cell Metabolism 33, 818-832).

The additional therapeutic agent may be a senomorphic molecule targeting SASP-proinflammatory signalling networks. For example, the senomorphic molecule may be a neutralising antibody against either IL-1a or its receptor to reduce NF-κB transcriptional activities (Orjalo et al., PNAS, Oct. 6, 2009; vol. 106, no. 40:17031-17036).

The additional therapeutic agent may be an autophagy inducer, which can promote autophagy and reduce inflammation. For example, an shRNA for mTOR inhibition (Lee et al., Invest Ophthalmol Vis Sci. 2020; 61 (2): 45.).

The additional therapeutic agent may be a pigment epithelium-derived factor (PEDF), one of the serpin superfamily proteins and neuroprotective factors, which has been found to be significantly reduced in expression level in Bruch's membrane and RPE in patients with AMD (Bhutto et al. Exp Eye Res. 2006 January; 82 (1): 99-110. doi: 10.1016/j.exer.2005.05.007) and diabetic retinopathy (DR) (Ogata et al., 2002, American Journal of Ophthalmology, Vol. 134 (3): 348-353).

The additional therapeutic agent may be a small molecule, a peptide, a polypeptide, an antibody or antibody-like fragment, or a nucleic acid (e.g., an shRNA).

In this specification “antibody” includes a fragment or derivative of an antibody, a synthetic antibody, or a synthetic antibody fragment. In view of today's techniques in relation to monoclonal antibody technology, antibodies can be prepared to most antigens. The antigen-binding portion may be a part of an antibody (for example a Fab fragment) or a synthetic antibody fragment (for example a single chain Fv fragment [ScFv]). Suitable monoclonal antibodies to selected antigens may be prepared by known techniques, for example those disclosed in “Monoclonal Antibodies: A manual of techniques”, H Zola (CRC Press, 1988) and in “Monoclonal Hybridoma Antibodies: Techniques and Applications”, J G R Hurrell (CRC Press, 1982). Chimeric antibodies are discussed by Neuberger et al (1988, 8th International RICMP7164916 Biotechnology Symposium Part 2, 792-799).

The additional therapeutic agent may be administered at the same time as an agent for increasing IRAK-M expression and/or increasing IRAK-M activity as described herein. For example, a composition comprising (i) the agent for increasing IRAK-M expression and/or increasing IRAK-M activity and (ii) the additional therapeutic agent may be administered to a subject.

The additional therapeutic agent (e.g., the peptide, polypeptide, antibody or antibody-like fragment, or RNA molecule as described herein) may be encoded by a nucleic acid sequence.

Where the agent for increasing IRAK-M expression and/or increasing IRAK-M activity is a nucleic acid, the nucleic acid may further comprise a nucleic acid sequence encoding the additional therapeutic agent. The nucleic acid may be capable of driving expression of the additional therapeutic agent. The nucleic acid comprising at least two nucleic acid sequences (e.g., one encoding IRAK-M and the other encoding the additional therapeutic agent) may comprise a separate promoter for each nucleic acid sequence.

Alternatively, the nucleic acid sequence encoding the therapeutic agent may be delivered to a subject via a separate nucleic acid to the nucleic acid comprising a nucleic acid sequence encoding the agent for increasing IRAK-M expression and/or increasing IRAK-M activity (i.e., two different nucleic acids).

The nucleic acids may be delivered to a target cell via viral delivery systems or non-viral delivery systems

Where the additional therapeutic agent is an antibody (or antibody-like molecule), the antibody (or Ab-like molecule) can be encoded in a single vector via the use of a 2A self-processing peptide sequence to express a heavy chain (HC) and a light chain (LC) separately. Two promoters can be used to separate IRAK-M from the HC and the LC. Alternatively, each of IRAK-M agent, the HC and the LC may be separated by a 2A-self processing (or self-cleaving) peptide. The use of a nucleic acid sequence encoding a 2A peptide is described in detail in Fuchs et al., 2016., PLOS ONE, DOI: 10.1371/journal.pone.0158009 and Lin and Balazs, Retrovirology (2018) 15:66. In some embodiments, the 2A sequence is a foot-and-mouth disease virus 2A sequence (F2A). In some embodiments, the 2A sequence is a picornavirus 2A sequence. In some embodiments, the 2A sequence is an equine rhinitis A virus 2A sequence (E2A). In some embodiments, the 2A sequence is a porcine teschovirus-1 2A sequence (P2A). In some embodiments, the 2A sequence is a thosea asigna virus 2A sequence (T2A). Without wishing to be bound by theory, it is believed that the separation of the HC and LC (or any two sequences) occurs through a ribosomal skip mechanism which prevents the formation of the peptide bond during translation. A furin cleavage sequence may also be used to remove the 2A peptide during processing in the Golgi.

### Pharmaceutical Composition and Routes of Administration

The agents and additional therapeutic agents described herein can be formulated in pharmaceutical compositions.

Methods for administering gene therapy vectors are well known to the skilled person. IRAK-M expression vectors may be introduced systemically (e.g., intravenously or by infusion). IRAK-M expression vectors may be introduced locally (i.e., directly to a particular tissue or organ, e.g., liver). IRAK-M expression vectors may be introduced directly into the eye (e.g., by ocular injection). For recent reviews see, e.g., Dinculescu et al., 2005, “Adeno-associated virus-vectored gene therapy for retinal disease” Hum Gene Ther. 16:649-63; Rex et al., 2004, “Adenovirus-mediated delivery of catalase to retinal pigment epithelial cells protects neighbouring photoreceptors from photo-oxidative stress” Hum Gene Ther. 15:960-7; Bennett, 2004, “Gene therapy for Leber congenital amaurosis” Novartis Found Symp. 255:195-202; Hauswirth et al., “Range of retinal diseases potentially treatable by AAV-vectored gene therapy” Novartis Found Symp. 255:179-188, and references cited therein.

Administration may be peripheral, e.g. intravenous, cutaneous, subcutaneous, nasal, intramuscular or intraperitoneal. Typically, though, in the context of the invention, administration to a subject may be intraocular. In some embodiments, administration to a subject may be intravitreal, subretinal, suprachoroidal, or periocular. In some embodiments, administration is by injection or infusion. In some embodiments, administration is by subretinal injection. In some embodiments, administration is topical. In other embodiments, administration by electroporation.

Typically, the retina can be accessed via three distinct routes: intravitreal, subretinal, and suprachoroidal. The subretinal injection is typically an invasive surgical procedure in which the therapeutic composition is delivered between the photoreceptors and the RPE. This vitro-retinal technique can require an operating room and is usually performed under general anaesthesia. Intravitreal injections (IVIs) on the other hand, do not need to be performed in an operating room. Suprachoroidal injections are less invasive than subretinal injection and involve accessing the retina by injecting into the space between the choroid (overlaying the RPE) and the sclera (Sahu B et al. Biomolecules 2021, 11, 1135).

Administration is preferably in a “prophylactically effective amount” or a “therapeutically effective amount”, this being sufficient to show benefit to the individual. The actual amount administered, and rate and time-course of administration, may depend on the individual subject and the nature and severity of their condition.

Pharmaceutical compositions may comprise, in additional to one of the above substances, a pharmaceutically acceptable excipient, carrier, buffer, stabiliser or other material well known to those skilled in the art. Such substances should be non-toxic and should not interfere with the efficacy of the active ingredient.

The nucleic acid-containing compositions of the invention can be stored and administered in a sterile physiologically acceptable carrier, where the nucleic acid is dispersed in conjunction with any agents which aid in the introduction of the DNA into cells.

Various sterile solutions may be used for administration of the composition, including water, PBS, ethanol, lipids, etc. The concentration of the DNA will be sufficient to provide a therapeutic dose, which will depend on the efficiency of transport into the cells.

Gene therapy vectors must be produced in compliance with the Good Manufacturing Practice (GMP) requirements rendering the product suitable for administration to patients. Disclosed herein are gene therapy vectors suitable for administration to patients including gene therapy vectors that are produced and tested in compliance with the GMP requirements. Gene therapy vectors subject to regulatory approval must be tested for potency and identity, be sterile, be free of extraneous material, and all ingredients in a product (i.e., preservatives, diluents, adjuvants, and the like) must meet standards of purity, quality, and not be deleterious to the patient. For example, the nucleic acid preparation is demonstrated to be mycoplasma-free. See, e.g., Islam et al., 1997, An academic centre for gene therapy research and clinical grade manufacturing capability, Ann Med 29, 579-583.

Pharmaceutical compositions may be prepared using a pharmaceutically acceptable “carrier” composed of materials that are considered safe and effective. The term “carrier” refers to diluents, binders, lubricants and disintegrants. Those with skill in the art are familiar with such pharmaceutical carriers and methods of compounding pharmaceutical compositions using such carriers. “Pharmaceutically acceptable” refers to molecular entities and compositions that are “generally regarded as safe”, e.g., that are physiologically tolerable and do not typically produce an allergic or similar untoward reaction, such as gastric upset and the like, when administered to a human. In some embodiments, this term refers to molecular entities and compositions approved by a regulatory agency of the US federal or a state government, as the GRAS list under section 204(s) and 409 of the Federal Food, Drug and Cosmetic Act, that is subject to premarket review and approval by the FDA or similar lists, the U.S. Pharmacopeia or another generally recognised pharmacopeia for use in animals, and more particularly in humans.

The pharmaceutical compositions provided herein may include one or more excipients, e.g., solvents, solubility enhancers, suspending agents, buffering agents, isotonicity agents, antioxidants or antimicrobial preservatives. When used, the excipients of the compositions will not adversely affect the stability, bioavailability, safety, and/or efficacy of the active ingredients. Thus, the skilled person will appreciate that compositions are provided wherein there is no incompatibility between any of the components of the dosage form. Excipients may be selected from the group consisting of buffering agents, solubilizing agents, tonicity agents, chelating agents, antioxidants, antimicrobial agents, and preservatives.

A composition may be administered alone or in combination with other treatments, either simultaneously or sequentially dependent upon the condition to be treated.

The terms “treatment”, “treat”, or “treating” are used herein to refer to the reduction in severity of a disease or condition, the reduction in the duration of a disease; the amelioration or elimination of one or more symptoms associated with a disease or condition, or the provision of beneficial effect to a subject with a disease or condition. The term also encompasses prophylaxis of a disease or condition or its symptoms thereof. “Prophylaxis” is known in the art to mean decreasing or reducing the occurrence or severity of a particular disease outcome. For example, delaying progression of cancer in a subject.

As used herein, the term “subject” refers to a human or any non-human animal (e g, mouse, rat, rabbit, dog, cat, cattle, swine, sheep, horse or primate). In many embodiments, a subject is a human being. A subject can be a patient, which refers to a human presenting to a medical provider for diagnosis or treatment of a disease. The term “subject” is used herein interchangeably with “individual” or “patient.” In some embodiments, the subject is human. A subject can be afflicted with or is susceptible to a disease or disorder but may or may not display symptoms of the disease or disorder. In some embodiments, the subject is affected or is likely to be affected with a retinal disease, in particular macular degeneration.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### EXAMPLES

### Example 1

Dry age-related macular degeneration (AMD) represents a major and growing unmet clinical need—it is currently untreatable, affects hundreds of millions of people and is a significant burden on society and healthcare budgets. A low-grade, chronic inflammation plays a critical role in the progression of AMD, but molecular mechanisms that initiate the dysregulation in immune responses and drive the pro-degenerative cues remain poorly understood.

As demonstrated below, expression of an anti-inflammatory molecule, named interleukin-1 receptor associated kinase (IRAK)-M, was identified in the retinal pigment epithelium (RPE) of both humans and mice. IRAK-M expression level was reduced with age and by oxidative stress, and also decreased in AMD donor eyes compared to age-matched controls. Mice deficient in IRAK-M displayed AMD-like phenotypes at earlier ages and were more susceptible to various oxidative insults than wildtype mice. Mechanistically, the absence of IRAK-M disrupted RPE cell homeostasis and function, as evidenced by altered mitochondrial metabolism, accelerated cellular senescence, and elevated inflammatory cytokine production. Conversely, augmentation of IRAK-M expression in RPE cells protects against oxidative or immune stress. The data reveal an underlying mechanism of neurodegeneration in the eye, due to pro-inflammatory processes which are compounded further by ageing and oxidative stress. IRAK-M is therefore a critical immunoregulatory molecule in the maintenance of RPE metabolic health and function.

### Materials and methods

### Mice

Irak3^{-/-} mice were obtained from Jackson Laboratory (B6.129S1-Irak3tm1Flv/J, stock #007016). Not reported previously (90), we found Rd8 mutation of Crb1 gene within this strain using established PCR genotyping protocol (90). Therefore, the mice were backcrossed with C57BL/6J (wildtype or WT, Charles River Laboratories, Portishead, UK) and Rd8-negative Irak3−/−genotype was established (not shown). The breeding colonies were maintained in the Animal Services Unit of the University of Bristol. All mice were kept in the animal house facilities of the university according to the Home Office Regulations. Treatment of the animals conformed to the Association for Research in Vision and Ophthalmology (ARVO) statement for the Use of Animals in Ophthalmic and Vision Research. The methods were carried out in accordance with the approved University of Bristol institutional guidelines and all experimental protocols under Home Office Project Licences 30/2745 and pp 9783,504 were approved by the University of Bristol Ethical Review Group.

### Human Ocular Tissues and Sample Processing

Human donor eyes or ocular tissue surpluses to corneal transplantation (without recorded ocular disease) were obtained from National Health Service (NHS) Blood and Transplant Services after research ethics committee approval (20/LO/0336), with experiments conducted according to the Declaration of Helsinki and in compliance with UK law. The age and sex of human eye samples are indicated in figures. For immunohistochemistry, the tissue was fixed in 4% formaldehyde in PBS, embedded in optimum cutting temperature compound and frozen in dry ice followed by storage at −80° C. before preparation of cryosections. For western blotting, dissected RPE/choroid tissue was crushed in 400 μL Pierce® RIPA buffer containing Protease and Phosphatase Inhibitors (Thermofisher Scientific, Paisley, UK) for protein extraction.

### Data Mining

Potential datasets for analyses of AMD-related changes were chosen according to availability of processed RNA-Seq data via the NCBI GEO Datasets, using search terms ‘AMD retina’ and ‘AMD RPE’. The Kim et al. RNA-Seq dataset in retinal and RPE-Choroid-Scleral (RCS) Homo Sapiens tissues (GEO accession GSE99248) was used, which includes both antisense and sense transcriptome data from 7 donor eyes without recorded ocular diseases (age range 83-92y, 3 females and 5 males), and 8 AMD donor eyes (age range 83-95y, 5 females and 2 males). The AMD eyes were characterized at various stages, including 2 early AMD, 1 dry AMD with RPE atrophy, 3 late dry AMD, and 1 late wet AMD. The dataset samples were sorted according to tissue-type (either retina or RCS) and phenotype (AMD or normal); normal samples corresponding to each tissue-type were used as controls. Geometric means were calculated for each group to calculate fold change for each gene, and unpaired two-tailed t-tests were used to calculate p-values. Genes with significant p-values (<0.05) were compiled into gene lists for analyses (fold change >2 for upregulation, <0.5 for downregulation). The same procedure was conducted for both the mRNA and antisense datasets. Compiled gene-lists were uploaded to the Metascape online analysis resource (91) for pathway enrichment analyses. Gene-sets where members are significantly overrepresented in the input gene-lists were reported. Heatmaps of significantly enriched clusters were produced.

### Antibodies

Rabbit polyclonal anti-IRAK-M, rabbit polyclonal anti-c-Fos (phospho T325), rabbit monoclonal anti-c-Fos antibody, rabbit polyclonal anti-HMGB1, rabbit monoclonal anti-c-Jun, rabbit monoclonal anti-c-Jun (phosphor S63), rabbit monoclonal anti-p21, rabbit monoclonal anti-Lamin B1, mouse monoclonal anti-RPE65, mouse monoclonal anti-Rhodopsin and goat polyclonal anti-8 hydroxyguanosine were all purchased from Abcam (Cambridge, UK). Rabbit polyclonal anti-ZO-1 was from Thermofisher Scientific. Rat monoclonal anti-CD11b (M1/70) was from BD Biosciences (Wokingham, UK). Secondary antibodies used in western blotting, including HRP-conjugated goat anti-rabbit and anti-mouse IgG were from New England Biolabs (Hitchin, UK). Secondary antibodies used in immunostaining including Alexa Fluor 488-goat anti-rabbit or anti-mouse IgG, Alexa Fluor 488-rabbit anti-rat IgG, Alexa Fluor 555-goat anti-rabbit IgG, and Alexa Fluor 488-donkey anti-goat IgG were from Thermofisher Scientific.

RPE Cell Lines, Primary Cells, iPSC Derived RPE Cells and Treatment

A human RPE cell line ARPE-19 (American Type Culture Collection) and a mouse RPE cell line B6-RPE07 (a gift from Prof. Heping Xu, Belfast) (92) were maintained in DMEM medium supplemented with 10% FBS, 1% L-glutamine, 1 mM sodium pyruvate, 60 μM 2-mercaptoethanol, 1% penicillin/streptomycin (complete medium) at 37° C. in an atmosphere of 5% CO2. Cells were passaged with a split ratio of 1:5 using trypsin/EDTA and allowed to recover for 2 days in complete medium prior to treatment.

Primary murine RPE cells were isolated and cultured as previously reported (79). Briefly, eyes from WT or Irak3−/− mice were enucleated and cleaned using angles scissors to ensure no connective tissue remained. After cornea and lens were removed, the eyes were incubated at 37° C. in hyaluronidase for 45 min, and in Hank's balanced salt solution (HBSS) with 10 mM HEPES for a further 30 min before retinas removed via incision. Following incubation at 37° C. in trypsin/EDTA for 45 min, eyecups were then transferred into HBSS with 20% heat-inactivated FBS and shaken gently to allow the RPE to detach. The RPE sheets were further incubated in trypsin/EDTA for 1 min to allow the formation of single cell suspensions. The resulting RPE cells were resuspended in alpha MEM basal medium supplemented with 1% N1 Medium Supplement, 1% L-glutamine, 1% penicillin-streptomycin and 1% nonessential amino acid solution (NEAA), 20 μg/l hydrocortisone, 250 mg/l taurine, 0.013 μg/l triiodo-thyronin and 5% FBS. Cell purity was confirmed by immunoblotting for RPE65 and rhodopsin as we described before (23). The cells were seeded to laminin-precoated Seahorse XF cell culture plates (Agilent Technologies, Santa Clara, California, USA) or 8-well chamber slides (Corning GmbH, Wiesbaden, Germany) at a density of 25,000/cm2. After the first 72 h of incubation, serum was removed from the medium. The culture medium was changed twice a week. The cells were used for experiments 7-10 days post isolation.

Primary human RPE cells (H-RPE) were purchased from Lonza (Slough, UK). The cells were maintained in RPE Basal Medium supplemented with 2% L-glutamine, 0.5% FGF-B and 0.1% GA-1000, and sub-cultured at ratio of 1:3 using trypsin/EDTA for no more than 4 passages. The H-RPE cells were plated to 24-well plates at a density of 10,000 cells/cm2. After overnight incubation, the cells were used for induction of oxidative stress.

Human fibroblast-induced pluripotent stem cells (IPSCs) derived from a healthy donor were a kind gift from Prof. Peter Coffey from UCL (78). iPSC colonies were cultured on Matrigel hESC-qualified matrix (BD Biosciences, Wokingham, UK) in E8 (Thermofisher Scientific). Once 80% confluent, the media was changed to Differentiation Media containing Knockout-DMEM, 20% Knockout Serum Replacement, 1% non-essential amino acids (NEAA), 1% Glutamax and 0.2% 2-mercaptoethanol (all from Thermofisher Scientific). Cultures were fed twice weekly and for at least a further 8 weeks until pigmented foci were observed. These pigmented foci were isolated manually and seeded to Matrigel-precoated 96-well plates or Seahorse XF plates with a density of 50,000 cells/cm2 for induction of oxidative or immune stress and analyses of metabolic function.

The cells from different sources were either continuously treated with different concentrations of paraquat (PQ), hydrogen peroxide (H₂O₂) or lipopolysaccharide (LPS, all from Sigma-Aldrich, Poole, UK) for up to 72 h, or under a repeated exposure to PQ or H₂O₂ for 2 h every day for a total of 7 days (21). In some experiments, RPE cells were pretreated with a c-Jun inhibitor (SP600125, Sigma-Aldrich) or c-Fos inhibitor (T-5224, Cambridge Bioscience, Cambridge, UK) for 2 h before addition of PQ.

### Immunohistochemistry

To examine the expression pattern of IRAK-M in human and mouse retinas, human eye tissue from a 20-year-old donor (no recorded ocular disease) or enucleated eyes from 8-week-old WT mice were fixed with 4% (for human tissue) or 2% (for murine tissue) paraformaldehyde (PFA) before 12-μm thick cryosections prepared on a cryostat. The sections were permeabilized with 0.1% Triton X-100, blocked with 10% normal donkey serum, 5% BSA plus 0.3 M glycine before incubation with rabbit anti-IRAK-M (1:1000) and mouse anti-Rhodopsin (1:500) or mouse anti-RPE65 (1:100) overnight at 4° C. After wash, sections were incubated with donkey anti-rabbit IgG conjugated with Alexa Fluor 555 and donkey anti-mouse IgG with Alexa Fluor 647 (both 1:1000). DAPI counterstain was used to show nuclei in sections. Tissues were washed and mounted in Vectashield antifade medium and examined by confocal microscopy.

To prepare mouse retinal and RPE/choroid wholemounts, enucleated eyes were initially fixed in 2% PFA overnight. After dissection of the eyes, the retinal and RPE/choroidal tissues were blocked and permeabilized in 10% normal goat serum, 5% BSA, 0.3 M glycine with 0.3% Triton X-100 in PBS for 2 hours, followed by incubation with the rat anti-CD11b (1:200) in 1% BSA with 0.15% Triton X-100 at 40C overnight. After thorough wash, samples were further incubated with Alexa Fluor 488-goat anti-rat IgG (1:400). Tissues were counterstained with DAPI and flat-mounted for observation by confocal microscopy.

Cell apoptosis in the retinal and RPE/choroid wholemounts was determined by TUNEL staining using an In Situ Cell Death Detection Kit, TMR Red (Roche Diagnostics, Burgess Hill, UK) as previously described (14).

Human iPSC derived RPE cell cultures were fixed with 2% PFA and permeabilization with 0.1% Triton X-100. After blocking with 10% normal goat serum, cells were incubated with a polyclonal rabbit anti-ZO-1 (1:200) overnight at 4° C., followed by labelling with goat anti-rabbit conjugated with Alexa Fluor 488 (1:400). Nuclei were detected with DAPI.

### Western Blot

Protein extraction from tissue or cells was prepared using Pierce® RIPA lysis buffer containing Halt™ Protease and Phosphatase Inhibitors (Thermofisher Scientific). Protein concentrations were measured using Pierce™ BCA protein assay kit (Thermofisher Scientific). 5-15 μg protein for each sample was mixed with Tris-Glycine SDS Sample Buffer (1:2) and reducing reagent (1:10, Thermofisher Scientific), followed by denaturing at 80° C. for 2 min. After separation using Novex™ 4-20% Tris-Glycine Mini Gels (Thermofisher Scientific), proteins were transferred to PVDF membrane (Thermofisher Scientific), before blocking with 5% w/v milk in Tris-Buffered Saline (TBS)+Tween 20 (TBS-T; 0.1% v/v). Blots were incubated with primary antibodies for IRAK-M (1:2000), c-Jun (1:1000), phosphor-c-Jun (1:1000), c-Fos (1:1000), phosphor-c-Fos (1:1000), p21 (1:1000), lamin B1 (1:1000) or β-actin (1:2000) at 4° C. overnight. After thorough washing, blots were incubated with appropriate secondary antibody, anti-rabbit HRP (1:2000) or anti-mouse HRP (1:2000; both from Cell Signaling Technologies, London, UK). Chemiluminescence was detected by Amersham ECL reagents (Sigma-Aldrich) and developed using Hyperfilm™ ECL film (Sigma-Aldrich) and X developer.

### Multiplex Cytokine Array and Enzyme-Linked Immunosorbent Assay (EIA)

Supernatants of ARPE-19 or BMMϕ cultures, or mouse sera prepared from lateral tail vein sampling were examined for the concentration of inflammatory cytokines using the LEGENDplex Human or Mouse Cytokine Array Kit (BioLegend, London, UK) according to the manufacturer's instructions. The concentration of HMGB1 in ARPE-19 cell culture supernatant was determined by a direct EIA using a polyclonal anti-HMGB1 (Abcam) as the manufacturer's protocol.

### Seahorse Metabolic Analysis

Seahorse XFp or XFe96 cell culture miniplates, sensor cartridges with utility plates, and all reagents for Mito Stress tests were obtained from Agilent Technologies. Different RPE cells were incubated in Seahorse XF DMEM (pH 7.4) containing 25 mM glucose, 1 mM pyruvate, and 2 mM glutamine in 37° C. incubator without CO2 for 45 min (23). Oligomycin (ATPase inhibitor, 1 μM), FCCP (protonophoric uncoupler, 0.5 μM) and antimycin A/rotenone (electron transport inhibitors, 1 μM) were injected where indicated and the oxygen consumption rate (OCR, pmol O2/min) and extracellular acidification rate (ECAR, mpH/min) were measured in real time. The measurement rates were normalized by total protein content analyzed using a BCA assay. Metabolic parameters were calculated using the following formulae: nonmitochondrial respiration (minimum OCR after antimycin A/rotenone injection), basal respiration (difference between OCR before oligomycin and nonmitochondrial respiration), maximal respiration (difference between maximum OCR after FCCP injection and nonmitochondrial respiration), H+ (proton) leak (difference between minimum OCR after oligomycin injection and nonmitochondrial respiration), ATP production (difference between OCR before oligomycin injection and minimum OCR after Oligomycin), spare respiratory capacity (difference between maximal respiration and basal respiration), glycolysis (maximum EACR before oligomycin injection), maximal glycolytic capacity (maximum ECAR after oligomycin injection) and glycolytic reserve (difference between maximal glycolytic capacity and glycolysis) (93).

### Senescence Associated β-Galactosidase Staining

Primary murine RPE cells cultured in laminin-precoated 8-well chamber slides (Corning GmbH, Wiesbaden, Germany) were treated with 0.25 mM PQ or H₂O₂ for 2 hours each day for a total of 7 days for induction of senescence (21). A fluorescence-based live cell senescence β-galactosidase (SA-β-Gal) assay kit (Enzo Life Sciences, Exeter, UK) was used to quantify cellular senescence according to the manufacturer's instructions. Briefly, the cells were incubated with Pretreatment Solution at 37° C. for 2 hours, followed by addition of SA-β-Gal Substrate Solution (1:200). After a further 4 hours incubation, the cells were thoroughly rinsed with PBS and captured via the GFP channel (480 nm excitation, 520 nm emission) on an Evos FL Fluorescence Microscope.

### Chromatin Immunoprecipitation (ChIP)

A One-step ChIP kit (Abcam) was used to identify whether AP-1 subunits c-Jun and c-Fos are transcription factors of IRAK-M in RPE cells. Subconfluent ARPE-19 cells were fixed with 1% formaldehyde for 15 min and quenched with 0.125 M glycine. Chromatin was isolated by adding chromatin lysis buffer, followed by disruption with a Dounce homogenizer. Lysates were sonicated to shear the DNA to an average length of 300-500 bp. Cross-linked chromatin fragments (from 1×106 cells) were immunoprecipitated with ChIP-grade antibodies against c-Jun or c-Fos (Cell Signaling, London, UK), or irrelevant IgG control. The precipitated DNA was amplified by PCR to amplify the IRAK-M Transcription Start Site (TSS). The primer sequences (38) are as follows: forward 5′-TGTGGCCAGGCGGACGCAG-3′ (SEQ ID NO: 34); reverse: 5′-AGGTCGAACAGCAGCGTGT-3′ (SEQ ID NO: 35).

### Cytotoxicity Assay

At various time points, RPE cell culture supernatants were collected and chemical-induced RPE cytotoxicity was assessed using a Lactate Dehydrogenase (LDH) detection kit (Abcam) according to manufacturer's instructions. Activity of released LDH was normalized to the LDH value of RPE lysates (100% cytotoxicity).

### Detection of Autophagy Flux

The formation of autophagosome and autolysosome in RPE cells was monitored through LC3B protein localization using the Premo™ Autophagy Tandem sensor RFP-GFP-LC3B Kit (Thermofisher Scientific) according to the manufacturer's instructions. The RFP-GFP-LC3B sensor kit uses the high transduction efficiency and minimal toxicity of BacMam 2.0 expression technology, enabling the detection of LC3B positive, neutral pH autophagosomes in green fluorescence (GFP) and LC3B positive acidic pH autolysosome in red fluorescence (RFP). The RFP and the GFP genes included in this chimera are TagRFP and Emerald GFP, respectively. Briefly, ARPE-19 cells were transduced with a mixture of TagRFP-LC3B and Emerald GFP-LC3B at a MOI of 30 in cell culture medium overnight, before the addition of chemicals for 24 hours. LC3B-positive puncta (green for autophagosomes and red for autolysosomes) were analyzed using fluorescence microscopy and quantified using Image J.

### Quantitative RT-PCR (QRT-PCR)

Total RNA was isolated using TRIzol reagent (Thermofisher Scientific). One μg of total RNA was treated with RQ1 RNase-free DNase before reverse-transcription using the ImProm-II™ Reverse Transcription System (Promega). cDNA was amplified using the PowerUp SYBR® Green PCR Master Mix Reagent (Thermofisher Scientific) on a QuantStudio Real-Time PCR System. Primer sequences were designed using the Primer-BLAST (NCBI): Mouse Irak3, forward 5′-GACCAGCTCCAACCCAAACT (SEQ ID NO: 36), reverse 5′-GCCACCGCCGGTCATATTTA (SEQ ID NO: 37); Human Irak3, forward 5′-CCCACTCCCTTGGCACATTC (SEQ ID NO: 38), reverse 5′-AGCATGGTTGAACGTTGTGC (SEQ ID NO: 39); Mouse Irak1, forward 5′-CAGAGGTGGAACAGCTATCAAG (SEQ ID NO: 40), reverse 5′-CATTGGGCAAGAAGCCATAAAC (SEQ ID NO: 41); Mouse Irak4, forward 5′-AAAGGACAGGACATCCGTAATG (SEQ ID NO: 42), reverse 5′-TCGCTGGACTCTACACTTCT (SEQ ID NO: 43); mouse Rps29, forward 5′-ACGGTCTGATCCGCAAATAC (SEQ ID NO: 44), reverse 5′-ATCCATTCAAGGTCGCTTAGTC (SEQ ID NO: 45).

### In Vivo Induction of Retinal Degeneration

Retinal degeneration in WT or Irak3−/− mice were induced by paraquat (PQ, a prooxidant) or light-induced oxidative damage as previously described (22, 35, 36). Mice aged 6-8 weeks were anesthetized by intraperitoneal injection of 200 μl of Vetelar (ketamine hydrochloride 100 mg/ml, Pfizer, Sandwich, UK) and Rompun (xylazine hydrochloride 20 mg/ml, Bayer, Newbury, UK) mixed with sterile water in the ratio 0.6:1:84. Pupils were dilated using 1% tropicamide and 2.5% phenylephrine (both from Chauvin, Essex, UK). A drop of Viscotears (Novartis, London, UK) was then applied to cover the surface of the eye before the following procedures.

For PQ-induced retinal degeneration (22, 36), administration of PQ (0.375-1.5 mM, Sigma-Aldrich, Poole, UK) or PBS in contralateral eye, was delivered by 2 μl intravitreal injection conducted under a surgical microscope.

For light-induced retinal degeneration (LIRD) (35), fundus camera-delivered intense light was delivered to retinas through a Nikon D80 digital camera that was connected to an endoscope with a 5-cm long teleotoscope. The position of the mouse was adjusted using stage controls to allow the cornea to contact the end of teleotoscope and the optic disk at the centre of the fundus image. Light was applied to one eye at an intensity of 100 klux for a one-time exposure of 20 minutes. The light intensity was regularly measured using a light meter to ensure equal illumination. The contralateral eye was left without light challenge as control.

### Optical Coherence Tomography (OCT)

At selected time-points during lifespan or after induction of retinal degeneration in mice, pupils were dilated, and animals anaesthetised for clinical assessment. The Micron IV retinal imaging microscope (Phoenix Research Laboratories, Pleasanton, CA) was used to capture OCT scans, and brightfield and fluorescence fundal images. Prior to imaging, the Micron IV CCD and OCT were calibrated in accordance with the manufacturer's protocol. The gain was set to +3 dB and the FPS to 15, or +12 dB and 2 for brightfield and GFP fluorescence imaging, respectively. OCT images were used to evaluate the retinal structure and thickness using ImageJ (47).

Plasmid Transfection and siRNA

To activate IRAK-M or c-Jun expression in ARPE-19 cells, the cells were plated in 48-well plates for reach 70-80% confluence. The CRISPR activation plasmid (Santa Cruz Biotechnology) was used to upregulate the expression of endogenous gene expression. For each transfection, 0.3 μg of plasmid DNA were mixed with 1.5 μl Lipofectamine 3000 and 1 μl P3000 reagent and left for 15 min. The transfection complex was then added to the cells and left for 48 hours with reduced serum (1% FBS), followed by western blot analysis of protein expression. Non-targeting CRISPR Plasmid (Santa Cruz Biotechnology) serves as a negative control.

To induce stable expression of exogenous IRAK-M gene in B6-RPE07 cells, 70% confluent cells in 48-well plates were transfected with 0.3 μg of control pUNO1 plasmid (cat. no. puno1-mcs) or pUNO1 plasmids bearing the human IRAK-M (cat. no. puno1-hirakm) or mouse IRAK-M (cat. no. puno1-mirakm) using Lipofectamine 3000 as above. The plasmids were all from InvivoGen, Toulouse, France. Two days post transfection, the media were replaced by selective culture media containing 10 μg/ml Blasticidin. The stable transfectants were selected and expanded over 3 weeks, and stable IRAK-M expression were determined by qRT-PCR or western blot.

To knockdown IRAK-M expression in ARPE-19 cells, the siRNA specific to human IRAK-M (Santa Cruz Biotechnology, Heidelberg, Germany) was utilized according to the manufacturer's instructions. The siRNAs were mixed with Lipofectamine 3000 reagent (Thermofisher Scientific) to form the transfection complex, prior to addition to RPE culture medium at a final concentration of 40 nM. Non-silencing siRNA was used as a negative control. IRAK-M expression was determined by western blot 48 hours post transfection.

### Statistics

Results are presented as means±standard deviation (SD). Statistical analysis was performed using an unpaired two-tailed Student's t-test between two groups. Tests for normal distribution and homogeneity of variance and comparisons between more than two groups were conducted using one-way ANOVA. A two-way ANOVA was used to assess the interrelationship of two independent variables on a dependent variable, followed by the Kruskal-Wallis test with Bonferroni correction for post hoc comparisons. Differences between groups were considered significant at P<0.05. Statistical analyses were conducted using GraphPad Prism 7.0.

### Results

### IRAK-M is Predominantly Expressed by RPE in the Retina

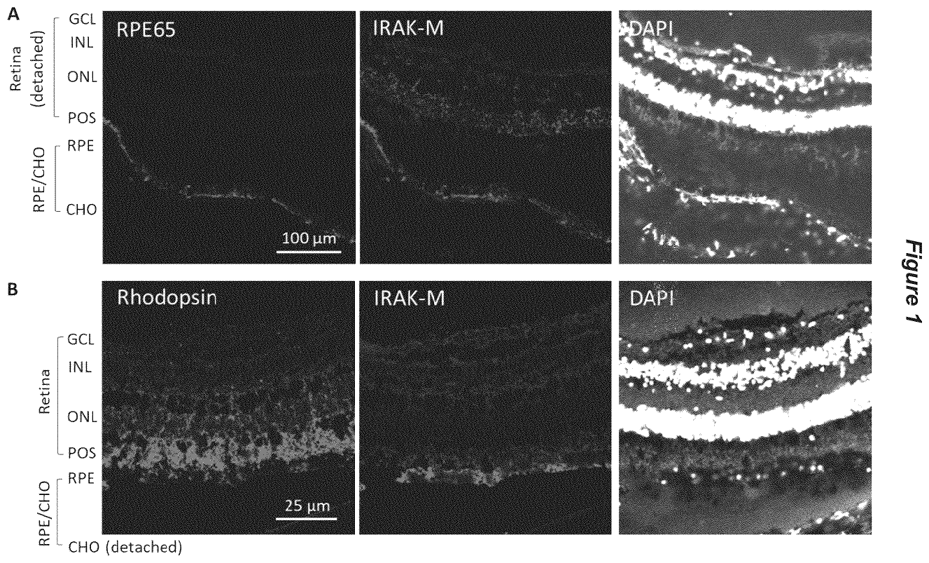

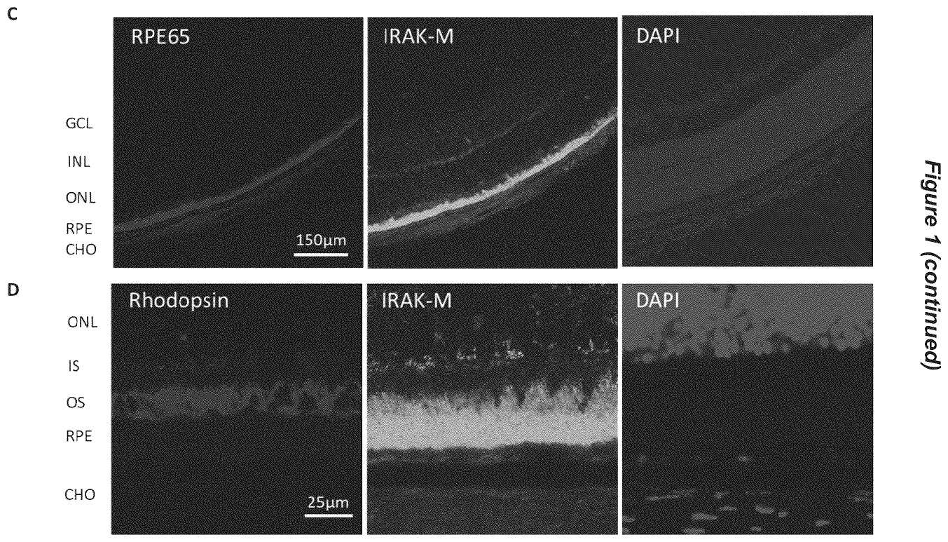

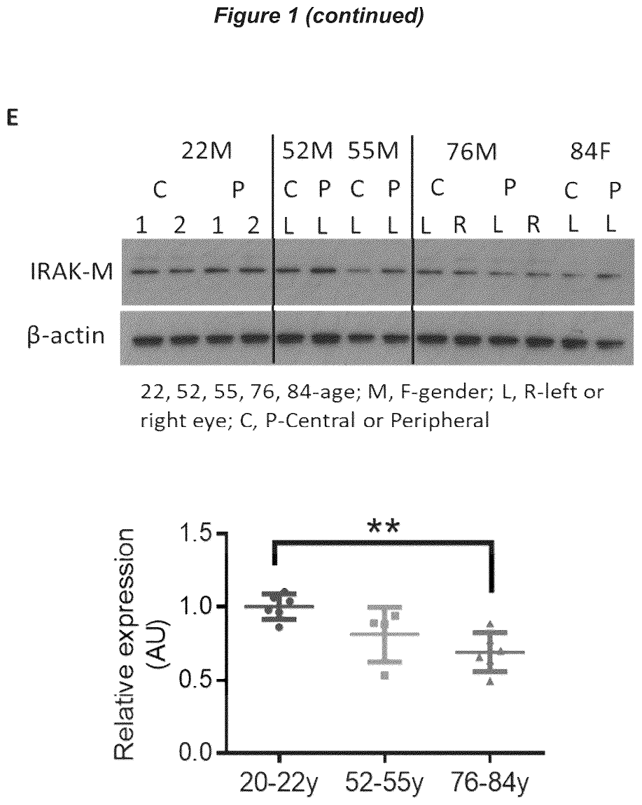

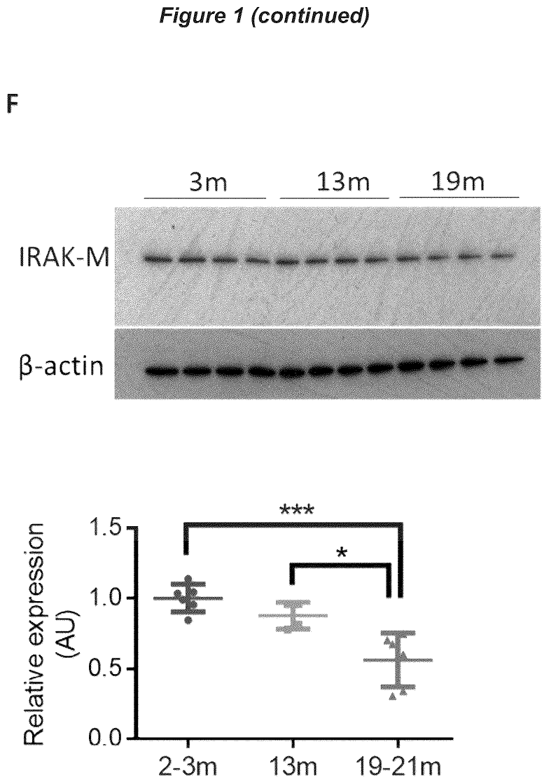

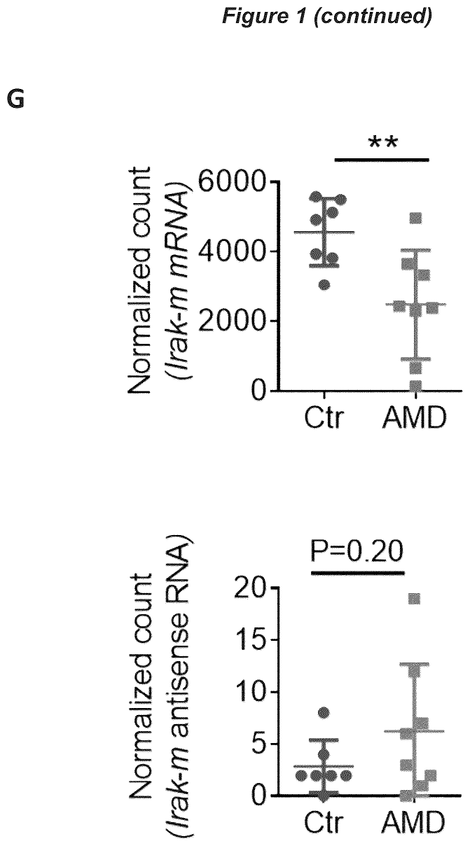

It was previously shown that IRAK-M transcripts were expressed in a murine RPE cell line (B6-RPE07) (13). To identify the tissue expression pattern in retinas, we performed immunohistochemistry on human retinal sections from a young donor eye (20y old) without recorded ocular disease, showing strong immunopositivity of IRAK-M localized at the RPE layer (stained using anti-RPE65, with counterstain of anti-Rhodopsin and DAPI) (FIGS. 1A and 1B). Weaker immunopositivity of IRAK-M was observed within the ganglion cell layer (GCL), inner plexiform layer (IPL), outer plexiform layer (OPL), outer nuclear layer (ONL), photoreceptor outer segment (POS) and choroid (FIGS. 1A and 1B). Negative controls with primary antibody omitted did not show significant fluorescence signal. Consistent with human samples, mouse retinal sections (8-weeks old) demonstrated expression of IRAK-M primarily in the RPE within the retina and choroid (FIGS. 1C and 1D).

### IRAK-M Expression is Reduced in Aged RPE and in AMD

Dysregulated inflammation is typical with age, the primary risk factor of AMD. Characteristics of immune activation in RPE, including lipofuscin and drusen formation and inflammasome activation, are evident during the progression of AMD (32). We hypothesized that IRAK-M, a vital immune regulator, alters in expression with age and in AMD.

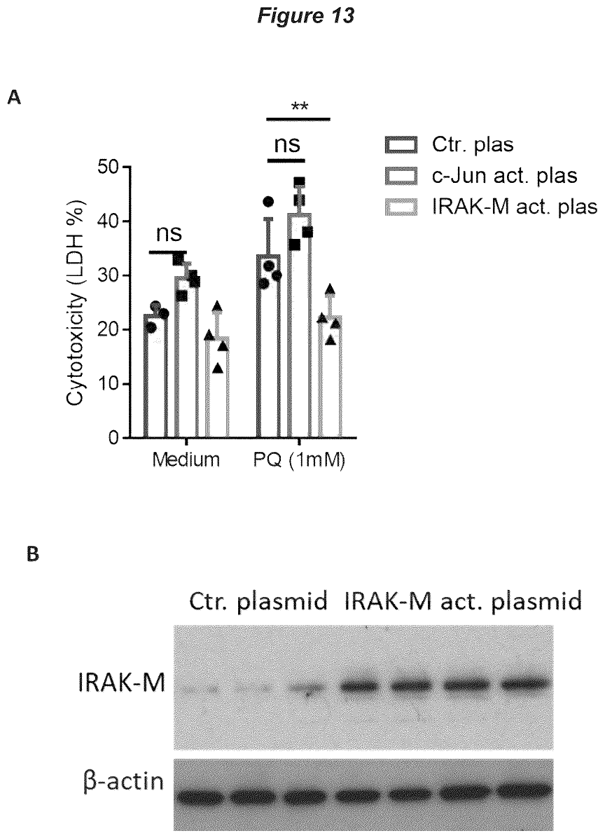

In human RPE/choroidal protein lysates from donor eyes (all without recorded ocular disease), western blot analyses demonstrated significantly decreased IRAK-M expression in elderly samples (76-84y; when AMD prevalence rate sharply increases (33)), compared to younger age (20-22y) (FIG. 1E and FIG. 9). In mid-years (52-55y) compared to young there was a reduction although not statistically significant (FIG. 1E). Similarly, 19m-old mice evinced reduced IRAK-M expression in RPE compared to young mice (2-3m, FIG. 1F) or 13m old mice.

We performed data mining on RNA-Seq datasets that include both sense and antisense transcripts (GEO accession number GSE99248) (34). There were more than 1000 genes changed at the mRNA level, and more than 3000 genes changed at the antisense RNA level in AMD-derived RPE/choroid/sclera complex compared with age-matched normal controls. The AMD-associated genes are involved in various biological processes, in which the top GO clusters are related to immune responses, cytoskeleton reorganization, extracellular matrix organization, regulation of MAPK cascade, lipid metabolism and cell apoptosis (FIG. 10). By focusing on IRAK family genes, we found that only the level of IRAK3 mRNA in AMD RPE/choroid/sclera showed a significant decrease, compared to age-matched normal controls (FIG. 1G), while antisense RNA specific to IRAK3 did not show significant difference (FIG. 1G). None of the mRNA or antisense RNA of other IRAK family members (IRAK1, IRAK2 and IRAK4) showed differences between AMD and controls (FIG. 11).

### Oxidative Stress Imitates Age-Related Decrease of IRAK-M Level in RPE

Age-associated accumulation of oxidative stress in the RPE is a recognised contributor to progression of AMD. To examine if additional oxidative stress could accelerate a reduction of IRAK-M expression, we induced oxidative insults both in vitro and in vivo.

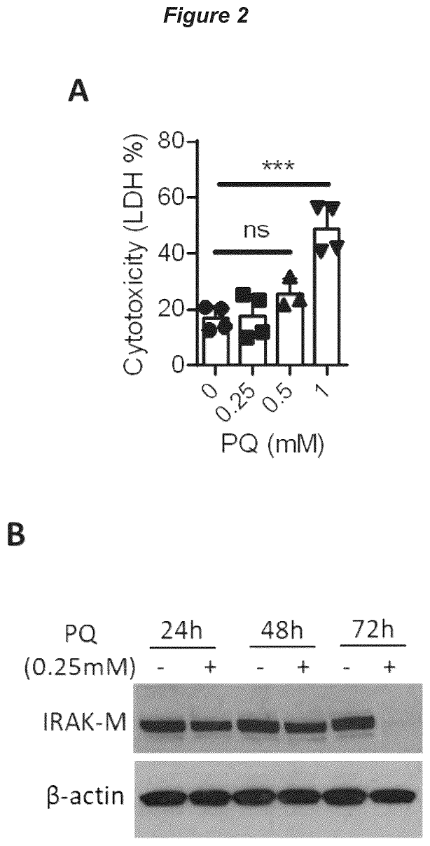

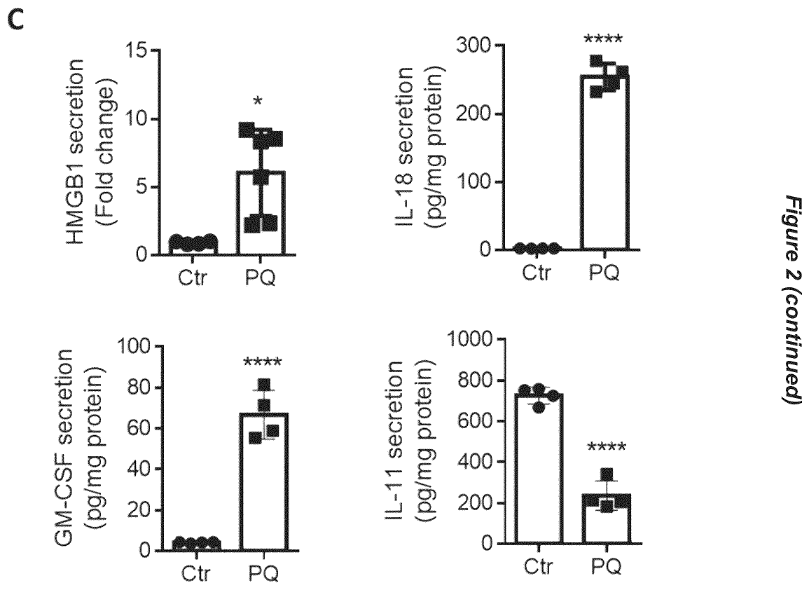

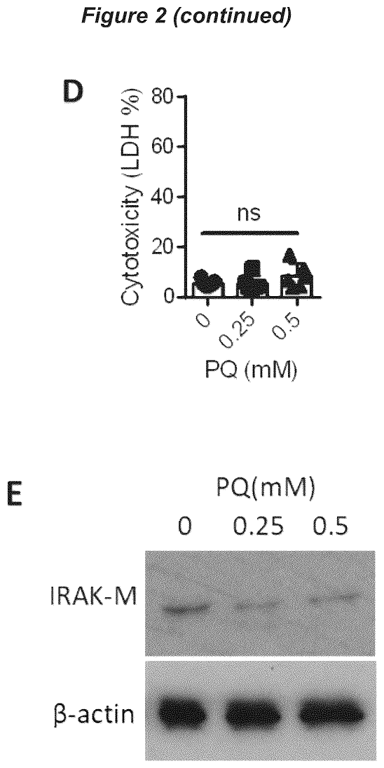

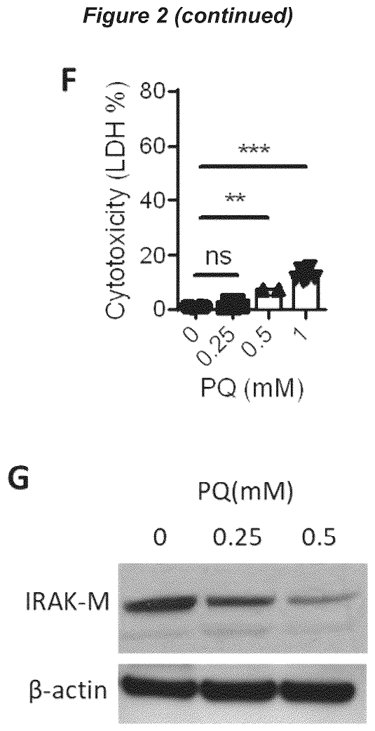

In vitro, treatment of a human RPE cell line (ARPE-19) with different concentrations of paraquat (PQ), a potent and stable chemical primarily inducing mitochondrial ROS, for up to 72 h. LDH cytotoxicity assay showed a dose-dependent cytotoxicity caused by PQ post 72 h (FIG. 2A). To define the potential regulation of IRAK-M expression by oxidative stress without inducing cell death, we tested a sub-toxic dose (0.25 mM) and observed it led to markedly diminished IRAK-M protein level after 72 h (FIG. 2B). Reduction in IRAK-M was accompanied by increased secretion of HMGB1, IL-18 and GM-CSF, and a decrease in IL-11, determined by EIA and multiplex cytokine array (FIG. 2C). Likewise, downregulated IRAK-M expression following 72 h treatment of sub-toxic doses of PQ (0.25-0.5 mM) occurred in human iPSC derived RPE (FIGS. 2D and 2E) and human primary RPE cells (FIGS. 2F and 2G).

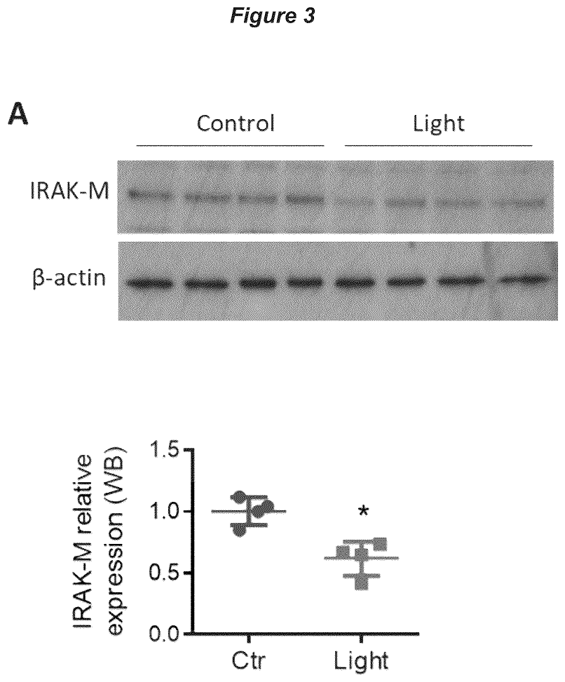

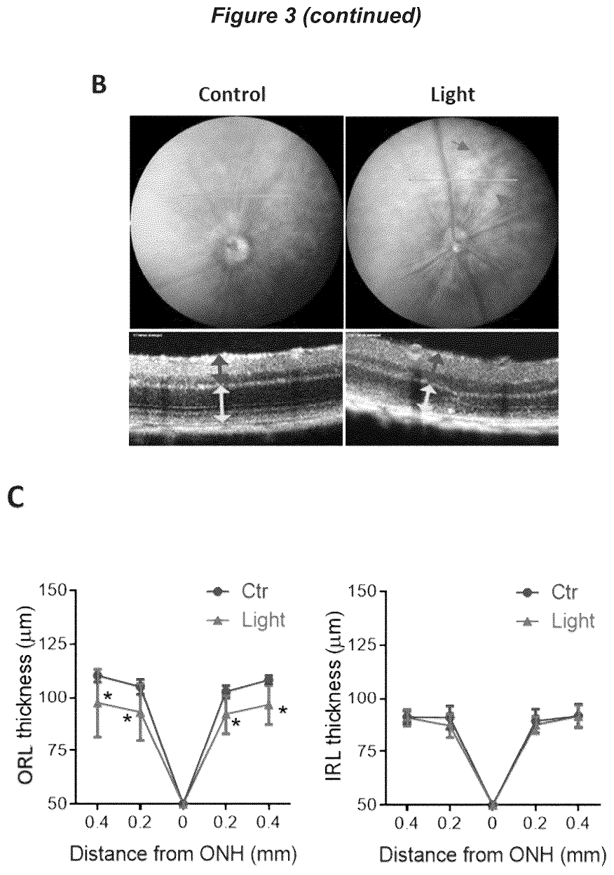

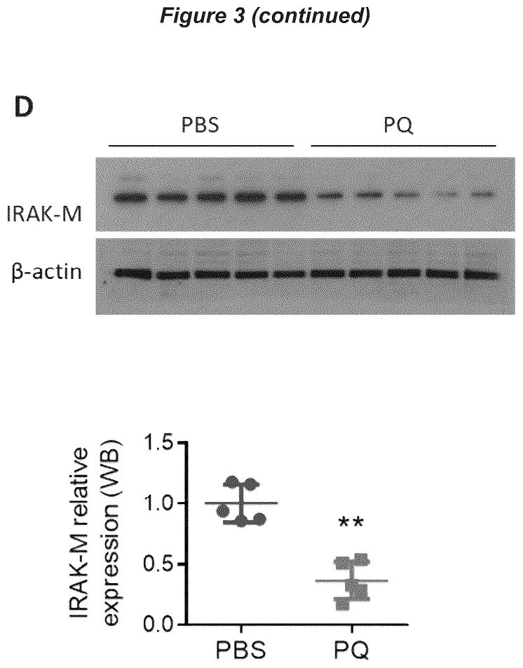

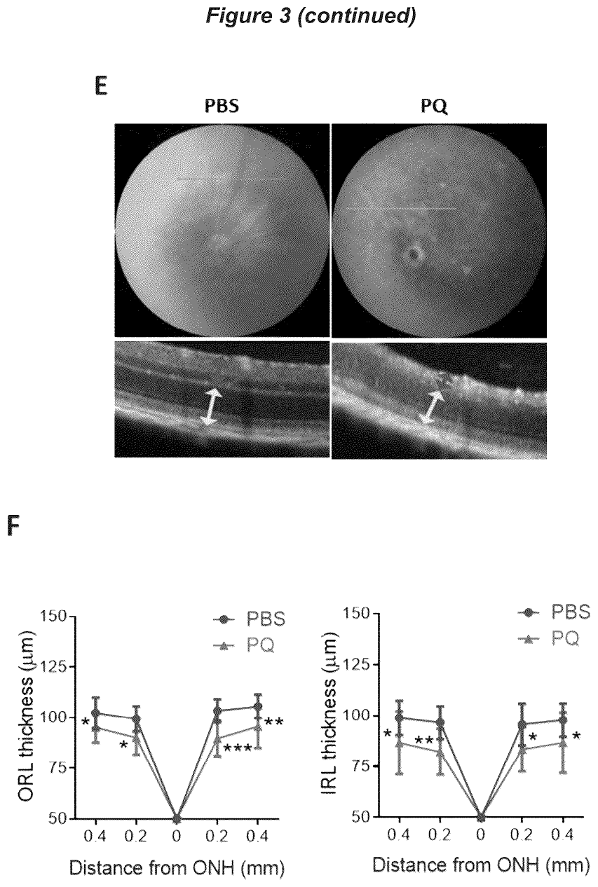

In vivo, retinal oxidative damage was introduced in C57BL/6J WT mice aged 8w by fundus camera-directed light exposure (100kLux for 20 min) (35) or intravitreal administration of PQ (2 μl at 1.5 mM) (36). Western blot analyses showed that IRAK-M expression in the RPE lysate was significantly abated on day 7 post light induction in both models (FIGS. 3A and 3D). Fundoscopy and OCT photographs obtained on day 14 displayed the fundal appearance of white spots (red arrows, FIGS. 3B and 3E) indicative of accumulated microglia/macrophages inside the ONL (37), alongside thinning of the outer retina indicative of cell loss in the light challenge model (FIG. 3C), and reduced thickness in both outer and inner retina in the PQ model (FIG. 3F).

### IRAK-M Expression in RPE Cells is Regulated by AP-1

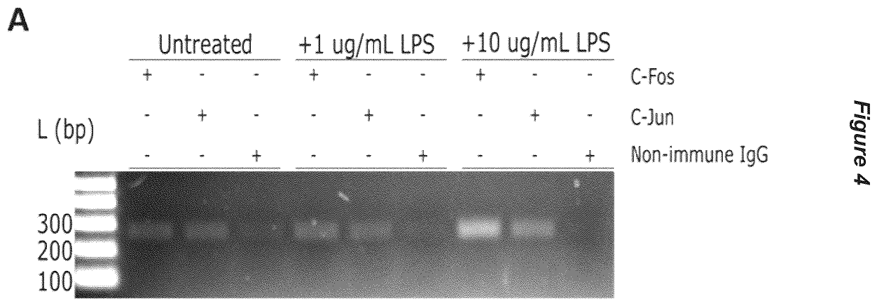

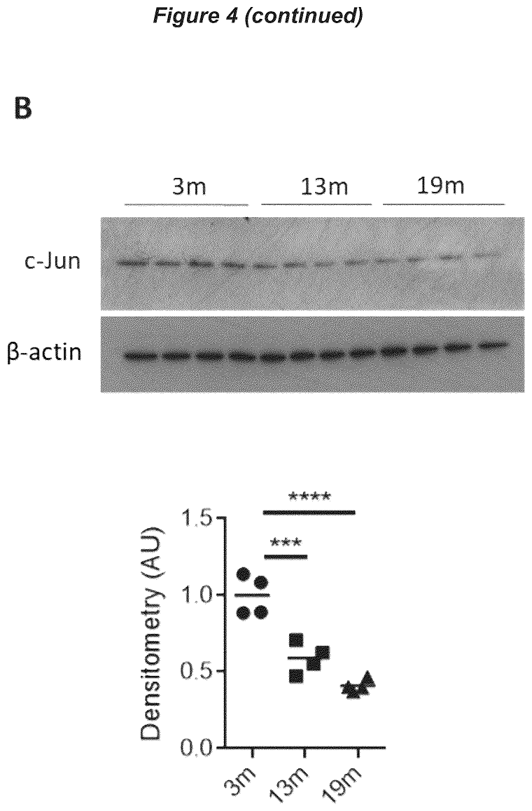

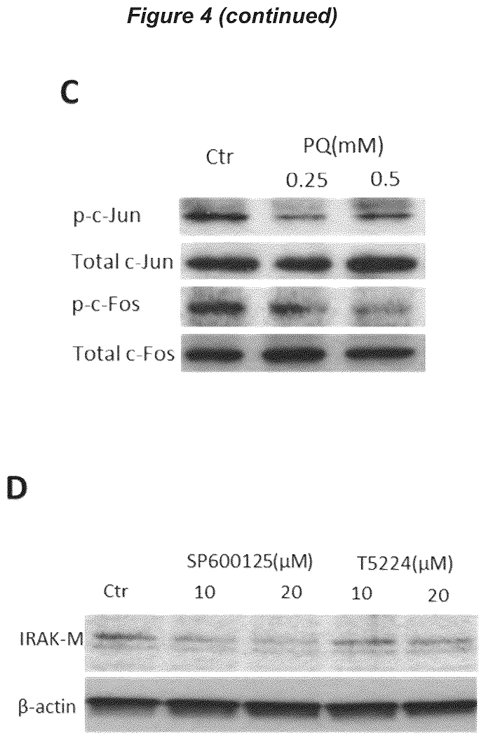

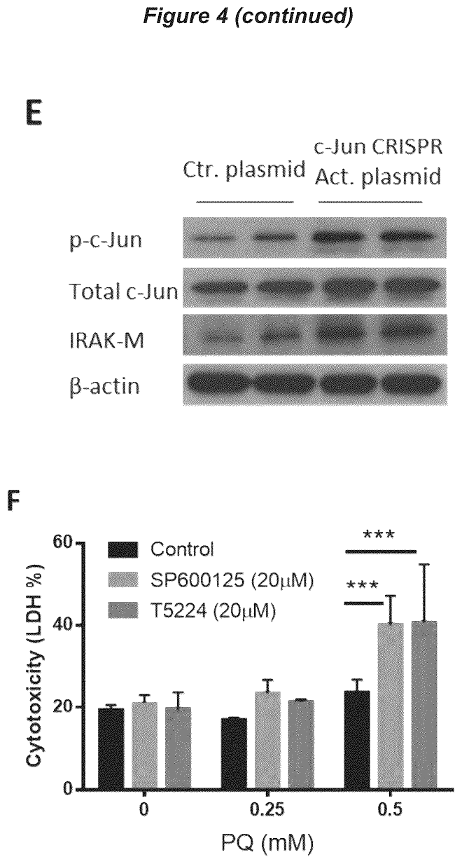

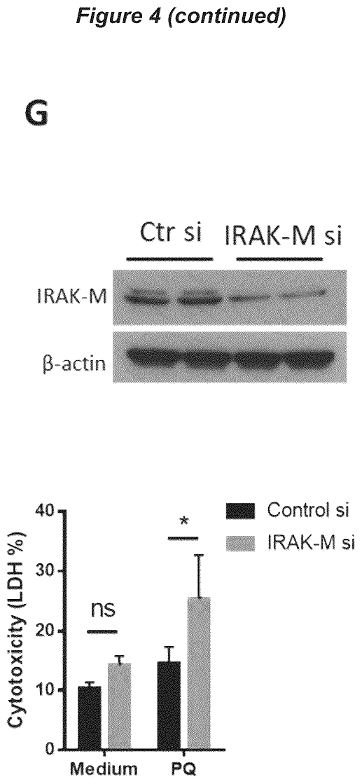

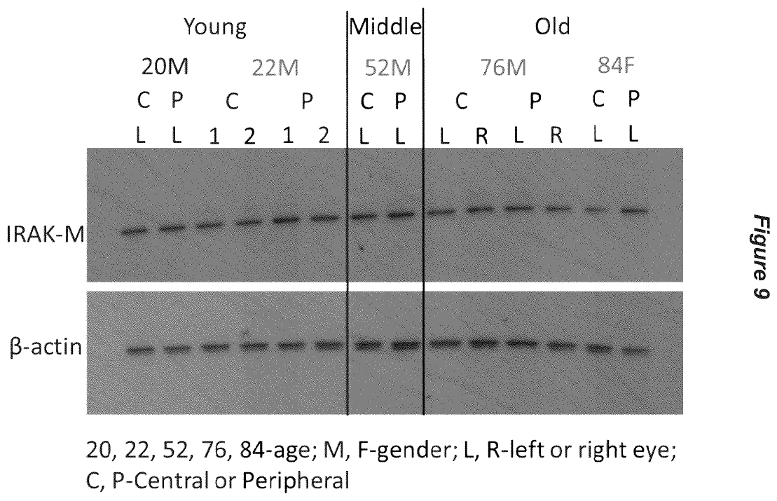

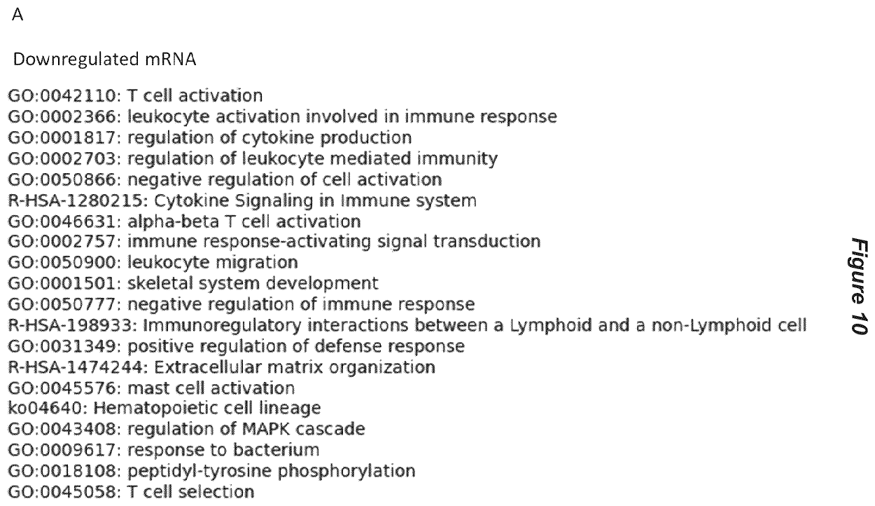

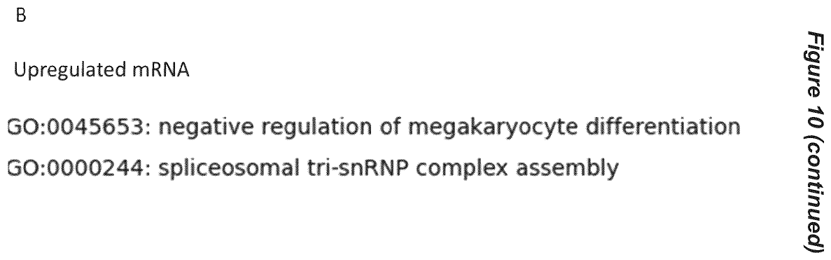

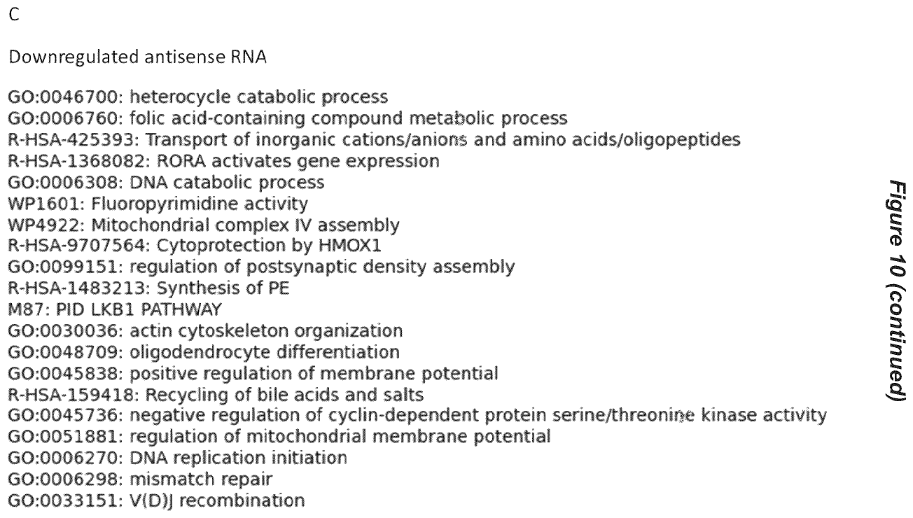

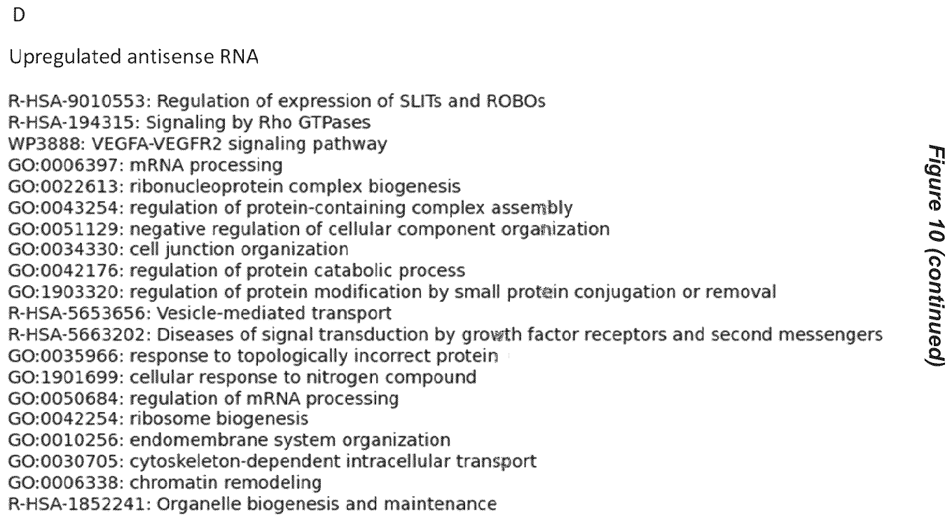

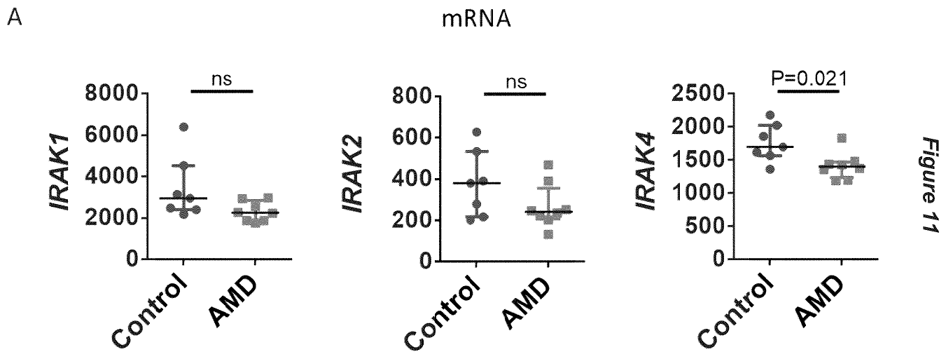

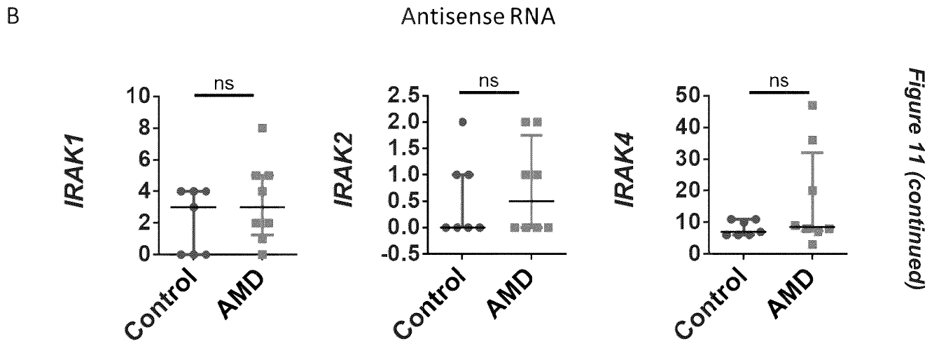

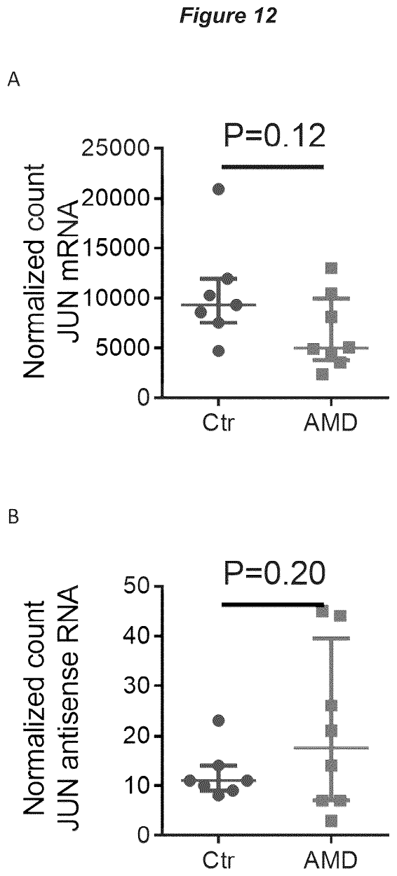

AP-1 transcription factor is one of the downstream effectors of TLR/IL-1R-mediated signalling pathways. It has been shown that AP-1 regulates IRAK-M in monocytes and lung epithelial cells, acting as an inhibitory loop (38). To investigate if AP-1 regulates IRAK-M in the RPE, we performed ChIP on ARPE-19 using antibodies against c-Jun and c-Fos (AP-1 subunits) for immunoprecipitation. The results demonstrated the occupancies of c-Jun and c-Fos in IRAK-M promoter in untreated cells, which was more pronounced with cells treated with LPS for 24 h (39) (FIG. 4A). In tandem with IRAK-M expression, the expression level of c-Jun was age-dependently reduced in ageing mouse RPE (FIG. 4B). The reduction started from 13m, earlier than the changes observed with IRAK-M (FIG. 4B vs. FIG. 1F). Data mining of RNA-Seq datasets (GSE99248) in AMD showed a non-significant decrease (P=0.12) in JUN mRNA level in AMD RPE/choroid/sclera compared to age-matched normal tissues (FIG. 12).

To study if oxidative stress also impacts on AP-1 activity or expression in the RPE, we treated ARPE-19 with PQ and demonstrated a downregulation of phosphorylation of both c-Jun and c-Fos after 72 h, and total c-Jun and c-Fos remained the same (FIG. 4C). Two AP-1 inhibitors, SP600125 (for c-Jun) and T5224 (for c-Fos) decreased IRAK-M expression in ARPE-19 (FIG. 4D). Conversely, by increasing c-Jun expression via CRISPR/Cas9 activation plasmid the IRAK-M expression was upregulated (FIG. 4E). Furthermore, treatment with AP-1 inhibitors resulted in enhanced ARPE-19 susceptibility to PQ-induced cytotoxicity (FIG. 4F), and like the detrimental effect induced by IRAK-M siRNA (FIG. 4G). We also performed experiments to test if by augmenting c-Jun expression using CRISPR/Cas9-based activation plasmid, which effectively activated c-Jun promoter to enhance expression, could inhibit oxidative stress-induced cell damage. However, over-expression of c-Jun itself showed a tendency of cytotoxic effect (P>0.05) and did not protect the cells from oxidative treatment (FIG. 13A), perhaps not surprising due to diverse functions of c-Jun/AP-1 signalling in stress response and apoptosis (40, 41).

### IRAK-M Deficient Mice Exhibit Advanced AMD-Like Pathologies

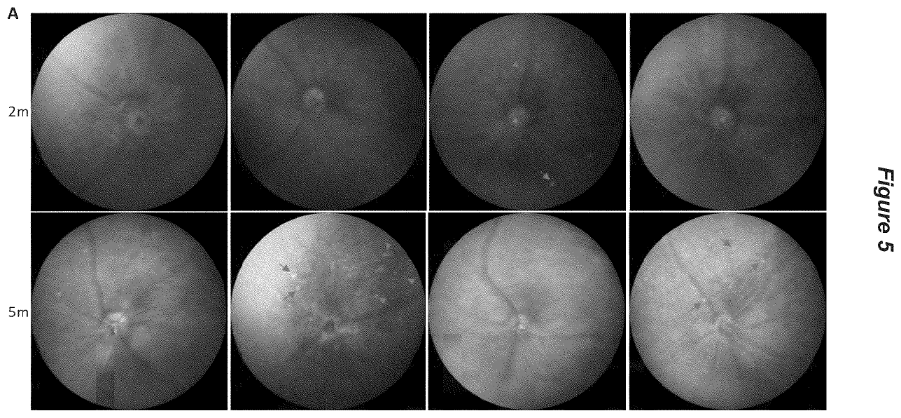

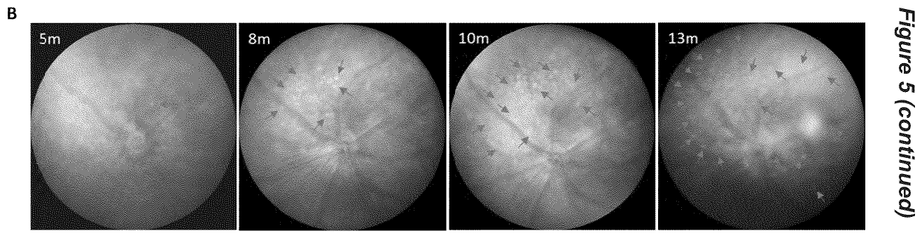

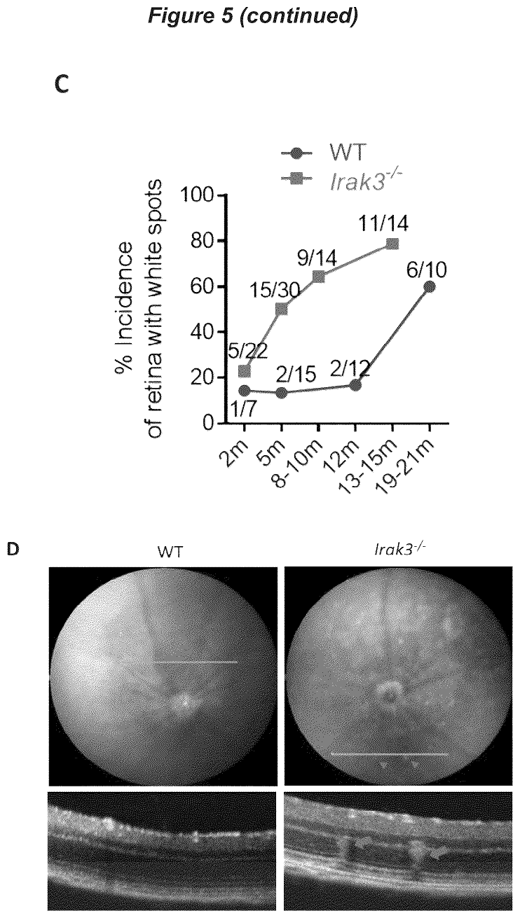

Having shown reduced IRAK-M expression with age and in AMD, we questioned if a lack of IRAK-M could accelerate retinal ageing and pathologies. To this end, Irak3−/− mice (without Rd8 mutation) were followed for 15 months using fundoscopy and OCT. Between 2 and 5m of age, there was a sharp increase in the incidence of retinas displaying variable number of fundus white spots, from 22.7% (5 out of 22 eyes) to 50% (15 out of 30) (FIGS. 5A and 5C). The incidence of abnormal retinal appearance increased continuously and reached 78.6% of eyes (11 out of 14) at 15m (FIG. 5C). We also noted that the white spots in those affected retinas developed whilst aging (FIG. 5B). In comparison, WT mice retained normal retinal appearance (i.e., no progression of white spots) at 12m, however a substantial incidence of WT retinas displayed white spots at 19-21m (60% or 6 out of 10, FIG. 5C). Notably, we did not apply fundoscopy for WT mice aged between 12 and 19m due to limited availability of aged mice. However, the findings (FIG. 5C) clearly indicate far earlier onset of retinal abnormalities in Irak3−/− mice than WT. The early appearance of white spots was associated with outer retinal lesions with OCT imaging (FIG. 5D).

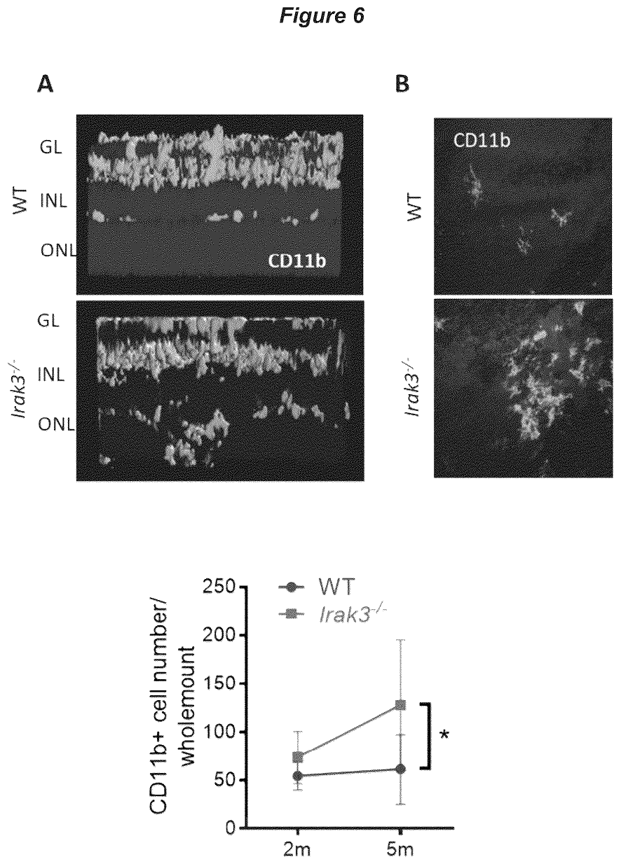

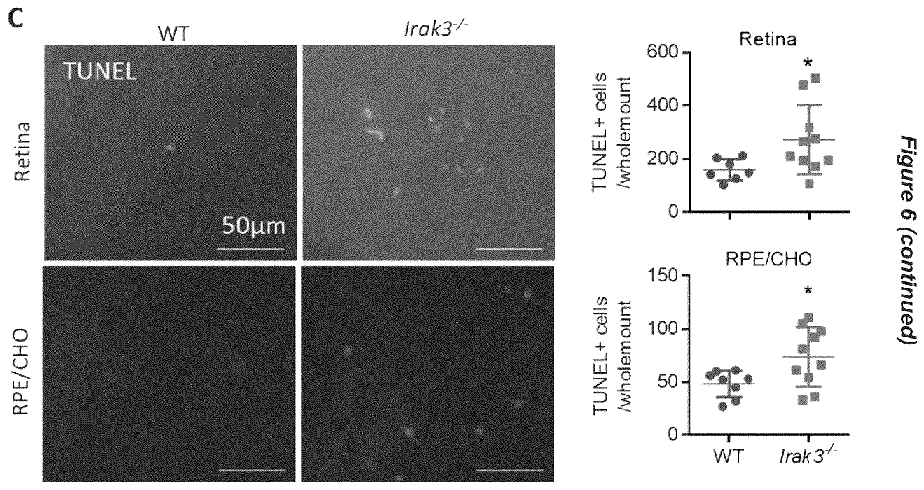

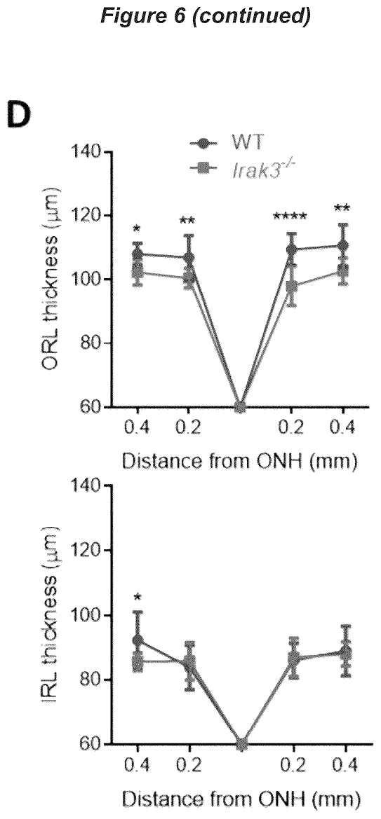

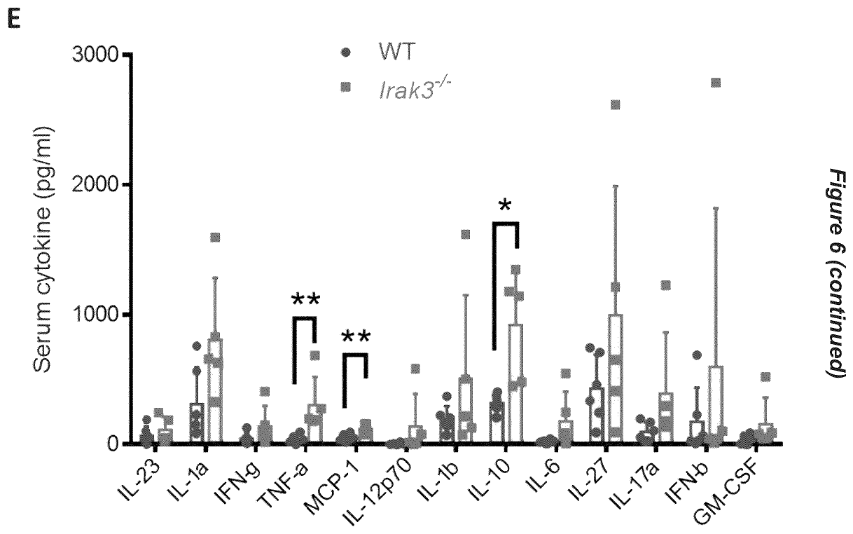

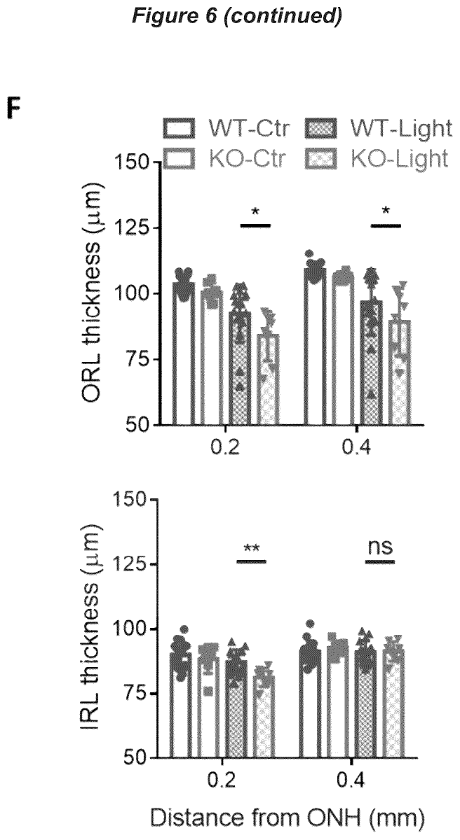

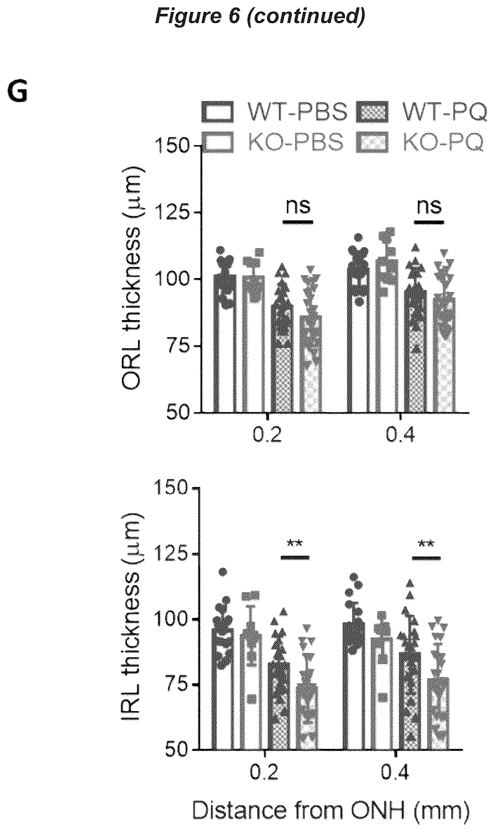

Alongside the early clinical changes in Irak3−/− mice, we noted the presence of increased numbers of CD11b+ myeloid cell populations in the outer nuclear layer (ONL), which is not seen in the retina of wildtype mice (FIG. 6A), and increased CD11b+ cell number in the subretinal space (FIG. 6B), together with more apoptotic cells within the retinal and RPE/choroidal tissues (FIG. 6C). Although no difference in retinal thickness was found at 5m between WT and KO mice, the outer retina of KO mice was thinner by 12-13m (FIG. 6D). In parallel, by 12-13m serum cytokine levels from KO mice increased compared to WT mice (significant increase in TNF-α, MCP-1 and IL-10; FIG. 6E).

Given age-dependent increase of retinal pathology in absence of IRAK-M, we next explored whether additional oxidative stress would exaggerate the pathology. Acute retinal oxidative stress was induced in adult WT and Irak3−/−animals (8w old) by light induction or PQ administration. KO mice exhibited amplified retinal damage compared to WT, particularly a greater reduction in outer and inner retinal thickness following light challenge (FIG. 6F), and a further reduction in inner retinal thickness by PQ administration (FIG. 6G).

### Loss of IRAK-M Disrupts RPE Cell Homeostasis

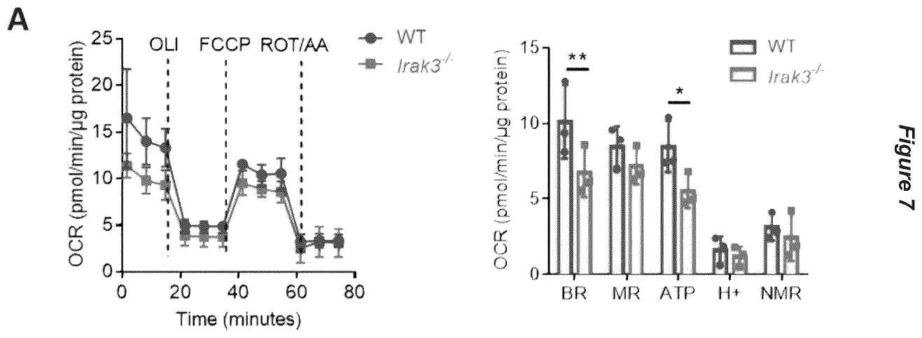

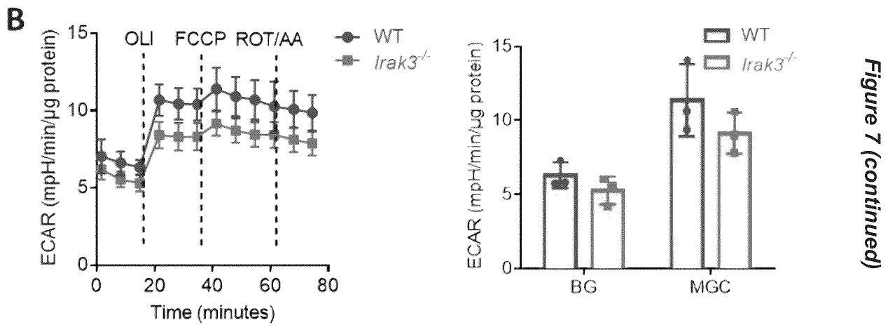

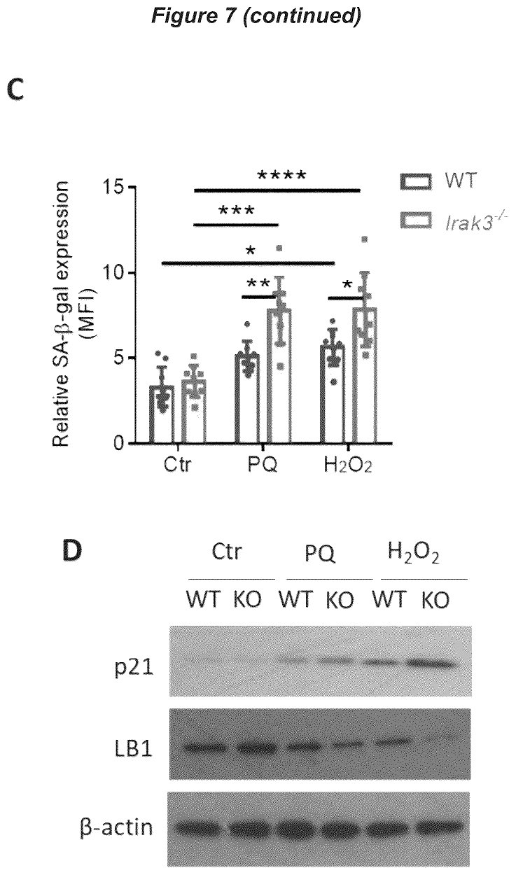

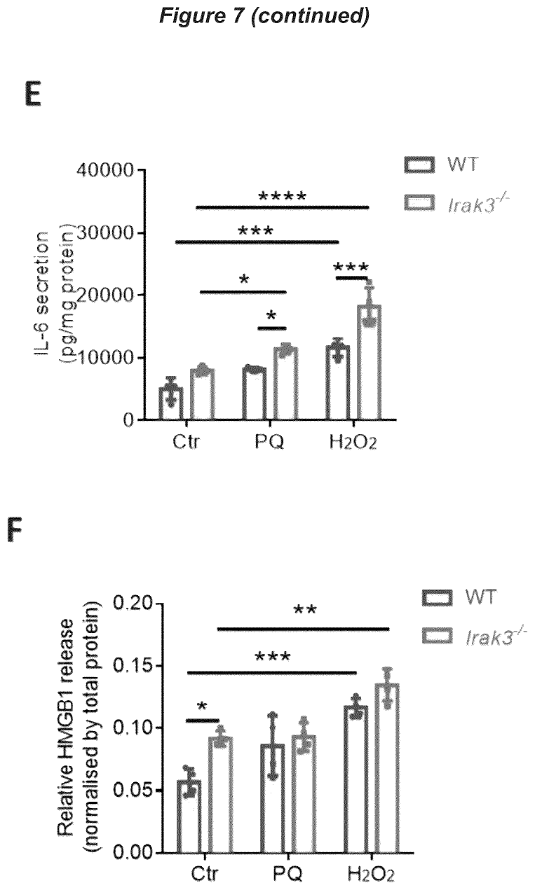

Within a retinal “metabolic ecosystem”, RPE primarily exploits mitochondria-dependent oxidative phosphorylation (OXPHOS) for energy synthesis and transports glucose to the outer retina, in particular photoreceptors that mainly rely on aerobic glycolysis (42). To elucidate metabolic mechanisms involved in IRAK-M deficiency-induced retinal degeneration, we examined RPE cell metabolism and senescence using primary mouse RPE cells. The cells without IRAK-M showed reduced levels of basal mitochondrial respiration (BR) and ATP production compared to WT cells as assessed by OCR analyses (FIG. 7A), while no significant differences in basal glycolysis (BG) and maximal glycolytic capacity (MGC) were observed between genotypes by ECAR examination (FIG. 7B). The data infers a role of IRAK-M to maintain mitochondrial function in RPE cells. In support, Irak3−/−RPE cells were more prone to oxidative stressor (PQ or H₂O₂)-induced senescence, demonstrated by increased SA-β-Gal activity (FIG. 7C), enhanced expression of cyclin-dependent kinase inhibitor p21CIP1, decreased nuclear lamina protein LB1 (FIG. 7D), and elicited secretion of IL-6 (a known SASP-cytokine released by RPE) (16, 21) (FIG. 7E). The basal secretion level of proinflammatory cytokine HMGB1 of Irak3−/− RPE cells was significantly higher than WT cells but only in absence of oxidative stressors (FIG. 7F).

### Overexpression of IRAK-M Protects RPE Cells

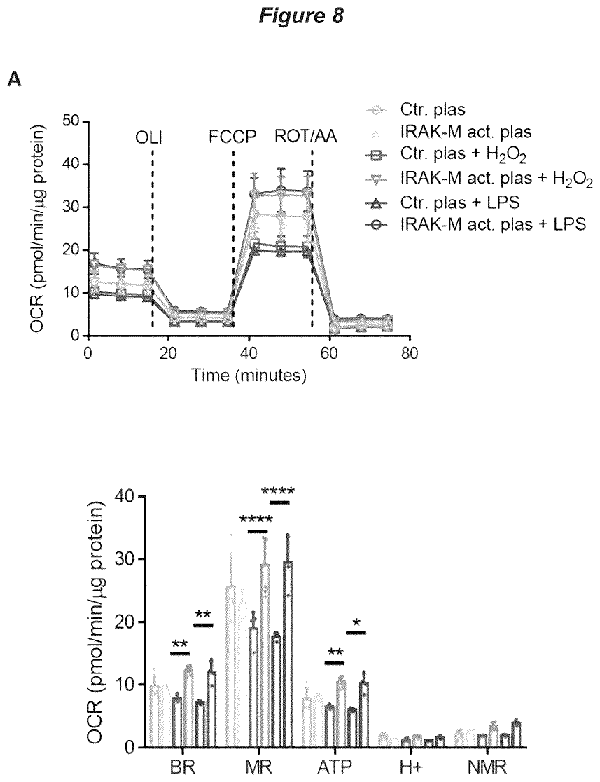

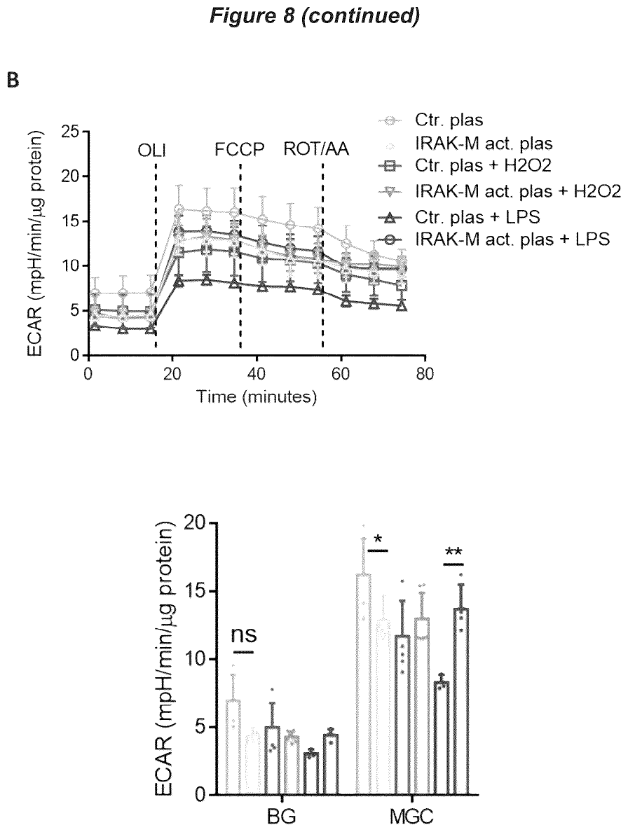

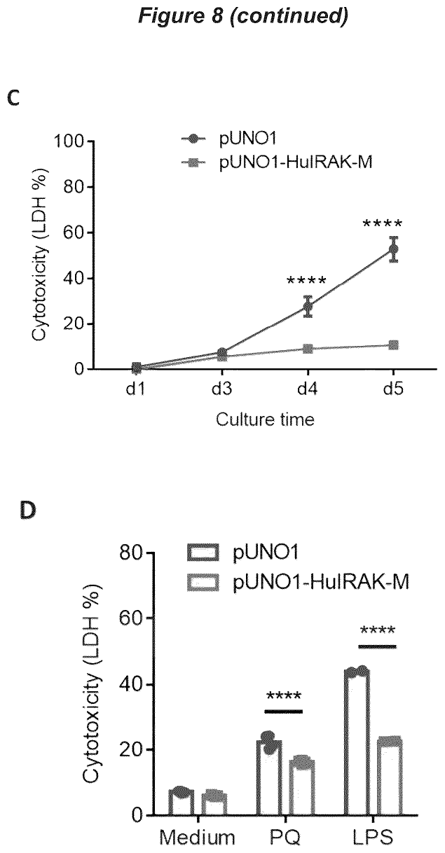

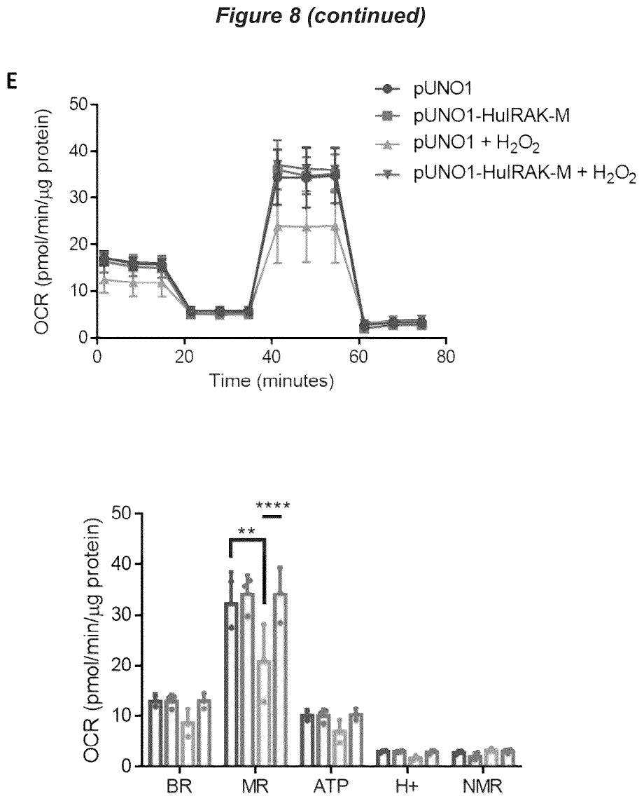

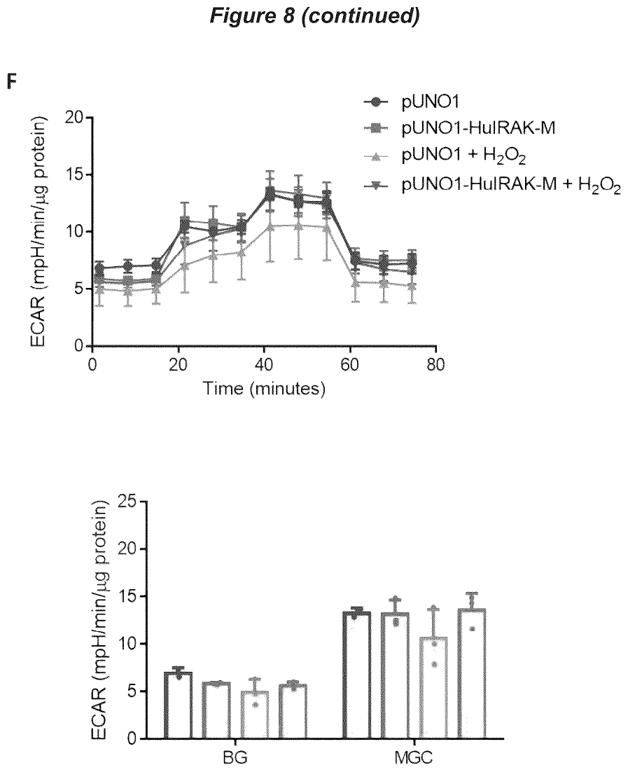

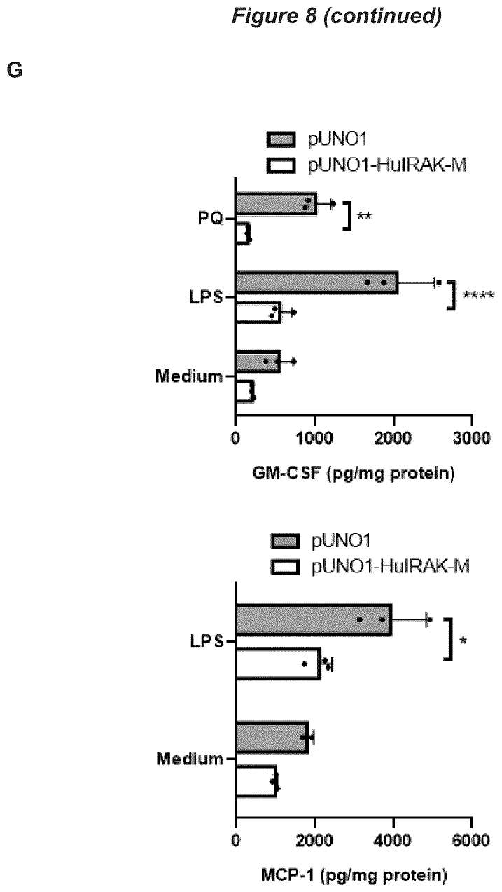

Given the data supporting a role for IRAK-M in maintaining RPE function and health in presence of oxidative stress, we wished to address whether an overexpression of IRAK-M could protect RPE cells. We augmented native IRAK-M expression in human iPSC-RPE cells via transfection with a CRISPR/Cas9-based activation plasmid (FIG. 13B). After 48 h of transfection, the cells were treated with H₂O₂ or LPS for a further 24 h. OCR analysis demonstrated that basal and maximal mitochondrial respiration were sustained by IRAK-M overexpression, but impaired in control transfected cells following either oxidative or immune stresses (FIG. 8A). Although untreated IRAK-M-overexpressing iPSC-RPE cells displayed lower maximal glycolytic activity than control plasmid-transfected cells, the level remained unchanged when stressed with H₂O₂ or LPS (FIG. 8B). In contrast, glycolytic activity in control cells was significantly reduced by both H₂O₂ and LPS (FIG. 8B). The lower level of glycolysis in resting iPSC-RPE with overexpressed IRAK-M suggests less dependency on glucose for energy metabolism, which could be beneficial to photoreceptors primarily relying on glycolysis (42).

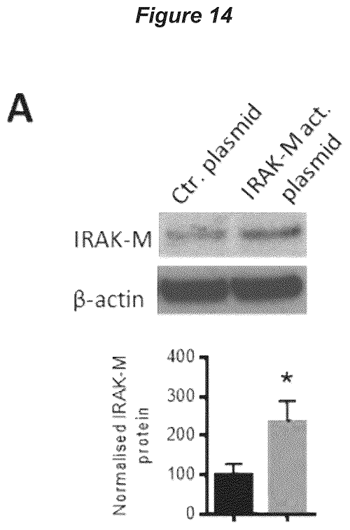

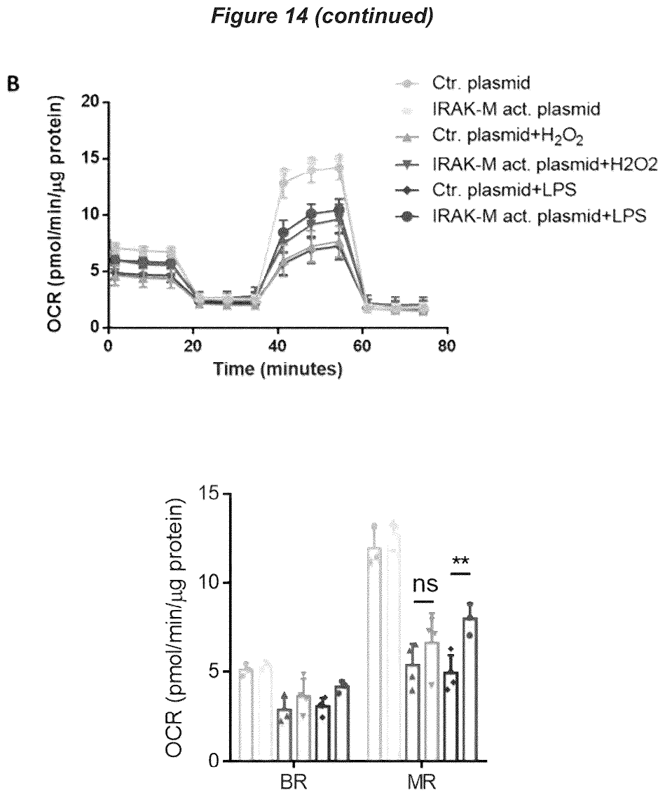

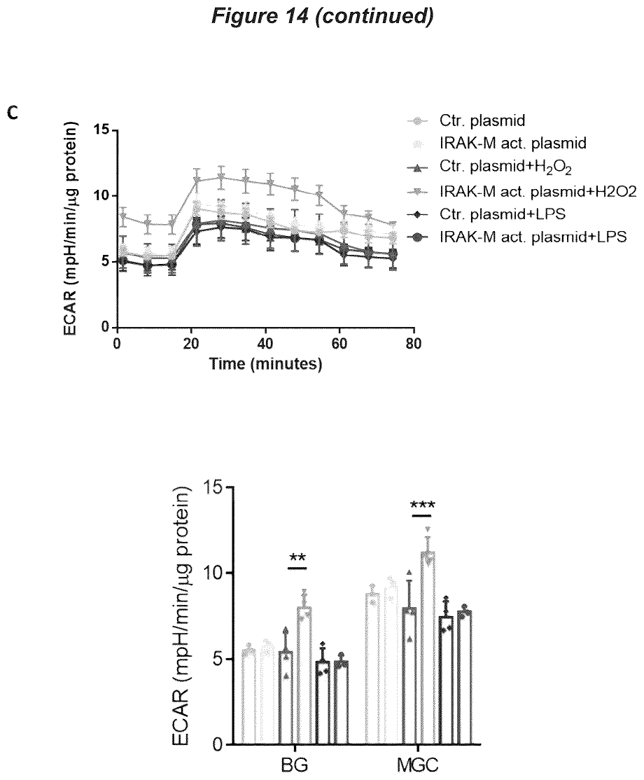

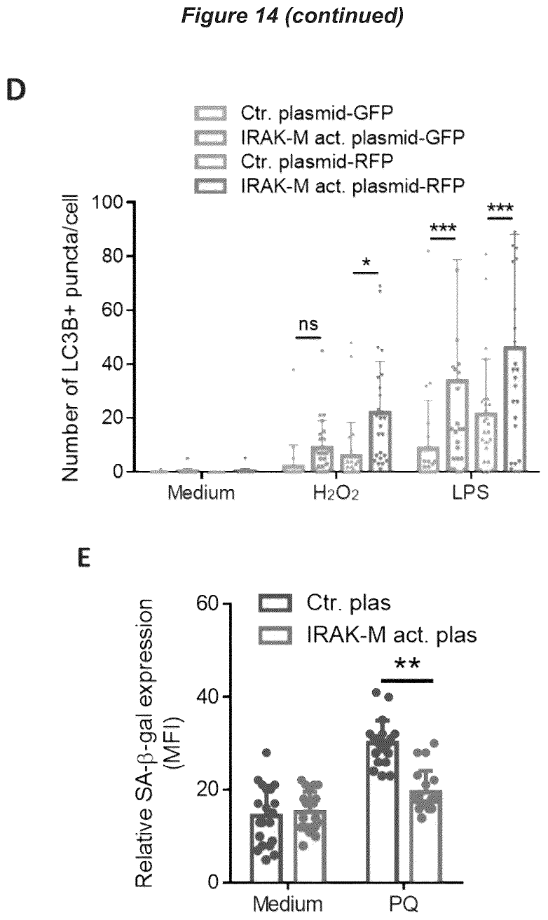

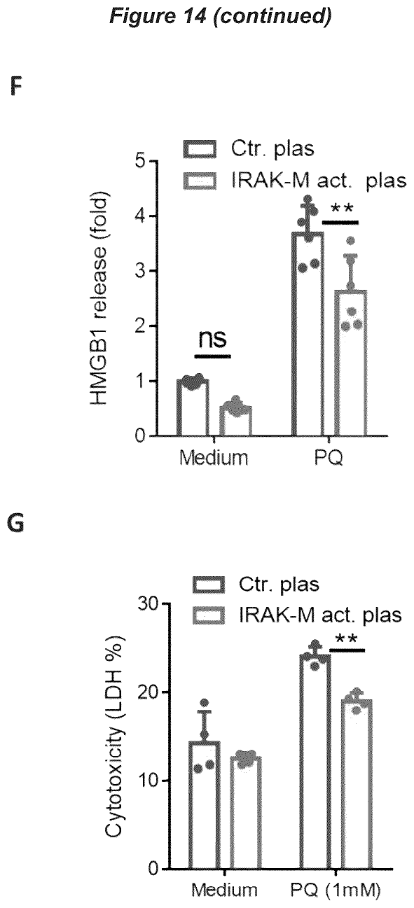

Utilising ARPE-19 cells, enhanced IRAK-M expression by CRISPR/Cas9 also partly reversed LPS-induced reduction in maximal mitochondrial respiration (FIGS. 14A and B), partly supporting our findings in iPSC-RPE (FIG. 8A). Given similarities we continued to interrogate, however ARPE-19 was able to maintain glycolytic activity after H₂O₂ or LPS exposure in control cells, and enhanced IRAK-M expression resulted in an increased glycolysis in response to H₂O₂ (FIG. 14C). Taken further, overexpression of IRAK-M in ARPE-19 induced the formation of autophagosomes (LC3B-GFP) and autolysosomes (LC3B-RFP) under H₂O₂ or LPS treatment, representing an upregulated autophagy flux, as shown by a tandem sensor RFP-GFP-LC3B kit (FIG. 14D). Moreover, ARPE-19 senescence induced by subtoxic dose of PQ (0.25 mM) was inhibited by IRAK-M overexpression, evidenced by decreased SA-β-Gal activity and HMGB1 secretion (FIGS. 14E and F). Finally, a toxic dose of PQ (1 mM) inducing marked LDH release (FIG. 2A), was significantly inhibited by increasing IRAK-M expression (FIG. 14G).

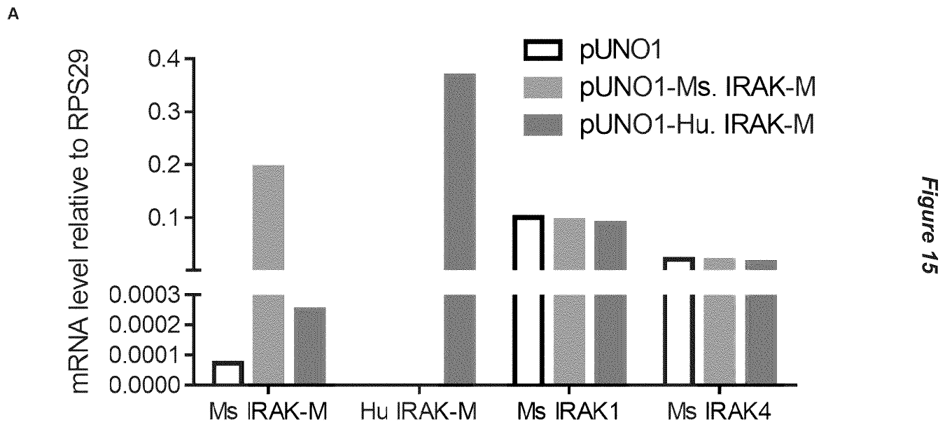

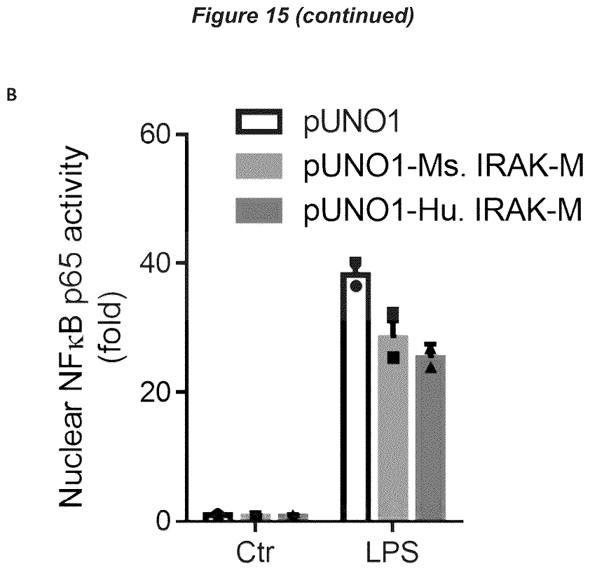

Gene therapy approaches to deliver human genes into mouse eyes have been pivotal for preclinical assessment (43-45). Here we created stable transfected RPE cell lines from parent mouse B6-RPE07 cell line using pUNO1 vectors (see Materials and Methods). The newly established cell lines were kept and subcultured in Blasticidin-containing medium and had stable and strong expression of human or mouse IRAK-M mRNA (FIG. 15A). Expressions of IRAK1 and IRAK4 mRNA were not affected, indicating the specificity of the gene delivery. Next, a NF-κB activity assay showed a decrease in DNA-binding activity of nuclear NF-κB in human IRAK-M-expressing mouse cells after acute LPS stimulation (30 min), similar to the inhibitory effect of murine IRAK-M overexpression (FIG. 15B), which confirms the functionality of human IRAK-M to suppress TLR/NF-κB signalling cascade in mouse RPE cells. As stable transfection allows longer-term research on mechanism and consequence of genetic regulation and pharmacology studies, we kept the cell monolayers after confluence in serum-free condition for up to 5 days. The cell viability of RPE cells expressing human IRAK-M was dramatically maintained, while control cells presented elevating toxicity between day 3 and 5 (FIG. 8C). When newly confluent cells (day 0) were treated by PQ (0.125 mM) or LPS (40 ng/ml) for 3 days, transduction with human IRAK-M significantly inhibited stresses-induced cytotoxicity (FIG. 8D). To exclude the possible implication of endogenous mouse IRAK-M in cell responsiveness observed above, we performed transient transfection on primary RPE cells isolated from adult Irak3^{-/-} mice. In this regard, Seahorse Metabolic Flux assay was applied to examine metabolic alterations in response to shorter period of treatment with H₂O₂ (24 h, FIGS. 8E and F). In support to the findings from human iPSC-RPE cells when transfected with CRISPR/Cas9 activation plasmid (FIGS. 8A and B), maximal mitochondrial respiration in primary Irak3^{-/-} RPE cells was retained by human IRAK-M transduction after H₂O₂ treatment, in contrast to the control transfection that showed a marked reduction in mitochondrial activity (FIG. 8E). H₂O₂-induced oxidative stress did not significantly alter glycolytic activities in Irak3^{-/-} RPE cells (FIG. 8F). The stable expression of human IRAK-M in transfected B6-RPE07 cells suppressed the production of proinflammatory cytokines under stresses, including LPS or PQ-induced GM-CSF, and LPS-induced MCP-1 (FIG. 8G). This set of data using cell models implies human IRAK-M is functional in mouse RPE, facilitating in vivo assessment and clinical translation.

### SUMMARY

AMD is a progressive, polygenic, and multifactorial eye disease. Here we reveal a molecular mechanism that highlights IRAK-M in modulating response to stressors and maintaining cell health with a potential to prevent progressive degeneration and cell loss.

Diminished IRAK-M level was observed with increasing age or following oxidative stress, each alone or compounding, precipitating an unchecked immune response in RPE (FIGS. 1-3). The RPE is vital to nourish the retina through the visual cycle and maintain photoreceptor function. Constantly exposed to insults caused by high metabolic rate, light exposure, heterophagy and free radical formation, RPE health is prone to age-associated defects and mitochondrial dysfunction (8, 46, 47). As an early clinical indication and risk determinant of AMD, drusen contain a comparable protein profile as degenerating RPE, which is therefore considered as the main source releasing drusen components via exosomes (48, 49).

Dynamic crosstalk between signalling cascades and an intact feedback system ensures immune homeostasis at the level of the tissue (autophagy or inflammasome activation) or the local immune network (para-inflammation) (50), where immune suppression executes a crucial controlling mechanism in the equation. RPE cells actively participate in regional innate and adaptive immune activation and suppression by expression of a host of immune molecules and serving as a gateway for systemic leukocyte trafficking (27, 51, 52). Apart from the known immunosuppressive factors produced by the RPE, such as anti-inflammatory cytokines (TGF-β, IL-11 and IFN-β), chemokine CX3CL1 (fractalkine), IL-1R antagonist (IL-1Ra), IL-1R2 (CD121b), and membrane glycoprotein CD200 (13, 27, 53-55), we identified IRAK-M as a key intracellular anti-inflammatory molecule localized in the retina and predominantly expressed by the RPE (FIG. 1A-D). Our finding therefore complements and extends the understanding of the essential role of RPE in immunoregulation at the posterior segment of the eye (51).

Oxidative stress triggers TLR-mediated inflammatory response either directly by ROS (e.g., H2O2) or indirectly by autocrine or paracrine secretion of products from oxidative damage (e.g., HMGB1) (25), as verified previously (56) and confirmed by the present work (FIG. 2C). Other oxidative damages include DNA break, mitochondrial disturbance and impairment of intracellular RPE processing pathways (autophagy, phagolysosome and protein trafficking). At least ten functional TLRs (TLR1-10) have been identified in humans, of which TLR1-7, 9, and 10 are found in the RPE (57). As a primary inhibitor for TLR/IL-1R-transduced, NF-κB/AP-1-mediated inflammatory responses, IRAK-M expression is regulated by numerous endogenous or exogenous factors including adiponectin, TGF-β1, GM-CSF, and cell surface or intracellular molecules including TREM-1 and PI3K (30). For instance, whilst acute alcohol intake increases IRAK-M in human monocytes, chronic alcohol exposure leads to decreased IRAK-M expression and hyperresponsiveness to LPS, associated with overactivation of NF-κB and increased TNF-α secretion (58). Reduced IRAK-M levels in monocytes and adipose tissues of obese subjects constitute a causative factor of systemic inflammation and mitochondrial stress (31). We demonstrate that old age and oxidative stress lead to IRAK-M downregulation in the RPE and the ensuing degenerative cytokine response (FIG. 1E, 1F, 2B, 2D, 2F, 3A, 3D). In support, transcriptome data mining suggests that the expression level of IRAK-M further subsides during AMD compared to age-matched controls, which other IRAK family members have no marked change in AMD (FIG. 1G, FIG. 11). This indicates that IRAK-M may serve as a harbinger molecule of degeneration into AMD.

We show that IRAK-M in the RPE is regulated by AP-1 (FIG. 4A, 4D, 4E), acting as a negative feedback control of inflammation. AP-1 is a dimeric transcription factor assembled from Jun and Fos family proteins, of which c-Jun and c-Fos are among the most important regulators of genes involved in cell function, proliferation, differentiation, apoptosis and immunity (59). Decreased transcription activity of AP-1 has been linked to tissue and cell ageing (60, 61), in opposed to NF-κB that was increased in activity in aged tissues and age-related conditions such as Alzheimer's disease, diabetes and osteoporosis (61, 62). In mice, depletion of c-Jun, but not c-Fos, causes embryonic death (63). We found that c-Jun expression or activity declined in tandem with IRAK-M reduction in ageing or under oxidative stress (FIG. 4B, 4C), which agrees with early studies showing impaired AP-1 activity or reduced AP-1 subunit expression in aged rodent tissues (64). Interestingly, the reduction of c-Jun phosphorylation did not occur until 72 h (FIG. 16A), which may reconcile with previous reports of increased AP-1 transcription upon oxidative stress in RPE cells within 24 h (65, 66) and implies a requirement of time to allow oxidative stress to accumulate during ageing (60, 64). Notably, although inhibition of AP-1 activity sensitized RPE cells to oxidative damage (FIG. 4F), over-expression of c-Jun failed in protection (FIG. 13A), possibly due to diverse functions of c-Jun/AP-1 signalling in cell response and apoptosis (40, 41).

Dysregulation of TLR-mediated signalling components has been focused on as a critical mediator in the initiation and progression of inflammation-associated degenerative diseases (67). There is increasing evidence of abnormal IRAK-M expression or IRAK signalling in human diseases such as chronic alcoholic liver disease, inflammatory bowel disease, insulin resistance and features of metabolic syndrome (28, 30, 31). Knockout IRAK-M in mice wreaks havoc on systemic or local immune activities, incurring susceptibility to endotoxin shock, autoimmune diabetes, osteoporosis and neuronal vascular injury (29, 30, 68-70). We show that Irak3−/− mice spontaneously develop AMD-like characteristics of retinal lesions and increased cell death, at early as 5m of age (FIG. 5A-D, 6A-C). They are more susceptible to oxidative insults, as demonstrated in two different retinal degeneration models (FIGS. 6F and 6G), suggesting a pathway convergency of immune dysregulation and excessive oxidative stress. Furthermore, the absence of IRAK-M in RPE cells with different origins (iPSC derived, primary cells or cell lines) leads to destructive cell homeostasis, evidenced by reduced mitochondrial energetics, increased cellular senescence and SASP cytokine secretion (FIG. 7A, 7C-F).

A gene therapy approach of delivering and restoring IRAK-M expression in RPE has clinical significance to prevent AMD progression at the early stage. The retinal degeneration in AMD is a collective outcome of aberrance in inflammation, mitochondrial function, lipid metabolism, autophagy and cellular senescence (8, 16, 71, 72), where immune regulators have emerged as the crux of the interplays and hold promise to break the vicious cycle (16, 73). Numerous non-steroidal anti-inflammatory drugs (NSAIDs), neutralizing antibodies against IL-1a or IL-1R, and anti-inflammatory IL-10 have been shown to regulate cell metabolism and autophagy (74-77). We have recently found the protective effects of IL-33 in the retina using an immune-mediated insidious retinal degeneration model (Cfh+/− with high-fat diet) (17, 23). In the present study, increasing IRAK-M in the RPE via boosting endogenous gene expression or exogenous gene delivery helps to maintain cell functions (mitochondrial activity and autophagy) and inhibit senescence/SASP, thereby promoting cell survival (FIG. 8A, 8C, 8D, 8E and FIG. 14B, 14D, 14E-G), implying that IRAK-M is a master regulator in the immunoregulatory hierarchy unfolding in RPE cells and AMD etiology. Notably, we found that increased IRAK-M expression is more protective in human iPSC-RPE and murine primary RPE cells compared to ARPE-19 cells against oxidative and/or immune stresses (FIGS. 8A and E, and FIG. 14B). Furthermore, glycolytic responses in these RPE cell cultures were different, where increased IRAK-M expression did not alter glycolysis in human iPSC-RPE (FIG. 8B) and murine primary RPE (FIG. 8F), but induced glycolysis in ARPE-19 (FIG. 14C). Despite variabilities between cell models (78, 79), the results demonstrate a protective role of IRAK-M expression in RPE mitochondrial health, which is essential for the RPE with high metabolic demand (42).

Experimental approaches have been used to introduce human genes, such as RPE65 and NADH dehydrogenase subunit 4 (ND4) to mouse models for preclinical assessment (43-45). It has been shown that human and mouse IRAK-M have a comparable cellular expression, and functional similarities with respects to signal transduction activities (29, 80). The IRAK-M gene is located on chromosome 12 in human (Uniprot ID Q9Y616, 596 amino acid (aa) in length) and chromosome 10 in mice (Q8K4B2, 609 aa), respectively. Regardless of species, full length of IRAK-M contains a death domain (DD, aa 41-106 for both human and mouse IRAK-M) involved in binding to other IRAK family members, a pseudokinase domain (aa 165-452 for human and aa 178-463 for mouse) and an unstructured C-terminal domain with a TRAF6 binding motif (81). We performed BLAST search and revealed that human IRAK-M shares 74.55% aa sequence identity with the murine homologue, where there are 91.67% identity in DD sequence and 83.22% identity in pseudokinase domain sequence. Dot plot of BLAST sequence alignment showed a close similarity in the domain sequences between human and mouse IRAK-M (FIG. 16B). Additionally, the in vitro functional studies using newly created stable transfectant cell lines and primary cells both confirmed the functionality of human IRAK-M gene delivery in mouse RPE cells (FIG. 8C-F).

Approaches of gene therapy, which induces persistent therapeutic transgene expression, have been utilized to treat chronic diseases (82, 83) where pathologic cues sustained over time could not be abolished by other solutions (84). The eye is an ideal organ for gene therapy because of its facile access and compartmentalization, relative immune privilege and a small size to reduce the viral load required. Ocular gene therapy has been successful applied in a variety of diseases (84, 85). There are two ongoing Phase 1 gene therapy trials for dry AMD, including GT005 that induces complement factor I (CFI) expression (86) and HMR59 (AAVCAGsCD59) that expresses C59 to prevent the formation of membrane attack complex (MAC) (87). As our data pinpointed the age-related and oxidative stress-induced decline of IRAK-M in immune activity to be at the root of AMD, our ongoing work is to explore a novel approach of targeted immunotherapy to restore RPE health and function by augmentation of IRAK-M expression using viral gene therapy. We are utilizing subretinal administration of AAV2 vector, hitherto the most characterised AAV serotype in clinical trials to treat RPE-related ocular diseases with durable gene expression and no deleterious side-effects (43, 88, 89). Proof of concept will be tested through augmentation of IRAK-M in the RPE in a light-induced retinal degeneration model (LIRD) using Irak3−/− mice. Future research is also warranted to assess tissue and humoral responses in mice following delivery of human IRAK-M transgene. With the continuously advancing safer viral vector techniques, our results potentialize further development towards an effective gene therapy to treat dry AMD. The study outcome may steer the development of gene therapies for not just AMD, but other age-related diseases where chronic inflammation plays a critical role.

### Example 2

### Materials and methods

### Immunohistochemistry of Human Eye Sections

Paraffin-embedded human eye sections from AMD and non-AMD subjects were obtained from the Lions Gift of Sight (Minnesota, USA) after research ethics committee approval (20/LO/0336), with experiments conducted according to the Declaration of Helsinki and in compliance with UK law. The slides were deparaffinized and rehydrated, followed by antigen retrieval with citrate buffer (pH 6.0) at 90° C. for 20 minutes. After three washes in PBS, and blocked and permeabilized in 5% normal goat serum (NGS), 5% BSA and 0.1% Triton X-100, specimen were incubated with rabbit anti-IRAK-M antibody (1:250, Cat. ab8116, Abcam, Cambridge, UK) at 4° C. overnight. The secondary antibody, a biotinylated goat anti-rabbit IgG (1:1000, Thermofisher Scientific, Paisley, UK) visualized via the avidin-biotin-alkaline phosphatase complex (ABC-AP) method (Vectastain ABC-AP Kit, 2Bscientific, Upper Heyford, UK) using the Vector Red Substrate (2Bscientific). Slides were then counterstained by Hematoxylin, dehydrated, and mounted with Histomount medium. The absence of staining when the primary antibody omitted was used as negative control. IHC images at the macular area and peripheral retina were captured using an Evos XL Core microscope (Thermofisher Scientific). Images were processed using Colour Deconvolution plugin in Fiji to separate Hematoxylin (blue), IRAK-M (AP-Red) and pigment (brown). The ROI of pigmented RPE was carefully identified in the pigment/brown picture, which was copied-and-pasted into the identical area of the AP-Red picture. The mean staining intensity of IRAK-M for RPE was measured using Fiji. The ROI for retina or choroid was selected based on the nuclear staining (blue).

### Subretinal Injection

Male C57BL/6J mice (Charles River Laboratories, Portishead, UK) aged 8 weeks or Irak3^{-/-} mice aged 2-4 months were anesthetized using an intraperitoneal injection of 200 μl of Vetelar (ketamine hydrochloride 100 mg/ml, Pfizer, Sandwich, UK) and Rompun (xylazine hydrochloride 20 mg/ml, Bayer, Newbury, UK) mixed with sterile water in the ratio 0.6:1:84. Pupils were dilated using 1% tropicamide and 2.5% phenylephrine (both from Chauvin, Essex, UK). A drop of Viscotears (Novartis, London, UK) was then applied to cover the surface of the eye before the following procedures. All trans-scleral subretinal injections used were 2 μL in volume at the titres indicated and were delivered using an operating microscope and a 33G needle on a microsyringe under direct visualization (Hamilton Company, Reno, NV, USA). 1% Chloramphenicol ointment (Martindale Pharma, Wooburn Green, UK) was applied topically immediately following injection.

### Light-Induced Retinal Degeneration (LIRD)

Two weeks post subretinal administration of AAV vectors, mice were subjected to LIRD as described in Example 1.

Optical Coherence Tomography (OCT) As described in Example 1.

### Quantitative RT-PCR (QRT-PCR)

Gene expression of exogenous human IRAK3 and endogenous mouse IRAK3 was analyzed using QRT-PCR as described in our filed application. The primer sequences were mouse IRAK3, forward 5′-GACCAGCTCCAACCCAAACT (SEQ ID NO: 36), reverse 5′-GCCACCGCCGGTCATATTTA (SEQ ID NO: 37); human IRAK3, forward 5′-CCCACTCCCTTGGCACATTC (SEQ ID NO: 38), reverse 5′-AGCATGGTTGAACGTTGTGC (SEQ ID NO: 39); mouse RPS29, forward 5′-ACGGTCTGATCCGCAAATAC (SEQ ID NO: 44), reverse 5′-ATCCATTCAAGGTCGCTTAGTC (SEQ ID NO: 45).

### Mouse Retinal Sections and Fluorescence Staining for IRAK-M, Mitochondria and Apoptosis

To evaluate whether subretinal administration of AAV2 vectors augments the expression of human IRAK-M within mouse retinas, eyes were enucleated 2 weeks post injection and fixed with 2% paraformaldehyde (PFA) before cryosections prepared. The sections were permeabilized with 0.1% Triton X-100, blocked with 10% normal donkey serum, 5% BSA plus 0.3 M glycine before incubation with either rabbit anti-human IRAK-M (1:500, Cat. HPA043097, Merck, Gillingham, UK) or rabbit anti-IRAK-M (1:500, Cat. ab8116, Abcam) overnight at 4° C. After wash, sections were incubated with donkey anti-rabbit IgG conjugated with Alexa Fluor 488 (1:1000, ThermoFisher Scientific). DAPI counterstain was used to show nuclei in sections. Tissues were washed and mounted in Vectashield antifade medium and examined by confocal microscopy.

MitoView Green is a mitochondrial membrane potential-insensitive dye that can be used to stain mitochondria in live as well as formaldehyde-fixed cells. The fluorescence of cells stained with this dye is directly proportional to the mitochondrial content. The fixed mouse retinal sections were washed in PBS and incubated with 100 nM of MitoView Green for 30 minutes at RT. After wash and counterstaining with DAPI, the samples were mounted for observation under confocal microscopy.

Retinal cell apoptosis was determined by TUNEL staining using an In Situ Cell Death Detection Kit (Roche Diagnostics, Burgess Hill, UK) according to the manufacturer's instructions.

### LDH Cytotoxicity Assay

As described in Example 1.

### Statistics

Results are presented as means±SD. Two-way ANOVA was used to assess the interrelationship of two independent variables on a dependent variable, followed by the Kruskal-Wallis test with Bonferroni correction for post hoc comparisons. Unpaired two-tailed Student's t-test was performed between two groups. Differences between groups were considered significant at P<0.05. Statistical analyses were conducted using GraphPad Prism 7.0.

### Results

Histological Analysis Demonstrates Reduced IRAK-M Expression in RPE with Age and in AMD

To determine the tissue spatial expression of IRAK-M protein associated with ageing and AMD, we performed IHC analysis on paraffin-embedded retinal sections collected from 2 “young” (aged 30 and 59y) and 5 “old” (76-97y) individuals without history of AMD, and 11 AMD patients (76-95y). In young samples, IRAK-M were present across different layers of inner and outer retina, RPE and choroid (data not shown, Ctr 59y). In aged control or AMD samples, the pattern and strength of signal immunopositivity altered variably, for example with a heightened signal in outer plexiform layer (OPL)/outer nuclear layer (ONL) (data not shown, Ctr 97y), nerve fiber layer (NFL) (data not shown, Early AMD 95y) or inner nuclear layer (INL)/ONL/inner segment (IS) (data not shown, mild AMD 76y). Through color deconvolution, we were able to separate IRAK-M immunopositivity from the RPE pigment for quantification of IRAK-M expression in the RPE, along with analyses for choroid and retina. We identified a markedly reduced IRAK-M expression at the macula in both RPE and choroid with old age (FIG. 17). Furthermore, in AMD patients IRAK-M level of expression was lower in macular RPE compared to age-matched non-AMD subjects, which was however not observed in macular choroid (FIG. 17). Reduction in IRAK-M expression in peripheral RPE, choroid and retina was only evident in aged choroid in comparison to young counterpart (FIG. 17). Additionally, a nonspecific staining of Bruch's membrane (BM) for Hematoxylin and IRAK-M was noted in AMD samples (data not shown), as this was also observed in negative control staining. The intensified BM was not evident in control eyes, which is in agreement with the finding that BM is markedly thickened in AMD (101).

### Subretinal Administration Induces AAV2-Mediated Transgene Expression of Human IRAK-M in Mouse RPE

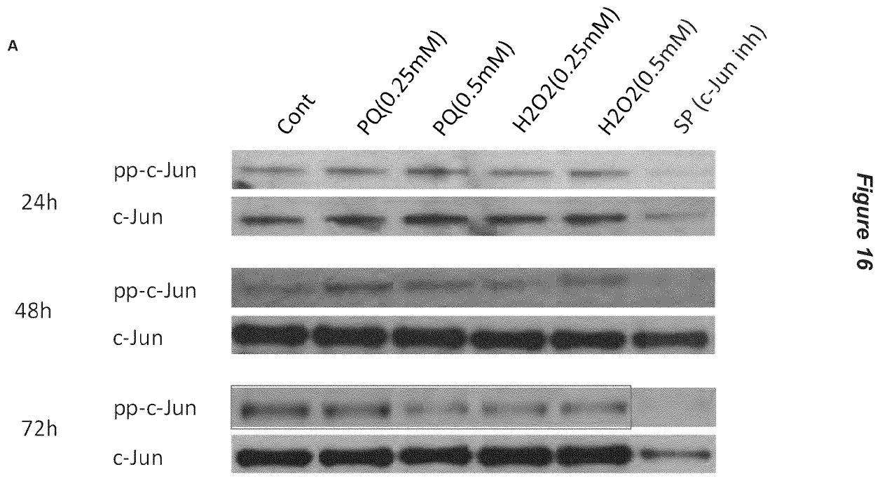

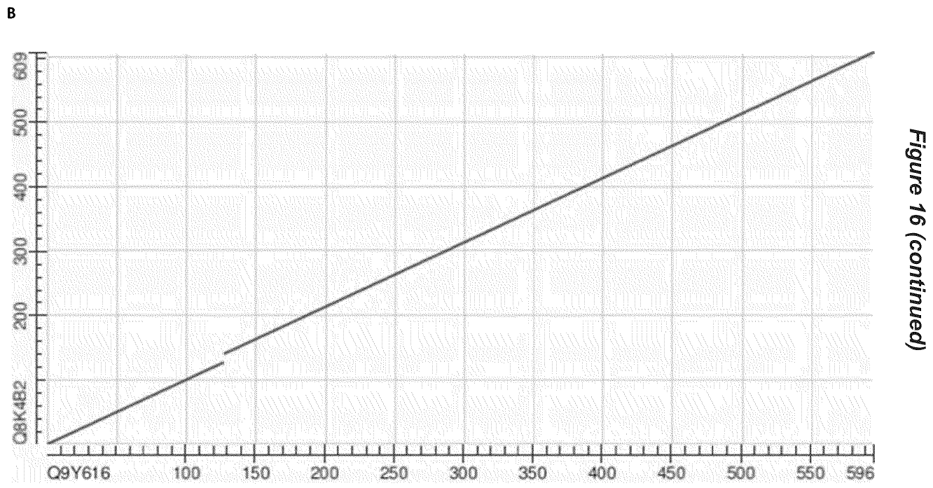

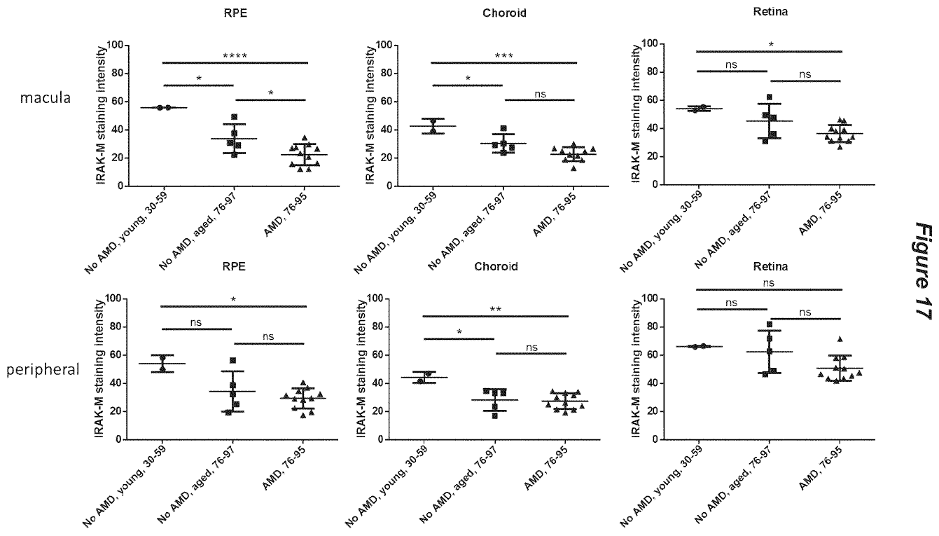

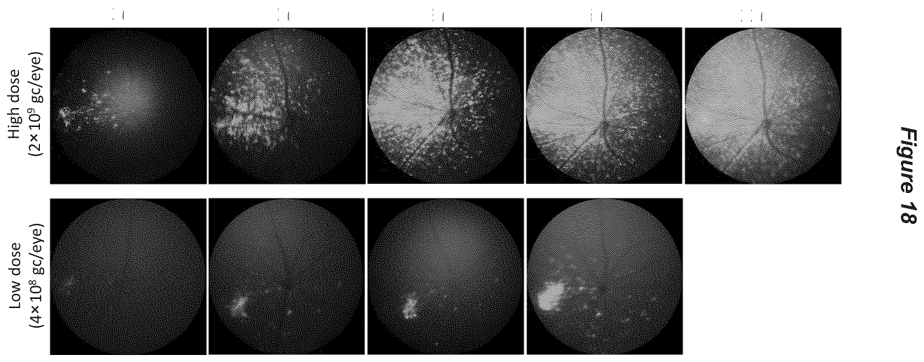

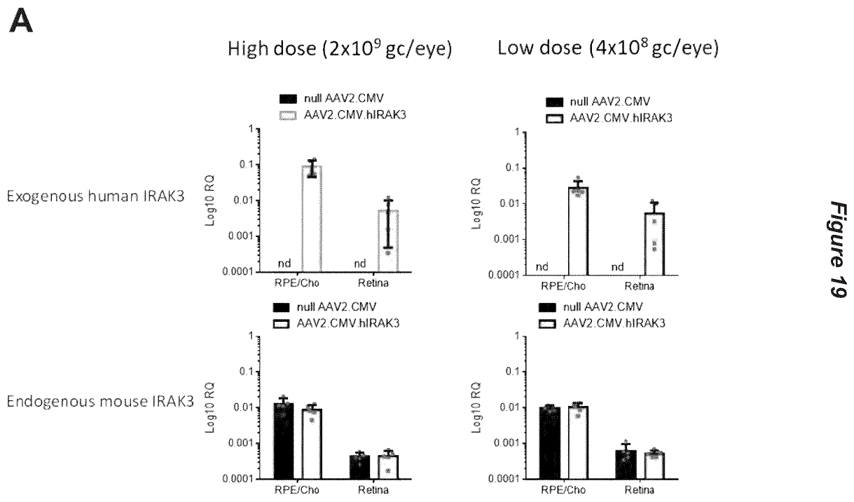

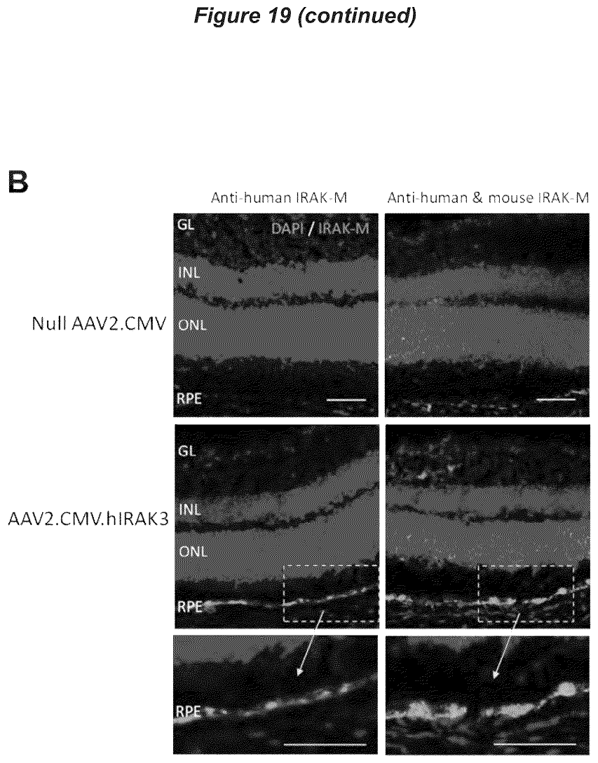

To date, AAV2 is the most well-characterised AAV serotype in clinical trials to treat RPE-related eye diseases (88). To identify the dose-dependent transduction efficacy, 2 μl AAV2 encoding EGFP under the control of constitutive cytomegalovirus (CMV) promoter (AAV2.CMV.EGFP) at either 1×10¹² or 2×10¹¹ gc/ml were delivered into mouse eyes via subretinal route. The high dose (1×10¹² gc/ml in 2 μl, or 2×10⁹ gc/eye) induced a more pronounced EGFP expression 2-11 weeks post the injection than the low dose (2×10¹¹ gc/ml or 4×10⁸ gc/eye) (FIG. 18). Administration with the high dose of AAV2.CMV.hIRAK3, compared to null AAV2.CMV, resulted in a higher hIRAK3 mRNA expression in RPE/choroid two weeks after injection (FIG. 19A). AAV-induced hIRAK3 mRNA expression in the retina was lower compared to that in the RPE/choroid. Notably, endogenous mouse IRAK3 mRNA expression in both RPE/choroid and retina tissues did not change following the introduction of exogenous hIRAK3 (FIG. 19A). The induced hIRAK-M protein expression was detected in the RPE, confirmed by immunohistochemistry using two independent IRAK-M antibodies (FIG. 19B).

### IRAK-M Gene Therapy Suppresses Light-Induced Retinal Degeneration

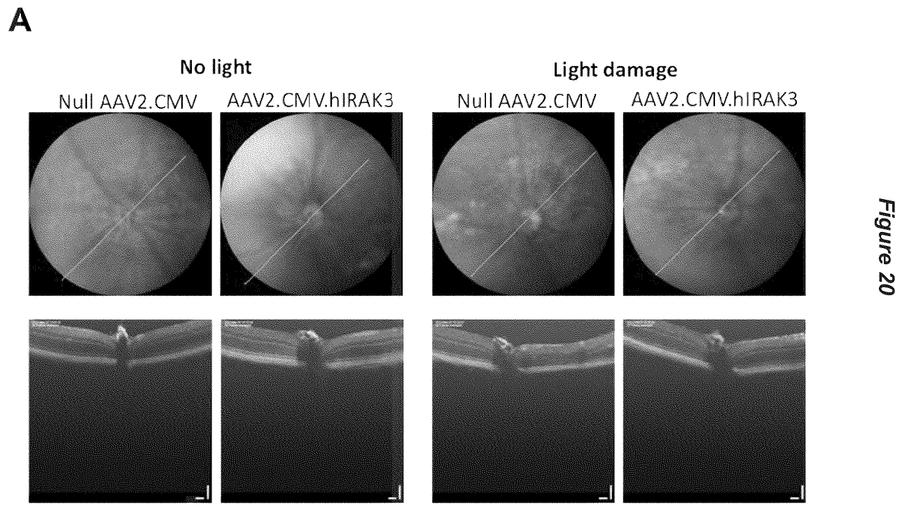

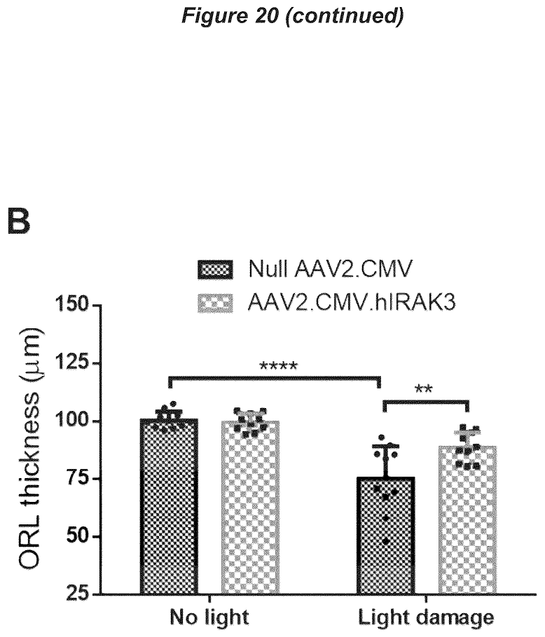

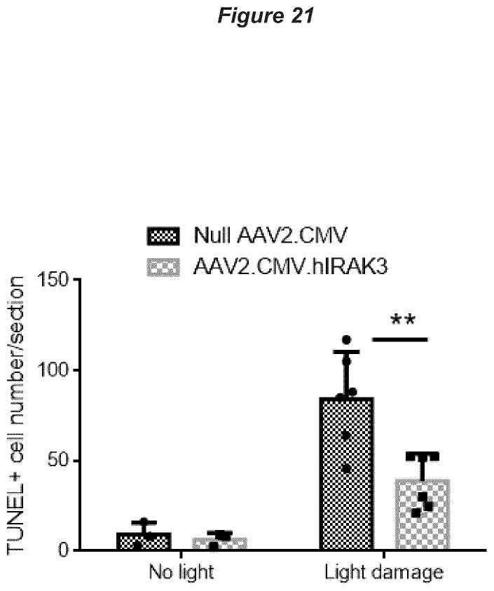

To evaluate the protective effects of IRAK-M transgene expression in vivo, we applied light-induced retinal degeneration (LIRD) in mice 2 weeks after the subretinal injection of the AAV2.CMV.hIRAK3 or null AAV2.CMV at the dose of 2×10⁹ gc/eye. Optical Coherence Tomography (OCT) was employed to detect retinal pathology in response to therapy. In control eyes without light challenge, hIRAK3 transduction did not alter the gross morphology of OCT sections 4 weeks after the injection (FIG. 20A). In the LIRD model, only outer retina thickness was reduced, whilst inner retina thickness remained unchanged as expected in this model. Light exposure of the null AAV2-injected eyes resulted in a decrease in outer retinal thickness, indicative of the PR loss. The protective effect of AAV2.CMV.hIRAK3 treatment from PR injury was conspicuous, as demonstrated by suppression of light-induced outer retinal thinning (FIG. 20B). Contemporaneous with the retaining of retinal thickness by IRAK-M gene therapy was a reduction in light-induced TUNEL-positive apoptosis within the retina (FIG. 21).

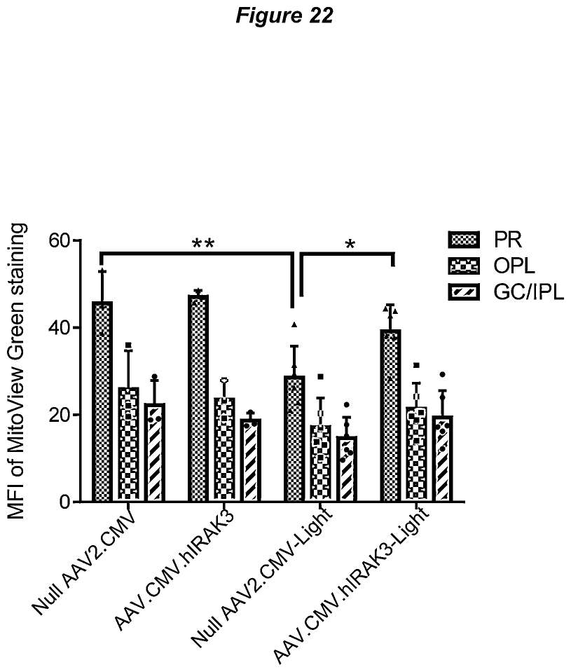

The retina is one of the most energy-demanding organs in the body. Alongside aerobic glycolysis, mitochondria activity in retinal cells such as PR, is essential for tissue function and normal vision (102). Within the retina, mitochondria are abundantly distributed at the ganglion cells (GC), inner plexiform layer (IPL), outer plexiform layer (OPL), and inner segments (IS) of the PR (103). In the LIRD model, mitochondrial staining using MitoView Green Dye in the retinal sections demonstrated impairment of mitochondria in PR cells by light damage (FIG. 22). This impairment was significantly reversed by AAV2-mediated IRAK-M gene delivery, as the mitochondria in GL, IPL and OPL were less affected in the light damage model (FIG. 22).

### IRAK-M Gene Therapy Prevents Age-Associated Retinal Degeneration in Irak3-Mice

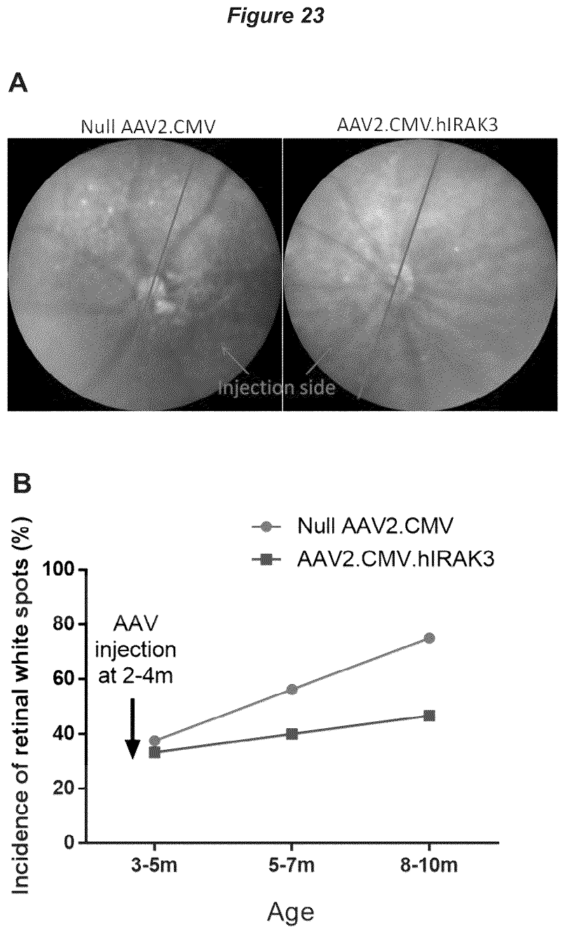

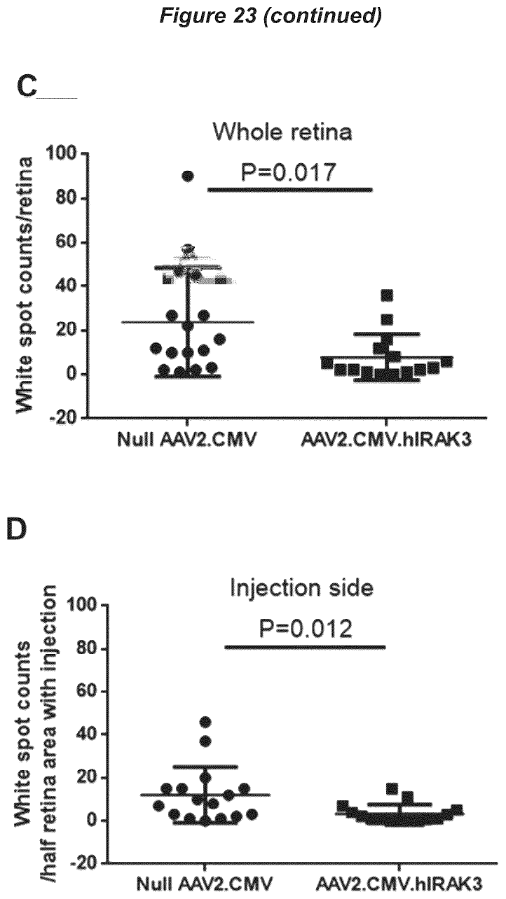

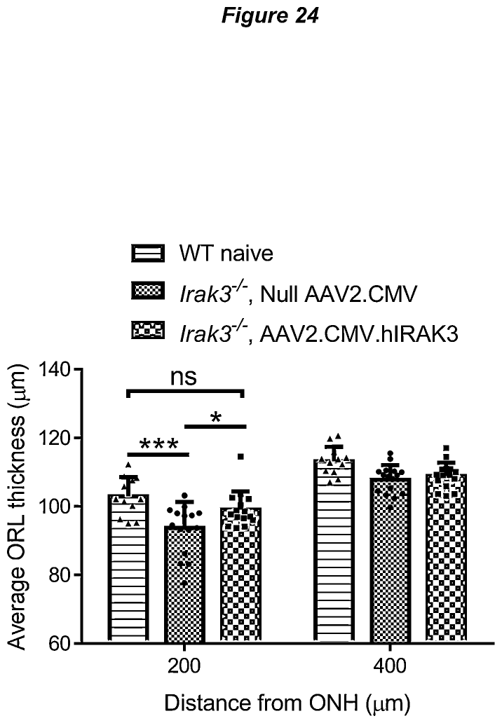

Based on the finding that Irak3^{-/-} mice develop signs of retinal degeneration earlier than WT controls, we asked whether AAV-mediated IRAK-M augmentation could prevent the retinal pathologies caused by IRAK-M deficiency. To this end, we performed subretinal administration of AAV2.CMV.hIRAK3 or null AAV2.CMV (2×10⁹ gc/eye) in young Irak3^{-/-} mice (2-4m old). Following the subretinal delivery of IRAK3 transgene, not only the age-dependent occurrence of retinal spots was markedly inhibited during ageing (FIGS. 23A and B), but also the number of retinal spots in aged KO mice (8-10m old) was significantly reduced in the retina (FIG. 23C), which is more pronounced at the virus administration side (FIG. 23D). The therapeutic effect of IRAK3 gene therapy was further demonstrated by an inhibition of outer retinal thinning in the KO mice (8-10m old) (FIG. 24).

### Comparison of Various Promoters for Efficient IRAK3 Gene Transduction

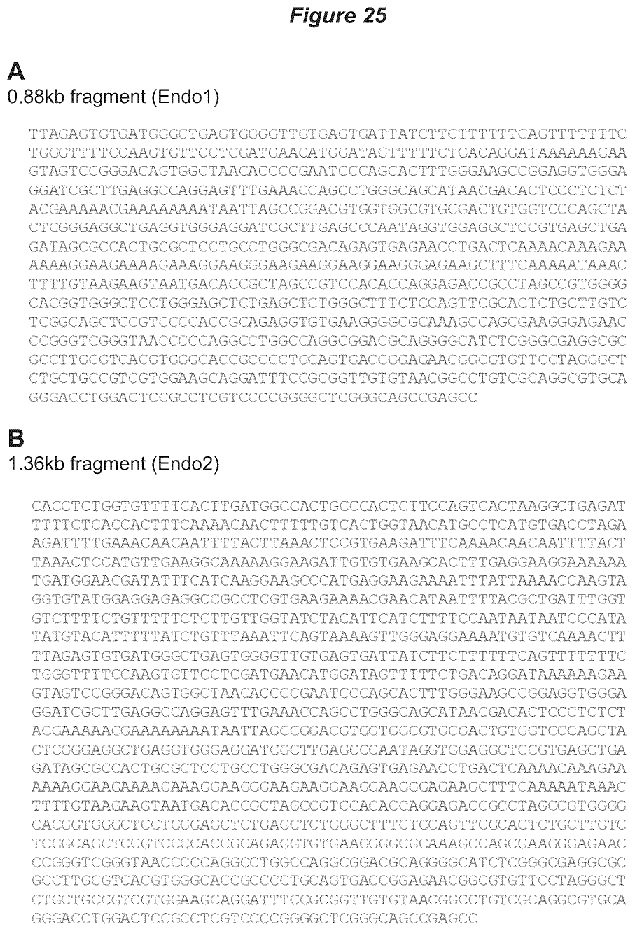

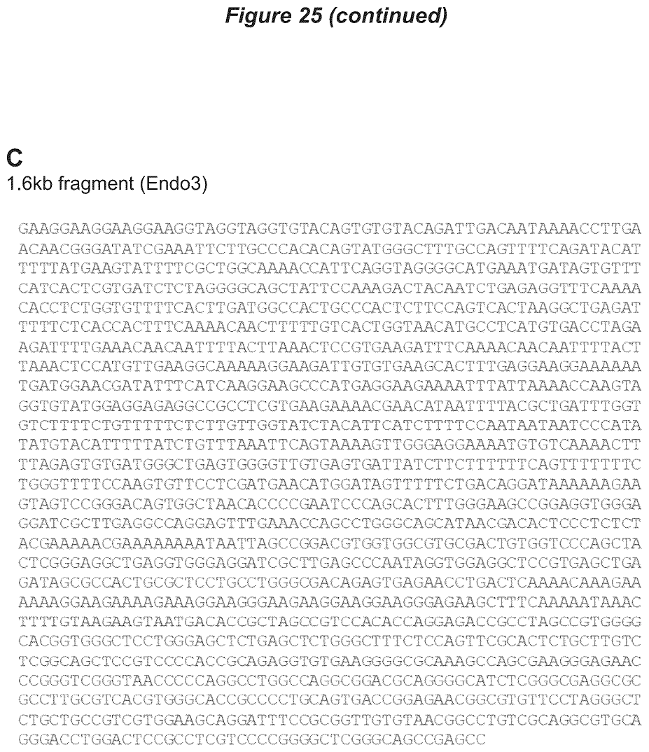

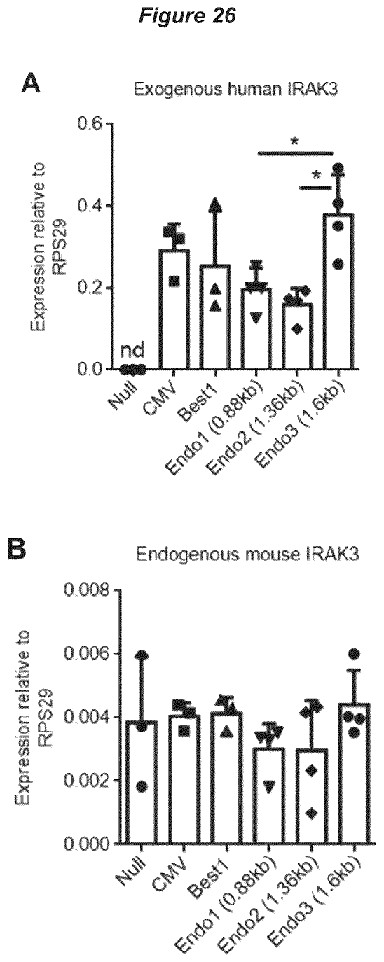

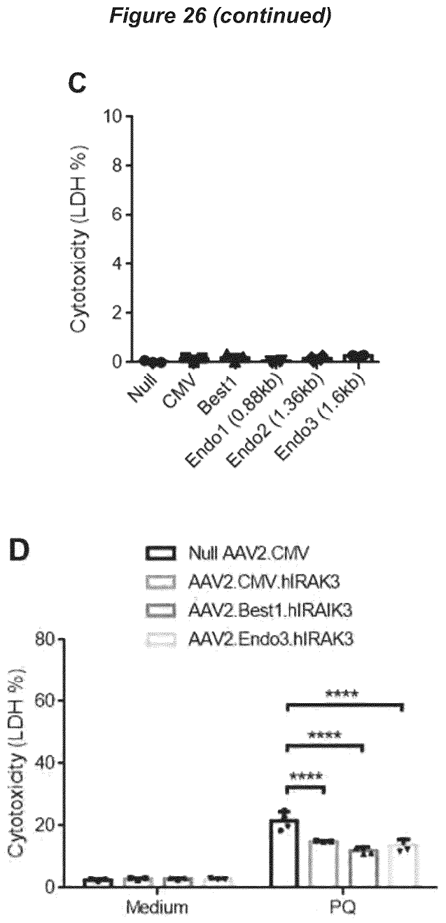

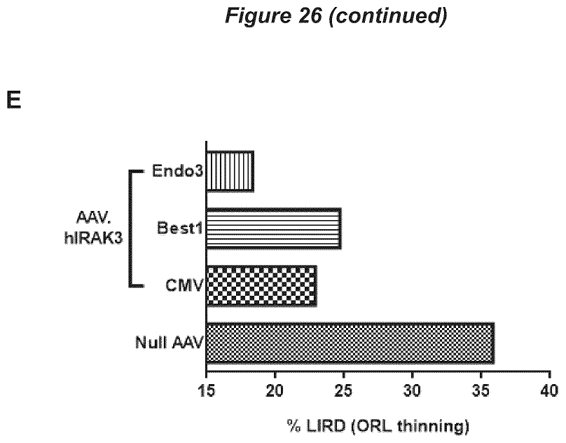

The promoter of a viral vector is the major regulatory element that dictates the efficiency and specificity of transgene expression. We compared the transduction efficiency of human IRAK-M gene transduction in mouse B6-RPE07 cells using AAV2 under control of five different promoters, including a ubiquitous CMV promoter, an RPE-specific Bestrophin 1 (Best1) promoter, and three putative endogenous (Endo) promoters of human IRAK3 gene. As shown in FIG. 25, three fragments in front of the first exon of human IRAK3 gene (Ensembl ID: ENSG00000090376) were selected as putative Endo promoters. The 0.88 kb fragment (Endo1) is our predicted “core promoter” which includes the CpG island and H3K methylation marks; the 1.36 kb fragment (Endo2) is predicted the maximum promoter size which the AAV backbone CMV.GFP.WPRE.io2 can accommodate given IRAK3 gene size plus WPRE and io2 elements; the 1.6 kb fragment (Endo3) is approx. the maximum promoter size that AAV vectors in general can accommodate given IRAK3 gene size. FIG. 26A demonstrated comparable transgene expressions induced by different AAV2 vectors with CMV, Best1 or Endo3 (1.6 kb) promoters, whereas the other two endogenous promoters with shorter sequences were less efficient but nevertheless capable of driving robust expression. Notably, the human IRAK3 gene transduction had no effects on the expression of endogenous mouse IRAK3 gene (FIG. 26B) and did not induce cell death (FIG. 26C). Following defining the transduction efficiency of different promoters, we then assessed protective effects of AAV2. hIRAK3 with the promoters in human RPE cells (ARPE-19) in response to oxidative stress. The data demonstrate that AAV2.CMV.hIRAK3, AAV2.Best1.hIRAK3 and AAV2.Endo3.hIRAK3 can all significantly prevent paraquat-induced cell death (FIG. 26D). In LIRD model, the AAVs with ubiquitous, RPE-specific, or native promoters also showed potential in protecting retinal degeneration against light damage (FIG. 26E).

### AAV5 Transduces IRAK-M Gene Expression

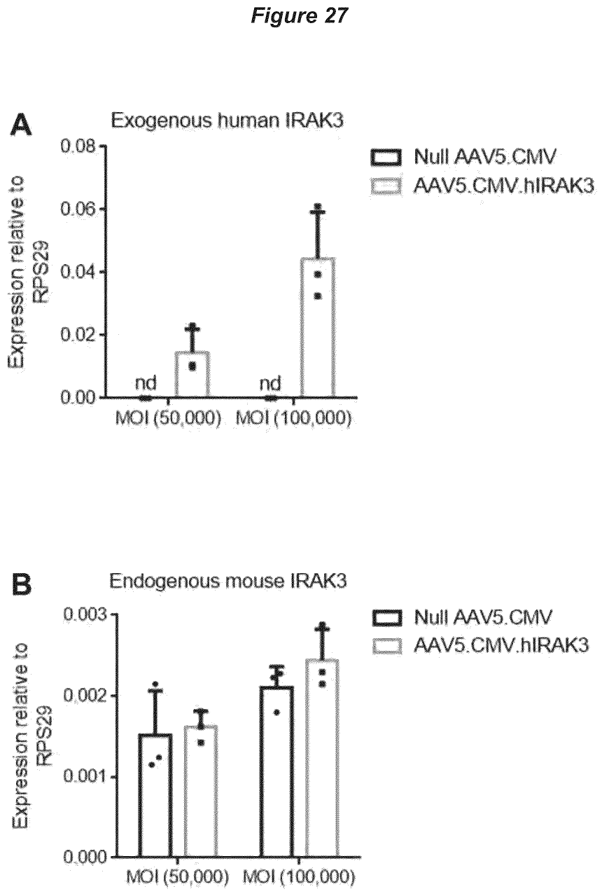

Apart from AAV2, the predominant serotype used in ongoing ocular gene therapy trials, other AAV capsid types such as AAV5, have also been under intensive investigation. Here we showed that AAV5.CMV.hIRAK3 transduction in mouse RPE cells enhanced human IRAK3 gene expression in a dose-dependent manner (FIG. 27A), without affecting the expression of endogenous mouse IRAK3 (FIG. 27B). Therefore, demonstrating the other AAV capsid serotypes may be used in context of the present invention.

### SUMMARY

As an in vivo proof of concept (POC), the proposed gene therapy modality to augment IRAK-M expression is effective. We demonstrate the prevention of AMD-like phenotype in two animal models, light-induced retinal damage (LIRD) in wild-type mice and age-related retinal damage in IRAK-M KO mice.

Apart from the mechanism-based and functional investigation, IHC staining of human donor eyes reveals significant down-regulation of IRAK-M in macular RPE by ageing, which is even lower in elderly people with AMD. Whether a more rapid decrease of IRAK-M triggers AMD initiation or AMD accelerates the decline of IRAK-M expression, remains to be identified. Nonetheless, as our data pinpoints an age-related and oxidative stress-induced decline of IRAK-M expression to be at the root of AMD, augmenting IRAK-M expression by gene therapy holds the potential to prevent or slow down the progression from early stage to late dry AMD, which has not been achieved currently.

The AAV2 vector we utilize for subretinal administration is hitherto the most characterised AAV serotype in clinical trials to treat RPE-related ocular diseases with durable gene expression and no deleterious side effects observed (88, 89, 43). Among other AAV capsid variants, AAV5 possesses the known advantage of low seroprevalence and high transduction efficiency. Gene therapy product using AAV5 in treatment for X-linked retinitis pigmentosa (XLRP) has recently entered phase 3 clinical trials (ClinicalTrials.gov Identifier: NCT04671433). In our test, AAV5 displayed a capability to deliver targeted human IRAK3 expression in mouse RPE, which expanded our vehicle choices for more comprehensive studies.

Another determinator of transduction specificity and efficiency is the promotor used to drive the target gene expression. In addition to CMV, both the RPE-specific Best1 promoter and all three versions of the promotor for the endogenous human IRAK3 gene (Endo1, 2, and 3) achieved robust transgene expression.

The two main strategies currently under investigation for dry AMD treatment are stem cell replacement (105) by RPE transplantation and immune regulation (106). Clinical trials along these two arms of strategies are ongoing at different phases. Other therapeutic innovations focusing on immune regulation are gene therapies targeting complement signal cascade, such as gene therapy trials GT005 that induces complement factor I (CFI) expression (107) and HMR59 (AAVCAGsCD59) that expresses C59 to prevent the formation of membrane attack complex (MAC) (108). The present invention is co-targeting both cell bioenergetic health, inflammation and oxidative stress, by restoring IRAK-M expression, to redress homeostatic control and treat AMD.

### REFERENCES

A number of publications are cited above in order to more fully describe and disclose the invention and the state of the art to which the invention pertains. Full citations for these references are provided below. The entirety of each of these references is incorporated herein.
1. J. Moretti, J. M. Blander, Cell-autonomous stress responses in innate immunity. J Leukoc Biol 101, 77-86 (2017).
2. Y. Dong et al., Stress-induced NLRP3 inflammasome activation negatively regulates fear memory in mice. J Neuroinflammation 17, 205 (2020).
3. M. M. Giarmarco et al., Daily mitochondrial dynamics in cone photoreceptors. Proc Natl Acad Sci USA 117, 28816-28827 (2020).
4. M. Chen, H. Xu, Parainflammation, chronic inflammation, and age-related macular degeneration. J Leukoc Biol 98, 713-725 (2015).
5. C. Franceschi, P. Garagnani, P. Parini, C. Giuliani, A. Santoro, Inflammaging: a new immune-metabolic viewpoint for age-related diseases. Nat Rev Endocrinol 14, 576-590 (2018).
6. J. T. Handa et al., A systems biology approach towards understanding and treating non-neovascular age-related macular degeneration. Nat Commun 10, 3347 (2019).
7. N. Congdon et al., Causes and prevalence of visual impairment among adults in the United States. Arch Ophthalmol 122, 477-485 (2004).
8. D. A. Copland, S. Theodoropoulou, J. Liu, A. D. Dick, A Perspective of AMD Through the Eyes of Immunology. Invest Ophthalmol Vis Sci 59, AMD83-AMD92 (2018).
9. J. Wu, X. Sun, Complement system and age-related macular degeneration: drugs and challenges. Drug Des Devel Ther 13, 2413-2425 (2019).
10. L. G. Fritsche et al., A large genome-wide association study of age-related macular degeneration highlights contributions of rare and common variants. Nat Genet 48, 134-143 (2016).
11. M. J. Geerlings, E. K. de Jong, A. I. den Hollander, The complement system in age-related macular degeneration: A review of rare genetic variants and implications for personalized treatment. Mol Immunol 84, 65-76 (2017).
12. A. Armento, M. Ueffing, S. J. Clark, The complement system in age-related macular degeneration. Cell Mol Life Sci 78, 4487-4505 (2021).
13. J. Liu et al., Impairing autophagy in retinal pigment epithelium leads to inflammasome activation and enhanced macrophage-mediated angiogenesis. Sci Rep 6, 20639 (2016).
14. J. Liu et al., Myeloid cells expressing VEGF and arginase-1 following uptake of damaged retinal pigment epithelium suggests potential mechanism that drives the onset of choroidal angiogenesis in mice. PLOS One 8, e72935 (2013).
15. W. K. Wu et al., IL-4 regulates specific Arg-1 (+) macrophage sFlt-1-mediated inhibition of angiogenesis. Am J Pathol 185, 2324-2335 (2015).
16. K. S. Lee, S. Lin, D. A. Copland, A. D. Dick, J. Liu, Cellular senescence in the aging retina and developments of senotherapies for age-related macular degeneration. J Neuroinflammation 18, 32 (2021).
17. A. J. Clare et al., Treatment with interleukin-33 is non-toxic and protects retinal pigment epithelium in an ageing model of outer retinal degeneration. J Cell Mol Med 24, 13546-13550 (2020).
18. C. A. Curcio, N. E. Medeiros, C. L. Millican, Photoreceptor loss in age-related macular degeneration. Invest Ophthalmol Vis Sci 37, 1236-1249 (1996).
19. S. Wang et al., Deciphering primate retinal aging at single-cell resolution. Protein Cell (2020).
20. J. G. Hollyfield et al., Oxidative damage-induced inflammation initiates age-related macular degeneration. Nat Med 14, 194-198 (2008).
21. M. C. Marazita, A. Dugour, M. D. Marquioni-Ramella, J. M. Figueroa, A. M. Suburo, Oxidative stress-induced premature senescence dysregulates VEGF and CFH expression in retinal pigment epithelial cells: Implications for Age-related Macular Degeneration. Redox Biol 7, 78-87 (2016).
22. M. Chen, C. Luo, R. Penalva, H. Xu, Paraquat-induced retinal degeneration is exaggerated in CX3CR1-deficient mice and is associated with increased retinal inflammation. Invest Ophthalmol Vis Sci 54, 682-690 (2013).
23. L. M. Scott et al., Interleukin-33 regulates metabolic reprogramming of the retinal pigment epithelium in response to immune stressors. JCI Insight 6 (2021).
24. N. Piippo et al., Oxidative Stress is the Principal Contributor to Inflammasome Activation in Retinal Pigment Epithelium Cells with Defunct Proteasomes and Autophagy. Cell Physiol Biochem 49, 359-367 (2018).
25. R. Gill, A. Tsung, T. Billiar, Linking oxidative stress to inflammation: Toll-like receptors. Free Radic Biol Med 48, 1121-1132 (2010).
26. R. B. Nussenblatt et al., Immune responses in age-related macular degeneration and a possible long-term therapeutic strategy for prevention. Am J Ophthalmol 158, 5-11.e12 (2014).
27. B. Detrick, J. J. Hooks, Immune regulation in the retina. Immunol Res 47, 153-161 (2010).
28. A. Jain, S. Kaczanowska, E. Davila, IL-1 Receptor-Associated Kinase Signaling and Its Role in Inflammation, Cancer Progression, and Therapy Resistance. Front Immunol 5, 553 (2014).
29. K. Kobayashi et al., IRAK-M is a negative regulator of Toll-like receptor signaling. Cell 110, 191-202 (2002).
30. L. L. Hubbard, B. B. Moore, IRAK-M regulation and function in host defense and immune homeostasis. Infect Dis Rep 2 (2010).
31. M. Hulsmans et al., Interleukin-1 receptor-associated kinase-3 is a key inhibitor of inflammation in obesity and metabolic syndrome. PLOS One 7, e30414 (2012).
32. W. A. Tseng et al., NLRP3 inflammasome activation in retinal pigment epithelial cells by lysosomal destabilization: implications for age-related macular degeneration. Invest Ophthalmol Vis Sci 54, 110-120 (2013).
33. S. Wang, X. Lai, Y. Deng, Y. Song, Correlation between mouse age and human age in anti-tumor research: Significance and method establishment. Life Sci 242, 117242 (2020).
34. E. J. Kim et al., Complete Transcriptome Profiling of Normal and Age-Related Macular Degeneration Eye Tissues Reveals Dysregulation of Anti-Sense Transcription. Sci Rep 8, 3040 (2018).
35. Y. Ding, B. Aredo, X. Zhong, C. X. Zhao, R. L. Ufret-Vincenty, Increased susceptibility to fundus camera-delivered light-induced retinal degeneration in mice deficient in oxidative stress response proteins. Exp Eye Res 159, 58-68 (2017).
36. C. Cingolani et al., Retinal degeneration from oxidative damage. Free Radic Biol Med 40, 660-669 (2006).
37. N. K. Wang et al., Cellular origin of fundus autofluorescence in patients and mice with a defective NR2E3 gene. Br J Ophthalmol 93, 1234-1240 (2009).
38. P. Jin et al., Activator protein 1 promotes the transcriptional activation of IRAK-M. Biomed Pharmacother 83, 1212-1219 (2016).
39. C. van't Veer et al., Induction of IRAK-M is associated with lipopolysaccharide tolerance in a human endotoxemia model. J Immunol 179, 7110-7120 (2007).
40. S. Leppä, D. Bohmann, Diverse functions of JNK signaling and c-Jun in stress response and apoptosis. Oncogene 18, 6158-6162 (1999).
41. B. J. Kim, D. J. Zack, The Role of c-Jun N-Terminal Kinase (JNK) in Retinal Degeneration and Vision Loss. Adv Exp Med Biol 1074, 351-357 (2018).
42. M. A. Kanow et al., Biochemical adaptations of the retina and retinal pigment epithelium support a metabolic ecosystem in the vertebrate eye. Elife 6 (2017).
43. S. E. Barker et al., Subretinal delivery of adeno-associated virus serotype 2 results in minimal immune responses that allow repeat vector administration in immunocompetent mice. J Gene Med 11, 486-497 (2009).
44. J. Guy et al., Efficiency and safety of AAV-mediated gene delivery of the human ND4 complex I subunit in the mouse visual system. Invest Ophthalmol Vis Sci 50, 4205-4214 (2009).
45. X. Qi, L. Sun, A. S. Lewin, W. W. Hauswirth, J. Guy, The mutant human ND4 subunit of complex I induces optic neuropathy in the mouse. Invest Ophthalmol Vis Sci 48, 1-10 (2007).
46. D. A. Ferrington, D. Sinha, K. Kaarniranta, Defects in retinal pigment epithelial cell proteolysis and the pathology associated with age-related macular degeneration. Prog Retin Eye Res 51, 69-89 (2016).
47. S. Theodoropoulou et al., Interleukin-33 regulates tissue remodelling and inhibits angiogenesis in the eye. J Pathol 241, 45-56 (2017).
48. J. W. Crabb, The proteomics of drusen. Cold Spring Harb Perspect Med 4, a017194 (2014).
49. A. L. Wang et al., Autophagy and exosomes in the aged retinal pigment epithelium: possible relevance to drusen formation and age-related macular degeneration. PLOS One 4, e4160 (2009).
50. R. Medzhitov, T. Horng, Transcriptional control of the inflammatory response. Nat Rev Immunol 9, 692-703 (2009).
51. S. Sugita, M. Mandai, H. Kamao, M. Takahashi, Immunological aspects of RPE cell transplantation. Prog Retin Eye Res, 100950 (2021).
52. I. Benhar, K. Reemst, V. Kalchenko, M. Schwartz, The retinal pigment epithelium as a gateway for monocyte trafficking into the eye. EMBO J 35, 1219-1235 (2016).
53. H. B. Juel, C. Faber, M. S. Udsen, L. Folkersen, M. H. Nissen, Chemokine expression in retinal pigment epithelial ARPE-19 cells in response to coculture with activated T cells. Invest Ophthalmol Vis Sci 53, 8472-8480 (2012).
54. A. Weber, P. Wasiliew, M. Kracht, Interleukin-1 (IL-1) pathway. Sci Signal 3, cm1 (2010).
55. S. Sugita et al., Retinal pigment epithelium-derived CTLA-2alpha induces TGFbeta-producing T regulatory cells. J Immunol 181, 7525-7536 (2008).
56. N. Arimura et al., Intraocular expression and release of high-mobility group box 1 protein in retinal detachment. Lab Invest 89, 278-289 (2009).
57. M. V. Kumar, C. N. Nagineni, M. S. Chin, J. J. Hooks, B. Detrick, Innate immunity in the retina: Toll-like receptor (TLR) signaling in human retinal pigment epithelial cells. J Neuroimmunol 153, 7-15 (2004).
58. P. Mandrekar, S. Bala, D. Catalano, K. Kodys, G. Szabo, The opposite effects of acute and chronic alcohol on lipopolysaccharide-induced inflammation are linked to IRAK-M in human monocytes. J Immunol 183, 1320-1327 (2009).
59. N. Wang et al., Adenovirus-mediated overexpression of c-Jun and c-Fos induces intercellular adhesion molecule-1 and monocyte chemoattractant protein-1 in human endothelial cells. Arterioscler Thromb Vasc Biol 19, 2078-2084 (1999).
60. K. Riabowol, J. Schiff, M. Z. Gilman, Transcription factor AP-1 activity is required for initiation of DNA synthesis and is lost during cellular aging. Proc Natl Acad Sci USA 89, 157-161 (1992).
61. M. Helenius, M. Hänninen, S. K. Lehtinen, A. Salminen, Changes associated with aging and replicative senescence in the regulation of transcription factor nuclear factor-kappa B. Biochem J 318 (Pt 2), 603-608 (1996).
62. J. S. Tilstra, C. L. Clauson, L. J. Niedernhofer, P. D. Robbins, NF-κB in Aging and Disease. Aging Dis 2, 449-465 (2011).
63. A. Sheerin, K. S. Thompson, M. H. Goyns, Altered composition and DNA binding activity of the AP-1 transcription factor during the ageing of human fibroblasts. Mech Ageing Dev 122, 1813-1824 (2001).
64. R. Medicherla, S. Leers-Sucheta, Y. Luo, S. Azhar, Impaired activation of AP-1 and altered expression of constituent proteins in rat adrenal during ageing. Mech Ageing Dev 122, 1169-1186 (2001).
65. J. Yin, F. Thomas, J. C. Lang, E. Chaum, Modulation of oxidative stress responses in the human retinal pigment epithelium following treatment with vitamin C. J Cell Physiol 226, 2025-2032 (2011).
66. E. Chaum, J. Yin, H. Yang, F. Thomas, J. C. Lang, Quantitative AP-1 gene regulation by oxidative stress in the human retinal pigment epithelium. J Cell Biochem 108, 1280-1291 (2009).
67. I. Jialal, H. Kaur, S. Devaraj, Toll-like receptor status in obesity and metabolic syndrome: a translational perspective. J Clin Endocrinol Metab 99, 39-48 (2014).
68. H. Li et al., IL-1 receptor-associated kinase M is a central regulator of osteoclast differentiation and activation. J Exp Med 201, 1169-1177 (2005).
69. C. Lyu et al., IRAK-M Deficiency Exacerbates Ischemic Neurovascular Injuries in Experimental Stroke Mice. Front Cell Neurosci 12, 504 (2018).
70. Q. Tan et al., IRAK-M deficiency promotes the development of type 1 diabetes in NOD mice. Diabetes 63, 2761-2775 (2014).
71. F. M. Pool, C. Kiel, L. Serrano, P. J. Luthert, Repository of proposed pathways and protein-protein interaction networks in age-related macular degeneration. NPJ Aging Mech Dis 6, 2 (2020).
72. K. Kaarniranta et al., Mechanisms of mitochondrial dysfunction and their impact on age-related macular degeneration. Prog Retin Eye Res 79, 100858 (2020).
73. N. Zmora, S. Bashiardes, M. Levy, E. Elinav, The Role of the Immune System in Metabolic Health and Disease. Cell Metab 25, 506-521 (2017).
74. W. K. E. Ip, N. Hoshi, D. S. Shouval, S. Snapper, R. Medzhitov, Anti-inflammatory effect of IL-10 mediated by metabolic reprogramming of macrophages. Science 356, 513-519 (2017).
75. X. Fu, T. Tan, P. Liu, Regulation of Autophagy by Non-Steroidal Anti-Inflammatory Drugs in Cancer. Cancer Manag Res 12, 4595-4604 (2020).
76. A. Salminen, A. Kauppinen, K. Kaarniranta, Emerging role of NF-κB signaling in the induction of senescence-associated secretory phenotype (SASP). Cell Signal 24, 835-845 (2012).
77. M. Borghesan, W. M. H. Hoogaars, M. Varela-Eirin, N. Talma, M. Demaria, A Senescence-Centric View of Aging: Implications for Longevity and Disease. Trends Cell Biol (2020).
78. C. M. Ramsden et al., Rescue of the MERTK phagocytic defect in a human iPSC disease model using translational read-through inducing drugs. Sci Rep 7, 51 (2017).
79. R. Fernandez-Godino, D. L. Garland, E. A. Pierce, Isolation, culture and characterization of primary mouse RPE cells. Nature protocols 11, 1206-1218 (2016).
80. O. Rosati, M. U. Martin, Identification and characterization of murine IRAK-M. Biochem Biophys Res Commun 293, 1472-1477 (2002).
81. J. Du et al., The structure function of the death domain of human IRAK-M. Cell Commun Signal 12, 77 (2014).
82. N. Davidsohn et al., A single combination gene therapy treats multiple age-related diseases. Proc Natl Acad Sci USA 116, 23505-23511 (2019).
83. J. Kaiser, Gene therapy beats premature-aging syndrome in mice. Science 371, 114 (2021).
84. J. Wu et al., Gene Therapy for Glaucoma by Ciliary Body Aquaporin 1 Disruption Using CRISPR-Cas9. Mol Ther 28, 820-829 (2020).
85. A. L. Askou, T. S. Jakobsen, T. J. Corydon, Retinal gene therapy: an eye-opener of the 21st century. Gene Ther (2020).
86. ClinicalTrials.gov (2019) First in Human Study to Evaluate the Safety and Efficacy of GT005 Administered in Subjects With Dry AMD.
87. ClinicalTrials.gov (2017) Treatment of Advanced Dry Age Related Macular Degeneration With AAVCAGsCD59.
88. S. Russell et al., Efficacy and safety of voretigene neparvovec (AAV2-hRPE65v2) in patients with RPE65-mediated inherited retinal dystrophy: a randomised, controlled, open-label, phase 3 trial. Lancet 390, 849-860 (2017).
89. E. A. Pierce, J. Bennett, The Status of RPE65 Gene Therapy Trials: Safety and Efficacy. Cold Spring Harb Perspect Med 5, a017285 (2015).
90. B. Chang, R. Hurd, J. Wang, P. Nishina, Survey of common eye diseases in laboratory mouse strains. Invest Ophthalmol Vis Sci 54, 4974-4981 (2013).
91. Y. Zhou et al., Metascape provides a biologist-oriented resource for the analysis of systems-level datasets. Nat Commun 10, 1523 (2019).
92. M. Chen et al., Characterization of a spontaneous mouse retinal pigment epithelial cell line B6-RPE07. Invest Ophthalmol Vis Sci 49, 3699-3706 (2008).
93. J. Van den Bossche, J. Baardman, M. P. de Winther, Metabolic Characterization of Polarized M1 and M2 Bone Marrow-derived Macrophages Using Real-time Extracellular Flux Analysis. J Vis Exp (2015).
94. Y. Tong, S. Wang, Not All Stressors Are Equal: Mechanism of Stressors on RPE Cell Degeneration. Front Cell Dev Biol. 8, 591067 (2020).
95. S. Somasundaran, I. J. Constable, C. B. Mellough, L. S. Carvalho, Retinal pigment epithelium and age-related macular degeneration: A review of major disease mechanisms. Clin Exp Ophthalmol. 48 (8), 1043-1056 (2020).
96. F. Bandello, R. Sacconi, L. Querques, E. Corbelli, M. V. Cicinelli, G. Querques, Recent advances in the management of dry age-related macular degeneration: A review. F1000Res. 6, 245 (2017).
97. W. Tan, J. Zou, S. Yoshida, B. Jiang, Y. Zhou, The Role of Inflammation in Age-Related Macular Degeneration. Int J Biol Sci. 16 (15), 2989-3001 (2020).
98. S. Romero-Vazquez, V. Llorens, A. Soler-Boronat, M. Figueras-Roca, A. Adan, B. Molins, Interlink between Inflammation and Oxidative Stress in Age-Related Macular Degeneration: Role of Complement Factor H. Biomedicines. 9 (7) (2021).
99. C. Ng Yin Ling, S. C. Lim, J. B. Jonas, C. Sabanayagam, Obesity and risk of age-related eye diseases: a systematic review of prospective population-based studies. Int J Obes (Lond). 45 (9), 1863-1885 (2021).
100. H. Ghaem Maralani, B. C. Tai, T. Y. Wong, et al., Metabolic syndrome and risk of age-related macular degeneration. Retina. 35 (3), 459-66 (2015).
101. J. Tode, E. Richert, S. Koinzer, et al., Selective Retina Therapy Reduces Bruch's Membrane Thickness and Retinal Pigment Epithelium Pathology in Age-Related Macular Degeneration Mouse Models. Transl Vis Sci Technol. 8 (6), 11 (2019).
102. W. W. Pan, T. J. Wubben, C. G. Besirli, Photoreceptor metabolic reprogramming: current understanding and therapeutic implications. Commun Biol. 4 (1), 245 (2021).
103. G. Chidlow, J. P. M. Wood, P. I. Sia, R. J. Casson, Distribution and Activity of Mitochondrial Proteins in Vascular and Avascular Retinas: Implications for Retinal Metabolism. Invest Ophthalmol Vis Sci. 60 (1), 331-344 (2019).
104. K. L. Pietersz, R. M. Martier, M. S. Baatje, et al., Transduction patterns in the CNS following various routes of AAV-5-mediated gene delivery. Gene Ther. 28 (7-8), 435-446 (2021).
105. T. Maeda, S. Sugita, Y. Kurimoto, M. Takahashi, Trends of Stem Cell Therapies in Age-Related Macular Degeneration. J Clin Med. 10 (8) (2021).
106. T. A. Cabral de Guimaraes, M. Daich Varela, M. Georgiou, M. Michaelides, Treatments for dry age-related macular degeneration: therapeutic avenues, clinical trials and future directions. Br J Ophthalmol. 106 (3), 297-304 (2022).
107. S. Ellis, A. Buchberger, J. Holder, E. Orhan, J. Hughes, GT005, a gene therapy for the treatment of dry age-related macular degeneration (AMD). ARVO Annual Meeting Abstract. 61 (7), 2295 (2020).
108. P. U. Dugel. CLINICAL TRIAL DOWNLOAD: Data on a Gene Therapy for Dry and Wet AMD. A phase 1 clinical trial program is targeting both disease states. Retinal Physician. 17 (April 2020), 16-17 (2020).

For standard molecular biology techniques, see Sambrook, J., Russel, D. W. Molecular Cloning, A Laboratory Manual. 3 ed. 2001, Cold Spring Harbor, New York: Cold Spring Harbor Laboratory Press

### Numbered Paragraphs

The following numbered paragraphs set out various aspects and features of the invention.

1. A nucleic acid for use in a method of treatment or prophylaxis of macular degeneration in a subject, wherein the nucleic acid comprises a nucleic acid sequence encoding IRAK-M and wherein the nucleic acid is capable of driving expression of IRAK-M in a target cell.

2. The nucleic acid for use according to paragraph 1, wherein a promoter is operably linked to the nucleic acid sequence.

3. The nucleic acid for use according to paragraph 2, wherein the promoter is an RPE-specific promoter.

4. The nucleic acid for use according to paragraph 3, wherein the RPE-specific promoter is selected from the group consisting of a RPE65 promoter, a NA65 promoter, a VMD2 promoter, and a Synpiii promoter.

5. The nucleic acid for use according to paragraph 2, wherein the promoter is a ubiquitous promoter.

6. The nucleic acid for use according to any one of the preceding paragraphs, wherein autophagic flux is maintained or increased in the target cell comprising the nucleic acid compared to an equivalent cell not comprising the nucleic acid.

7. The nucleic acid for use according to any one of the preceding paragraphs, wherein mitochondrial activity is maintained or increased in the target cell comprising the nucleic acid compared to an equivalent cell not comprising the nucleic acid.

8. The nucleic acid for use according to any one of the preceding paragraphs, wherein the nucleic acid is suitable for integration into the genome of the target cell by an RNA-guided endonuclease system.

9. The nucleic acid for use according to any one of the preceding paragraphs, wherein the nucleic acid is DNA.

10. The nucleic acid for use according to paragraph 9, wherein the nucleic acid is a plasmid or a minicircle.

11. The nucleic acid for use according to any one of paragraphs 1 to 7, wherein the nucleic acid is RNA.

12. The nucleic acid for use according to paragraph 11, wherein the nucleic acid is messenger RNA or circular RNA.

13. The nucleic acid for use according to any one of the preceding paragraphs, wherein the nucleic acid is delivered to a target cell via a viral vector.

14. The nucleic acid for use according to paragraph 13, wherein the viral vector is selected from the group consisting of an adeno-associated virus vector, an adenovirus vector, a retrovirus vector, an orthomyxovirus vector, a paramyxovirus vector, a papovavirus vector, a picornavirus vector, a lentivirus vector, a herpes simplex virus vector, a vaccinia virus vector, a pox virus vector, an anellovirus vector, and an alphavirus vector.

15. The nucleic acid for use according to any one of paragraphs 1 to 12, wherein the nucleic acid is delivered to a target cell via a non-viral carrier.

16. The nucleic acid for use according to paragraph 15, wherein the non-viral carrier is selected from the group consisting of nanoparticles, liposomes, cationic polymer, and calcium phosphate particles.

17. The nucleic acid for use according to any one of paragraphs 1 to 14, wherein the nucleic acid is a viral vector genome.

18. The nucleic acid for use according to paragraph 17, wherein the viral vector genome is selected from the group consisting of an adeno-associated virus vector genome, an adenovirus vector genome, a retrovirus vector genome, an orthomyxovirus vector genome, a paramyxovirus vector genome, a papovavirus vector genome, a picornavirus vector genome, a lentivirus vector genome, a herpes simplex virus vector genome, a vaccinia virus vector genome, a pox virus vector genome, an anellovirus vector genome, and an alphavirus vector genome.

19. The nucleic acid for use according any one of the preceding paragraphs, wherein the macular degeneration is age-related macular degeneration (AMD).

20. The nucleic acid for use according to paragraph 19, wherein the age-related macular degeneration is dry AMD.

21. The nucleic acid for use according any one of the preceding paragraphs, wherein the target cell is a cell of the retina or the choroid.

22. The nucleic acid for use according to paragraph 21, wherein the target cell is a cell of the retina.

23. The nucleic acid for use according to paragraph 22, wherein the target cell is a cell of the ganglion cell layer (GCL), the inner plexiform layer (IPL), the inner nuclear layer (INL), the outer plexiform layer (OPL), the outer nuclear layer (ONL), the photoreceptor outer segment (POS), or the retinal pigmental epithelium (RPE).

24. The nucleic acid for use according to paragraph 23, wherein the target cell is a cell of the RPE.

25. The nucleic acid for use according any one of paragraphs 1 to 20, wherein the target cell is a myeloid cell.

26. The nucleic acid for use according any one of the preceding paragraphs, wherein the nucleic acid is administered intraocularly, intravitreally, subretinally, or periocularly to a subject.

27. The nucleic acid for use according to paragraph 26, wherein the nucleic acid is administered subretinally.

28. The nucleic acid or vector for use according to any one of the preceding paragraphs, wherein the nucleic acid is administered by injection or infusion.

29. The nucleic acid for use according to paragraph 28, wherein the nucleic acid is administered by subretinal injection.

30. The nucleic acid for use according to any one of the preceding paragraphs, wherein the subject is human.

31. The nucleic acid for use according to any one of the preceding paragraphs, wherein the nucleic acid sequence encodes a polypeptide comprising an amino acid sequence having at least 60% sequence identity to the amino acid sequence of SEQ ID NO: 1.

32. The nucleic acid for use according to any one of the preceding paragraphs, wherein the nucleic acid sequence encodes a polypeptide capable of preventing dissociation of IRAK-1 and/or IRAK-4 from MyD88 in a target cell.

33. A vector virion for use in a method of treatment or prophylaxis of macular degeneration in a subject, wherein the vector virion comprises a nucleic acid comprising a nucleic acid sequence encoding IRAK-M and wherein the nucleic acid is capable of driving expression of IRAK-M in a target cell.

34. The vector virion for use according to paragraph 33, wherein a promoter is operably linked to the nucleic acid sequence.

35. The vector virion for use according to paragraph 34, wherein the promoter is an RPE-specific promoter.

36. The vector virion for use according to paragraph 35, wherein the RPE-specific promoter is selected from the group consisting of a RPE65 promoter, a NA65 promoter, a VMD2 promoter, and a Synpiii promoter.

37. The vector virion for use according to paragraph 34, wherein the promoter is a ubiquitous promoter.

38. The vector virion for use according to any one of paragraphs 33 to 37, wherein autophagic flux is maintained or increased in the target cell comprising the vector virion compared to an equivalent cell not comprising the vector virion.

39. The vector virion for use according to any one of paragraphs 33 to 38, wherein mitochondrial activity is maintained or increased in the target cell comprising the vector virion compared to an equivalent cell not comprising the vector virion.

40. The vector virion for use according to any one of paragraphs 33 to 39, wherein the nucleic acid is suitable for integration into the genome of the target cell by an RNA-guided endonuclease system.

41. The vector virion for use according to any one of paragraphs 33 to 40, wherein the vector virion is selected from the group consisting of adeno-associated virus, adenovirus, retrovirus, orthomyxovirus, paramyxovirus, papovavirus, picornavirus, lentivirus, herpes simplex virus, vaccinia virus, pox virus, anellovirus, and alphavirus.

42. The vector virion for use according to paragraph 41, wherein the vector virion is an adeno-associated virus (AAV).

43. The vector virion for use according to paragraph 42, wherein the AAV is selected from the group consisting of AAV type 1 (AAV-1), AAV type 2 (AAV-2), AAV type 3 (AAV-3), AAV type 4 (AAV-4), AAV type 5 (AAV-5), AAV type 6 (AAV6), AAV type 7 (AAV-7), AAV type 8 (AAV-8), and AAV type 9 (AAV9).

44. The vector virion for use according to paragraph 43, wherein the AAV is AAV2.

45. The vector virion for use according any one of paragraphs 33 to 44, wherein the macular degeneration is age-related macular degeneration (AMD).

46. The vector virion for use according to paragraph 45, wherein the age-related macular degeneration is dry AMD.

47. The vector virion for use according any one of paragraphs 33 to 46, wherein the target cell is a cell of the retina or the choroid.

48. The vector virion for use according to paragraph 47, wherein the target cell is a cell of the retina.

49. The vector virion for use according to paragraph 48, wherein the target cell is a cell of the ganglion cell layer (GCL), the inner plexiform layer (IPL), the inner nuclear layer (INL), the outer plexiform layer (OPL), the outer nuclear layer (ONL), the photoreceptor outer segment (POS), or the retinal pigmental epithelium (RPE).

50. The vector virion for use according to paragraph 49, wherein the target cell is a cell of the RPE.

51. The vector virion for use according any one of paragraphs 33 to 46, wherein the target cell is a myeloid cell.

52. The vector virion for use according any one of paragraphs 33 to 51, wherein the vector virion is administered intraocularly, intravitreally, subretinally, or periocularly to a subject.

53. The vector virion for use according to paragraph 52, wherein the vector virion is administered subretinally.

54. The vector virion for use according to any one of paragraphs 33 to 53, wherein the vector virion is administered by injection or infusion.

55. The vector virion for use according to paragraph 54, wherein the vector virion is administered by subretinal injection.

56. The vector virion for use according to any one of paragraphs 33 to 55, wherein the subject is human.

57. The vector virion for use according to any one of paragraphs 33 to 56, wherein the nucleic acid sequence encodes a polypeptide comprising an amino acid sequence having at least 60% sequence identity to the amino acid sequence of SEQ ID NO: 1.

58. The vector virion for use according to any one of paragraphs 33 to 57, wherein the nucleic acid sequence encodes a polypeptide capable of preventing dissociation of IRAK-1 and/or IRAK-4 from MyD88 in a target cell.

59. An IRAK-M polypeptide for use in a method of treatment or prophylaxis of macular degeneration in a subject.

60. The IRAK-M polypeptide for use according to paragraph 59, wherein the IRAK-M polypeptide comprises an amino acid sequence having at least 60% sequence identity to the amino acid sequence of SEQ ID NO: 1.

61. The IRAK-M polypeptide for use according to paragraph 59 or paragraph 60, wherein the IRAK-M polypeptide is capable of preventing dissociation of IRAK-1 and/or IRAK-4 from MyD88 in a target cell.

62. The IRAK-M polypeptide for use according to any one of paragraphs 59 to 61, wherein the IRAK-M polypeptide further comprises a cell penetrating peptide (CPP).

63. The IRAK-M polypeptide for use according to paragraph 62, wherein the IRAK-M polypeptide further comprises a peptide-based cleavable linker (PCL).

64. The IRAK-M polypeptide for use according to paragraphs 63, wherein the CPP is conjugated to the N-terminus of the PCL and wherein the amino acid sequence having at least 60% sequence identity to the amino acid sequence of SEQ ID NO: 1 is conjugated to the C-terminus of the PCL.

65. The IRAK-M polypeptide of paragraph 63 or paragraph 64, wherein the PCL is a peptide sequence that is cleavable by cathepsin D.

66. The IRAK-M polypeptide for use according to any one of paragraphs 59 to 65, wherein autophagic flux is maintained or increased in the target cell comprising the IRAK-M polypeptide compared to an equivalent cell not comprising the IRAK-M polypeptide.

67. The IRAK-M polypeptide for use according to any one of paragraphs 59 to 66, wherein mitochondrial activity is maintained or increased in the target cell comprising the IRAK-M polypeptide compared to an equivalent cell not comprising the IRAK-M polypeptide.

68. A nucleic acid system, comprising one or more nucleic acids, comprising:
a) a nucleic acid sequence encoding an RNA-guided endonuclease;
b) a nucleic acid sequence encoding a guide RNA complementary to a target sequence associated with an insertion site in the genome of the target cell and capable of directing said RNA-guided endonuclease to said target sequence; and
c) a nucleic acid sequence encoding IRAK-M,
   for use in a method of treatment or prophylaxis of macular degeneration in a subject, wherein the nucleic acid sequence encoding IRAK-M is capable of driving expression of IRAK-M in a target cell of the subject and wherein the nucleic acid system is suitable for directed insertion of the nucleic acid sequence encoding IRAK-M at the insertion site in the genome of the target cell.

69. The nucleic acid system for use according to paragraph 68, wherein the nucleic acid sequence encoding IRAK-M is flanked by a 5′ homology arm and a 3′ homology arm, wherein the 5′ homology arm is homologous to a DNA sequence 5′ of the target sequence from the insertion site and the 3′ homology arm is homologous to a DNA sequence 3′ of the target sequence from the insertion site.

70. The nucleic acid system for use according to paragraph 69, wherein the nucleic acid sequence encoding IRAK-M further comprises a 5′ flanking sequence comprising a target sequence and a 3′ flanking sequence comprising a target sequence.

71. The nucleic acid system for use according to paragraph 70, wherein the 5′ flanking sequence is 5′ of the 5′ homology arm and wherein the 3′ flanking sequence is 3′ of the 3′ homology arm.

72. The nucleic acid system for use according to paragraph 68, wherein the nucleic acid sequence encoding IRAK-M is flanked by a 5′ target sequence and a 3′ target sequence.

73. The nucleic acid system for use according to paragraph 72, wherein the 5′ target sequence and the 3′ target sequence are identical to the target sequence from an insertion site in the genome.

74. A nucleic acid system, comprising one or more nucleic acids, comprising:
a) a nucleic acid sequence encoding a deactivated RNA-guided endonuclease fused to one or more transcriptional activators; and
b) a nucleic acid sequence encoding a guide RNA complementary to a target sequence in the promoter or regulatory sequences of the IRAK3 gene and capable of directing said RNA-guided endonuclease to said target sequence,
   for use in a method of treatment or prophylaxis of macular degeneration in a subject, wherein the nucleic acid system increases IRAK-M expression in a target cell of the subject.

75. A nucleic acid system, comprising one or nucleic acids, comprising:
a) a nucleic acid sequence encoding a deactivated RNA-guided endonuclease; and
b) a nucleic acid sequence encoding a guide RNA complementary to a target sequence in the promoter or regulatory sequences of the IRAK3 gene and capable of directing said RNA-guided endonuclease to said target sequence, wherein the guide RNA is fused to one or more transcriptional activators,
   for use in a method of treatment or prophylaxis of macular degeneration in a subject, wherein the nucleic acid system increases IRAK-M expression in a target cell of the subject.

76. The nucleic acid system of paragraph 74 or paragraph 75, wherein the transcriptional activator is the transactivation domain, VP64.

77. The nucleic acid system for use according to any one of paragraphs 74 to 76, wherein the one or more nucleic acids are one or more viral vector genomes.

78. The nucleic acid system for use according to paragraph 77, wherein the one or more viral vector genomes are one or more adeno-associated virus vector genomes.

79. A viral vector system comprising the nucleic acid system for use according to any one of paragraphs 68 to 78.

80. A pharmaceutical composition comprising the nucleic acid for use according to any one of paragraphs 1 to 32, the vector virion for use according to any one of paragraphs 33 to 58, the IRAK-M polypeptide for use according to any one of paragraphs 59 to 67, the nucleic acid system for use according to any one of paragraphs 68 to 78, or the viral vector system comprising the nucleic acid system according to paragraph 79.

81. The pharmaceutical composition for use according to paragraph 80, wherein the pharmaceutical composition is formulated for ocular delivery.

82. A system comprising:
a) an RNA-guided endonuclease;
b) a guide RNA complementary to a target sequence associated with an insertion site in the genome of the target cell and capable of directing said RNA-guided endonuclease to said target sequence; and
c) a nucleic acid sequence encoding IRAK-M,
   for use in a method of treatment of prophylaxis of macular degeneration in a subject, wherein the nucleic acid sequence encoding IRAK-M is capable of driving expression of IRAK-M in a target cell of the subject and the system is suitable for directed insertion of the nucleic acid sequence encoding IRAK-M at the insertion site in the genome of the target cell.

83. A nucleic acid system comprising one or more nucleic acids, comprising:
a) a nucleic acid sequence encoding a deactivated RNA-guided endonuclease; and
b) a nucleic acid sequence encoding a guide RNA complementary to a target sequence in the promoter or regulatory sequences for IRAK3 gene and capable of directing said RNA-guided endonuclease to said target sequence, wherein said guide RNA further comprises an aptamer capable of specifically binding to a transcriptional activator,
   for use in a method of treatment or prophylaxis of macular degeneration in a subject, wherein the nucleic acid system increases IRAK-M expression in a target cell of the subject.

84. The nucleic acid system for use according to paragraph 83, wherein the aptamer is an RNA aptamer.

85. A nucleic acid system comprising one or more nucleic acids, comprising:
a) a nucleic acid sequence encoding a deactivated RNA-guided endonuclease;
b) a nucleic acid sequence encoding an RNA binding protein fused to one or more transcriptional activators; and
c) a nucleic acid sequence encoding a guide RNA complementary to a target sequence in the promoter or regulatory sequences for the IRAK3 gene and capable of directing said RNA-guided endonuclease to said target sequence, wherein said guide RNA further comprises an RNA aptamer capable of specifically binding to the RNA binding protein,
   for use in a method of treatment or prophylaxis of macular degeneration in a subject, wherein the nucleic acid system increases IRAK-M expression in a target cell of the subject.

86. The nucleic acid system for use according to paragraph 85, wherein the one or more transcriptional activators is selected from the group consisting of VP64, p65 and HSF1.

87. The nucleic acid system for use according to paragraph 85 or paragraph 86, wherein the RNA aptamer is capable of binding to an RNA binding protein dimer.

88. The nucleic acid system for use according to any one of paragraphs 85 to 87, wherein the RNA binding protein is MS2.

89. The nucleic acid system for use according to any one of paragraphs 85 to 88, wherein the deactivated RNA-guided endonuclease is fused to an additional transcriptional activator.

90. The nucleic acid system for use according to paragraph 89, wherein the additional transcriptional activator is VP64.

91. A nucleic acid system comprising one or more nucleic acids, comprising:
a) a nucleic acid sequence encoding a deactivated RNA-guided endonuclease fused to an epitope repeat array comprising one or more epitopes;
b) one or more nucleic acid sequences encoding an epitope binding molecule fused to one or more transcriptional activators, wherein said epitope binding molecule is capable of specifically binding to an epitope of the epitope repeat array; and
c) a nucleic acid sequence encoding a guide RNA complementary to a target sequence in the promoter or regulatory sequences for the IRAK3 gene and capable of directing said RNA-guided endonuclease to said target sequence,
   for use in a method of treatment or prophylaxis of macular degeneration in a subject, wherein the nucleic acid system increases IRAK-M expression in a target cell of the subject.

92. The nucleic acid system for use according to paragraph 91, wherein the epitope binding molecule comprises a nuclear localisation sequence (NLS).

93. The nucleic acid system for use according to paragraph 91 or paragraph 92, wherein the epitope binding molecule is an antibody or antibody-like molecule.

94. The nucleic acid system for use according to any one of paragraphs 91 to 93, wherein the one or more transcriptional activators are selected from the group consisting of VP64, p65 and Rta.

95. The nucleic acid system for use according to any one of paragraphs 83 to 94, wherein the one or more nucleic acids are one or more viral vector genomes.

96. The nucleic acid system for use according to paragraph 95, wherein the one or more viral vector genomes are one or more adeno-associated virus vector genomes.

97. A system comprising:
a) a deactivated RNA-guided endonuclease fused to one or more transcriptional activators; and
b) a guide RNA complementary to a target sequence in the promoter or regulatory sequences for the IRAK3 gene and capable of directing said RNA-guided endonuclease to said target sequence,
   for use in a method of treatment or prophylaxis of macular degeneration in a subject, wherein the system increases IRAK-M expression in a target cell of the subject.

98. A system comprising:
a) a deactivated RNA-guided endonuclease; and
b) a guide RNA complementary to a target sequence in the promoter or regulatory sequences for IRAK3 gene and capable of directing said RNA-guided endonuclease to said target sequence, wherein said guide RNA further comprises an aptamer capable of specifically binding to a transcriptional activator,
   for use in a method of treatment or prophylaxis of macular degeneration in a subject, wherein the system increases IRAK-M expression in a target cell of the subject.

99. A system comprising:
a) a deactivated RNA-guided endonuclease;
b) an RNA binding protein fused to one or more transcriptional activators;
c) a guide RNA complementary to a target sequence in the promoter or regulatory sequences for the IRAK3 gene and capable of directing said RNA-guided endonuclease to said target sequence, wherein said guide RNA comprises an RNA aptamer capable of specifically binding to the RNA binding protein,
   for use in a method of treatment or prophylaxis of macular degeneration in a subject, wherein the system increases IRAK-M expression in a target cell of the subject 100. A system comprising:
a) a deactivated RNA-guided endonuclease fused to an epitope repeat array comprising one or more epitopes;
b) one or more epitope binding molecules fused to one or more transcriptional activators, wherein said epitope binding molecule is capable of specifically binding to an epitope of the epitope repeat array; and
c) a guide RNA complementary to a target sequence in the promoter or regulatory sequences for the IRAK3 gene and capable of directing said RNA-guided endonuclease to said target sequence,
   for use in a method of treatment or prophylaxis of macular degeneration in a subject, wherein the system increases IRAK-M expression in a target cell of the subject.

101. A nucleic acid system comprising one or more nucleic acids, comprising:
a) a nucleic acid sequence encoding a deactivated RNA-guided endonuclease fused to one or more DNA demethylating agents; and
b) a nucleic acid sequence encoding a guide RNA complementary to (i) a target sequence in the promoter sequence for the IRAK3 gene, (ii) a target sequence in the regulatory sequences for the IRAK3 gene or (iii) a target sequence in the IRAK3 gene and capable of directing said RNA-guided endonuclease to said target sequence,
   for use in a method of treatment or prophylaxis of macular degeneration in a subject, wherein the nucleic acid system increases IRAK-M expression in a target cell of the subject.

102. A nucleic acid system comprising one or more nucleic acids, comprising:
a) a nucleic acid sequence encoding a deactivated RNA-guided endonuclease; and
b) a nucleic acid sequence encoding a guide RNA complementary to (i) a target sequence in the promoter sequence for the IRAK3 gene, (ii) a target sequence in the regulatory sequences for the IRAK3 gene or (iii) a target sequence in the IRAK3 gene and capable of directing said RNA-guided endonuclease to said target sequence, wherein said guide RNA further comprises an aptamer capable of specifically binding to a DNA demethylating agent,
   for use in a method of treatment or prophylaxis of macular degeneration in a subject, wherein the nucleic acid system increases IRAK-M expression in a target cell of the subject.

103. The nucleic acid system for use according to paragraph 102, wherein the aptamer is an RNA aptamer.

104. A nucleic acid system comprising one or more nucleic acids, comprising:
a) a nucleic acid sequence encoding a deactivated RNA-guided endonuclease;
b) a nucleic acid sequence encoding an RNA binding protein fused to one or more DNA demethylating agents;
c) a nucleic acid sequence encoding a guide RNA complementary to (i) a target sequence in the promoter sequence for the IRAK3 gene, (ii) a target sequence in the regulatory sequences for the IRAK3 gene or (iii) a target sequence in the IRAK3 gene and capable of directing said RNA-guided endonuclease to said target sequence, wherein said guide RNA further comprises an RNA aptamer capable of specifically binding to the RNA binding protein,
   for use in a method of treatment or prophylaxis of macular degeneration in a subject, wherein the nucleic acid system increases IRAK-M expression in a target cell of the subject.

105. The nucleic acid system for use according to paragraph 104, wherein the RNA aptamer is capable of binding to an RNA binding protein dimer.

106. The nucleic acid system for use according to paragraphs 105, wherein the RNA binding protein is MS2.

107. The nucleic acid system for use according to any one of paragraphs 104 to 106, wherein the deactivated RNA-guided endonuclease is fused to an additional DNA demethylating agent.

108. A nucleic acid system comprising one or more nucleic acids, comprising:
a) a nucleic acid sequence encoding a deactivated RNA-guided endonuclease fused to an epitope repeat array comprising one or more epitopes;
b) one or more nucleic acid sequences encoding an epitope binding molecule fused to one or more DNA demethylating agents, wherein said epitope binding molecule is capable of specifically binding to an epitope of the epitope repeat array; and
c) a nucleic acid sequence encoding a guide RNA complementary to (i) a target sequence in the promoter sequence for the IRAK3 gene, (ii) a target sequence in the regulatory sequences for the IRAK3 gene or (iii) a target sequence in the IRAK3 gene and capable of directing said RNA-guided endonuclease to said target sequence,
   for use in a method of treatment or prophylaxis of macular degeneration in a subject, wherein the nucleic acid system increases IRAK-M expression in a target cell of the subject.

109. The nucleic acid system for use according to paragraph 108, wherein the epitope binding molecule comprises a nuclear localisation sequence (NLS).

110. The nucleic acid system for use according to paragraph 109 or paragraph 110, wherein the epitope binding molecule is an antibody or antibody-like molecule.

111. The nucleic acid system for use according to any one of paragraphs 101 to 110, wherein the DNA demethylating agent is TET1.

112. The nucleic acid system for use according to any one of paragraphs 101 to 110, wherein the DNA demethylating agent is LESD1.

113. The nucleic acid system for use according to any one of paragraphs 101 to 112, wherein the one or more nucleic acids are one or more viral vector genomes.

114. The nucleic acid system for use according to paragraph 113, wherein the one or more viral vector genomes are one or more adeno-associated virus vector genomes.

115. A system comprising:
a) a deactivated RNA-guided endonuclease fused to one or more DNA demethylating agents; and
b) a guide RNA complementary to (i) a target sequence in the promoter sequence for the IRAK3 gene, (ii) a target sequence in the regulatory sequences for the IRAK3 gene or (iii) a target sequence in the IRAK3 gene and capable of directing said RNA-guided endonuclease to said target sequence,
   for use in a method of treatment or prophylaxis of macular degeneration in a subject, wherein the system increases IRAK-M expression in a target cell of the subject.

116. A system comprising:
a) a deactivated RNA-guided endonuclease; and
b) a guide RNA complementary to (i) a target sequence in the promoter sequence for the IRAK3 gene, (ii) a target sequence in the regulatory sequences for the IRAK3 gene or (iii) a target sequence in the IRAK3 gene and capable of directing said RNA-guided endonuclease to said target sequence, wherein said guide RNA further comprises an aptamer capable of specifically binding to a DNA demethylating agent,
   for use in a method of treatment or prophylaxis of macular degeneration in a subject, wherein the system increases IRAK-M expression in a target cell of the subject.

117. A system comprising:
a) a deactivated RNA-guided endonuclease;
b) an RNA binding protein fused to one or more DNA demethylating agents;
c) a guide RNA complementary to (i) a target sequence in the promoter sequence for the IRAK3 gene, (ii) a target sequence in the regulatory sequences for the IRAK3 gene or (iii) a target sequence in the IRAK3 gene and capable of directing said RNA-guided endonuclease to said target sequence, wherein said guide RNA further comprises an RNA aptamer capable of specifically binding to the RNA binding protein,
   for use in a method of treatment or prophylaxis of macular degeneration in a subject, wherein the system increases IRAK-M expression in a target cell of the subject.

118. A system comprising:
a) a deactivated RNA-guided endonuclease fused to an epitope repeat array comprising one or more epitopes;
b) one or more epitope binding molecules fused to one or more DNA demethylating agents, wherein said epitope binding molecule is capable of specifically binding to an epitope of the epitope repeat array; and
c) a guide RNA complementary to (i) a target sequence in the promoter sequence for the IRAK3 gene, (ii) a target sequence in the regulatory sequences for the IRAK3 gene or (iii) a target sequence in the IRAK3 gene and capable of directing said RNA-guided endonuclease to said target sequence,
   for use in a method of treatment or prophylaxis of macular degeneration in a subject, wherein the system increases IRAK-M expression in a target cell of the subject.

119. A nucleic acid comprising a nucleic acid sequence encoding a fusion protein, the fusion protein comprising:
(a) a nucleic acid binding molecule capable of binding to a target sequence in the promoter or regulatory sequences of the IRAK3 gene; and
(b) one or more transcriptional activators,
   for use in a method of treatment or prophylaxis of macular degeneration in a subject, wherein the fusion protein is capable of increasing IRAK-M expression in a target cell of the subject.

120. A nucleic acid system comprising:
a) a nucleic acid sequence encoding a fusion protein, wherein the fusion protein comprises (i) a nucleic acid binding molecule capable of binding to a target sequence in the promoter or regulatory sequences of the IRAK3 gene and (ii) an epitope repeat array; and
b) one or more nucleic acid sequences encoding an epitope binding molecule fused to one or more transcriptional activators, wherein said epitope binding molecule is capable of specifically binding to an epitope of the epitope repeat array,
   for use in a method of treatment or prophylaxis of macular degeneration in a subject, wherein the nucleic acid system is capable of increasing IRAK-M expression in a target cell of the subject.

121. A nucleic acid comprising a nucleic acid sequence encoding a fusion protein, the fusion protein comprising:
a) a nucleic acid binding molecule capable of binding to (i) a target sequence in the promoter sequence for the IRAK3 gene, (ii) a target sequence in the regulatory sequences for the IRAK3 gene or (iii) a target sequence in the IRAK3 gene; and
b) one or more DNA demethylating agents,
   for use in a method of treatment or prophylaxis of macular degeneration in a subject, wherein the fusion protein is capable of increasing IRAK-M expression in a target cell of the subject.

122. A nucleic acid system comprising:
a) a nucleic acid sequence encoding a fusion protein, wherein the fusion protein comprising (i) nucleic acid binding molecule capable of binding to (1) a target sequence in the promoter sequence for the IRAK3 gene, (2) a target sequence in the regulatory sequences for the IRAK3 gene or (3) a target sequence in the IRAK3 gene and (ii) an epitope repeat array; and
b) one or more nucleic acid sequences encoding an epitope binding molecule fused to one or more DNA demethylating agents, wherein said epitope binding molecule is capable of specifically binding to an epitope of the epitope repeat array,
   for use in a method of treatment or prophylaxis of macular degeneration in a subject, wherein the nucleic acid system is capable of increasing IRAK-M expression in a target cell of the subject.

123. The nucleic acid or nucleic acid system for use according to paragraph 119 or paragraph 120, wherein the transcriptional activator is the transactivation domain, VP64.

124. The nucleic acid or nucleic acid system for use according to paragraph 121 or paragraph 122, wherein the DNA demethylating agent is TET1.

125. The nucleic acid or nucleic acid system for use according to paragraph 121 or paragraph 122, wherein the DNA demethylating agent is LESD1.

126. The nucleic acid or nucleic acid system for use according to any one of paragraphs 119 to 125, wherein the nucleic acid binding molecule is a TAL effector repeat array.

127. The nucleic acid or nucleic acid system for use according to any one of paragraphs 119 to 125, wherein the nucleic acid binding molecule is zinc finger array.

128. The nucleic acid or nucleic acid system for use according to any one of the paragraphs 119 to 127, wherein the nucleic acid is delivered to a target cell via a viral vector.

129. The nucleic acid for or nucleic acid system use according to any one of paragraphs 119 to 127, wherein the nucleic acid is delivered to a target cell via a non-viral carrier.

130. The nucleic acid or nucleic acid system for use according to any one of paragraphs 119 to 127, wherein the nucleic acid is a viral vector genome.

131. A fusion protein comprising:
(a) a nucleic acid binding molecule capable of binding to a target sequence in the promoter or regulatory sequences of the IRAK3 gene; and
(b) one or more transcriptional activators,
   for use in a method of treatment or prophylaxis of macular degeneration in a subject, wherein the fusion protein is capable of increasing IRAK-M expression in a target cell of the subject.

132. A fusion protein comprising:
a) a nucleic acid binding molecule capable of binding to (i) a target sequence in the promoter sequence for the IRAK3 gene, (ii) a target sequence in the regulatory sequences for the IRAK3 gene or (iii) a target sequence in the IRAK3 gene; and
b) one or more DNA demethylating agents,
   for use in a method of treatment or prophylaxis of macular degeneration in a subject, wherein the fusion protein is capable of increasing IRAK-M expression in a target cell of the subject.

133. A system comprising:
a) a fusion protein, wherein the fusion protein comprises (i) a nucleic acid binding molecule capable of binding to a target sequence in the promoter or regulatory sequences of the IRAK3 gene and (ii) an epitope repeat array; and
b) one or more epitope binding molecules fused to one or more transcriptional activators, wherein said epitope binding molecule is capable of specifically binding to an epitope of the epitope repeat array,
   for use in a method of treatment or prophylaxis of macular degeneration in a subject, wherein the system is capable of increasing IRAK-M expression in a target cell of the subject.

134. A system comprising:
a) a fusion protein, wherein the fusion protein comprising (i) nucleic acid binding molecule capable of binding to (1) a target sequence in the promoter sequence for the IRAK3 gene, (2) a target sequence in the regulatory sequences for the IRAK3 gene or (3) a target sequence in the IRAK3 gene and (ii) an epitope repeat array; and
b) one or more epitope binding molecules fused to one or more DNA demethylating agents, wherein said epitope binding molecule is capable of specifically binding to an epitope of the epitope repeat array,
   for use in a method of treatment or prophylaxis of macular degeneration in a subject, wherein the system is capable of increasing IRAK-M expression in a target cell of the subject.

135. A small molecule for use in a method of treatment or prophylaxis of macular degeneration in a subject, wherein the small molecule increases endogenous IRAK-M expression in a target cell of the subject.

136. The small molecule for use according to paragraph 135, wherein the small molecule reduces DNA methylation in the promoter sequence for the IRAK3 gene and/or reduces DNA methylation in the IRAK3 gene.

137. The small molecule for use according to paragraph 136, wherein the small molecule is EPZ-6438.

138. The small molecule for use according to paragraph 136, wherein the small molecule is azacytidine.

139. The small molecule for use according to paragraph 135, wherein the small molecule is ibudilast.

140. The small molecule for use according to paragraph 135, wherein the small molecule is capable of recruiting one or more polypeptides that promote transcription to the IRAK3 promoter.

141. The small molecule for use according to paragraph 140, wherein the small molecule is a glucocorticoid.

142. The small molecule for use according to paragraph 141, wherein the small molecule is cortisol.

143. The small molecule for use according to paragraph 135, wherein the small molecule is a METTL3 inhibitor.

144. The small molecule for use according to paragraph 143, wherein the inhibitor is selected from the group consisting of STM2457, Cpd-564 and UZH2.

145. A nucleic acid for use in a method of treatment or prophylaxis of macular degeneration in a subject, wherein the nucleic acid increases endogenous IRAK-M expression in a target cell of the subject.

146. The nucleic acid for use according to paragraph 145, wherein the nucleic acid inhibits expression of METTL3.

147. The nucleic acid for use according to paragraph 146, wherein the nucleic acid is an siRNA, an shRNA, a miRNA, or an ASO.

148. A polypeptide for use in a method of treatment or prophylaxis of macular degeneration in a subject, wherein the polypeptide increases endogenous IRAK-M expression in a target cell of the subject.

149. The polypeptide for use according to paragraph 148, wherein the polypeptide activates ERK1/2 and/or activates PI3K and Akt1.

150. The polypeptide for use according to paragraph 149, wherein the polypeptide is adiponectin.

151. A small molecule for use in a method of treatment or prophylaxis of macular degeneration in a subject, wherein the small molecule increases IRAK-M activity in a target cell of the subject.

152. The small molecule for use according to paragraph 151, wherein the small molecule promotes

IRAK-M binding to IRAK-1 and/or IRAK-4.

153. The small molecule for use according to paragraph 151 or paragraph 152, wherein the small molecule promotes IRAK-M binding to MyD88.

154. The small molecule for use according to any one of paragraphs 151 to 153, wherein the small molecule increases cellular cGMP.

155. The small molecule for use according to paragraph 154, wherein the small molecule is a nitric oxide donor.

156. The small molecule for use according to paragraph 154, wherein the small molecule is riociguat.

157. A polypeptide for in a method of treatment or prophylaxis of macular degeneration in a subject, wherein the polypeptide increases IRAK-M activity in a target cell of a subject.

158. The polypeptide for use according to paragraph 157, wherein the polypeptide promotes IRAK-M binding to IRAK-1 and/or IRAK-4.

159. The polypeptide for use according to paragraph 157 or paragraph 158, wherein the polypeptide promotes IRAK-M binding to MyD88.

160. The polypeptide for use according to any one of paragraphs 157 to 159, wherein the polypeptide is α-MSH or a fragment thereof.

161. A pharmaceutical composition comprising the system for use according to paragraph 82, nucleic acid system for use according to any one of paragraphs 83 to 96, the system for use according to any one of paragraphs 97 to 100, the nucleic acid system for use according to any one of paragraphs 101 to 114, the system for use according to paragraphs 115 to 118, the nucleic acid or nucleic acid system for use according to any one of paragraphs 119 to 130, a fusion protein or system for use according to 131 to 134, the small molecule for use according to any one of paragraphs 135 to 144, the nucleic acid for use according to any one of paragraphs 145 to 147, the polypeptide for use according to any one of paragraphs 148 to 150, the small molecule for use according to any one of paragraphs 151 to 156, or the polypeptide for use according to any one of paragraphs 157 to 160.

162. The pharmaceutical composition for use according to paragraph 161, wherein the pharmaceutical composition is formulated for ocular delivery.

163. The pharmaceutical composition for use according to paragraph 161 or paragraph 162, wherein the pharmaceutical composition further comprises an additional therapeutic agent.

164. The pharmaceutical composition for use according to paragraph 80 or paragraph 81, wherein the pharmaceutical composition further comprises an additional therapeutic agent.

1. A method of treatment or prophylaxis of macular degeneration comprising administering a nucleic acid to a subject in need thereof, wherein the nucleic acid comprises a nucleic acid sequence encoding IRAK-M and wherein the nucleic acid is capable of driving expression of IRAK-M in a target cell.

2. The method of claim 1, wherein a promoter is operably linked to the nucleic acid sequence, optionally wherein the promoter is selected from the group consisting of a CMV promoter, a CAG promoter, a CB7 promoter, a Best1 promoter, and the native promoter for IRAK3 or a functional fragment thereof.

3. (canceled)

4. The method according to claim 1, wherein:
(i) autophagic flux is maintained or increased in the target cell comprising the nucleic acid compared to an equivalent cell not comprising the nucleic acid;
(ii) mitochondrial activity is maintained or increased in the target cell comprising the nucleic acid compared to an equivalent cell not comprising the nucleic acid; and/or
(iii) proinflammatory cytokine production is reduced in the target cell comprising the nucleic acid compared to an equivalent target cell not comprising the nucleic acid.

5. The method according to claim 1, wherein the nucleic acid is delivered to a target cell via a viral vector, optionally wherein, wherein the viral vector is selected from the group consisting of an adeno-associated virus vector, an adenovirus vector, a retrovirus vector, an orthomyxovirus vector, a paramyxovirus vector, a papovavirus vector, a picornavirus vector, a lentivirus vector, a herpes simplex virus vector, a vaccinia virus vector, a pox virus vector, an anellovirus vector, and an alphavirus vector.

6. (canceled)

7. The method according to claim 1, wherein the nucleic acid is a viral vector genome, optionally wherein the viral vector genome is selected from the group consisting of an adeno-associated virus vector genome, an adenovirus vector genome, a retrovirus vector genome, an orthomyxovirus vector genome, a paramyxovirus vector genome, a papovavirus vector genome, a picornavirus vector genome, a lentivirus vector genome, a herpes simplex virus vector genome, a vaccinia virus vector genome, a pox virus vector genome, an anellovirus vector genome, and an alphavirus vector genome.

8. (canceled)

9. The method according to claim 1, wherein the macular degeneration is selected from the group comprising age-related macular degeneration (AMD) and dry AMD.

10. (canceled)

11. The method according to claim 1, wherein the target cell is selected from the group consisting of a cell of the retina, a cell of the choroid and a cell of the RPE.

12. (canceled)

13. The method according to claim 1, wherein the nucleic acid is administered intraocularly, intravitreally, subretinally, suprachoroidally or periocularly to a subject, optionally wherein the nucleic acid is administered by subretinal injection.

14. (canceled)

15. The method according to claim 1, wherein the nucleic acid sequence encodes a polypeptide selected from the group consisting of a polypeptide comprising an amino acid sequence having at least 60% sequence identity to the amino acid sequence of SEQ ID NO: 1 and a polypeptide capable of preventing dissociation of IRAK-1 and/or IRAK-4 from MyD88 in a target cell.

16. (canceled)

17. A method of treatment or prophylaxis of macular degeneration comprising administering a vector virion to a subject in need thereof, wherein the vector virion comprises a nucleic acid comprising a nucleic acid sequence encoding IRAK-M and wherein the nucleic acid is capable of driving expression of IRAK-M in a target cell.

18. The method according to claim 17, wherein a promoter is operably linked to the nucleic acid sequence optionally wherein the promoter is selected from the group consisting of a CMV promoter, a CAG promoter, a CB7 promoter, a Best 1 promoter, and the native promoter for IRAK3 or a functional fragment thereof.

19. (canceled)

20. The method according to claim 17, wherein:
(i) autophagic flux is maintained or increased in the target cell comprising the vector virion compared to an equivalent cell not comprising the vector virion;
(ii) mitochondrial activity is maintained or increased in the target cell comprising the vector virion compared to an equivalent cell not comprising the vector virion; and/or
(iii) proinflammatory cytokine production is reduced in the target cell comprising the vector virion compared to an equivalent target cell not comprising the vector virion.

21. The method according to claim 17, wherein the nucleic acid is suitable for integration into the genome of the target cell by an RNA-guided endonuclease system.

22. The method according to claim 17, wherein the vector virion is selected from the group consisting of adeno-associated virus, adenovirus, retrovirus, orthomyxovirus, paramyxovirus, papovavirus, picornavirus, lentivirus, herpes simplex virus, vaccinia virus, pox virus, anellovirus, and alphavirus.

23. The method according to claim 22, wherein the vector virion is an adeno-associated virus (AAV) selected from the group consisting of AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), and AAV type 9 (AAV-9).

24.-26. (canceled)

27. The method according to claim 17, wherein the macular degeneration is selected from the group consisting of age-related macular degeneration (AMD) and dry AMD.

28. (canceled)

29. The method according to claim 17, wherein the target cell is selected from the group consisting of a cell of the retina, a cell of the choroid, and a cell of the RPE.

30. (canceled)

31. The method according to claim 17, wherein the vector virion is administered intraocularly, intravitreally, subretinally, suprachoroidally or periocularly to a subject, optionally wherein the vector virion is administered by subretinal injection.

32. (canceled)

33. The method according to claim 17, wherein the nucleic acid sequence encodes a polypeptide selected from the group consisting of a polypeptide comprising an amino acid sequence having at least 60% sequence identity to the amino acid sequence of SEQ ID NO: 1 and a polypeptide capable of preventing dissociation of IRAK-1 and/or IRAK-4 from MyD88 in a target cell.

34. (canceled)

35. An method of treatment or prophylaxis of macular degeneration comprising administering an IRAK-M polypeptide to a subject in need thereof.

36. The method according to claim 35, wherein the IRAK-M polypeptide is selected from the group consisting of a polypeptide comprising an amino acid sequence having at least 60% sequence identity to the amino acid sequence of SEQ ID NO: 1 and an IRAK-M polypeptide capable of preventing dissociation of IRAK-1 and/or IRAK-4 from MyD88 in a target cell.

37. (canceled)

38. The method according to claim 35, wherein the IRAK-M polypeptide further comprises a cell penetrating peptide (CPP).

39. The method according to claim 35, wherein:
(i) autophagic flux is maintained or increased in the target cell comprising the IRAK-M polypeptide compared to an equivalent cell not comprising the IRAK-M polypeptide;
(ii) mitochondrial activity is maintained or increased in the target cell comprising the IRAK-M polypeptide compared to an equivalent cell not comprising the IRAK-M polypeptide; and/or
(iii) proinflammatory cytokine production is reduced in the target cell comprising the IRAK-M polypeptide compared to an equivalent target cell not comprising the IRAK-M polypeptide.

40. A method of treatment or prophylaxis of macular degeneration comprising administering a nucleic acid system to a subject in need thereof, wherein the nucleic acid system comprises one or more nucleic acids, comprising:
a) a nucleic acid sequence encoding an RNA-guided endonuclease;
b) a nucleic acid sequence encoding a guide RNA complementary to a target sequence associated with an insertion site in the genome of the target cell and capable of directing said RNA-guided endonuclease to said target sequence; and
c) a nucleic acid sequence encoding IRAK-M,
wherein the nucleic acid sequence encoding IRAK-M is capable of driving expression of IRAK-M in a target cell of the subject and wherein the nucleic acid system is suitable for directed insertion of the nucleic acid sequence encoding IRAK-M at the insertion site in the genome of the target cell.

41. A method of treatment or prophylaxis of macular degeneration comprising administering a nucleic acid system to a subject in need thereof, wherein the nucleic acid system comprises one or more nucleic acids, comprising:
a) a nucleic acid sequence encoding a deactivated RNA-guided endonuclease fused to one or more transcriptional activators; and
b) a nucleic acid sequence encoding a guide RNA complementary to a target sequence in the promoter or regulatory sequences for the IRAK3 gene and capable of directing said RNA-guided endonuclease to said target sequence, wherein the nucleic acid system increases IRAK-M expression in a target cell of the subject.

42. A method of treatment or prophylaxis of macular degeneration comprising administering a nucleic acid system to a subject in need thereof, wherein the nucleic acid system comprises one or more nucleic acids, comprising:
a) a nucleic acid sequence encoding a deactivated RNA-guided endonuclease; and
b) a nucleic acid sequence encoding a guide RNA complementary to a target sequence in the promoter or regulatory sequences for IRAK3 gene and capable of directing said RNA-guided endonuclease to said target sequence, wherein said guide RNA further comprises an aptamer capable of specifically binding to a transcriptional activator,
wherein the nucleic acid system increases IRAK-M expression in a target cell of the subject.

43. A method of treatment or prophylaxis of macular degeneration comprising administering a nucleic acid system to a subject in need thereof, wherein the nucleic acid system comprises one or more nucleic acids, comprising:
a) a nucleic acid sequence encoding a deactivated RNA-guided endonuclease;
b) a nucleic acid sequence encoding an RNA binding protein fused to one or more transcriptional activators; and
c) a nucleic acid sequence encoding a guide RNA complementary to a target sequence in the promoter or regulatory sequences for the IRAK3 gene and capable of directing said RNA-guided endonuclease to said target sequence, wherein said guide RNA further comprises an RNA aptamer capable of specifically binding to the RNA binding protein,
wherein the nucleic acid system increases IRAK-M expression in a target cell of the subject.

44. The method according to claim 43, wherein (i) the RNA aptamer is capable of binding to an RNA binding protein dimer; (ii) the RNA binding protein is MS2;
and/or (iii) the deactivated RNA-guided endonuclease is fused to an additional transcriptional activator.

45.-46. (canceled)

47. A method of treatment or prophylaxis of macular degeneration comprising administering nucleic acid system to a subject in need thereof, wherein the nucleic acid system comprises one or more nucleic acids, comprising:
a) a nucleic acid sequence encoding a deactivated RNA-guided endonuclease fused to an epitope repeat array comprising one or more epitopes;
b) one or more nucleic acid sequences encoding an epitope binding molecule fused to one or more transcriptional activators, wherein said epitope binding molecule is capable of specifically binding to an epitope of the epitope repeat array; and
c) a nucleic acid sequence encoding a guide RNA complementary to a target sequence in the promoter or regulatory sequences for the IRAK3 gene and capable of directing said RNA-guided endonuclease to said target sequence,
wherein the nucleic acid system increases IRAK-M expression in a target cell of the subject.

48. The method according to claim 47, wherein the epitope binding molecule comprises a nuclear localisation sequence (NLS) and/or wherein the epitope binding molecule is an antibody or antibody-like molecule.

49. (canceled)

50. A method of treatment or prophylaxis of macular degeneration comprising administering a nucleic acid system to a subject in need thereof, wherein the nucleic acid system comprises one or more nucleic acids, comprising:
a) a nucleic acid sequence encoding a deactivated RNA-guided endonuclease fused to one or more DNA demethylating agents; and
b) a nucleic acid sequence encoding a guide RNA complementary to (i) a target sequence in the promoter sequence for the IRAK3 gene, (ii) a target sequence in the regulatory sequences for the IRAK3 gene or (iii) a target sequence in the IRAK3 gene and capable of directing said RNA-guided endonuclease to said target sequence,
wherein the nucleic acid system increases IRAK-M expression in a target cell of the subject.

51. A method of treatment or prophylaxis of macular degeneration comprising administering a nucleic acid system to a subject in need thereof, wherein the nucleic acid system comprises one or more nucleic acids, comprising:
a) a nucleic acid sequence encoding a deactivated RNA-guided endonuclease; and
b) a nucleic acid sequence encoding a guide RNA complementary to (i) a target sequence in the promoter sequence for the IRAK3 gene, (ii) a target sequence in the regulatory sequences for the IRAK3 gene or (iii) a target sequence in the IRAK3 gene and capable of directing said RNA-guided endonuclease to said target sequence, wherein said guide RNA further comprises an aptamer capable of specifically binding to a DNA demethylating agent,
wherein the nucleic acid system increases IRAK-M expression in a target cell of the subject.

52. A method of treatment or prophylaxis of macular degeneration comprising administering a nucleic acid system to a subject in need thereof, wherein the nucleic acid system comprises one or more nucleic acids, comprising:
a) a nucleic acid sequence encoding a deactivated RNA-guided endonuclease;
b) a nucleic acid sequence encoding an RNA binding protein fused to one or more DNA demethylating agents;
c) a nucleic acid sequence encoding a guide RNA complementary to (i) a target sequence in the promoter sequence for the IRAK3 gene, (ii) a target sequence in the regulatory sequences for the IRAK3 gene or (iii) a target sequence in the IRAK3 gene and capable of directing said RNA-guided endonuclease to said target sequence, wherein said guide RNA further comprises an RNA aptamer capable of specifically binding to the RNA binding protein,
wherein the nucleic acid system increases IRAK-M expression in a target cell of the subject.

53. The method according to claim 52, wherein (i) the RNA aptamer is capable of binding to an RNA binding protein dimer; (ii) the RNA binding protein is MS2; and/or (iii) wherein the deactivated RNA-guided endonuclease is fused to an additional DNA demethylating agent.

54.-55. (canceled)

56. A method of treatment or prophylaxis of macular degeneration comprising administering a nucleic acid system to a subject in need thereof, wherein the nucleic acid system comprises one or more nucleic acids, comprising:
a) a nucleic acid sequence encoding a deactivated RNA-guided endonuclease fused to an epitope repeat array comprising one or more epitopes;
b) one or more nucleic acid sequences encoding an epitope binding molecule fused to one or more DNA demethylating agents, wherein said epitope binding molecule is capable of specifically binding to an epitope of the epitope repeat array; and
c) a nucleic acid sequence encoding a guide RNA complementary to (i) a target sequence in the promoter sequence for the IRAK3 gene, (ii) a target sequence in the regulatory sequences for the IRAK3 gene or (iii) a target sequence in the IRAK3 gene and capable of directing said RNA-guided endonuclease to said target sequence,
wherein the nucleic acid system increases IRAK-M expression in a target cell of the subject.

57. The method according to claim 56, wherein the epitope binding molecule comprises a nuclear localisation sequence (NLS) and/or wherein the epitope binding molecule is an antibody or antibody-like molecule.

58. (canceled)

59. The method according to claim 50, wherein the DNA demethylating agent is selected from the group consisting of TET1 and Lysine-specific demethylase 1.

60. (canceled)

61. The method according to claim 40, wherein the one or more nucleic acids are one or more viral vector genomes, optionally wherein the one or more viral vector genomes are one or more adeno-associated virus vector genomes.

62. (canceled)

63. A method of treatment or prophylaxis of macular degeneration comprising administering a nucleic acid to a subject in need thereof, wherein the nucleic acid comprises a nucleic acid sequence encoding a fusion protein, the fusion protein comprising:
(a) a nucleic acid binding molecule capable of binding to a target sequence in the promoter or regulatory sequences of the IRAK3 gene; and
(b) one or more transcriptional activators,
wherein the fusion protein is capable of increasing IRAK-M expression in a target cell of the subject, optionally wherein the transcriptional activator is the transactivation domain, VP64.

64. (canceled)

65. A method of treatment or prophylaxis of macular degeneration comprising administering a nucleic acid to a subject in need thereof, wherein the nucleic acid comprises a nucleic acid sequence encoding a fusion protein, the fusion protein comprising:
a) a nucleic acid binding molecule capable of binding to (i) a target sequence in the promoter sequence for the IRAK3 gene, (ii) a target sequence in the regulatory sequences for the IRAK3 gene or (iii) a target sequence in the IRAK3 gene; and
b) one or more DNA demethylating agents,
wherein the fusion protein is capable of increasing IRAK-M expression in a target cell of the subject.

66. The method according to claim 65, wherein the DNA demethylating agent is selected from the group consisting of TET1 and Lysine-specific demethylase 1.

67. (canceled)

68. The method according to claim 63, wherein (a) the nucleic acid binding molecule is selected from the group consisting of a TAL effector repeat array and a zinc finger array; (b) the nucleic acid is delivered to a target cell via a viral vector and/or (c) the nucleic acid is a viral vector genome.

69.-73. (canceled)

74. The method according to claim 65, wherein (a) the nucleic acid binding molecule is selected from the group consisting of a TAL effector repeat array and a zinc finger array; (b) the nucleic acid is delivered to a target cell via a viral vector and/or (c) the nucleic acid is a viral vector genome.
